SEQUENCE LISTING (113 sequences)

SEQ ID NO: 1 - DNA, 62 bp, Artificial Sequence
  5′-UTR of human peroxisomal multifunctional enzyme type 2 lacking the 5′-terminal oligopyrimidine tract (HSD17B4)
gtcccgcagtcggcgtccagcggctctgcttgttcgtgtgtgtgtcgttgcaggccttattc

SEQ ID NO: 2 - RNA, 62 nt, Artificial Sequence
  5′-UTR of human peroxisomal multifunctional enzyme type 2 lacking the 5′-terminal oligopyrimidine tract (HSD17B4) mRNA
gucccgcagucggcguccagcggcucugcuuguucgugugugugucguugcaggccuuauuc

SEQ ID NO: 3 - DNA, 42 bp, Artificial Sequence
  5′-UTR of human acid ceramidase lacking the 5′-terminal oligopyrimidine tract (ASAH1)
gcctctgctggagtccggggagtggcgttggctgctagagcg

SEQ ID NO: 4 - RNA, 42 nt, Artificial Sequence
  5′-UTR of human acid ceramidase lacking the 5′-terminal oligopyrimidine tract (ASAH1) mRNA
gccucugcuggaguccggggaguggcguuggcugcuagagcg

SEQ ID NO: 5 - DNA, 75 bp, Artificial Sequence
  5′-UTR of human mitochondrial ATP synthase subunit alpha lacking the 5′-terminal oligopyrimidine tract (ATP5A1)
gcggctcggccattttgtcccagtcagtccggaggctgcggctgcagaagtaccgcctgcggagtaactgcaaag

SEQ ID NO: 6 - RNA, 75 nt, Artificial Sequence
  5′-UTR of human mitochondrial ATP synthase subunit alpha lacking the 5′-terminal oligopyrimidine tract (ATP5A1) mRNA
gcggcucggccauuuugucccagucaguccggaggcugcggcugcagaaguaccgccugcggaguaacugcaaag

SEQ ID NO: 7 - DNA, 54 bp, Artificial Sequence
  5′-UTR of mouse RIKEN cDNA 2010107E04 gene (Mp68, 2010107E04Rik)
ctttcccattctgtagcagaatttggtgttgcctgtggtcttggtcccgcggag

SEQ ID NO: 8 - RNA, 54 nt, Artificial Sequence
  5′-UTR of mouse RIKEN cDNA 2010107E04 gene (Mp68, 2010107E04Rik) mRNA
cuuucccauucuguagcagaauuugguguugccuguggucuuggucccgcggag

SEQ ID NO: 9 - DNA, 81 bp, Artificial Sequence
  5′-UTR of mouse NADH dehydrogenase (ubiquinone) 1 alpha subcomplex, 4 (Ndufa4)
gtccgctcagccaggttgcagaagcggcttagcgtgtgtcctaatcttctctctgcgtgtaggtaggcctgtgccgcaaac

SEQ ID NO: 10 - RNA, 81 nt, Artificial Sequence
  5′-UTR of mouse NADH dehydrogenase (ubiquinone) 1 alpha subcomplex, 4 (Ndufa4) mRNA
guccgcucagccagguugcagaagcggcuuagcguguguccuaaucuucucucugcguguagguaggccugugccgcaaac

SEQ ID NO: 11 - DNA, 108 bp, Artificial Sequence
  5′-UTR of mouse nitric oxide synthase interacting protein (Nosip)
ctcctgtcgggcggaagtaggaggagtagagtttaaaaacagtactctttttccggttcgggacgtagttgaagcaacgacaagccggataaccgctcttgagacagg

SEQ ID NO: 12 - RNA, 108 nt, Artificial Sequence
  5′-UTR of mouse nitric oxide synthase interacting protein (Nosip) mRNA
cuccugucgggcggaaguaggaggaguagaguuuaaaaacaguacucuuuuuccgguucgggacguaguugaagcaacgacaagccggauaaccgcucuugagacagg

SEQ ID NO: 13 - DNA, 84 bp, Artificial Sequence
  5′-UTR of mouse ribosomal protein L31 (Rpl31)
cccgtgacccggaagttgtacggctacgcgactttccctcccacaaaccctcgcgcccttcctttcctacttgggcccggcaga

SEQ ID NO: 14 - RNA, 84 nt, Artificial Sequence
  5′-UTR of mouse ribosomal protein L31 (Rpl31) mRNA
cccgugacccggaaguuguacggcuacgcgacuuucccucccacaaacccucgcgcccuuccuuuccuacuugggcccggcaga

SEQ ID NO: 15 - DNA, 159 bp, Artificial Sequence
  5′-UTR of mouse solute carrier family 7 (cationic amino acid transporter, y+ system), member 3 (Slc7a3)
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 16 - RNA, 159 nt, Artificial Sequence
  5′-UTR of mouse solute carrier family 7 (cationic amino acid transporter, y+ system), member 3 (Slc7a3) mRNA
gggcgcuuggcuugcaaggacccugagcugcggcauugaagcacacccaacccaacucgacugaagucagccucacugaaccggaucugagaaucuucucucucugggcuugccagggcucuccgaaccuagcuagcauccucuucaauuccaacuaga

SEQ ID NO: 17 - DNA, 102 bp, Artificial Sequence
  5′-UTR of human tubulin beta 4B class Ivb (TUBB4B)
atataagcgttggcggagcgtcggttgtagcactctgcgcgcccgctcttctgctgctgtttgtctacttcctcctgcttccccgccgccgccgccgccatc

SEQ ID NO: 18 - RNA, 102 nt, Artificial Sequence
  5′-UTR of human tubulin beta 4B class Ivb (TUBB4B) mRNA
auauaagcguuggcggagcgucgguuguagcacucugcgcgcccgcucuucugcugcuguuugucuacuuccuccugcuuccccgccgccgccgccgccauc

SEQ ID NO: 19 - DNA, 222 bp, Artificial Sequence
  5′-UTR of mouse ubiquilin 2 (Ubqln2)
cggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgcc

SEQ ID NO: 20 - RNA, 222 nt, Artificial Sequence
  5′-UTR of mouse ubiquilin 2 (Ubqln2)  mRNA
cggagacggccugcaggaccugcucucucagcccucagccgaggccuacgccgagccgagugcgcagccgacgaccgggaggagccgcagccuucaacucugagguacugugauccgcgcugcccgccgggccgccccaguccgcugcugcggcaccuccuucccucgcgcccucuucgcucgccagcgccuucccugugagccugcgucaccgcggccgcc

SEQ ID NO: 21 - DNA, 42 bp, Artificial Sequence
  5′-UTR of human ribosomal protein Large 32 lacking the 5′-terminal oligopyrimidine tract (RPL32, 32L4)
ggcgctgcctacggaggtggcagccatctccttctcggcatc

SEQ ID NO: 22 - RNA, 42 nt, Artificial Sequence
  5′-UTR of human ribosomal protein Large 32 lacking the 5′-terminal oligopyrimidine tract (RPL32, 32L4) mRNA
ggcgcugccuacggagguggcagccaucuccuucucggcauc

SEQ ID NO: 23 - DNA, 57 bp, Artificial Sequence
  3′-UTR of human proteasome subunit beta 3 (PSMB3)
ccctgttcccagagcccacttttttttctttttttgaaataaaatagcctgtctttc

SEQ ID NO: 24 - RNA, 57 nt, Artificial Sequence
  3′-UTR of human proteasome subunit beta 3 (PSMB3) mRNA
cccuguucccagagcccacuuuuuuuucuuuuuuugaaauaaaauagccugucuuuc

SEQ ID NO: 25 - DNA, 121 bp, Artificial Sequence
  3′-UTR of human caspase 1 (CASP1)
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 26 - RNA, 121 nt, Artificial Sequence
  3′-UTR of human caspase 1 (CASP1) mRNA
aauaaggaaacuguaugaaugucugugggcaggaagugaagagauccuucuguaaagguuuuuggaauuaugucugcugaauaauaaacuuuuuugaaauaauaaaucugguagaaaaaug

SEQ ID NO: 27 - DNA, 137 bp, Artificial Sequence
  3′-UTR of human cytochrome c oxidase subunit 6B1 (COX6B1)
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 28 - RNA, 137 nt, Artificial Sequence
  3′-UTR of human cytochrome c oxidase subunit 6B1 (COX6B1) mRNA
acuggcugcaucucccuuuccucuguccuccauccuucucccaggauggugaagggggaccugguacccagugauccccaccccaggauccuaaaucaugacuuaccugcuaauaaaaacucauuggaaaagugaga

SEQ ID NO: 29 - DNA, 353 bp, Artificial Sequence
  3′-UTR of mouse GNAS (guanine nucleotide binding protein, alpha stimulating) complex locus (Gnas)
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 30 - RNA, 353 nt, Artificial Sequence
  3′-UTR of mouse GNAS (guanine nucleotide binding protein, alpha stimulating) complex locus (Gnas) mRNA
gaagggaacacccaaauuuaauucagccuuaagcacaauuaauuaagagugaaacguaauuguacaagcaguuggucacccaccauagggcaugaucaacaccgcaaccuuuccuuuuucccccagugauucugaaaaaccccucuucccuucagcuugcuuagauguuccaaauuuaguaagcuuaaggcggccuacagaagaaaaagaaaaaaaaggccacaaaaguucccucucacuuucaguaaauaaaauaaaagcagcaacagaaauaaagaaauaaaugaaauucaaaaugaaauaaauauuguguugugcagcauuaaaaaaucaauaaaaauuaaaaaugagca

SEQ ID NO: 31 - DNA, 133 bp, Artificial Sequence
  3′-UTR of mouse NADH dehydrogenase (ubiquinone) 1 alpha subcomplex, 1 (Ndufa1)
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 32 - RNA, 133 nt, Artificial Sequence
  3′-UTR of mouse NADH dehydrogenase (ubiquinone) 1 alpha subcomplex, 1 (Ndufa1) mRNA
ggaagcauuuuccuggcugauuaaaagaaauuacucagcuauggucaucuguuccuguuagaaggcuaugcagcauauuauauacuaugcgcauguuaugaaaugcauaauaaaaaauuuuaaaaaaucuaaa

SEQ ID NO: 33 - DNA, 70 bp, Artificial Sequence
  3′-UTR of human ribosomal protein S9 (RPS9)
gtccacctgtccctcctgggctgctggattgtctcgttttcctgccaaataaacaggatcagcgctttac

SEQ ID NO: 34 - RNA, 70 nt, Artificial Sequence
  3′-UTR of human ribosomal protein S9 (RPS9) mRNA
guccaccugucccuccugggcugcuggauugucucguuuuccugccaaauaaacaggaucagcgcuuuac

SEQ ID NO: 35 - DNA, 187 bp, Artificial Sequence
  3′-UTR ALB7
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 36 - RNA, 187 nt, Artificial Sequence
  3′-UTR ALB7 mRNA
gcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccu

SEQ ID NO: 37 - DNA, 44 bp, Artificial Sequence
  Center, alpha-complex-binding portion of the 3′-UTR of an alpha-globin gene (muag)
gcccgatgggcctcccaacgggccctcctcccctccttgcaccg

SEQ ID NO: 38 - RNA, 44 nt, Artificial Sequence
  Center, alpha-complex-binding portion of the 3′-UTR of an alpha-globin gene (muag) mRNA
gcccgaugggccucccaacgggcccuccuccccuccuugcaccg

SEQ ID NO: 39 - DNA, 24 bp, Artificial Sequence
  histone stem-loop sequence (histoneSL, hSL)
caaaggctcttttcagagccacca

SEQ ID NO: 40 - RNA, 24 nt, Artificial Sequence
  histone stem-loop sequence (histoneSL, hSL) mRNA
caaaggcucuuuucagagccacca

SEQ ID NO: 41 - DNA, 13 bp, Artificial Sequence
  Kozak
gccgccaccatgg

SEQ ID NO: 42 - RNA, 13 nt, Artificial Sequence
  Kozak mRNA
gccgccaccaugg

SEQ ID NO: 43 - RNA, 6 nt, Artificial Sequence
  mRNA 5′-end
gggaga

SEQ ID NO: 44 - RNA, 6 nt, Artificial Sequence
  mRNA 5′-end
aggaga

SEQ ID NO: 45 - RNA, 128 nt, Artificial Sequence
  mRNA 3′-end A64-N5-C30-histoneSL-N5
aaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 46 - RNA, 129 nt, Artificial Sequence
  mRNA 3′-end histoneSL-A100-N5
caaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 47 - RNA, 93 nt, Artificial Sequence
  mRNA 3′-end histoneSL-A64-N5
caaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 48 - RNA, 105 nt, Artificial Sequence
  mRNA 3′-end A100-N5
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 49 - RNA, 69 nt, Artificial Sequence
  mRNA 3′-end A64-N5
aaaaaaaaaaaaaaaaaaaaaaaaaaaaaaaaaaaaaaaaaaaaaaaaaaaaaaaaaaaaaaaagaauu

SEQ ID NO: 50 - RNA, 124 nt, Artificial Sequence
  mRNA 3′-end histoneSL-A100
caaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 51 - RNA, 88 nt, Artificial Sequence
  mRNA 3′-end histoneSL-A64
caaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 52 - RNA, 100 nt, Artificial Sequence
  mRNA 3′-end A100
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 53 - RNA, 64 nt, Artificial Sequence
  mRNA 3′-end A64
aaaaaaaaaaaaaaaaaaaaaaaaaaaaaaaaaaaaaaaaaaaaaaaaaaaaaaaaaaaaaaaa

SEQ ID NO: 54 - RNA, 93 nt, Artificial Sequence
  mRNA 3′-end A64-histoneSL-N5
aaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 55 - RNA, 134 nt, Artificial Sequence
  mRNA 3′-end N6-A64-N5-C30-histoneSL-N5
auuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 56 - RNA, 134 nt, Artificial Sequence
  mRNA 3′-end N6-A64-N5-C30-histoneSL-N5
agaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 57 - RNA, 135 nt, Artificial Sequence
  mRNA 3′-end N6-histoneSL-A100-N5
auuaaucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 58 - RNA, 135 nt, Artificial Sequence
  mRNA 3′-end N6-histoneSL-A100-N5
agaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 59 - RNA, 99 nt, Artificial Sequence
  mRNA 3′-end N6-histoneSL-A64-N5
auuaaucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 60 - RNA, 99 nt, Artificial Sequence
  mRNA 3′-end N6-histoneSL-A64-N5
agaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 61 - RNA, 111 nt, Artificial Sequence
  mRNA 3′-end N6-A100-N5
auuaauaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 62 - RNA, 111 nt, Artificial Sequence
  mRNA 3′-end N6-A100-N5
agaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 63 - RNA, 75 nt, Artificial Sequence
  mRNA 3′-end N6-A64-N5
auuaauaaaaaaaaaaaaaaaaaaaaaaaaaaaaaaaaaaaaaaaaaaaaaaaaaaaaaaaaaaaaaaaagaauu

SEQ ID NO: 64 - RNA, 75 nt, Artificial Sequence
  mRNA 3′-end N6-A64-N5
agaucuaaaaaaaaaaaaaaaaaaaaaaaaaaaaaaaaaaaaaaaaaaaaaaaaaaaaaaaaaaaaaaaagaauu

SEQ ID NO: 65 - RNA, 130 nt, Artificial Sequence
  mRNA 3′-end N6-histoneSL-A100
auuaaucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - RNA, 130 nt, Artificial Sequence
  mRNA 3′-end N6-histoneSL-A100
agaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - RNA, 94 nt, Artificial Sequence
  mRNA 3′-end N6-histoneSL-A64
auuaaucaaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 68 - RNA, 94 nt, Artificial Sequence
  mRNA 3′-end N6-histoneSL-A64
agaucucaaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 69 - RNA, 106 nt, Artificial Sequence
  mRNA 3′-end N6-A100
auuaauaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 70 - RNA, 106 nt, Artificial Sequence
  mRNA 3′-end N6-A100
agaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 71 - RNA, 70 nt, Artificial Sequence
  mRNA 3′-end N6-A64
auuaauaaaaaaaaaaaaaaaaaaaaaaaaaaaaaaaaaaaaaaaaaaaaaaaaaaaaaaaaaaaaaaaa

SEQ ID NO: 72 - RNA, 70 nt, Artificial Sequence
  mRNA 3′-end N6-A64
agaucuaaaaaaaaaaaaaaaaaaaaaaaaaaaaaaaaaaaaaaaaaaaaaaaaaaaaaaaaaaaaaaaa

SEQ ID NO: 73 - RNA, 99 nt, Artificial Sequence
  mRNA 3′-end N6-A64-histoneSL-N5
auuaauaaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 74 - RNA, 99 nt, Artificial Sequence
  mRNA 3′-end N6-A64-histoneSL-N5
agaucuaaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 75 - protein, 14 aa, Artificial Sequence
  CR12C carrier peptide
CRRRRRRRRRRRRC

SEQ ID NO: 76 - protein, 13 aa, Artificial Sequence
  CR12 carrier peptide
CRRRRRRRRRRRR

SEQ ID NO: 77 - protein, 14 aa, Artificial Sequence
  WR12C carrier peptide
WRRRRRRRRRRRRC

SEQ ID NO: 78 - protein, 18 aa, Artificial Sequence
  CH6R4H6C carrier peptide
CHHHHHHRRRRHHHHHHC

SEQ ID NO: 79 - protein, 18 aa, Artificial Sequence
  CGH5R4H5GC carrier peptide
CGHHHHHRRRRHHHHHGC

SEQ ID NO: 80 - protein, 235 aa, Artificial Sequence
  LC protein (R8534)
METPAQLLFLLLLWLPDTTGEIVLTQSPGTLSLSPGERATLSCRASQSVSSSYLAWYQQKPGQAPRLLIYGASTRATGIPARFSGSGSGTDFTLTISRLEPEDLAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 476 aa, Artificial Sequence
  HC protein (R8535)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 476 aa, Artificial Sequence
  HC-HC_PP3_HC-A protein (R8544)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 476 aa, Artificial Sequence
  HC-HC_PP3_HC-B protein (R8545)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 84 - protein, 476 aa, Artificial Sequence
  HC-HC_PP4_HC-A protein (R8536)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVHLTCLVKGFYPSDIAVEWESNGQPENNYKTTPPVLDSDGSFALYSKLTVDKSRWQQGNVFSCSVMHEALHNHYTQKSLSLSPGK

SEQ ID NO: 85 - protein, 476 aa, Artificial Sequence
  HC-HC_PP4_HC-B protein (R8537)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTTLPPSRDELTKNQVSLTCLVKGFYPSDIAVEWESNGQPENNYKTFPPVLDSDGSFFLYSKLTVDKSRWQQGNVFSCSVMHEALHNHYTQKSLSLSPGK

SEQ ID NO: 86 - protein, 476 aa, Artificial Sequence
  HC-HC_PP5_HC-A protein (R8538)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 87 - protein, 476 aa, Artificial Sequence
  HC-HC_PP5_HC-B protein (R8539)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 88 - protein, 476 aa, Artificial Sequence
  HC-HC_PP16_HC-A protein (R8540)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 89 - protein, 476 aa, Artificial Sequence
  HC-HC_PP16_HC-B protein (R8541)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 90 - protein, 476 aa, Artificial Sequence
  HC-HC_PP18_HC-A protein (R8542)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVSTLPPSRDELTKNQVSLMCLVYGFYPSDIAVEWESNGQPENNYKTTPPVLDSDGSFFLYSVLTVDKSRWQQGNVFSCSVMHEALHNHYTQKSLSLSPGK

SEQ ID NO: 91 - protein, 476 aa, Artificial Sequence
  HC-HC_PP18_HC-B protein (R8543)
MELGLSWIFLLAILKGVQCEVQLVETGGGLVQPGRSLRLSCAASGFSFDEYTMHWVRQAPGKGLEWVAGINWKGNFMGYADSVQGRFTISRDNGKNSLYLQMNSLRAEDTALYYCAKDRLESSAMDILEGGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRGDLTKNQVQLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 92 - RNA, 980 nt, Artificial Sequence
  LC mRNA product R8534
aggagagucccgcagucggcguccagcggcucugcuuguucgugugugugucguugcaggccuuauucaagcuuaccauggagacccccgcccagcugcuguuccugcugcuccuguggcugccggacaccacgggcgagaucgugcucacccagagccccgggacccugucccugagccccggcgagcgcgccacccucuccugccgggcgagccaguccgucagcuccagcuaccuggccugguaccagcagaagcccgggcaggccccgcgccugcucaucuacggcgccuccacgcgggccaccgggauccccgcgcgcuucagcggcuccggcagcgggaccgacuucacccugacgaucucccggcuggagcccgaggaccucgccguguacuacugccagcaguacggcagcuccccguggaccuucgggcagggcaccaagguggagaucaagcgcaccgucgccgcccccagcguguucaucuucccgcccuccgacgagcagcucaagagcgggacggcguccguggucugccugcucaacaacuucuacccccgggaggccaaggugcaguggaagguggacaacgcccugcagagcggcaacagccaggaguccgucaccgagcaggacagcaaggacuccaccuacagccuguccagcacccucacgcuguccaaggccgacuacgagaagcacaagguguacgccugcgaggugacccaccagggccugagcuccccggucaccaagagcuucaaccgcggggagugcugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 93 - RNA, 1703 nt, Artificial Sequence
  HC mRNA product R8535
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucuacacgcugccgcccagccgggacgagcugaccaagaaccaggugucccugaccugccucgugaagggguucuaccccagcgacaucgcggucgagugggagagcaacggccagcccgagaacaacuacaagaccacgccgcccgugcuggacuccgacggcagcuucuuccucuacuccaagcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 94 - RNA, 1703 nt, Artificial Sequence
  HC-HC_PP3_HC-A mRNA product R8544
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucuacacgcugccgcccugccgggacgagcugaccaagaaccaggugucccuguggugccucgugaagggguucuaccccagcgacaucgcggucgagugggagagcaacggccagcccgagaacaacuacaagaccacgccgcccgugcuggacuccgacggcagcuucuuccucuacuccaagcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 95 - RNA, 1703 nt, Artificial Sequence
  HC-HC_PP3_HC-B mRNA product R8545
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucugcacgcugccgcccagccgggacgagcugaccaagaaccaggugucccuguccugcgccgugaagggguucuaccccagcgacaucgcggucgagugggagagcaacggccagcccgagaacaacuacaagaccacgccgcccgugcuggacuccgacggcagcuucuuccucgucuccaagcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 96 - RNA, 1703 nt, Artificial Sequence
  HC-HC_PP4_HC-A mRNA product R8536
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucuacacgcugccgcccagccgggacgagcugaccaagaaccaggugcaccugaccugccucgugaagggguucuaccccagcgacaucgcggucgagugggagagcaacggccagcccgagaacaacuacaagaccacgccgcccgugcuggacuccgacggcagcuucgcccucuacuccaagcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 97 - RNA, 1703 nt, Artificial Sequence
  HC-HC_PP4_HC-B mRNA product R8537
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucaccacgcugccgcccagccgggacgagcugaccaagaaccaggugucccugaccugccucgugaagggguucuaccccagcgacaucgcggucgagugggagagcaacggccagcccgagaacaacuacaagaccuucccgcccgugcuggacuccgacggcagcuucuuccucuacuccaagcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 98 - RNA, 1703 nt, Artificial Sequence
  HC-HC_PP5_HC-A mRNA product R8538
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucuacguguacccgcccagccgggacgagcugaccaagaaccaggugucccugaccugccucgugaagggguucuaccccagcgacaucgcggucgagugggagagcaacggccagcccgagaacaacuacaagaccacgccgcccgugcuggacuccgacggcagcuucgcccucgucuccaagcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 99 - RNA, 1703 nt, Artificial Sequence
  HC-HC_PP5_HC-B mRNA product R8539
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucuacgugcugccgcccagccgggacgagcugaccaagaaccaggugucccugcucugccucgugaagggguucuaccccagcgacaucgcggucgagugggagagcaacggccagcccgagaacaacuaccugaccuggccgcccgugcuggacuccgacggcagcuucuuccucuacuccaagcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 100 - RNA, 1703 nt, Artificial Sequence
  HC-HC_PP16_HC-A mRNA product R8540
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucuacacgcugccgcccagccgggacgagcugaccaagaaccaggugucccugaccugccucgugaagggguucuaccccagcgacaucgcggucgagugggagagcaacggccagcccgagaacaacuacaagaccacgccgcccgugcuggacuccgacggcagcuuccuccucuacuccaagcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 101 - RNA, 1703 nt, Artificial Sequence
  HC-HC_PP16_HC-B mRNA product R8541
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucuacacgcugccgcccagccgggacgagcugaccaagaaccaggugucccugaccugccucgugaagggguucuaccccagcgacaucgcggucgagugggagagcaacggccagcccgagaacaacuacaagaccacgccgcccgugcuggacuccgacggcagcuucuuccucuacucccggcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 102 - RNA, 1703 nt, Artificial Sequence
  HC-HC_PP18_HC-A mRNA product R8542
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucagcacgcugccgcccagccgggacgagcugaccaagaaccaggugucccugaugugccucguguacggguucuaccccagcgacaucgcggucgagugggagagcaacggccagcccgagaacaacuacaagaccacgccgcccgugcuggacuccgacggcagcuucuuccucuacuccgugcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 103 - RNA, 1703 nt, Artificial Sequence
  HC-HC_PP18_HC-B mRNA product R8543
aggagagucccgcagucggcguccagcggcucugcuuguucgugugugugucguugcaggccuuauucaagcuuaccauggagcugggccucagcuggaucuuccugcuggccauccucaagggggugcagugcgagguccagcugguggagaccggcgggggccuggugcagcccgggcgcucccuccggcugagcugcgccgcguccggcuucagcuucgacgaguacaccaugcacuggguccgccaggccccgggcaaggggcuggaguggguggccggcaucaacuggaaggggaacuucaugggcuacgccgacuccgugcaggggcgguucacgaucagccgcgacaacggcaagaacucccucuaccugcagaugaacagccugcgggccgaggacaccgcgcucuacuacugcgccaaggaccgccuggaguccagcgccauggacauccuggagggcgggaccuucgacaucuggggccaggggaccauggucacgguguccagcgccuccaccaagggccccagcguguucccccucgcgcccuccagcaaguccaccagcggcgggaccgccgcccugggcugccuggucaaggacuacuucccggagcccgugacgguguccuggaacagcggggcccucaccagcggcguccacaccuuccccgccgugcugcaguccagcgggcuguacucccucagcuccguggucaccgugcccagcuccagccugggcacgcagaccuacaucugcaacgugaaccacaagcccuccaacaccaaggucgacaagcggguggagcccaagagcugcgacaagacccacacgugcccgcccugcccggcgcccgagcugcucggcgggcccuccguguuccuguucccgccgaagcccaaggacacccugaugaucagccggacccccgaggugaccugcgucguggucgacgugucccacgaggaccccgaggucaaguucaacugguacguggacggcguggagguccacaacgccaagacgaagccccgcgaggagcaguacaacagcaccuaccgggugguguccguccucaccgugcugcaccaggacuggcugaacgggaaggaguacaagugcaaggugagcaacaaggcccuccccgcccccaucgagaagaccaucuccaaggccaagggccagccccgcgagccgcaggucuacacgcugccgcccagccggggcgaccugaccaagaaccaggugcagcugaccugccucgugaagggguucuaccccagcgacaucgcggucgagugggagagcaacggccagcccgagaacaacuacaagaccacgccgcccgugcuggacuccgacggcagcuucuuccucgccuccaagcugaccguggacaagagccgcuggcagcaggggaacgucuucuccugcagcgugaugcacgaggcccugcacaaccacuacacccagaagucccucagccuguccccgggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 104 - protein, 66 aa, Artificial Sequence
  HC-A stretch aaE345-L410 comprising assembly promoter (of PP3)
EPQVYTLPPCRDELTKNQVSLWCLVKGFYPSDIAVEWESNGQPENNYKTTPPVLDSDGSFFLYSKL

SEQ ID NO: 105 - protein, 66 aa, Artificial Sequence
  HC-B stretch aaE345-L410 comprising assembly promoter (of PP3)
EPQVCTLPPSRDELTKNQVSLSCAVKGFYPSDIAVEWESNGQPENNYKTTPPVLDSDGSFFLVSKL

SEQ ID NO: 106 - protein, 66 aa, Artificial Sequence
  HC-A stretch aaE345-L410 comprising assembly promoter (of PP4)
EPQVYTLPPSRDELTKNQVHLTCLVKGFYPSDIAVEWESNGQPENNYKTTPPVLDSDGSFALYSKL

SEQ ID NO: 107 - protein, 66 aa, Artificial Sequence
  HC-B stretch aaE345-L410 comprising assembly promoter (of PP4)
EPQVTTLPPSRDELTKNQVSLTCLVKGFYPSDIAVEWESNGQPENNYKTFPPVLDSDGSFFLYSKL

SEQ ID NO: 108 - protein, 66 aa, Artificial Sequence
  HC-A stretch aaE345-L410 comprising assembly promoter (of PP5)
EPQVYVYPPSRDELTKNQVSLTCLVKGFYPSDIAVEWESNGQPENNYKTTPPVLDSDGSFALVSKL

SEQ ID NO: 109 - protein, 66 aa, Artificial Sequence
  HC-B stretch aaE345-L410 comprising assembly promoter (of PP5)
EPQVYVLPPSRDELTKNQVSLLCLVKGFYPSDIAVEWESNGQPENNYLTWPPVLDSDGSFFLYSKL

SEQ ID NO: 110 - protein, 66 aa, Artificial Sequence
  HC-A stretch aaE345-L410 comprising assembly promoter (of PP16)
EPQVYTLPPSRDELTKNQVSLTCLVKGFYPSDIAVEWESNGQPENNYKTTPPVLDSDGSFLLYSKL

SEQ ID NO: 111 - protein, 66 aa, Artificial Sequence
  HC-B stretch aaE345-L410 comprising assembly promoter (of PP16)
EPQVYTLPPSRDELTKNQVSLTCLVKGFYPSDIAVEWESNGQPENNYKTTPPVLDSDGSFFLYSRL

SEQ ID NO: 112 - protein, 66 aa, Artificial Sequence
  HC-A stretch aaE345-L410 comprising assembly promoter (of PP18)
EPQVSTLPPSRDELTKNQVSLMCLVYGFYPSDIAVEWESNGQPENNYKTTPPVLDSDGSFFLYSVL

SEQ ID NO: 113 - protein, 66 aa, Artificial Sequence
  HC-B stretch aaE345-L410 comprising assembly promoter (of PP18)
EPQVYTLPPSRGDLTKNQVQLTCLVKGFYPSDIAVEWESNGQPENNYKTTPPVLDSDGSFFLASKL

(19) United States
(12) Patent Application Publication
(10) Pub. No.: US 2023/0272052 A1
(43) Pub. Date: Aug. 31, 2023

(54) NUCLEIC ACID ENCODED ANTIBODY MIXTURES

(71) Applicant: CureVac SE, Tübingen (DE)

(72) Inventors: Hans Wolfgang GROßE, Tübingen (DE); Patrick BAUMHOF, Tübingen (DE)

(73) Assignee: CureVac SE, Tübingen (DE)

(21) Appl. No.: 18/007,468

(22) PCT Filed: Jul. 30, 2021
(86) PCT No.: PCT/EP2021/071479

§ 371 Date: Jan. 30, 2023

(30) Foreign Application Priority Data
Jul. 31, 2020 (WO) PCT/EP2020/071678

(51) Int. Cl. C07K 16/10; A61K 9/51; A61K 48/00

(52) U.S. Cl. CPC C07K 16/1018; A61K 9/5123; A61K 48/005; A61K 2039/505

(57) ABSTRACT

The invention relates inter alia to a nucleic acid composition for the expression of at least two antibodies, preferably a mixture of assembled antibodies in a cell or subject, wherein at least one coding sequence of the nucleic acid composition encodes at least one antibody chain assembly promoter. Further, the invention relates to a nucleic acid sequence set for expression of at least one assembled antibody and to a combination of different nucleic acid sequence sets. Additionally, first and second medical uses, methods of treating or preventing diseases, disorders or conditions, and methods for the production of antibody mixtures are provided.

### INTRODUCTION

The invention relates inter alia to a nucleic acid composition for the expression of at least two, preferably a mixture of different assembled antibodies in a cell or subject, wherein at least one coding sequence of the nucleic acid composition encodes at least one antibody chain assembly promoter. Further, the invention relates to a nucleic acid sequence set for expression of at least one assembled antibody and to a combination of different nucleic acid sequence sets. Additionally, first and second medical uses, methods of treating or preventing diseases, disorders or conditions, and methods for the production of antibody mixtures are provided.

Antibodies are powerful therapeutic molecules, and are currently used for various therapeutic treatments including cancer, autoimmune diseases, cardiovascular disease, or passive vaccination. A combination of different therapeutic antibodies opens up a broad variety of new treatment options. However, a combination of multiple antibodies increases costs and complexity, in particular when produced by classical recombinant technologies.

Nucleic acid based therapeutics provide alternative approaches to reduce costs and complexity of antibody therapies. For example, coding nucleic acid (e.g. mRNA) can be administered for delivering large amounts of antibodies in vivo. Moreover, nucleic-acid based therapeutics, e.g. mRNA therapeutics have the potential to encode a plurality of different antibodies in one single nucleic acid composition. Unfortunately, the provision of such a therapeutic nucleic acid composition encoding a plurality of antibodies is associated with various fundamental technical problems, particularly problems associated with the correct assembly of the encoded antibodies.

A typical antibody comprises two identical heavy chains (HC) and two identical light chains (LC) which are combined to form Y-shaped antibody molecules. In a B-cell clone, HCs and LCs are co-translationally translocated into the ER, and folding begins before the polypeptide chains are completely translated. The assembly of such a Y-shaped antibody molecule takes place in one specific B-cell clone and involves steps including homo dimerization of the fragment crystallizable (Fc) regions of two identical heavy chains (HCs) and the subsequent assembly of two identical light chains (LCs) via disulfide linkages between each HC and LC. Correct antibody assembly is unproblematic due to the fact that only one type of antibody is produced by a one type of B-cell clone.

However, the administration of a nucleic acid composition encoding more than one antibody (e.g. an antibody mixture or cocktail) to a cell or preferably to a subject (for in vivo applications) requires the correct assembly of all the encoded heavy chains (HC) and, optionally, all the encoded light chains (LC). As cells that get transfected with such a composition could express multiple different HCs and LCs simultaneously, a correct assembly of the antibodies is more complex, and totally different to the “natural” situation where one specific B-cell produces only one type of antibody.

Approaches to generate more than one antibody encoded by nucleic acids have been described in regards of in vitro antibody production and subsequent antibody recovery from the cells and purification of the antibodies: WO2013157953 relates to the expression of at least two different Ig-like molecules from a single host cell, wherein the IgG-like molecules are provided by plasmid DNA. After production of the antibodies in vitro, the antibodies are harvested which also involves steps of antibody recovery and purification.

WO2004009618 relates to the expression of a mixture of antibodies in cell culture, wherein the different antibodies of the mixture can form various different heterodimeric by-products. After production of the antibodies in vitro, the antibodies are harvested which also involves steps of antibody recovery and purification.

Yu, Jie, et al (Journal of Biological Chemistry 292.43 (2017): 17885-17896) relates to the in vitro production of antibody mixtures in a single cell line, wherein the antibody mixture is provided by mammalian expression vectors. After production of the antibodies in vitro, the antibodies are harvested which also involves steps of antibody recovery and purification.

EP2889313 relates to the in vitro production of antibody mixtures in a single cell line, wherein the antibody mixture is provided by mammalian expression vectors. After production of the antibodies in vitro, the antibodies are harvested which also involves steps of antibody recovery and purification.

In particular for in vivo applications, it is of paramount importance that a correct assembly of the more than one antibodies is achieved. For example, already in simple case scenario where only two monospecific antibodies are provided, the administration of such a nucleic acid composition would generate multiple unwanted by-products e.g. heterodimeric HC-HC by-products, and only a small portion would assemble correctly. A further complexity may be introduced if a plurality of monospecific antibodies and/or bispecific antibodies are to be administered via a nucleic acid based composition.

Accordingly, such an approach would eventually generate a large portion of mismatched by-products e.g. heterodimeric HC-HC by-products, which would then reduce or minimize the therapeutic efficacy e.g. for in vivo use. Furthermore, the production of mismatched, by-products could induce dramatic unwanted side-effects in a subject (e.g., in case where the misassembled antibodies show off-target binding activity).

The provided technical solution as described in detail herein is a prerequisite for the provision of nucleic acid based medicaments, preferably RNA based medicaments, encoding a mixture of correctly assembled antibodies without generating mis-assembled by-products, and therefore opens up a plethora of novel therapeutic treatment options.

The objects outlined above are inter alia solved by the claimed subject matter of the invention.

### Definitions

For the sake of clarity and readability the following definitions are provided. Any technical feature mentioned for these definitions may be read on each and every embodiment of the invention. In particular, each definition provided in the following may be read on embodiments of the first aspect (“composition”), second aspect (“nucleic acid sequence set”), the third aspect (combination), the fourth aspect (“kit or kit of parts”), and all further aspects (medical uses, method of treatment, method for expressing/producing antibodies).

Percentages in the context of numbers should be understood as relative to the total number of the respective items. In other cases, and depending on the context, percentages should be understood as percentages by weight (wt.-%).

About or approximately: The terms “about” or “approximately” are used herein when parameters or values do not necessarily need to be identical, i.e. 100% the same. Accordingly, “about” means, that a parameter or value may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, or 20%. The skilled person will know that e.g. certain parameters or values may slightly vary based on the method how the parameter is determined. For example, if a certain parameter or value is defined herein to have e.g. a length of “about 1000 nucleotides”, the length may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. Accordingly, the skilled person will know that in that specific example, the length may diverge by 1 to 200 nucleotides, preferably by 1 to 100 nucleotides; in particular, by 5, 10, 20, 30, 40, 50, 60, 70, 80, 90, 100, 110, 120, 130, 140, 150, 160, 170, 180, 190, 200 nucleotides.

Allotype, immunoglobulin allotype: The term “allotype” or “immunoglobulin allotype” as used herein refers to an antibody chain, e.g. antibody heavy chain or antibody light chain found in an individual. The term relates to the allele of the antibody chains found in the individual. Typically, each immunoglobulin has unique sequences particular to the individual's genome that manifest in its constant region. An “allotype” may have unique sequences particular to the individual's genome. These differences may be on amino acid level, and may be manifested in the amino acid sequence in its constant region of an antibody chain, in particular, of an antibody heavy chain or antibody light chain. The most important types are Gm (allotypes of the IgG heavy chain) and km (allotypes of the kappa light chain). For example, the allotypes of the human heavy gamma chains of the IgG are designated as Gm (for gamma marker). The allotypes G1m, G2m, and G3m are carried by the constant region of the gamma1, gamma2, and gamma3 chains, encoded by the IGHG1, IGHG2, and IGHG3 genes, respectively. On the immunoglobulin heavy gamma 1 chains (H-gamma1), the following serological markers have been characterized: four G1m allotypes: G1m17, G1m3, G1m1 and G1m2, two G1m alloallotypes: G1m27 and G1m28 (first characterized and defined as G3m allotypes), and two G1m isoallotypes: nG1m17 and nG1 m1.

Antigen: The term “antigen” as used herein will be recognized and understood by the person of ordinary skill in the art, and is e.g. intended to refer to any substance which may be recognized by components of the immune system, preferably by components of the adaptive immune system. Typically, an antigen is capable of triggering an antigen-specific immune response, e.g. by formation of antibodies and/or antigen-specific T cells as part of an adaptive immune response. As defined above, an antigen can be any target that an antibody or antigen-binding molecule is capable to bind to, e.g. a peptide, a protein, a carbohydrate, a lipid, or any combination thereof.

Antibody, antibody fragment: In the context of the invention, an “antibody” is a polypeptide that specifically recognizes and/or binds to a particular target. The term “target” encompasses all molecules, structures, or agents that an antibody is capable to bind to. Typically, the target is e.g. a peptide, a protein, a carbohydrate, a lipid, or any combination thereof. Most targets of an antibody are considered to be antigens. Accordingly, the term “antibody” refers in the broadest sense to any type of antigen-binding molecule. The term “antibody” may encompass various forms of antigen-binding molecules and antibodies, preferably monoclonal antibodies, including but not being limited to whole antibodies, antibodies of any (recombinant or naturally occurring) antibody format, human antibodies, chimeric antibodies, humanized antibodies and genetically engineered antibodies (variant or mutant antibodies) as long as the characteristic properties of an antibody are retained.

Typically, antibodies are immunoglobulins or can be derived from immunoglobulins. Immunoglobulins can in turn be differentiated into five main classes on the basis of their heavy chain (HC), the IgM (μ), IgD (δ), IgG (γ), IgA (α) and IgE (ε) antibodies, of those IgG antibodies making up the largest proportion. Immunoglobulins can moreover be differentiated into the isotypes K and A on the basis of their light chains.

IgG antibodies are typically built up by two identical light and two identical heavy chain proteins which are bonded to one another via disulfide bridges. The light chain (LC) comprises the N-terminal variable domain VL (also referred to as “light chain variable region”) and the C-terminal constant domain CL (also referred to as “light chain constant region”). The heavy chain (HC) of an IgG antibody can be divided into an N-terminal variable domain VH (also referred to as “heavy chain variable region”) and three constant domains C_{H}1, C_{H}2 and C_{H}3 (all three constant domains together are also referred to as “heavy chain constant region”). While the amino acid sequence is largely the same in the region of the constant domains, wide differences in sequence are typically found within the variable domains.

An antibody recognizes a unique target of e.g. an antigen via its variable domains. In particular, the antibody mediates this function by binding to the target or antigen. The term “antibody” refers to both, glycosylated and non-glycosylated immunoglobulins of any isotype or subclass (e.g., IgG, IgG, IgM, IgE, IgA and IgD). A typical antibody is a tetramer. Each tetramer consists of two pairs of polypeptide chains, each pair having a “light chain” (LC) and a “heavy chain” (HC) as defined above.

Typical examples of antibodies include monoclonal antibodies, monospecific antibodies, bispecific antibodies, multispecific antibodies, minibodies, domain antibodies, synthetic antibodies, antibody mimetic, chimeric antibodies, humanized antibodies, human antibodies, antibody fusions, antibody conjugates, single chain antibodies, antibody derivatives, intrabodies, antibody analogues, and functional antibody fragments. Unless otherwise indicated, the term “antibody” includes, in addition to antibodies comprising two full-length heavy chains and two full-length light chains, derivatives, variants, and antibodies of any formats, which do not comprise two full-length heavy chains and/or two full-length light chains. In some instances an “antibody” may thus include fewer chains, for example a single chain or two chains only. Especially preferred are human or humanized monoclonal antibodies and/or recombinant antibodies, especially as recombinant human monoclonal antibodies.

Typically, an antibody recognizes (and binds to) an antigen or a target. To this end, an antibody usually comprises at least one target binding site (or “antigen binding moiety”), which is also referred to as “paratope” and which recognizes (and binds to) an epitope on the antigen or target. A paratope typically comprises a set of complementary determining regions (CDRs) and usually contains parts of the light chain and parts of the heavy chain of the antibody.

For example, a paratope of native IgG comprises three CDRs of the heavy chain (CDRH1, CDRH2 and CDRH3) and three CDRs of the light chain (CDRL1, CDRL2, and CDRL3). The CDRs of an antibody are arranged in the antibody's variable region: CDRH1, CDRH2 and CDRH3 in the heavy chain variable region (VH) and CDRL1, CDRL2, and CDRL3 in the light chain variable region (VL). In addition, an antibody may comprise a constant region (on heavy and light chain: CH and CL, respectively). In native IgGs, the heavy chain constant region comprises three domains (CH1, CH2 and CH3), whereas the light chain constant region comprises one domain only. Accordingly, an antibody is typically an immunoglobulin or is derived from an immunoglobulin.

An antibody (or an antibody fragment) may fulfill various different functions by recognizing (and binding to) a target, e.g. an antigen, such as neutralization, agglutination, precipitation and/or complement activation. Further, antibodies may recruit one or more effector cells or molecules, e.g. immune effector cells (e.g. in the case of bispecific antibodies), or e.g. selectively engage distinct trigger molecules. Further effector functions may include fixation of complement, binding of phagocytic cells, lymphocytes, platelets, mast cells, and basophils which have immunoglobulin receptors.

Antibody fragments or variants, fragment or a variant of an antibody: The term “fragment or a variant of an antibody” is preferably to be understood as a functional fragment or a functional variant, which comprises at least one functional CDR of the corresponding antibody capable of recognizing (and binding to) an antigen or target. Examples of such antibody fragments are any antibody fragments known to a person skilled in the art, e.g. Fab, Fab′, F(ab′)2, Fc, Facb, pFc′, Fd, und Fv fragments of the above mentioned antibodies etc. For example, a Fab (fragment antigen binding) fragment typically comprises the variable and a constant domain of a light and a heavy chain, e.g. the CH1 and the VH domain of the heavy chain and the complete light chain. The two chains are bonded to one another via a disulfide bridge. A Fab fragment thus conventionally contains the complete antigen-binding region of the original antibody and usually has the same affinity for the antigen, the immunogen or an epitope of a protein. Moreover, antibody fragments consisting of the minimal binding subunit of antibodies are usually known as single-chain antibodies (scFvs) and typically have excellent binding specificity and affinity for their ligands. An scFv fragment (single chain variable fragment) typically comprises the variable domain of the light and of the heavy chain, which are bonded to one another via an artificial polypeptide linker.

The term “variants of an antibody” has to be understood as (i) having the same or similar biological function as the corresponding full length antibody or of the corresponding antibody fragment, or (ii) the same or similar activity of the corresponding full length antibody or of the corresponding antibody fragment, e.g. the specific binding to particular antigens as defined herein.

A fragment or a variant of an antibody according to the invention may typically comprise an amino acid sequence having a sequence identity of at least 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective reference full-length antibody or a fragment thereof.

A fragment of an antibody according to the invention may typically comprise an amino acid sequence having a sequence length of at least 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence length of the respective reference full-length antibody or a fragment thereof.

Antibody light chain fragment: The term “antibody light chain fragment” as used herein, e.g. in the context of antibody light chain A (LC-A) or the antibody light chain B (LC-B) relates to a fragment of an antibody light chain. A typical antibody light chain comprises a variable domain (VL), and a constant domain (CL). Accordingly, in the context of the invention, the term “antibody light chain fragment” may relate to a fragment comprising or consisting of at least a fragment of VL and/or CL. A fragment of a antibody light chain may be N-terminally truncated (e.g. lacking the VL domain or parts of the VL domain), or C-terminally truncated (e.g. lacking the CL domain, or parts of the CL domain), or may be N- and C-terminally truncated. A fragment of an antibody light chain in the context of the invention comprises an amino acid sequence having a sequence length of at least 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence length of the respective reference full-length antibody light chain.

Antibody light chain variant: The term “antibody light chain variant” has to be understood as (i) having the same or similar biological function as the corresponding full length antibody light chain or of the corresponding antibody light chain fragment or, respectively, (ii) the same or similar activity of the corresponding full length antibody light chain or of the corresponding antibody light chain fragment, e.g. the specific binding of particular antigens as defined herein. A variant of an antibody light chain according to the invention may typically comprise an amino acid sequence having a sequence identity of at least 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective reference full length antibody light chain or a fragment thereof.

Antibody heavy chain fragment: The term “antibody heavy chain fragment” as used herein, e.g. in the context of the antibody heavy chain A (HC-A provided by the nucleic acid sequence A) or the antibody heavy chain B (HC-B provided by nucleic acid sequence B) relates to a fragment of an antibody heavy chain. A typical antibody heavy chain comprises a variable domain (VH), and a constant region comprises three domains (CH1, CH2 and CH3). Accordingly, in the context of the invention, the term “antibody heavy chain fragment” may relate to a fragment comprising or consisting of at least a fragment of VH, CH1, CH2, and/or CH3. A fragment of a antibody heavy chain may be N-terminally truncated (e.g. lacking the VH domain or parts of the VH domain), or C-terminally truncated (e.g. lacking the CH3 domain, or parts of the CH3 domain), or may be N- and C-terminally truncated. A typical fragment of an antibody heavy chain may comprise a heavy chain Fab region (comprising to VH and CH1 and a hinge region), and/or an FC region (comprising a hinge region and CH2 and CH3). Accordingly, a typical fragment of an antibody heavy chain may comprise a VH, CH1 and a hinge region, and/or optionally an Fc region (comprising a hinge region and CH2 and CH3). A fragment of a antibody heavy chain in the context of the invention comprises an amino acid sequence having a sequence length of at least 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence length of the respective reference full-length antibody heavy chain.

Antibody heavy chain variant: The term “antibody heavy chain variant” has to be understood as (i) having the same or similar biological function as the corresponding full length antibody heavy chain or of the corresponding antibody heavy chain fragment or, respectively, (ii) the same or similar activity of the corresponding full length antibody heavy chain or of the corresponding antibody heavy chain fragment, e.g. the specific binding of particular antigens as defined herein. A variant of an antibody heavy chain according to the invention may typically comprise an amino acid sequence having a sequence identity of at least 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective reference full length antibody heavy chain or a fragment thereof.

Artificial nucleic acid, artificial DNA, artificial RNA, artificial nucleic acid sequence: The term “artificial nucleic acid” as used herein is intended to refer to a nucleic acid that does not occur naturally. In other words, an artificial nucleic acid may be understood as a non-natural nucleic acid molecule. Such nucleic acid molecules may be non-natural due to its individual sequence (e.g. G/C content modified coding sequence, UTRs) and/or due to other modifications, e.g. structural modifications of nucleotides. An artificial nucleic acid sequence may be a DNA sequence, an RNA sequence, or a hybrid-sequence comprising DNA and RNA portions. An artificial nucleic acid sequence may also comprise or consist of PNA, LNA or other modified nucleotides or nucleotide analogs.

Typically, artificial nucleic acid may be designed and/or generated by genetic engineering to correspond to a desired artificial sequence of nucleotides. In this context, an artificial nucleic acid is a sequence that may not occur naturally, i.e. a sequence that differs from the wild type sequence/the naturally occurring sequence/the reference sequence by at least one nucleotide (via e.g. codon modification). The term “artificial nucleic acid” is not restricted to mean “one single molecule” but is understood to comprise an ensemble of essentially identical nucleic acid molecules. Accordingly, it may relate to a plurality of essentially identical nucleic acid molecules. The term “artificial nucleic acid” as used herein may for example relate to an artificial DNA or, preferably, to an artificial RNA.

Assembled antibody: The terms “intact antibody” or “fully assembled antibody” or “assembled antibody” are used in reference to an antibody to mean that it contains two heavy chains and, optionally two light chains, optionally associated by disulfide bonds as occurs with naturally-produced antibodies. Accordingly, an “intact antibody” or “fully assembled antibody” or “assembled antibody” exerts its function, e.g. binding of at least one antigen. Correct assembly depends on the desired configuration of the encoded antibody. Methods to determine assembly or mis-assembly of an antibody exists in the art, and may suitably be used in the context of the invention to determine the percentage of assembled antibodies and misassembled antibodies. Preferably, mass spectrometry (MS) can be used to determine the percentage of assembled antibodies and misassembled antibodies. For example, the nucleic acid composition encoding antibodies can be administered to cells in vitro (e.g. BHK cells in a cell culture) using a transfection agent (e.g. lipofectamine) to allow expression and secretion of the antibodies. The secreted antibodies can be purified from the cell-culture supernatant using a purification matrix (e.g., protein A plus agarose). Further, the purified antibodies can be subjected to treatment with a cysteine protease that digests IgG antibodies (e.g., FabALACTICA (IgdE) (Genovis)) to yield the disulphide-bridged Fc-portion of the antibodies. Further, the disulphide-bridged Fc-portion may be deglycosylated (e.g. using PNGase). The enzymatic treatment can reduce a full-length antibody (150 kDa plus Glycan pattern) to an Fc portion of 50 kDa without glycan pattern. Afterwards, the samples can be analyzed using HPLC-MS to observe mass differences and to determine the ratio of assembled and misassembled antibodies. An example of such a procedure is provided in the example section (see Example 2). To analyze assembly of antibodies in vivo, the nucleic acid composition encoding antibodies may be administered to animal models e.g. to mice or rats using a suitable delivery system e.g. liposomes or LNPs. Produced antibodies can be purified and analyzed using MS as described above. An example of such a procedure is provided in the example section (see Example 4).

Bispecific antibody, bifunctional antibody: The term “bispecific antibody” or “bifunctional antibody” relates to antibodies that comprise specificities to two antigens (bi+specific) in any of several ways: antibodies that have affinities for two antigens; antibodies that are specific to two antigens or two epitopes; or antibodies specific to two types of cell or tissues. Bispecific antibody can simultaneously bind to two different types of antigen. Accordingly, a bispecific antibody has specificities for at least two different, typically non-overlapping, epitopes. Such epitopes may be on the same or different targets. If the epitopes are on different targets, such targets may be on the same cell or different cells or cell types. Bispecific antibodies may be in the IgG-like configuration or format. This format retains the traditional monoclonal antibody (mAb) structure of two Fab arms and one Fc region, except the two Fab sites bind different antigens. There are other bispecific antibodies that lack an Fc region entirely. These include chemically linked Fabs, consisting of only the Fab regions, and various types of bivalent single-chain variable fragments (scFvs). There are also fusion proteins mimicking the variable domains of two antibodies, or formats e.g. bi-specific T-cell engagers (BiTEs). According to the invention, a bispecific antibody would comprise two different target binding sites.

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In other embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Carrier/polymeric carrier: A carrier in the context of the invention may typically be a compound that facilitates transport and/or complexation of another compound (cargo). A polymeric carrier is typically a carrier that is formed of a polymer. A carrier may be associated to its cargo by covalent or non-covalent interaction. A carrier in the context of the invention may transport nucleic acids, e.g. RNA or DNA, to the target cells. The carrier may—for some embodiments—be a cationic or polycationic compound.

Cationic compound, polycationic compound: The term “cationic compound” typically refers to a charged molecule, which is positively charged (cation) at a pH value typically from 1 to 9, preferably at a pH value of or below 9 (e.g. from 5 to 9), of or below 8 (e.g. from 5 to 8), of or below 7 (e.g. from 5 to 7), most preferably at a physiological pH, e.g. from 7.3 to 7.4. Accordingly, a cationic compound may be any positively charged compound or polymer, preferably a cationic peptide or protein, or a lipid or lipidoid, which is positively charged under physiological conditions, particularly under physiological conditions in vivo. A “cationic peptide or protein” may contain at least one positively charged amino acid, or more than one positively charged amino acid, e.g. selected from Arg, His, Lys or Orn. Accordingly, “polycationic” compounds are also within the scope exhibiting more than one positive charge under the conditions given, e.g. polycationic peptide or protein, or a polycationic lipid or lipidoid.

Cap, 5′-cap structure, 5-cap, cap: The term “5′-cap structure” as used herein will be recognized and understood by the person of ordinary skill in the art, and is e.g. intended to refer to a 5′ modified nucleotide, particularly a guanine nucleotide, positioned at the 5′-end of a nucleic acid, e.g. an RNA or mRNA. Preferably, the 5′-cap structure is connected via a 5′-5′-triphosphate linkage to a nucleic acid. 5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

Cap analoque, tri-nucleotide cap analogue: The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of a nucleic acid molecule, particularly of an RNA molecule, when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogues in that context are described in WO2017/066793, WO2017/066781, WO2017/066791, WO2017/066789, WO2017/053297, WO2017/066782, WO2018/075827 and WO2017/066797 wherein the disclosures referring to cap analogues are incorporated herewith by reference. Suitable in the context of the invention are tri-nucleotide cap analogue for the co-transcriptional generation of a cap1 structure (as defined herein).

Chimeric antibody: The term “chimeric antibody”, as used herein, refers to an antibody in which both chain types are chimeric as a result of antibody engineering. A chimeric chain is a chain that contains a foreign variable domain (originating from a non-human species, or synthetic or engineered from any species including human) linked to a constant region of e.g. human origin. The variable domain of a chimeric chain has a V region amino acid sequence which, analyzed as a whole, is closer to non-human species than to human.

Circular RNA, circRNAs: As used herein, the terms “circular RNA” or “circRNAs” has to be understood as a circular polynucleotide constructs that encode at least one antibody chain as defined herein. Preferably, such a circRNA is a single stranded RNA molecule. In the context of the invention, circRNA comprises at least one coding sequence encoding at least one antibody or antibody, or a fragment or a variant thereof.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be a DNA sequence, preferably an RNA sequence, consisting of a number of nucleotides that may be divided by three, which starts with a start codon and which preferably terminates with a stop codon. In embodiments, the cds of the DNA or RNA may terminate with one or two or more stop codons.

Codon modified coding sequence: The term “codon modified coding sequence” relates to coding sequences that differ in at least one codon (triplets of nucleotides coding for one amino acid) compared to the corresponding wild type (or reference) coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably (cf. Table 2) to optimize/modify the coding sequence for in vivo applications as outlined herein.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived.

The skilled person is aware that sequence identity is typically calculated for the same types of nucleic acids, i.e. for DNA sequences or for RNA sequences. Thus, it is understood, that if a DNA is “derived from” an RNA or if an RNA is “derived from” a DNA, in a first step, the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antibody chains) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

DNA, coding DNA: DNA is the usual abbreviation for deoxy-ribonucleic acid. It is a nucleic acid molecule, i.e. a polymer consisting of nucleotides. These nucleotides are usually deoxy-adenosine-monophosphate, deoxy-thymidine-monophosphate, deoxy-guanosine-monophosphate and deoxy-cytidine-monophosphate monomers which are—by themselves—composed of a sugar moiety (deoxyribose), a base moiety and a phosphate moiety, and polymerise by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing. In the context of the invention, in particular in the context of the nucleic acid sequence set of the invention, a DNA is preferably a coding DNA (encoding an antibody chain, or a fragment or variant thereof).

Epitope: The term “epitope” (also called “antigen determinant” in the art) as used herein will be recognized and understood by the person of ordinary skill in the art, and is e.g. intended to refer to T cell epitopes and B cell epitopes. T cell epitopes or parts of the antigenic peptides or proteins and may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 to about 20 or even more amino acids. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, preferably having 5 to 15 amino acids, more preferably having 5 to 12 amino acids, even more preferably having 6 to 9 amino acids, which may be recognized by antibodies, i.e. in their native form. Such epitopes of proteins or peptides may furthermore be selected from any of the herein mentioned variants of such proteins or peptides. In this context epitopes can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Expression: The term “expression” as used herein will be recognized and understood by the person of ordinary skill in the art, and is e.g. intended to refer to the production of a polypeptide (e.g. heavy chain or light chain of an antibody) or production of multiple polypeptides (e.g. assembled antibody), wherein said polypeptide/said multiple polypeptides are provided by a coding sequence of a nucleic acid sequence as defined herein. For example, “expression” of an RNA sequence refers to production of a protein via translation of the RNA into a polypeptide, or into multiple polypeptides. “Expression” of a DNA sequence refers to production of a protein via transcription of the DNA into RNA and subsequent translation into protein, or into assembled multiple polypeptides. The term “expression” and the term “production” may be used interchangeably herein. Further, the term “expression” preferably relates to production of a certain polypeptide (antibody chains) upon administration of a nucleic acid sequence set to a cell or an organism.

Fragment: The term “fragment” as used throughout the present specification in the context of a nucleic acid sequence (e.g. RNA or a DNA) or an amino acid sequence may typically be a shorter portion of a full-length sequence of e.g. a nucleic acid sequence or an amino acid sequence. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 25%, 30%, 35%, 40%, 50%, 60%, 70%, 80%, 90%, 95% of the total (i.e. full-length) molecule from which the fragment is derived (e.g. from an antibody chain, e.g. HC or LC). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide.

Heterologous: The terms “heterologous” or “heterologous sequence” as used throughout the present specification in the context of a nucleic acid sequence or an amino acid sequence refers to a sequence (e.g. RNA, DNA, amino acid) has to be understood as a sequence that is derived from another gene, another allele, or e.g. another species or virus. Two sequences are typically understood to be “heterologous” if they are not derivable from the same gene or from the same allele. I.e., although heterologous sequences may be derivable from the same organism or virus, in nature, they do not occur in the same nucleic acid or protein.

Histone stem-loop sequences/histone stem-loop structure: The term “histone stem-loop” (abbreviated as “hSL”) is intended to refer to nucleic acid sequences that form a stem-loop secondary structure predominantly found in histone mRNAs. In the context of the invention, histone stem-loop sequences/structures may suitably be selected from histone stem-loop sequences as disclosed in WO2012/019780, the disclosure relating to histone stem-loop sequences/histone stem-loop structures incorporated herewith by reference. A histone stem-loop sequence that may be used within the present invention may preferably be derived from formulae (I) or (II) of WO2012/019780.

Human antibody: The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations, insertions or deletions introduced by random or site-specific mutagenesis in vitro or by somatic mutation in vivo).

However, the term “human antibody”, as used herein, is not intended to include antibodies in which CDR sequences derived from the germline of another mammalian species, such as a mouse, have been grafted onto human framework sequences. In the context of the invention, a human antibody may be encoded by a nucleic acid sequence of the invention.

Humanized antibody: The term “humanized antibody”, as used herein, refers to an antibody in which both chain types are humanized as a result of antibody engineering. A humanized chain is typically a chain in which the complementarity determining regions (CDR) of the variable domains are foreign (originating from one species other than human, or synthetic) whereas the remainder of the chain is of human origin. Humanization assessment is based on the resulting amino acid sequence, and not on the methodology per se, which allows protocols other than grafting to be used. The variable domain of a humanized chain has a V region amino acid sequence which, analyzed as a whole, is closer to human than to other species. In the context of the invention, a humanized antibody may be encoded by a nucleic acid sequence of the invention.

Immunoglobulin isotype, isotype: The term “isotype” as used herein, refers to the immunoglobulin class, for instance IgG1, IgG2, IgG3, IgG4, IgD, IgAI, IgGA2, IgE, or IgM or any allotypes thereof such as IgGlm(za) and IgGlm(f)) that is encoded by heavy chain constant region genes. Further, each heavy chain isotype can be combined with either a kappa (K) or lambda (l) light chain. The expression of a specific isotype determines the function of an antibody via the specific binding to Fc receptor molecules on different immune effector cells. Isotype expression reflects the maturation stage of a B cell. Naive B cells express IgM and IgD isotypes with unmutated variable genes, which are produced from the same initial transcript following alternative splicing.

Intrabody: The term “intrabody”, as used herein are intracellularly expressed antibodies, i.e. antibodies which are coded by nucleic acids localized in the cell and are expressed there. Intrabodies can be localized and expressed at certain sites in the cell. For example, intrabodies can be expressed in the cytoplasm, the formation of disulfide bridges usually being decreased under the reducing conditions of the cytoplasm. It has been possible to demonstrate, however, that cytoplasmic intrabodies, and in particular scFv fragments, can be functional. Cytoplasmic expression opens up the possibility of also inhibiting cytoplasmic proteins. By expression of a signal peptide, intrabodies can be transported into the endoplasmic reticulum (ER) and then secreted as with regular antibodies. In this case, typically only secreted or membrane-located proteins are a target for these antibodies. By additional coding of a C-terminal ER retention signal (for example KDEL) by the RNA according to the invention, the intrabody can remain in the ER (where it may bind to specific antigen located in the ER) and prevent secretion of its antigen and/or transport of its antigen or its target molecule to the plasma membrane. Depending on the requirement, intrabodies can include full length antibodies or antibody fragments as described above. Intrabodies in the context of the present invention preferably initially include full length antibodies, which are retained in the cell and not secreted from the cell (by whatever technique, e.g. retention signal sequences etc.). However, if e.g. intracellular expression of full length antibodies is technically not possible or not appropriate, antibody fragments as described above can also be employed as intrabodies. In the context of the invention, a intrabody may be encoded by a nucleic acid sequence.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Lipidoid compound: A lipidoid compound, also simply referred to as lipidoid, is a lipid-like compound, i.e. an amphiphilic compound with lipid-like physical properties. In the context of the present invention, the term lipid is considered to also encompass lipidoid compounds.

MicroRNAs (or miRNA): The terms “MicroRNAs” or “miRNA” relate to 19-25 nucleotide long noncoding RNAs that bind to the 3-UTR of nucleic acid molecules (the respective miRNA binding sites) and down-regulate gene expression either by reducing nucleic acid molecule stability or by inhibiting translation. E.g., microRNAs are known to regulate RNA, and thereby protein expression, e.g. in liver (miR-122), heart (miR-Id, miR-149), endothelial cells (miR-17-92, miR-126), adipose tissue (let-7, miR-30c), kidney (miR-192, miR-194, miR-204), myeloid cells (miR-142-3p, miR-142-5p, miR-16, miR-21, miR-223, miR-24, miR-27), muscle (miR-133, miR-206, miR-208), and lung epithelial cells (let-7, miR-133, miR-126). An nucleic acid of the invention may comprise one or more microRNA target sequences, microRNA sequences, or microRNA seeds.

Mixed isotvpe: The term “mixed isotype” used herein refers to Fc region of an immunoglobulin generated by combining structural features of one isotype with the analogous region from another isotype thereby generating a hybrid isotype. A mixed isotype may comprise an Fc region having a sequence comprised of two or more isotypes selected from the following IgG1, IgG2, IgG3, IgG4, IgD, IgAI, IgGA2, IgE, or IgM thereby generating combinations such as e.g. IgG1/IgG3, IgG1/IgG4, IgG2/IgG3 or IgG2/IgG4.

Mixture of different antibodies: The term “mixture of different antibodies” denotes a composition comprising different antibody molecules which may differ with respect to their amino acid sequence. Accordingly, different antibodies in a mixture (e.g. at least two) represent different antibody species. Identical antibodies in the mixture belong to the same antibody molecule species. Antibodies of different species differ with respect to their sequence and/or their structure. Hence, a “species” denotes a group of essentially identical antibody molecules. Each of the different antibody species in the context of the invention are encoded by the n different nucleic acid sequence sets and, optionally, by the m additional nucleic acid sequences. For example, the nucleic acid composition of the invention comprising n different nucleic acid sequence sets and, optionally, by the m additional nucleic acid sequences may encode for a mixture of antibodies as defined herein, preferably to 2 to 40, preferably 2, 3, 4, 5, 6, 7, 8, 9, 10, 11, 12, 13, 14, 15, 16, 17, 18, 19, or 20 assembled antibodies. Accordingly, in the context of the invention, the term “mixture of different antibodies” relates to a composition comprising a plurality, e.g. 2, 3, 4, 5, 6, 7, 8, 9, 10, 11, 12, 13, 14, 15, 16, 17, 18, 19, or 20 different (preferably correctly) assembled antibody species.

Monocistronic. bicistronic, multicistronic: The term “monocistronic” will be recognized and understood by the person of ordinary skill in the art, and is e.g. intended to refer to a nucleic acid that comprises only one coding sequence. For example, a monocistronic nucleic acid of the invention may encode one protein, e.g. HC or LC, or a fragment thereof. The terms “bicistronic”, or “multicistronic” as used herein will be recognized and understood by the person of ordinary skill in the art, and are e.g. intended to refer to a nucleic acid that may comprise two (bicistronic) or more (multicistronic) coding sequences. For example, a bicistronic nucleic acid of the invention may encode two proteins, e.g. HC and LC, or a fragment thereof.

Monoclonal antibody: The terms “monoclonal antibody”, “monoclonal Ab”, “monoclonal antibody composition”, “mAb”, or the like, as used herein refer to a Ab molecule of single molecular composition. A monoclonal antibody displays a single binding specificity and affinity for a particular epitope. Accordingly, the term “human monoclonal antibody” refers to Abs displaying a single binding specificity which have variable and constant regions derived from human germline immunoglobulin sequences. Human mAbs can be generated by a hybridoma which includes a B cell obtained from a transgenic or trans-chromosomal non-human animal, such as a transgenic mouse, having a genome comprising a human heavy chain transgene repertoire and a light chain transgene repertoire, rearranged to produce a functional human antibody and fused to an immortalized cell. In the context of the invention, a monoclonal antibody may be encoded by a nucleic acid sequence of the invention.

Monospecific antibody: The term “monospecific antibody” relates to antibodies whose specificity to antigens is singular (mono-+specific) in any of several ways: antibodies that all have affinity for the same antigen; antibodies that are specific to one antigen or one epitope; or antibodies specific to one type of cell or tissue. The terms “monospecific” and “monovalent” may be used interchangeably; both can indicate specificity to one antigen, one epitope, or one cell type. In the context of the invention, a monospecific antibody suitably comprises two essentially identical target binding sites. In the context of the invention, a monospecific antibody may be encoded by a nucleic acid sequence of the invention.

Multispecific antibody: The term “multispecific antibody” relates to antibodies that comprise specificities to multiple antigens (multi-+specific) in any of several ways: antibodies that have affinities for multiple antigens; antibodies that are specific to multiple antigens or multiple epitopes; or antibodies specific to multiple types of cell or tissues. The terms “multispecific” and “multivalent” may be used interchangeably; both can indicate specificity to multiple antigens, one multiple epitopes, or multiple cell types. In the context of the invention, a multispecific antibody would comprise at least two different target binding sites. In the context of the invention, a multispecific antibody may be encoded by a nucleic acid sequence.

Nucleoside, Nucleotide: The term “nucleoside” generally refers to compounds consisting of a sugar, usually ribose or deoxyribose, and a purine or pyrimidine base. The term “nucleotide” generally refers to a nucleoside comprising a phosphate group attached to the sugar.

Nucleic acid, nucleic acid molecule: The terms “nucleic acid” or “nucleic acid molecule” as used herein, in particular as used herein will be recognized and understood by the person of ordinary skill in the art. The terms “nucleic acid” or “nucleic acid molecule” preferably refers to DNA (molecules) or RNA (molecules). The term is used synonymously with the term polynucleotide. Preferably, a nucleic acid or a nucleic acid molecule is a polymer comprising or consisting of nucleotide monomers that are covalently linked to each other by phosphodiester-bonds of a sugar/phosphate-backbone. The terms “nucleic acid” or “nucleic acid molecule” also encompasses modified nucleic acid (molecules), such as base-modified, sugar-modified or backbone-modified DNA or RNA (molecules) as defined herein. Accordingly, the nucleic acid of the invention may be a DNA or an RNA.

Nucleic acid sequence, DNA sequence, RNA sequence: The terms “nucleic acid sequence”, “DNA sequence”, “RNA sequence” will be recognized and understood by the person of ordinary skill in the art, and e.g. refer to a particular and individual order of the succession of its nucleotides.

Nucleic acid species: In the context of the invention, the term “nucleic acid species” is not restricted to mean “one single nucleic acid molecule” but is understood to comprise an ensemble of essentially identical nucleic acid molecules (e.g. DNA molecules or RNA molecules). Accordingly, it may relate to a plurality of essentially identical (coding) nucleic acid molecules. Said ensemble of essentially identical (coding) nucleic acid molecules typically encodes essentially the same protein, e.g. the same antibody chain.

Pentaspecific antibody, Hexaspecific antibody: The term “pentaspecific antibody” or “hexaspecific antibody” relates to antibodies that comprise specificities to five or six antigens in any of several ways: antibodies that have affinities for five or six antigens; antibodies that are specific to five or six antigens or five or six epitopes; or antibodies specific to five or six types of cell or tissues. In the context of the invention, a pentaspecific antibody or hexaspecific antibody may be encoded by a nucleic acid sequence.

Permanently cationic: The term “permanently cationic” as used herein will be recognized and understood by the person of ordinary skill in the art, and means, e.g., that the respective compound, or group, or atom, is positively charged at any pH value or hydrogen ion activity of its environment. Typically, the positive charge results from the presence of a quaternary nitrogen atom. Where a compound carries a plurality of such positive charges, it may be referred to as permanently polycationic.

Pharmaceutically effective amount: A pharmaceutically effective amount in the context of the invention is typically understood to be an amount that is sufficient to induce a pharmaceutical effect. For example, in the context of the invention, a pharmaceutically effective amount relates to the amount of nucleic acid that is required to obtain expression of at least two assembled antibodies, thereby induce a pharmaceutical effect.

Poly(A) sequence, poly(A) tail, 3′-poly(A) tail: The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of a linear nucleic acid (e.g. mRNA), of up to about 1000 adenosine nucleotides. Preferably, said poly(A) sequence is essentially homopolymeric, e.g. a poly(A) sequence of e.g. 100 adenosine nucleotides has essentially the length of 100 nucleotides. In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide—or a stretch of nucleotides—different from an adenosine nucleotide). It has to be understood that “poly(A) sequence” as defined herein typically relates to RNA—however in the context of the invention, the term may in some embodiments relate to sequences in a DNA molecule (e.g. a “poly(T) sequence”).

Poly(C) sequence, poly(C) tail, 3′-poly(C) tail: The term “poly(C) sequence” as used herein is intended to be a sequence of cytosine nucleotides of up to about 200 cytosine nucleotides. In preferred embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly preferred embodiment, the poly(C) sequence comprises about 30 cytosine nucleotides. It has to be understood that “poly(C) sequence” as defined herein typically relates to RNA—however in the context of the invention, the term may in some embodiments relate to sequences in a DNA molecule (e.g. a “poly(G) sequence”).

Purified nucleic acid, purified RNA: The term “purified nucleic acid” as used herein has to be understood as nucleic acid which has a higher purity after certain purification steps than the starting material. Typical impurities that are essentially not present in purified nucleic acid comprise peptides or proteins, spermidine, BSA, abortive nucleic acid sequences, nucleic acid fragments, free nucleotides, bacterial impurities, or impurities derived from purification procedures. Accordingly, it is desirable in this regard for the “degree of nucleic acid purity” to be as close as possible to 100%. It is also desirable for the degree of nucleic acid purity that the amount of full-length nucleic acid is as close as possible to 100%. Accordingly “purified nucleic acid” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target nucleic acid and the total area of all peaks representing the by-products. Alternatively, the degree of purity may for example be determined by an analytical agarose gel electrophoresis or capillary gel electrophoresis.

The term “purified RNA” or “purified mRNA” as used herein has to be understood as RNA which has a higher purity after certain purification steps (e.g. HPLC, TFF, Oligo d(T) purification, precipitation steps, AEX, cellulose-based purification) than the starting material (e.g. in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA fragments, abortive sequences etc.), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full-length RNA transcripts is as close as possible to 100%. Accordingly, “purified RNA” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target RNA and the total area of all peaks representing the by-products. Alternatively, the degree of purity may for example be determined by an analytical agarose gel electrophoresis or capillary gel electrophoresis.

RNA, messenger RNA (mRNA): The terms “RNA” and “mRNA” will be recognized and understood by the person of ordinary skill in the art, and are e.g. intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA (messenger RNA) provides the nucleotide coding sequence that may be translated into an aminoacid sequence of a particular peptide or protein. The term “messenger RNA” refers to one specific type of RNA molecule. Typically, an mRNA comprises a 5′-cap, a 5′-UTR, a coding sequence, a 3′-UTR and a poly(A).

RNA in vitro transcription, in vitro transcription, IVT: The terms “RNA in vitro transcription” or “in vitro transcription” relate to a process wherein RNA is synthesized in a cell-free system (in vitro). RNA may be obtained by DNA-dependent in vitro transcription of an appropriate DNA template, which according to the present invention is a linearized plasmid DNA template or a PCR-amplified DNA template. The promoter for controlling RNA in vitro transcription can be any promoter for any DNA-dependent RNA polymerase. Particular examples of DNA-dependent RNA polymerases are the T7, T3, SP6, or Syn5 RNA polymerases. In a preferred embodiment of the present invention the DNA template is linearized with a suitable restriction enzyme, before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein; optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, pyrophosphatase to degrade pyrophosphate, which may inhibit RNA in vitro transcription; MgCl2, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising TRIS-Citrate as disclosed in WO2017/109161. The nucleotide mixture used in RNA in vitro transcription may additionally comprise modified nucleotides as defined herein. In that context, preferred modified nucleotides may be selected from pseudouridine (ψ), N1-methylpseudouridine (m1y), 5-methylcytosine, and 5-methoxyuridine. In particular embodiments, uracil nucleotides in the nucleotide mixture are replaced (either partially or completely) by pseudouridine (y) and/or N1-methylpseudouridine (m1y) to obtain a modified RNA. The nucleotide mixture (i.e. the fraction of each nucleotide in the mixture) used for RNA in vitro transcription reactions may be optimized for the given RNA sequence, preferably as described WO2015/188933. Where more than one different RNA species as defined herein has to be produced, e.g. where 2, 3, 4, 5, 6, 7, 8, 9, 10 or even more different RNAs have to be produced, procedures as described in WO2017/109134 may suitably be used to allow simultaneous manufacturing of different RNAs.

Replicon RNA: The term “replicon RNA” will be recognized and understood by the person of ordinary skill in the art, and is e.g. intended to be an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the coding sequence encoding at least one antibody chain as defined herein). Alternatively, the replicase may be provided on an independent coding RNA construct or a coding DNA construct. Downstream of the replicase may be a sub-genomic promoter that controls replication of the replicon RNA. A replicon RNA may be linear or circular.

RNA species: In the context of the invention, the term “RNA species” is not restricted to mean “one single RNA molecule” but is understood to comprise an ensemble of essentially identical RNA molecules. Accordingly, it may relate to a plurality of essentially identical (coding) RNA molecules. Said ensemble of essentially identical (coding) RNA molecules typically encodes essentially the same protein, e.g. the same antibody chain.

Stabilized nucleic acid: The term “stabilized nucleic acid” refers to “stabilized RNA” or “stabilized DNA” and is intended to comprise nucleic acid that is modified such, e.g. that it is more stable to disintegration or degradation, e.g., by environmental factors or enzymatic digest, such as by exo- or endonuclease degradation, compared to an nucleic acid without such modification. Preferably, a stabilized nucleic acid (e.g. RNA or DNA) in the context of the present invention is stabilized in a cell, such as a prokaryotic or eukaryotic cell, preferably in a mammalian cell, such as a human cell. The stabilization effect may also be exerted outside of cells, e.g. in a buffer solution etc., e.g., for storage of a composition comprising the stabilized nucleic acid.

Single domain antibody: The term “single domain antibody” (sdAb), also known as a Nanobody®, is an antibody fragment consisting of a single monomeric variable antibody chain or domain. Like a whole antibody, a single domain antibody is able to bind selectively to a specific antigen or target. The first single-domain antibodies were engineered from heavy-chain antibodies found in camelids; these are called VHH fragments (also called VNAR-Fragment). Cartilaginous fishes also have heavy-chain antibodies (IgNAR, “immunoglobulin new antigen receptor”), from which single-domain antibodies called VNAR fragments can be obtained. An alternative approach is to split the dimeric variable domains from common immunoglobulin G (IgG) into monomers. Although most research into single-domain antibodies is currently based on heavy chain variable domains, nanobodies derived from light chains have also been shown to bind specifically to target epitopes. In the context of the invention, a single domain antibody may be encoded by a nucleic acid sequence.

Single chain antibody: The term single chain antibody also often called single-chain variable fragments (scFV) typically relates to a fusion protein of the variable regions of the heavy (VH) and light chains (VL) of immunoglobulins, typically connected with a short linker peptide of e.g. ten to about 25 amino acids. The linker may for example be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. In embodiments, a single chain antibody is suitably a fusion protein of HC and LC and typically needs to assemble to a dimer to be an active fully assembled antibody.

Tetraspecific antibody, tetrafunctional antibody: The term “tetraspecific antibody” or “tetrafunctional antibody” relates to antibodies that comprise specificities to four antigens (tetra-+specific) in any of several ways: antibodies that have affinities for four antigens; antibodies that are specific to four antigens or four epitopes; or antibodies specific to four types of cell or tissues. The terms “tetraspecific” and “tetravalent” may be used interchangeably; both can indicate specificity to four antigens, four epitopes, or four cell types. In the context of the invention, a tetraspecific antibody would comprise at least two different target binding sites and at least two further target binding sites. In the context of the invention, a tetraspecific antibody may be encoded by a nucleic acid sequence.

Tri-nucleotide cap analogue, cap1 analogue: A (modified) cap1 structure may be co-transcriptionally generated using tri-nucleotide cap analogue (cap1 analogue) as disclosed in WO2017/053297, WO2017/066793, WO2017/066781, WO2017/066791, WO2017/066789, WO2017/066782, WO2018/075827 and WO2017/066797. In particular, any cap structures derivable from the structure disclosed in claim 1-5 of WO2017/053297 may be suitably used to co-transcriptionally generate a (modified) cap1 structure. Further, any cap structures derivable from the structure defined in claim 1 or claim 21 of WO2018/075827 may be suitably used to co-transcriptionally generate a modified cap1.

Trispecific antibody, trifunctional antibody: The term “trispecific antibody” or “trifunctional antibody” relates to antibodies that comprise specificities to three antigens (tri+specific) in any of several ways: antibodies that have affinities for three antigens; antibodies that are specific to three antigens or three epitopes; or antibodies specific to three types of cell or tissues. The terms “trispecific” and “trivalent” may be used interchangeably; both can indicate specificity to three antigens, three epitopes, or three cell types. In the context of the invention, a trispecific antibody would comprise at least three different target binding sites. Trispecific antibodies typically have three unique binding sites on the antibody: the two Fab regions, and the Fc region. The Fc region made from the two heavy chains forms the third binding site. According to the invention, a trispecific antibody may be encoded by a nucleic acid sequence.

Untranslated region, UTR. UTR element: The term “untranslated region” or “UTR” or “UTR element” will be recognized and understood by the person of ordinary skill in the art, and are e.g. intended to refer to a part of a nucleic acid molecule typically located 5′ or 3′ of a coding sequence. An UTR is not translated into protein. An UTR may be part of a nucleic acid, e.g. a DNA or an RNA. An UTR may comprise elements for controlling gene expression, also called regulatory elements. Such regulatory elements may be, e.g., ribosomal binding sites, miRNA binding sites, promotor elements etc.

3′-untranslated region, 3′-UTR, 3′-UTR element: The term “3′-untranslated region” or “3′-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of a nucleic acid, e.g. a DNA or an RNA, located between a coding sequence and an (optional) terminal poly(A) sequence. A 3′-UTR may comprise elements for controlling gene expression, also called regulatory elements. Such regulatory elements may be, e.g., ribosomal binding sites, miRNA binding sites etc.

5′-untranslated region, 5′-UTR, 5′-UTR element: The terms “5′-untranslated region” or “5′-UTR” or “5′-UTR element” will be recognized and understood by the person of ordinary skill in the art, and are e.g. intended to refer to a part of a nucleic acid molecule located 5′ (i.e. “upstream”) of a coding sequence and which is not translated into protein. A 5′-UTR may be part of a nucleic acid located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′-cap structure (e.g. for mRNA as defined below).

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is preferably a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides will be recognized and understood by the person of ordinary skill in the art, and is e.g. intended to refer to a proteins or peptide variant having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific property. “Variants” of proteins or peptides as defined herein may comprise conservative amino acid substitution(s) compared to their native, i.e. non-mutated physiological, sequence. A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means that the variant exerts the same effect or functionality or at least 40%, 50%, 60%, 70%, 80%, 90%, or 95% of the effect or functionality as the protein it is derived from.

### Short Description of the Invention

Nucleic-acid based therapeutics, e.g. mRNA therapeutics have the potential to encode a plurality, e.g. a mixture of different antibodies in one single nucleic acid composition. However, the provision of such a therapeutic nucleic acid composition encoding a plurality of antibodies is associated with various fundamental problems, particularly associated with the correct assembly of the encoded antibodies, as further outlined below.

The administration of a nucleic acid composition encoding more than one antibody (e.g. an IgG antibody cocktail) to a cell or a subject requires the correct assembly of all the encoded heavy chains (HC) and, optionally, all the encoded light chains (LC) of each antibody. For example, already in simple case scenario where only two monospecific antibodies are provided (e.g. Antibody 1, Antibody 2), upon administration of such a nucleic acid composition only a small portion would assemble correctly (Antibody 1: LC1-HC1-HC1-LC1; Antibody 2: LC2-HC2-HC2-LC2), and multiple unwanted (e.g. heterodimeric or heterotetrameric) by-products would be generated (e.g. LC2-HC1-HC1-LC1; LC2-HC1-HC1-LC2; LC2-HC2-HC1-LC1; LC1-HC2-HC1-LC2; LC2-HC2-HC2-LC1; LC1-HC2-HC2-LC1m, etc.). A further complexity may be introduced if a plurality of monospecific antibodies and/or multispecific antibodies are to be administered via a nucleic acid based composition.

Accordingly, such an approach would eventually generate a large portion of mismatched (e.g. heterodimeric or heterotetrameric) by-products, which would then reduce or minimize the therapeutic efficacy. Furthermore, the production of mismatched, heterodimeric or heterotetrameric by-products could induce dramatic unwanted side-effects in a subject (e.g., in case where the misassembled antibodies show off-target binding activity).

The present invention is, in part, based on the surprising finding that the production of a plurality of fully assembled antibodies can be accomplished in vitro and in vivo by delivering a nucleic acid composition encoding said plurality of antibodies, wherein at least one coding sequence of the nucleic acid sequences encodes at least one antibody chain assembly promoter. The inventive approach is supported by experiments provided in the example section where the inventors identified suitable assembly promotors that allow the combination of different nucleic acid sequences (herein referred to as “nucleic acid sequence set”) for expression of a mixture of correctly assembled antibodies in one cell and in vivo (see Example section). An exemplary illustration how an assembly promoter of the invention can support assembly and, at the same time, can prevent mis-assembly is shown in FIGS. 1 to 3. These findings are the basis for novel treatment options for nucleic acid based compositions, in particular RNA based compositions encoding antibody mixtures, in particular for in vivo applications. The inventors have successfully demonstrated that antibody mixtures can be delivered by nucleic acid sequences and produced upon administration, which makes it possible to eliminate the highly expensive recombinant antibody manufacturing process. In addition, the antibody mixtures produced according to the teaching of the present invention show a high percentage of correctly assembled antibody (species), which is a prerequisite for therapeutic use of nucleic acid compositions encoding antibody mixtures. Moreover, mis-pairing to other antibody heavy chains could be reduced or prevented (e.g. to heavy chains that do not comprise assembly promoters, e.g. wild type (unmodified) heavy chains).

In a first aspect, the present invention relates to a composition comprising n nucleic acid sequence sets for expression of at least two different antibodies in a cell or subject. A nucleic acid set may comprise (a) nucleic acid sequence A comprising at least one coding sequence encoding at least one antibody heavy chain A (HC-A), or a fragment or variant thereof, and (b) nucleic acid sequence B comprising at least one coding sequence encoding at least one antibody heavy chain B (HC-B), or a fragment or variant thereof. The at least one coding sequence of the nucleic acid sequence A and/or the nucleic acid sequence B encodes at least one antibody chain assembly promoter.

Advantageously, administration of the composition of the first aspect to a cell or to a subject leads to expression of at least two assembled antibodies, optionally to expression of 2 to 40, preferably 2, 3, 4, 5, 6, 7, 8, 9, 10, 11, 12, 13, 14, 15, 16, 17, 18, 19, or 20 assembled antibodies in said cell or said subject. Suitably, administration of the composition of the first aspect to a subject leads to in vivo expression of at least two assembled antibodies, optionally to expression of 2 to 40, preferably 2, 3, 4, 5, 6, 7, 8, 9, 10, 11, 12, 13, 14, 15, 16, 17, 18, 19, or 20 assembled antibodies in said subject.

In a second aspect, the present invention relates to a nucleic acid sequence set, preferably as defined in the context of the first aspect.

In a third aspect, the present invention relates to a combination comprising at least two (different) nucleic acid sequence sets of the second aspect.

In a fourth aspect, the invention relates to a kit or kit of parts comprising at least one composition of the first aspect, or at least one nucleic acid sequence set of the second aspect, optionally comprising at least one liquid vehicle for solubilising, and, optionally, technical instructions providing information on administration and dosage of the kit components.

In further aspects, the invention relates to first/second medical uses, method of treatments, and methods of expressing at least two nucleic acid encoded antibodies in an organ or tissue or a subject, and to in vitro, in situ, or ex vivo methods of producing at least two nucleic acid encoded antibodies.

### DETAILED DESCRIPTION OF THE INVENTION

The present application is filed together with a sequence listing in electronic format, which is part of the description of the present application (WIPO standard ST.25). The information contained in the electronic format of the sequence listing filed together with this application is incorporated herein by reference in its entirety. For many sequences, the sequence listing also provides additional detailed information, e.g. regarding certain structural features, sequence modifications, GenBank identifiers, or additional detailed information. In particular, such information is provided under numeric identifier <223> in the WIPO standard ST.25 sequence listing. Accordingly, information provided under said numeric identifier <223> is explicitly included herein in its entirety and has to be understood as integral part of the description of the underlying invention.

Composition

In a first aspect, the present invention relates inter alia to a nucleic acid composition for expression of at least two different antibodies, preferably for expression of a plurality of different antibodies in a cell or a subject.

An antibody in the context of the invention may be without being limited thereto, any type of a monospecific antibody, a bispecific antibody, multispecific antibody, a minibody, a (single) domain antibody, a single chain antibody, a synthetic antibody, an antibody mimetic, a chimeric antibody, a humanized or human antibody, an antibody fusion protein, an antibody conjugate, an antibody derivative, an intrabody, or any antibody analogue or functional antibody fragment thereof.

Antibodies encoded by the nucleic acid composition can be chosen from all antibodies or antibody fragments as defined herein, in particular antibodies or antibody fragments which are or which can be employed for (any) therapeutic or for (any) diagnostic or for (any) research purposes or have been found or are employed in a particular diseases, e.g. cancer diseases, infectious diseases, autoimmune diseases, inflammatory diseases etc.

In a preferred embodiment of the first aspect, the composition encodes at least two different antibodies, preferably a plurality of different antibodies, e.g. 2, 3, 4, 5, 6, 7, 8, 9, 10, 11, 12, 13, 14, 15, 16, 17, 18, 19, 20.

In preferred embodiments, the composition comprises n nucleic acid sequence sets encoding at least one antibody or a fragment or variant thereof, wherein the n different nucleic acid sequence sets comprise
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody heavy chain B (HC-B), or a fragment or variant thereof,
wherein the at least one coding sequence of the nucleic acid sequence A and/or the nucleic acid sequence B encodes at least one antibody chain assembly promoter.

In preferred embodiments, the composition of the first aspect is for expression of at least two different antibodies in a cell. Advantageously, the composition of the first aspect is for expression of at least two (correctly) assembled antibodies in the same cell.

In particularly preferred embodiments, the composition of the first aspect is for expression of at least two different antibodies in vivo, e.g. in a subject, preferably a human subject. Advantageously, said composition is for expression of at least two (correctly) assembled antibodies in vivo, e.g. in a subject, preferably a human subject.

In the following, advantageous features and embodiments of the composition of the first aspect are defined and described. In particular, advantageous embodiments and features of nucleic acid sequence A, nucleic acid sequence B, and further, optional nucleic acid sequences are defined and described. Notably, all embodiments and features of said nucleic acid sequences provided in the context of the first aspect (the “composition”) are likewise be applicable to nucleic acid sequences provided in the context of the second aspect (“the nucleic acid sequence set”), the third aspect (“the combination”), the fourth aspect (“kit or kit of parts”) or to any further aspect described herein (e.g. “medical use”, “method of treatment”, “method of expressing antibodies”, etc.).

In the context of the present invention, the term “nucleic acid sequence set” as used herein preferably means a combined occurrence of nucleic acid sequence A, and nucleic acid sequence B, and, optionally, further nucleic acid sequences (e.g. nucleic acid sequence C, and nucleic acid sequence D) as defined herein. “Combined occurrence” means that the individual components of the nucleic acid sequence set may be provided as (physically) separate entities (e.g. as separate nucleic acid molecules, e.g. a DNA or RNA) or as a combined entity (e.g. as one nucleic acid molecule comprising nucleic acid sequence A and nucleic acid sequence B) or any combination thereof.

Accordingly, in the context of the invention, a “nucleic acid sequence set” comprises at least two nucleic acid sequences (e.g., nucleic acid sequence A and B), optionally, 3, 4, 5, 6, 7, 8, 9, 10 or even more nucleic acid sequences. Said at least two nucleic acid sequences, optionally, 3, 4, 5, 6, 7, 8, 9, 10 or even more nucleic acid sequences, may be provided by one nucleic acid molecule, e.g., DNA or RNA, or may be provide by 2, 3, 4, 5, 6, 7, 8, 9, 10 or more separate nucleic acid molecules as further specified herein. In the context of the invention, one nucleic acid sequence set encodes at least the heavy chains (e.g. HC-A and HC-B) of one antibody species.

The term “nucleic acid sequence A” as used herein has to be understood as any type of nucleic acid sequence, including DNA or RNA sequences, provided that said nucleic acid sequence comprises at least one coding sequence encoding at least one antibody heavy chain A (HC-A), or a fragment or variant thereof. Nucleic acid sequence A is part of the nucleic acid sequence set encoding an antibody. Said “nucleic acid sequence A” may be located on a separate nucleic acid molecule (e.g. a DNA molecule or an RNA molecule) or may be located on a nucleic acid molecule (e.g. a bicistronic—or multicistronic nucleic acid as defined herein) together with nucleic acid sequence B and/or together with an optional further nucleic acid sequence as defined herein. Accordingly, nucleic acid sequence A and nucleic acid sequence B, and, optionally, further nucleic acid sequences may be located on separate entities (e.g. different RNA or DNA molecules) or on the same entity (e.g. the same RNA molecule/the same DNA molecule).

The term “nucleic acid sequence B” as used herein has to be understood as any type of nucleic acid sequence, including DNA, RNA sequences, provided that said nucleic acid sequence comprises at least one coding sequence encoding at least one antibody heavy chain B (HC-B), or a fragment or variant thereof. Nucleic acid sequence B is part of the nucleic acid sequence set encoding an antibody. Said “nucleic acid sequence B” may be located on a separate nucleic acid molecule (e.g. a DNA molecule or an RNA molecule) or may be located on a nucleic acid molecule (e.g. a bicistronic—or multicistronic nucleic acid as defined herein) together with nucleic acid sequence A and/or together with an optional further nucleic acid sequence as defined herein. Accordingly, the nucleic acid sequence B and nucleic acid sequence A, and, optionally, further nucleic acid sequences may be located on separate entities (e.g. different RNA or DNA molecules) or on the same entity (e.g. the same RNA molecule/the same DNA molecule).

The term “antibody chain assembly promoter” as used herein relates to at least one moiety (e.g. an amino acid) that promotes, supports, forces, or directs the correct assembly of at least two antibody polypeptide chains (herein, provided by the nucleic acid sequence set). Further, antibody chain assembly promoter suppresses or reduces mis-assembly. In the context of the invention, such a moiety is typically at least one amino acid substitution capable of promoting, supporting, forcing, or directing a certain assembly of two antibody polypeptide chains. Preferably in the context of the invention, such an amino acid substitution is a substitution that does not occur naturally (in a position that does not occur naturally), suitably, a substitution that does not occur naturally in human antibody chains.

For example, an “antibody chain assembly promoter” may be located on an antibody heavy A and/or on an antibody heavy B to promote, support, force, or direct correct assembly between the two heavy chains, e.g. to promote, support, force, or direct e.g. a heterodimerization of e.g. HCs (if desired) or a homodimerization of e.g. HCs (if desired).

Suitably in the context of the invention, an antibody chain assembly promoter promotes, supports, forces, or directs (correct) assembly of at least two antibody polypeptide chains wherein said at least two antibody polypeptide chains have a corresponding or matching antibody chain assembly promoter. Further, antibody chain assembly promoter suppresses or reduces mis-assembly. Suitably, an antibody chain assembly promoter promotes, supports, forces, or directs assembly of at least two antibody polypeptide chains wherein said at least two antibody polypeptide chains have a corresponding or matching antibody chain assembly promoter, wherein assembly of at least two antibody polypeptide chains is promoted in the presence of an additional antibody polypeptide chain (or additional polypeptide chains) having a non-matching antibody chain assembly promoter or that is lacking an antibody chain assembly promoter.

Merely as an example, suitable antibody chain assembly promoters may promote, support, force, or direct (correct) assembly of at least two antibody polypeptide chains while, at the same time, avoiding assembly to other antibody polypeptide chains lacking an antibody chain assembly promoter or comprising a different antibody chain assembly promoter.

In the context of the invention, said at least one moiety of the antibody chain assembly promoter (e.g. at least one amino acid) is encoded by the at least one coding sequence of nucleic acid sequence A and/or nucleic acid sequence B. As an example, antibody chains comprising an “antibody chain assembly promoter” may show an increased occurrence of correctly assembled antibody chains under certain conditions, compared to naturally occurring antibody chains lacking such an “antibody chain assembly promoter”. An increased occurrence of correctly assembled antibody chains is suitably observed also in the presence of other antibody polypeptide chains (e.g. lacking an assembly promoter) that can provided by e.g. via additional nucleic acid sequences in the composition (see below).

It has to be understood that “correctly assembled” depends on the actual purpose, e.g. whether e.g. heterodimerization (for e.g. HCs of bispecific antibodies) or homodimerization (for e.g. HCs of monospecific antibodies) of heavy chains is preferred.

In a naturally occurring antibody or antibody chains, e.g. an IgG antibody, HCs and LCs are co-translationally translocated into the ER of a B-cell, and folding begins before the polypeptide chains are completely translated. Most IgGs assemble first as HC dimers to which LCs are added covalently via a disulphide bond between the CL and CH1 domains. Heavy chain assembly is mediated by the last domain (the C-terminal domain) of the constant region, i.e. CH3. Interaction of two heavy chains involves about 16 amino acid residues at the interface of the two heavy chains (CH3-CH3 interface). After correct assembly, disulphide bonds in the hinge region connect the two heavy chains to form a HC-HC homodimer. Accordingly, a typical antibody heavy chain comprises a natural antibody chain assembly sequence, forming a CH3-CH3 interface that mediates assembly. It has to be emphasized that such naturally occurring antibody chain assembly interfaces are not comprised by the term “antibody chain assembly promoter” as used herein.

Merely as an example, an “antibody chain assembly promoter” may be derived from any naturally occurring antibody chain assembly sequence, wherein at least one amino acid residue is mutated/changed/substituted to e.g. another amino acid residue. Further, the term “antibody chain assembly promoter” may have a sequence that is 100% identical to a naturally occurring antibody chain assembly sequence, wherein said “antibody chain assembly promoter” is in a position that does not occur in nature. Accordingly, the term “antibody chain assembly promoter” has to be understood as “non-naturally occurring” in terms of the amino acid sequence or the position in an antibody chain (specifically, “non-naturally occurring” has to be understood in comparison to wild-type or naturally occurring human antibody chains).

Typically, in the context of the invention, at least one antibody chain promoter may be located on one antibody chain (e.g. on heavy chain A), and one (preferably different) antibody chain promoter may be located on an antibody chain to which assembly is to be promoted (e.g. on heavy chain B). Suitably, the two antibody chain promoters interact to allow specific assembly of the antibody heavy chains (e.g. HC-A and HC-B). Accordingly, in some embodiments, a antibody chain promoter pair promotes assembly of antibody chains (herein referred to as “assembly promoter pair”).

Accordingly, an antibody chain assembly promoter pair of the invention may comprise a paired amino acid substitution (as further described in the context of the first aspect). A paired amino acid substitution of such a antibody chain assembly promoter pair has to be understood as a substitution pair (of at least two different substitutions), wherein one substitution is located on e.g. heavy chain A and one substitution is located on e.g. heavy chain B.

In preferred embodiments, the at least one antibody chain assembly promoter is a moiety that promotes, supports, forces, or directs (correct) assembly of at least two antibody chains, preferably wherein the moiety comprises at least one amino acid residue in a position that does not occur naturally or at least one amino acid sequence that does not occur naturally.

In embodiments, the at least one antibody chain assembly promoter is a moiety that prevents or reduces assembly of HC-A and/or HC-B to a wild-type (unmodified) antibody heavy chain, preferably to a wild-type (unmodified) antibody heavy chain selected or derived from a human. This is particularly advantageous in the context of in vivo applications, as a mis-pairing to endogenous antibody heavy chains can be prevented which reduces side-effects for medical applications.

In preferred embodiments, the composition comprises at least one nucleic acid sequence set encoding at least one antibody or a fragment or variant of an antibody, wherein the at least one antibody or antibody fragment or variant thereof is derived or selected from a monoclonal antibody or fragments thereof, a chimeric antibody or fragments thereof, a human antibody or fragments thereof, a humanized antibody or fragments thereof, an intrabody or fragments thereof, a single chain antibody or fragments thereof.

In preferred embodiments, the composition comprises at least one nucleic acid sequence set encoding at least one antibody or a fragment or variant of an antibody, wherein the at least one antibody or antibody fragment or variant thereof is derived or selected from an IgG1, IgG2, IgG3, IgG4, IgD, IgA1, IgA2, IgE, IgM, IgNAR, hclgG, BiTE, diabody, DART, VHH or VNAR-Fragment, TandAb, scDiabody; sc-Diabody-CH3, Diabody-CH3, Triple Body, mini antibody, minibody, nanobody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, Tandem scFv-Fc, Fab, Fab′, Fc, Facb, pFc′, Fd, Fv or scFv antibody fragment, scFv-Fc, scFab-Fc. Preferred in that context is IgG1, IgG3, scFv-Fc and scFab-Fc.

In preferred embodiments, the composition comprises at least one nucleic acid sequence set encoding at least one antibody or a fragment or variant of an antibody, wherein the at least one antibody or antibody fragment variant thereof is derived or selected from a single chain variable fragment (scFv antibody). Accordingly, in preferred embodiments, nucleic acid sequence A and/or nucleic acid sequence B comprise at least one coding sequence encoding at least one single chain variable fragment (or a fragment or variant thereof).

In preferred embodiments, the composition comprises at least one nucleic acid sequence set encoding at least one antibody or a fragment or variant of an antibody, wherein the at least one antibody or antibody fragment specifically recognizes and/or binds to at least one target. In preferred embodiments, a target may be selected from at least one epitope or at least one antigen.

In preferred embodiments, the composition comprises at least one nucleic acid sequence set encoding at least one antibody or a fragment or variant of an antibody, wherein the at least one antibody or antibody fragment specifically recognizes and/or binds to at least one target selected from at least one tumor antigen or epitope, at least one antigen or epitope of a pathogen, at least one viral antigen or epitope, at least one bacterial antigen or epitope, at least one protozoan antigen or epitope, at least one antigen or epitope of a cellular signalling molecule, at least one antigen or epitope of a component of the immune system, at least one antigen or epitope of an intracellular protein, or any combination thereof.

In particularly preferred embodiments, the composition comprises at least one nucleic acid sequence set encoding at least one antibody or a fragment or variant of an antibody, wherein the at least one antibody or antibody fragment specifically recognizes and/or binds to at least one target selected from at least one antigen or epitope of a pathogen, preferably a virus or a bacterium.

In preferred embodiments, the composition comprises at least one nucleic acid sequence set encoding at least one antibody or a fragment or variant of an antibody, wherein the at least one antibody or antibody fragment is derived or selected from a monospecific antibody or fragment or variant thereof, or a multispecific antibody or fragment or variant thereof.

In preferred embodiments, the multispecific antibody is derived or selected from a bispecific, trispecific, tetraspecific, pentaspecific, or a hexaspecific antibody or a fragment or variant of any of these.

In the context of the invention, antibody heavy chain A and/or antibody heavy chain B may be selected from a heavy chain that is or is derived from IgM (p), a heavy chain derived from IgD (6), a heavy chain derived from IgG (y), a heavy chain derived from IgA (a) and a heavy chain derived from IgE (s) antibodies.

In preferred embodiments, the at least one HC-A and/or the at least one HC-B is derived or selected from antibody heavy chains selected from IgG1, IgG2, IgG3, IgG4, IgD, IgA1, IgA2, IgE, or IgM, or an allotype, an isotype, or mixed isotype or a fragment or variant of any of these.

In preferred embodiments, the at least one HC-A and/or the at least one HC-B is derived or selected from antibody heavy chains selected from IgG1 and/or IgG3.

In some embodiments, at least one nucleic acid sequence set comprises antibody heavy chains derived from IgG1 and at least one nucleic acid sequence set comprises antibody heavy chains derived from IgG3. In such embodiments, the likelihood of mis-assembly (e.g. HC (of IgG1) to HC (of IgG3)) is further reduced.

In preferred embodiments, the at least one HC-A and/or the at least one HC-B is derived or selected from an antibody heavy chain of IgG, or an allotype or an isotype thereof, preferably an antibody heavy chain of IgG1 or an allotype or an isotype thereof.

Accordingly, in preferred embodiments, the at least one antibody is an IgG or is derived from an IgG. An antibody that is “derived from an IgG” has to be understood as an antibody that comprises two heavy chains (derived from an IgG heavy chain). Preferably, an antibody that is “derived from an IgG” additionally comprises at least a portion of a light chain, preferably at least two light chains.

In embodiments, specific allotypes of heavy chains, in particular IgG heavy chains are selected to e.g. improve protein half life e.g. after expression of the antibody in a cell or a subject (e.g., upon administration of the composition). Without wishing to be bound to theory, specific IgG heavy chains show improved or increase FcRn recycling which leads to longer half-life of the protein.

IgG-heavy chain allotypes are designated as natural genetic marker (Gm) together with the antibody subclass (e.g., G1m) and the allotype number (e.g., G1m3 or G1m1). A total of 4 G1m human allotypes: G1m17, G1m3, G1m1, and G1m2; two G1m alloallotypes: G1m27 and G1m28; and two G1m isoallotypes: nG1m17 and nG1m1 have been identified via serological typing. These define 7 G1m alleles: G1m17,1; G1m3; G1m17,1,27; G1m17,1,28; G1m17,1,27,28; G1m17,1,2; and G1m3,1; where the G1m1 allotype is common to all alleles except G1m3. Most Gm allotypes are located in the Fc-region (CH2 or CH3) of antibodies, with the exception of G1m3 which is linked to amino acid changes in the CH1-region: expressing Arg rather than Lys at position 120. G1m3 also expresses unique amino acids at positions 356 (Glu) and 358 (Met) in CH3 as opposed to Asp/Leu common to all G1m1 allotypes.

While allotypes are encoded by one given Ig gene, some amino acid variations can be found in antibody chains of other isotypes (isoallotypes). For example, the amino acid residue Arg120, which corresponds to G1m3, is also found in antibodies belonging to the IGHG3 and IGHG4 allele family. (Numbering of amino acid residues according to IMGT nomenclature).

In embodiments, the at least one HC-A and/or the at least one HC-B is derived or selected from an antibody heavy chain of IgG, preferably an antibody heavy chain of IgG1 or an allotype or an isotype thereof, wherein the antibody heavy chain of IgG, preferably IgG1, is selected from G1 m17, G1 m3, G1m1 and G1m2, G1m27, G1m28, nG1m17, nG1m1, or any combination thereof. In the context of the invention, also artificially generated IgG allotypes may be used.

In preferred embodiments, heavy chain A and/or heavy chain B is selected or is derived from heavy chain allotype G1m17.

Allotype G1m17 corresponds to the gene IGHG1 CH1 [K120, a359] according to the IMGT unique numbering for C-DOMAIN (Exon numbering 97, Eu numbering 214). The allotype G1m17 (CH1 K120) is found on alleles IGHG1*01, IGHG1*02, IGHG1*04, IGHG1*05, IGHG1*05p, IGHG1*06p and IGHG1*07p.

Accordingly, in particularly preferred embodiments, G1m17,1 (K120;D12/L14) and/or G1m17, -1 (K120; E12/M14) are selected as suitable heavy chains.

In preferred embodiments, heavy chain A and/or heavy chain B is selected or is derived from heavy chain allotype G1m1.

The allotype G1m1 corresponds to the gene IGHG1 CH3 [D12, t36; L14, c40] according to the IMGT unique numbering for C-DOMAIN (Exon numbering 16 and 18, Eu numbering 356 and 358). The allotype G1m1 (CH3 D12, L14) is found on alleles IGHG1*01, IGHG1*02, IGHG1*04, IGHG1*05, IGHG1*05p IGHG1*06p, IGHG1*07p and IGHG1*08p.

Accordingly, in particularly preferred embodiments, G1m3, 1 (R120; D12/L14) and/or G1m3, -1 (R120; E12/M14) are selected as suitable heavy chains of the invention.

In embodiments, the antibody heavy chain of IgG, preferably IgG1, is selected from the allotype G1m3,1 (R120, D12/L14). Without whishing to be bound to theory, G1m3,1 is suitably used as G1m3,1 shows a prolonged protein half-life.

In embodiments, at least one HC-A and/or the at least one HC-B of at least one nucleic acid sequence set is derived or selected from an antibody heavy chain of IgG1, and at least one HC-A and/or the at least one HC-B of at least one nucleic acid sequence set is derived or selected from an antibody heavy chain of IgG2, IgG3, IgG4, IgD, IgA1, IgA2, IgE, or IgM, or an allotype, an isotype, or mixed isotype or a fragment or variant of any of these.

In preferred embodiments, at least one HC-A and the at least one HC-B of at least one nucleic acid sequence set is derived or selected from an antibody heavy chain of IgG1, and at least one HC-A and the at least one HC-B of at least one nucleic acid sequence set is derived or selected from an antibody heavy chain of IgG3.

In various preferred embodiments, the at least one coding sequence nucleic acid sequence A and the nucleic acid sequence B encodes at least one antibody chain assembly promoter.

In preferred embodiments, the at least one antibody chain assembly promoter (e.g. encoded by the coding sequence of nucleic acid sequence A and/or the nucleic acid sequence B) is a heavy chain—heavy chain (HC-HC) assembly promoter and/or a heavy chain-light chain (HC-LC) assembly promoter.

The term “heavy chain-heavy chain assembly promoter” or “HC-HC assembly promoter” as used herein relates to a moiety (e.g. an amino acid) that promotes, supports, forces, or directs assembly of at least two antibody heavy chains (e.g. provided by the nucleic acid set). In the context of the invention, such a moiety is typically at least one amino acid capable of promoting, supporting, forcing, or directing a certain assembly of two antibody heavy chains. For example, an “HC-HC assembly promoter” may be located on an antibody heavy A and/or on an antibody heavy B to promote, support, force, or direct an assembly between the two heavy chains, e.g. to promote, support, force, or direct a heterodimerization (if desired) or a homodimerization of e.g. HCs (if desired).

In the context of the invention, said at least one moiety of the HC-HC assembly promoter (e.g. at least one amino acid) is encoded by the at least one coding sequence of the first nucleic acid sequence and/or the second nucleic acid sequence. As an example, two antibody chains comprising such an “HC-HC assembly promoter” may show an increased occurrence of correctly assembled antibody heavy chains under certain conditions, compared to naturally occurring antibody chains lacking such an “HC-HC assembly promoter”. It has to be understood that “correctly assembled” depends on the actual purpose, e.g. whether e.g. heterodimerization or homodimerization of heavy chains is preferred.

In naturally occurring (wild type or non-modified) antibodies, heavy chain assembly is typically mediated by the last domain (the C-terminal domain) of the constant region, i.e. CH3. For example, interaction of two IgG heavy chains involves about 14, 15, 16, 17, or 18 amino acid residues at the interface of the two heavy chains (CH3-CH3 interface). Said about sixteen amino acid residues on each CH3 domain are typically located on four anti-parallel p-strands. After assembly, disulphide bonds in the hinge region connect the two heavy chains to form a HC-HC homodimer. Accordingly, a typical antibody heavy chain comprises a natural antibody heavy chain assembly sequence interface, forming a CH3-CH3 interface that mediates assembly. It has to be emphasized that such naturally occurring antibody heavy chain assembly interfaces are not comprised by the term “HC-HC assembly promoter” as used herein.

Merely as an example, an “HC-HC assembly promoter” may be derived from any naturally occurring antibody heavy chain assembly sequence, wherein at least one amino acid residue is mutated/changed/substituted to e.g. another amino acid residue. Further, the term “HC-HC assembly promoter” may have a sequence that is 100% identical to a naturally occurring antibody chain assembly sequence, wherein said “HC-HC assembly promoter” is located in a position that does not occur in nature. Accordingly, the term “antibody chain assembly promoter” has to be understood as “non-naturally occurring” in terms of the amino acid sequence or the position in an antibody heavy chain.

In particularly preferred embodiments, the at least one antibody chain assembly promoter (encoded by the coding sequence of nucleic acid sequence A and/nucleic acid sequence B) is a HC-HC assembly promoter. As specified above, a HC-HC assembly promoter is suitable in the context of the invention, as such an element is for promoting, supporting, forcing, or directing the assembly of at least two antibody polypeptide chains that are provided by the nucleic acid sequence set comprised in the composition.

In preferred embodiments, the at least one HC-HC assembly promoter is located in the constant region of antibody heavy chain A and/or antibody heavy chain B. Preferably, at least one HC-HC assembly promoter is located in the constant region of antibody heavy chain A and antibody heavy chain B.

The term “constant region of antibody heavy chain” has to be understood as the region of an antibody chain that does (typically) not contribute to target (e.g. antigen or epitope) binding. Typically, the constant region of antibody heavy chain comprises of at least one of a CH1, a CH2, and/or a CH3 domain, or a fragment or a variant thereof. In embodiments, the constant region of antibody heavy chain comprises of at least a CH3 domain, or a fragment or a variant thereof. Preferably, the constant region of antibody heavy chain consists of a CH1, a CH2, and a CH3 domain.

In preferred embodiments, the at least one HC-HC assembly promoter is located in the Fc region of antibody heavy chain A and/or antibody heavy chain B. Preferably, at least one HC-HC assembly promoter is located in the Fc region of antibody heavy chain A and antibody heavy chain B.

The fragment crystallizable region (Fc region) is the tail region of an antibody that interacts with cell surface receptors called Fc receptors and some proteins of the complement system. This property allows antibodies to activate and/or interact with the immune system. In IgG, IgA and IgD antibody isotypes, the Fc region is composed of two identical protein fragments, derived from the second and third constant domains of the antibody's two heavy chains (CH2 and CH3). IgM and IgE Fc regions contain three heavy chain constant domains (CH domains 2-4) in each polypeptide chain.

In preferred embodiments, the at least one HC-HC assembly promoter is located in the CH3 domain of antibody heavy chain A and/or antibody heavy chain B. Preferably, at least one HC-HC assembly promoter is located in the CH3 domain of antibody heavy chain A and antibody heavy chain B.

For example a HC-HC assembly promoter may be located in a CH3 domain, or in a fragment or a variant of a CH3 domain, wherein the CH3 domain comprises at least one mutation or at least one amino acid substitution. In other words, the CH3 domain may comprise at least one mutation or at least one amino acid substitution compared to a naturally occurring CH3 domain.

More preferably, HC-HC assembly promoter may be located in a CH3 domain, preferably in the region or the amino acid sequence that generates/defines a CH3-CH3 interface between two different antibody heavy chains, e.g. two different heavy chains provided by the nucleic acid sequence set of the invention.

The CH3 domain of human IgG ranges from amino acid 342 to amino acid 446 (numbering according to EU numbering as derived from Edelman, Gerald M., et al. “The covalent structure of an entire γG immunoglobulin molecule.” Proceedings of the National Academy of Sciences 63.1 (1969): 78-85).

A typical CH3-CH3 interface in e.g. an IgG1 heavy chain is located in an amino acid element ranging from amino acid position aa E345 to amino acid position aa L410 (numbering according to EU numbering). Contact residues in the CH3-CH3 interface may include residues e.g. at positions 347, 349, 350, 351, 352, 353, 354, 355, 356, 357, 360, 364, 366, 368, 370, 390, 392, 394, 395, 397, 399, 400, 405, 407, 409, 439 according to the EU numbering system.

Accordingly, the CH3 domain of one heavy chain typically interacts in such a interface region with a second heavy chain to allow formation of a CH3-CH3 interface. A representative amino acid sequence stretch (spanning from aa E345 to amino acid position aa L410) involved in CH3-CH3 assembly is provided in SEQ ID NO: 81. Accordingly, all assembly promotor elements and all amino acid substitutions mentioned herein may be applied to that sequence stretch in the CH3 region (see for example Table A).

In preferred embodiments, the at least one HC-HC assembly promoter comprises at least one amino acid substitution that destroys or destabilize the naturally occurring CH3-CH3 interface of an antibody heavy chain, thereby preventing assembly of HC-A and/or HC-B to a non-modified or to a wild-type antibody heavy chain.

Accordingly, in preferred embodiments, the at least one HC-HC assembly promoter comprises at least one amino acid substitution in an amino acid sequence of a CH3-CH3 assembly interface of antibody heavy chain A and/or antibody heavy chain B.

In preferred embodiments, the at least one HC-HC assembly promoter comprises or consists of at least one selected from steric assembly element, electrostatic steering assembly element, SEED assembly element, DEEK assembly element, interchain disulfides assembly element, or any combination thereof. In particularly preferred embodiments, the at least one HC-HC assembly promoter does not comprises or consists an electrostatic steering assembly element.

Typically, different HC-HC assembly promoters are selected for antibody HC A and antibody HC B, wherein said different assembly promoters interact with each other to promote assembly of antibody HC A and antibody HC B (herein also referred to as “assembly promoter pair”). Preferably, as defined above, said different HC-HC assembly promoter elements are suitably located in the Fc region of antibody HC A and HC B, preferably in the CH3 region of antibody HC A and HC B, preferably in the region defining the CH3-CH3 interface. Preferably, said different HC-HC assembly promoters differs in at least one amino acid. Further, said HC-HC assembly promoter elements suitably prevent assembly to a wild-type (non-modified) antibody heavy chain.

In embodiments the at least one HC-HC assembly promoter comprises or consists of at least one SEED assembly element. As used herein, a SEED assembly element (strand-exchange engineered domain, SEED, IgG/IgA strand-exchange element) is at least one element designed to generate asymmetric antibody molecules (e.g. wherein the heavy chains of the asymmetric antibody are provided by the nucleic acid sequence set). In embodiments, alternating sequences from human IgA and IgG are assembled, preferably in the CH3 domain of the at least one antibody heavy chain A and/or the at least one antibody heavy chain B. It is preferred that antibody heavy chain A and antibody heavy chain B comprises a SEED assembly element (pair), wherein said SEED assembly elements allow specific assembly of the two antibody heavy chains. The concept of SEED assembly has been described in the art and may be applied to the nucleic acid sequence set of the invention.

In embodiments, the at least one HC-HC assembly promoter comprises or consists of at least one SEED assembly element, preferably antibody heavy chain A and antibody heavy chain B comprise at least one SEED assembly element.

In embodiments the at least one HC-HC assembly promoter comprises or consists of at least one DEEK assembly element. As used herein, a DEEK element is at least one amino acid residue, suitable to change the charge complementarity at the CH3 domain interface. The concept of DEEK assembly has been described in the art and may be applied to the nucleic acid sequence set of the invention.

In embodiments, the at least one HC-HC assembly promoter comprises or consists of at least one DEEK assembly element, preferably antibody heavy chain A and antibody heavy chain B comprise at least one DEEK assembly element.

In embodiments the at least one HC-HC assembly promoter comprises or consists of at least one electrostatic steering element. As used herein, an electrostatic steering element is at least one amino acid residue, suitable to change the charge complementarity at the CH3-CH3 domain interface.

In embodiments, the at least one antibody heavy chain A comprises K409D or K409E substitution in the CH3 domain, and the at least one antibody heavy chain B comprises a D399K or a D399R substitution in the CH3 domain (numbering according to EU numbering).

In embodiments, the at least one HC-HC assembly promoter comprises or consists of at least one electrostatic steering assembly element, preferably antibody heavy chain A and antibody heavy chain B comprise at least one Electrostatic steering assembly element.

In embodiments the at least one HC-HC assembly promoter comprises or consists of at least one interchain disulfides assembly element assembly element. As used herein, an interchain disulfides assembly element is at least one amino acid residue, suitably a Cysteine residue, that is integrated into the at least one antibody heavy chain A and/or antibody heavy chain B amino acid sequence to allow the formation of disulphide bonds. In that context it is preferred that antibody heavy chain A and antibody heavy chain B comprises at least one amino acid substitution, preferably a Cysteine substitution, to allow specific assembly and covalent connection (via C-C bonds) of the two antibody heavy chains.

In embodiments, the at least one antibody heavy chain A and/or the at least one antibody heavy chain B comprises at least one interchain disulfides assembly element comprising at least one of the following amino acid substitutions: S364C, F405C, L368C, Y349C, Y407C, K370C, D399C, L365C, K409C, T366C, L406C, T411C, L351C, P353C, S408C, V369C, V363C, E357C, L398C, P395C, K392C, N390C, T394C, Q347C, P352C, T393C, K439C, D356C, Q362C, S400C, K360C, S354C

In embodiments, the at least one antibody heavy chain A comprises S354C or Y349C substitution in the CH3 domain and the at least one antibody heavy chain B comprises a Y349C or S354C substitution in the CH3 domain (numbering according to EU numbering).

An interchain disulfides assembly element may preferably be combined with a steric assembly element, electrostatic steering assembly element, SEED assembly element, DEEK assembly element. An interchain disulfides assembly element may additionally stabilize (e.g. via covalent bond formation) correct assembly of two antibody chains (e.g. of antibody chain A and antibody chain B). In particularly preferred embodiments, interchain disulfides assembly element is combined with at least one steric assembly element.

In preferred embodiments, the at least one coding sequence of nucleic acid sequence A and nucleic acid sequence B encodes at least one antibody chain assembly promoter, wherein the at least one antibody chain assembly promoter is an HC-HC assembly promoter, wherein the HC-HC assembly promoter comprises or consists of at least one steric assembly element.

In the context of the invention it is particularly preferred that such a steric assembly element sterically forces the pairing or the assembly of two (different) antibody heavy chains, wherein the antibody heavy chains are provided by the nucleic acid sequence set of the composition. Further preferred is that such a steric assembly element sterically prevents the pairing or the assembly to e.g. wild-type antibody heavy chains or non-modified antibody heavy chains.

In preferred embodiments, the at least one steric assembly element as specified herein comprises a modification selected from at least one knob-modification and/or at least one hole modification.

The term “knob modification” has to be understood as a moiety, e.g. an amino acid substitution, wherein an amino acid with a small side chain volume (e.g. A, S, T, L, V etc.) is substituted with an amino acid with a larger side chain volume to generate a “knob”. Such a “knob” has to be understood as a protuberance in at least one antibody heavy chain (provided by the nucleic avid sequence set, e.g. antibody heavy chain A) that is suitable for sterically interacting with a compatible “hole” modification or cavity on a corresponding antibody heavy chain (provided by the nucleic sequence set, e.g. antibody heavy chain B). Accordingly, an antibody chain assembly promoter of the invention may comprise at least one knob modification.

Suitably, said amino acid residue having a larger side chain volume is selected from the group consisting of e.g. arginine (R), phenylalanine (F), tyrosine (Y), tryptophan (W). Accordingly, an R, F, Y, or W may be introduced (preferably by substituting another amino acid residue) to generate a “knob” or protuberance in at least one antibody heavy chain (e.g. antibody heavy chain A).

The term “hole modification” has to be understood as an amino acid substitution, wherein an amino acid with a large side chain volume (e.g. R, F, Y, W, T, L etc.) is substituted with an amino acid with a small side chain volume to generate a “hole”. Such a “hole” has to be understood as a cavity in at least one antibody heavy chain (provided by the artificial sequence set, e.g. antibody heavy chain B) that is suitable for sterically interacting with a compatible “knob” modification or protuberance on a corresponding antibody heavy chain (provided by the artificial sequence set, e.g. antibody heavy chain A). Accordingly, a antibody chain assembly promoter of the invention may comprise at least one hole modification.

Suitably, an amino acid residue having a smaller side chain volume is selected from the group consisting of alanine (A), serine (S), threonine (T), valine (V). Accordingly, an A, S, T, or V may be introduced (preferably by substituting another amino acid residue) to generate a “hole” or cavity in at least one antibody heavy chain (e.g. antibody heavy chain B).

In preferred embodiments, the at least one steric assembly element as specified herein comprises a modification selected from at least one knob-modification wherein, preferably, the at least one knob-modification is at least one amino acid substitution in a CH3-CH3 assembly interface.

A suitable knob-modification or protuberance modification may be selected from at least one of the following substitutions (numbering according to EU numbering of the CH3 domain):
Substitution of L in aa position 351 to a Y, R, F, or W, preferably L351Y
Substitution of T in aa position 366 to a Y, R, F, or W, preferably T366W or T366Y
Substitution of T in aa position 394 to a Y, R, F, or W, preferably T394F or T394W

In various embodiments, a knob-modification may correspond to multiple amino acid substitutions.

In preferred embodiments, the at least one steric assembly element as specified herein comprises a modification selected from at least one hole modification, wherein, preferably, the at least one hole-modification is at least one amino acid substitution in a CH3-CH3 assembly interface.

A suitable hole-modification or cavity modification may be selected from at least one of the following substitutions (numbering according to EU numbering of the CH3 domain):
Substitution of T in aa position 350 to an A, S, or V, preferably T350V
Substitution of T in aa position 366 to an A, S, or V, preferably T366S
Substitution of L in aa position 368 to an A, S, T, or V, preferably L368A
Substitution of F in aa position 405 to an A, S, T, or V, preferably F405A
Substitution of Y in aa position 407 to an A, S, T, or V, preferably Y407V or Y407T

In various embodiments, a hole-modification may correspond to multiple amino acid substitutions.

In preferred embodiments, the at least one coding sequence of nucleic acid sequence A encodes at least one HC-HC assembly promoter and the at least one coding sequence of nucleic acid sequence B encodes at least one HC-HC assembly promoter.

In particularly preferred embodiments, the at least one HC-HC assembly promoter of HC-A comprises at least one knob-modification and the at least one HC-HC assembly promoter of HC-B comprises at least one hole modification, preferably thereby forming an assembly promoter pair.

Accordingly, antibody heavy chain A and antibody heavy chain B are suitably modified to comprise at least one ‘knob-hole’ HC-HC assembly promoter pair.

Specifically, in one preferred embodiment of the invention, the CH3 domain of antibody heavy chain A and the CH3 domain of antibody heavy chain B can be altered in a way that one antibody heavy chain, e.g. antibody heavy chain A comprises at least one knob modification and one antibody heavy chain, e.g. antibody heavy chain B comprises at least one hole modification. Suitably, by expressing these two heavy chains (provided by the sequence set of the invention), high yields of heterodimer formation (‘knob-hole’) versus homodimer formation (‘hole-hole’ or ‘knob-knob’) may suitably be achieved. Each of the two CH3 domains (of the two heavy chains) can be the “knob”, while the other one is the “hole”.

In preferred embodiments, the CH3 domains of the two heavy chains (HC-A, HC-B) each meet at an interface which comprises an original interface between the antibody CH3 domains (the CH3-CH3 interface) wherein said interface is altered to promote the formation of an assembled antibody.

In embodiments it may be beneficial to introduce multiple (e.g. 2, 3, 4, or more) knob-hole modifications (or multiple knob-hole modification pairs) to improve heavy chain assembly. For example, it is possible and in the scope of the invention that one antibody heavy chain (e.g. heavy chain A) comprises a knob modification and a hole modification, whereas the other antibody heavy chain (e.g. heavy chain B) also comprises a knob modification and a hole modification, and that upon expression of the antibody chains two different steric knob-hole interactions for promote antibody chain assembly and a correctly assembled antibody is generated.

In preferred embodiments, the nucleic acid sequence set encodes HC-A and HC-B comprising at least one HC-HC assembly promoter pair (HC-HC PP) comprising the following amino acid substitutions (numbering according to EU numbering of the CH3 domain). Notably, the modification provided below may be adapted and transferred to different allotypes:
HC-HC-PP 1: T366Y on HC-A; Y407T on HC-B
HC-HC-PP 2: T366W on HC-A; 366S, L368A, Y407V on HC-B
HC-HC-PP 3: S354C, T366W on HC-A; Y349C, T366S, L368A, Y407V on HC-B
HC-HC-PP 4: S364H, F405A on HC-A; Y349T, T394F on HC-B
HC-HC-PP 5: T350V, L351Y, F405A, Y407V on HC-A; T350V, T366L, K392L, T394W on HC-B
HC-HC-PP 6: K409D on HC-A; D399K on HC-B
HC-HC-PP 7: K409D on HC-A; D399R on HC-B
HC-HC-PP 8: K409E on HC-A; D399R on HC-B
HC-HC-PP 9: K409E on HC-A; D399K on HC-B
HC-HC-PP 10: K392D, K409D on HC-A; E/D356K, D399K on HC-B
HC-HC-PP 11: D221E, P228E, L368E on HC-A; D221R, P228R, K409R on HC-B
HC-HC-PP 12: K360E, K409W on HC-A; Q347R, D399V, F405T on HC-B
HC-HC-PP 13: Y349C, K360E, K409W on HC-A; Q347R, S354C, D399V, F405T on HC-B
HC-HC-PP 14: L351L/K, T366K on HC-A; Y349D/E, R355D/E on HC-B
HC-HC-PP 15: L351L/K, T366K on HC-A; Y349D/E and/or L351D/E and/or R355D/E and/or L368D/E on HC-B
HC-HC-PP 16: F405L on HC-A; K409R on HC-B
HC-HC-PP 17: K360D, D399M, Y407A on HC-A; E345R, Q347R, T366V, K409V on HC-B
HC-HC-PP 18: Y349S, T366M, K370Y, K409V on HC-A; E/D356G, E357D, S364Q, Y407A on HC-B

Suitably, the assembly promoter pairs are designed and selected in a way that mis-assembly between different HC-HC promoter pairs is reduced or avoided. This is particularly important in the context of expressing antibody mixtures e.g. in vivo. Accordingly, the HC-HC promoters of HC-HC-PP 1 do preferably not assemble with any one of the HC-HC promoters of HC-HC-PP 2-18. HC-HC promoters of HC-HC-PP 2 do preferably not assemble with any one of the HC-HC promoters of HC-HC-PP 1, 3-18. HC-HC promoters of HC-HC-PP 3 do preferably not assemble with any one of the HC-HC promoters of HC-HC-PP 1-2, 4-18. HC-HC promoters of HC-HC-PP 4 do preferably not assemble with any one of the HC-HC promoters of HC-HC-PP 1-3, 5-18. HC-HC promoters of HC-HC-PP 5 do preferably not assemble with any one of the HC-HC promoters of HC-HC-PP 1-4, 6-18. HC-HC promoters of HC-HC-PP 6 do preferably not assemble with any one of the HC-HC promoters of HC-HC-PP 1-5, 7-18. HC-HC promoters of HC-HC-PP 7 do preferably not assemble with any one of the HC-HC promoters of HC-HC-PP 1-6, 8-18. HC-HC promoters of HC-HC-PP 8 do preferably not assemble with any one of the HC-HC promoters of HC-HC-PP 1-7, 9-18. HC-HC promoters of HC-HC-PP 9 do preferably not assemble with any one of the HC-HC promoters of HC-HC-PP 1-8, 10-18. HC-HC promoters of HC-HC-PP 10 do preferably not assemble with any one of the HC-HC promoters of HC-HC-PP 1-9, 11-18. HC-HC promoters of HC-HC-PP 11 do preferably not assemble with any one of the HC-HC promoters of HC-HC-PP 1-10, 12-18. HC-HC promoters of HC-HC-PP 12 do preferably not assemble with any one of the HC-HC promoters of HC-HC-PP 1-11, 13-18. HC-HC promoters of HC-HC-PP 13 do preferably not assemble with any one of the HC-HC promoters of HC-HC-PP 1-12, 14-18. HC-HC promoters of HC-HC-PP 14 do preferably not assemble with any one of the HC-HC promoters of HC-HC-PP 1-13, 18. HC-HC promoters of HC-HC-PP 15 do preferably not assemble with any one of the HC-HC promoters of HC-HC-PP 1-14, 16-18. HC-HC promoters of HC-HC-PP 16 do preferably not assemble with any one of the HC-HC promoters of HC-HC-PP 1-15, 17-18. HC-HC promoters of HC-HC-PP 17 do preferably not assemble with any one of the HC-HC promoters of HC-HC-PP 1-16, 18. HC-HC promoters of HC-HC-PP 18 do preferably not assemble with any one of the HC-HC promoters of HC-HC-PP 1-17. Moreover, HC-HC promoters of HC-HC-PP 1 to 18 do preferably not assemble with naturally occurring HCs (e.g. wild type (unmodified) heavy chains).

In preferred embodiments, the HC-HC promoters of HC-HC-PP 3 do preferably not assemble with any one of the HC-HC promoters of HC-HC-PP 4, HC-HC-PP 5, or HC-HC-PP 18. In preferred embodiments, the HC-HC promoters of HC-HC-PP 4 do preferably not assemble with any one of the HC-HC promoters of HC-HC-PP 3, HC-HC-PP 5, or HC-HC-PP 18. In preferred embodiments, the HC-HC promoters of HC-HC-PP 5 do preferably not assemble with any one of the HC-HC promoters of HC-HC-PP 3, HC-HC-PP 4, or HC-HC-PP 18. In preferred embodiments, the HC-HC promoters of HC-HC-PP 18 do preferably not assemble with any one of the HC-HC promoters of HC-HC-PP 3, HC-HC-PP 4, or HC-HC-PP 5. Moreover, HC-HC promoters of HC-HC-PP 3, 4, 5 and 18 do preferably not assemble with naturally occurring HCs (e.g. wild type (unmodified) heavy chains).

Accordingly, HC-HC promoter pairs HC-HC PP 1 to HC-HC PP 18 may be used to generated compositions comprising up to 18 different nucleic acid sequence sets, comprising up to 18 specific HC-HC promoter pairs. Administration of such a composition to a cell or a subject suitably leads to production of up to 18 different, correctly assembled antibodies. Accordingly, HC-HC promoter pairs HC-HC PP 1 to HC-HC PP 18 may be used to generated compositions comprising up to 2, 3, 4, 5, 6, 7, 8, 9, 10, 11, 12, 13, 14, 15, 16, 17, or 18 different nucleic acid sequence sets, comprising up to 2, 3, 4, 5, 6, 7, 8, 9, 10, 11, 12, 13, 14, 15, 16, 17, or 18 specific HC-HC promoter pairs. Administration of such a composition to a cell or a subject suitably leads to production of up to 2, 3, 4, 5, 6, 7, 8, 9, 10, 11, 12, 13, 14, 15, 16, 17, or 18 different, correctly assembled antibodies.

In preferred embodiments, antibody heavy chain A (HC-A) and antibody heavy chain B (HC-B) comprises at least one HC-HC assembly promoter pair comprising the following amino acid substitutions (numbering according to EU numbering of the CH3 domain):
HC-HC-PP1: T366Y on HC-A; Y407T on HC-B
HC-HC-PP3: S354C, T366W on HC-A; Y349C, T366S, L368A, Y407V on HC-B
HC-HC-PP4: S364H, F405A on HC-A; Y349T, T394F on HC-B
HC-HC-PP5: T350V, L351Y, F405A, Y407V on HC-A; T350V, T366L, K392L, T394W on HC-B
HC-HC-PP7: K409D on HC-A; D399R on HC-B
HC-HC-PP11: D221E, P228E, L368E on HC-A; D221R, P228R, K409R on HC-B
HC-HC-PP13: Y349C, K360E, K409W on HC-A; Q347R, S354C, D399V, F405T on HC-B
HC-HC-PP14: L351L, T366K on HC-A; Y349D, R355E on HC-B
HC-HC-PP16: F405L on HC-A; K409R on HC-B
HC-HC-PP18: Y349S, T366M, K370Y, K409V on HC-A; E/D356G, E357D, S364Q, Y407A on HC-B

In particularly preferred embodiments, antibody heavy chain A (HC-A) and antibody heavy chain B (HC-B) comprises at least one HC-HC assembly promoter pair comprising the following amino acid substitutions (numbering according to EU numbering of the CH3 domain):
HC-HC-PP3: S354C, T366W on HC-A; Y349C, T366S, L368A, Y407V on HC-B
HC-HC-PP4: S364H, F405A on HC-A; Y349T, T394F on HC-B
HC-HC-PP5: T350V, L351Y, F405A, Y407V on HC-A; T350V, T366L, K392L, T394W on HC-B
HC-HC-PP18: Y349S, T366M, K370Y, K409V on HC-A; E/D356G, E357D, S364Q, Y407A on HC-B

In Table 1, particularly suitable HC-HC assembly promoters and HC-HC assembly promoter pairs are provided. Therein, Column A indicates the identifier of the HC-HC assembly promoter pair as used herein. Column B indicates the concepts for of HC-HC assembly used. Column C indicates the amino acid substitutions of HC-A assembly promoter. Column D indicates the amino acid substitutions of HC-B assembly promoter (numbering according to EU numbering).

TABLE 1
Preferred HC-HC assembly promoters and promoter pairs of the invention

| | B | C | D |
|---|---|---|---|
| A | Concepts | HC-A assembly promoter | HC-B assembly promoter |
| HC-HC-PP1 | steric assembly | T366Y | Y407T |
| HC-HC-PP2 | steric assembly | T366W | T366S, L368A, Y407V |
| HC-HC-PP3 | steric assembly | S354C, T366W | Y349C, T366S, L368A, Y407V |
| | interchain disulfides assembly | | |
| HC-HC PP4 | steric assembly | S364H, F405A | Y349T, T394F |
| HC-HC PP5 | steric assembly | T350V, L351Y, F405A, Y407V | T350V, T366L, K392L, T394W |
| HC-HC PP6 | electrostatic steering | K409D | D399K |
| HC-HC PP7 | electrostatic steering | K409D | D399R |
| HC-HC PP8 | electrostatic steering | K409E | D399R |
| HC-HC PP9 | electrostatic steering | K409E | D399K |
| HC-HC PP10 | electrostatic steering | K392D, K409D | E/D356K, D399K |
| HC-HC PP11 | electrostatic steering | D221E, P228E, L368E | D221R, P228R, K409R |
| HC-HC PP12 | steric assembly | K360E, K409W | Q347R, D399V, F405T |
| | electrostatic steering | | |
| HC-HC PP13 | steric assembly | Y349C, K360E, K409W | Q347R, S354C, D399V, F405T |
| | electrostatic steering | | |
| | interchain disulfides assembly | | |
| HC-HC PP14 | electrostatic steering | L351L/K, T366K | Y349D/E, R355D/E |
| HC-HC PP15 | electrostatic steering | L351L/K, T366K | Y349D/E and/or L351D/E and/or |
| | | | R355D/E and/or L368D/E |
| HC-HC PP16 | steric assembly | F405L | K409R |
| HC-HC PP17 | steric assembly | K360D, D399M, Y407A | E345R, Q347R, T366V, K409V |
| HC-HC PP18 | steric assembly | Y349S, T366M, K370Y, K409V | E/D356G, E357D, S364Q, |
| | | | Y407A |

In preferred embodiments, HC-HC PP1 to HC-CH PP18 can be combined with at least one interchain disulfides assembly element as defined herein.

In particularly preferred embodiments, the composition comprises at least two, three or four nucleic acid sequence sets, wherein the at least two, three, four, or five nucleic acid sequence sets comprise a different HC-HC assembly promoter pair each selected from HC-HC-PP3, HC-HC-PP4, HC-HC-PP5, HC-HC-PP16, or HC-HC-PP18.

Preferably, each nucleic acid sequence set encodes a different antibody (that produces an assembled antibody upon administration, in particular upon in vivo administration).

As outlined above, a typical CH3-CH3 interface in e.g. an IgG1 heavy chain is located in an amino acid element ranging from amino acid position aa E345 to amino acid position aa L410 (numbering according to EU numbering). Particularly suitable amino acid sequence stretches (ranging from E345 to amino acid position aa L410) that can suitably be used and included in the respective HC-A and/or HC-B of the invention are provided in Table A. Column A provides a short description of HC-HC assembly promoters (compare with Table 1). Column B shows the amino acid sequence stretch from aa E345 to amino acid position aa L410 (numbering according to EU numbering), wherein for each row the amino acid substitution compared to a wild type (non-modified) HC is indicated. Column C provides the amino acid sequence SEQ ID NO for the respective stretch. Column D provides the amino acid sequence SEQ ID NO for a representative HC as e.g. used in the Example section.

TABLE A
CH3-CH3 assembly regions of preferred HC-HC
promoters of the invention

| A | B | C | D |
|---|---|---|---|
| HC | epqvytlppsrdeltknqvsltclvkgfypsdiavewes | | 81 |
| (wild type) | ngqpennykttppvldsdgsfflyskl | | |
| HC-HC-PP3 | epqvytlpp [symbol] rdeltknqvsl [symbol] clvkgfypsdiavew | 104 | 82 |
| | esngqpennykttppvldsdgsfflyskl | | |
| HC-HC-PP3 | epqv [symbol] tlppsrdeltknqvsl [symbol] c [symbol] vkgfypsdiavew | 105 | 83 |
| | esngqpennykttppvldsdgsffl [symbol] skl | | |
| HC-HC-PP4 | epqvytlppsrdeltknqv [symbol] ltclvkgfypsdiavewe | 106 | 84 |
| | sngqpennykttppvldsdgsf [symbol] lyskl | | |
| HC-HC PP4 | epqv [symbol] tlppsrdeltknqvsltclvkgfypsdiavewe | 107 | 85 |
| | sngqpennykt [symbol] ppvldsdgsfflyskl | | |
| HC-HC-PP5 | epqvy [symbol] ppsrdeltknqvsltclvkgfypsdiavewes | 108 | 86 |
| | ngqpennykttppvldsdgsf [symbol] l [symbol] skl | | |
| HC-HC-PP5 | epqvy [symbol] lppsrdeltknqvsl [symbol] clvkgfypsdiavewe | 109 | 87 |
| | sngqpenny [symbol] t [symbol] ppvldsdgsfflyskl | | |
| HC-HC-PP16 | epqvytlppsrdeltknqvsltclvkgfypsdiavewe | 110 | 88 |
| | sngqpennykttppvldsdgsf [symbol] lyskl | | |
| HC-HC-PP16 | epqvytlppsrdeltknqvsltclvkgfypsdiavewe | 111 | 89 |
| | sngqpennykttppvldsdgsfflys [symbol] l | | |
| HC-HC-PP18 | epqv [symbol] tlppsrdeltknqvsl [symbol] clv [symbol] gfypsdiave | 112 | 90 |
| | wesngqpennykttppvldsdgsfflys [symbol] l | | |
| HC-HC-PP18 | epqvytlppsr [symbol] ltknqv [symbol] ltclvkgfypsdiavewe | 113 | 91 |
| | sngqpennykttppvldsdgsffl [symbol] skl | | |

In particularly preferred embodiments, antibody heavy chain A (HC-A) and antibody heavy chain B (HC-B) encoded by the nucleic acid sequence set of the invention comprises at least one HC-HC assembly promoter pair comprising the following amino acid sequence stretch in the CH3 domain, being identical or at least 90%, 95%, 96%, 97%, 98%, 99% identical to the following amino acid sequences:
HC-HC-PP3: SEQ ID NO: 104 on HC-A; SEQ ID NO: 105 on HC-B
HC-HC-PP4: SEQ ID NO: 106 on HC-A; SEQ ID NO: 107 on HC-B
HC-HC-PP5: SEQ ID NO: 108 on HC-A; SEQ ID NO: 109 on HC-B
HC-HC-PP18: SEQ ID NO: 112 on HC-A; SEQ ID NO: 113 on HC-B

In particularly preferred embodiments, the composition comprises, at least one
(i) nucleic acid sequence set encoding HC-A and HC-B, comprising an assembly promoter pair HC-HC-PP3, and/or
(ii) nucleic acid sequence set encoding HC-A and HC-B, comprising an assembly promoter pair HC-HC-PP4, and/or
(iii) nucleic acid sequence set encoding HC-A and HC-B, comprising an assembly promoter pair HC-HC-PP5, and/or
(iv) nucleic acid sequence set encoding HC-A and HC-B, comprising an assembly promoter pair HC-HC-PP18.

In preferred embodiments, the coding sequence of nucleic acid sequence A additionally encodes at least one fragment selected or derived from an antibody light chain A (LC-A) or a variant thereof.

In preferred embodiments, the coding sequence of nucleic acid sequence B additionally encodes at least one fragment selected or derived from an antibody light chain A (LC-B) or a variant thereof.

In embodiments, the coding sequence of nucleic acid sequence A additionally encodes at least one fragment selected or derived from an antibody light chain A (LC-A) or a variant thereof and the coding sequence of nucleic acid sequence B additionally encodes at least one fragment selected or derived from an antibody light chain B (LC-B) or a variant thereof.

In preferred embodiments, the at least one LC-A and/or the at least one LC-B is selected or derived from a K light chain or λ light chain or a fragment or variant thereof.

In embodiments, the at least one LC-A fragment or variant is N-terminally or C-terminally fused to HC-A. In preferred embodiments, the at least one LC-A fragment or variant is N-terminally or C-terminally fused to the variable region of HC-A. In preferred embodiments, the at least one LC-A fragment or variant is N-terminally fused to HC-A as defined herein, preferably fused to the variable region of HC-A as defined herein. In preferred embodiments, the at least one LC-A fragment or variant is C-terminally fused to HC-A as defined herein, preferably fused to the variable region of HC-A as defined herein.

In embodiments, the at least one LC-B fragment or variant is N-terminally or C-terminally fused to HC-B. In preferred embodiments, the at least one LC-B fragment or variant is N-terminally or C-terminally fused the variable region of HC-B. In preferred embodiments, the at least one LC-B fragment or variant is N-terminally fused to HC-B as defined herein, preferably fused to the variable region of HC-B as defined herein. In preferred embodiments, the at least one LC-B fragment or variant is C-terminally fused to HC-B as defined herein, preferably fused to the variable region of HC-B as defined herein.

In preferred embodiments, the LC-A fragment or variant is a variable region of an antibody light chain or a fragment thereof. In preferred embodiments, the LC-B fragment or variant is a variable region of an antibody light chain or a fragment thereof.

In preferred embodiments, a variable region of LC-A is fused to the variable region of HC-A, optionally via a linker peptide element. In preferred embodiments, a variable region of LC-B is fused to the variable region of HC-B, optionally via a linker peptide element, e.g. a flexible linker peptide element.

In preferred embodiments, the nucleic acid sequence set of the composition comprises
a) nucleic acid sequence A comprising at least one coding sequence encoding
   at least one HC-A, or a fragment or variant thereof, and
   at least one HC-HC assembly promoter as defined herein, and
   at least one LC-A, or a fragment or variant thereof,

preferably, wherein the variable region of LC-A is fused to the variable region of HC-A;
b) nucleic acid sequence B comprising at least one coding sequence encoding
   at least one HC-B, or a fragment or variant thereof, and
   at least one HC-HC assembly promoter as defined herein, and
   at least one LC-B, or a fragment or variant thereof,

preferably, wherein the variable region of LC-B is fused to the variable region of HC-B.

Having the light-chain, in particular the variable domain of a light chain, fused to a heavy chain has the advantage that by introducing HC-HC promoters as defined herein, functional assembled antibodies can be generated, also in compositions expressing multiple different antibodies.

Embodiments where the light chain is provided via a separate coding sequence (that is, not as an HC-LC fusion protein as described above) may require the introduction of further antibody chain assembly promoters to facilitate correct HC-LC assembly (e.g. HC-LC assembly promotor, LC-HC assembly promotor). Such embodiments are described in the following.

In embodiments, the at least one coding sequence of the nucleic acid sequence A and/or the nucleic acid sequence B encodes at least one antibody chain assembly promoter, wherein the at least one antibody chain assembly promoter is selected from a heavy chain-light chain (HC-LC) assembly promoter.

Accordingly, in embodiments, antibody heavy chain A (HC-A) may comprise at least one HC-HC assembly promoter (as defined above) and, additionally or alternatively, at least one HC-LC assembly promoter. In embodiments, antibody heavy chain B (HC-A) may comprise at least one HC-HC assembly promoter (as defined herein) and, additionally or alternatively, at least one HC-LC assembly promoter. In various embodiments, HC-A and/or HC-B may comprise at least one HC-HC assembly promoter (preferably an HC-HC assembly promoter pair as defined above) and, additionally, at least one HC-LC assembly promoter (as defined in the following).

The term “heavy chain-light chain assembly promoter” or “HC-LC assembly promoter” as used herein relates to a moiety (e.g. an amino acid) that promotes, supports, forces, or directs assembly of at least one antibody heavy chain and at least one antibody light chain (herein, provided by the nucleic acid sequence set). In the context of the invention, such a moiety is typically at least one amino acid capable of promoting, supporting, forcing, or directing a certain assembly of the at least two antibody polypeptide chains. Preferably, in the context of the invention, such an amino acid substitution is a substitution that does not occur naturally, suitably, a substitution that does not occur naturally in human antibody chains.

For example, an “HC-LC assembly promoter” may be located on an antibody heavy chain A and/or on an antibody heavy chain B to promote, support, force, or direct an assembly between the two antibody chains, e.g. to promote, support, force, or direct a heterodimerization (if desired) or a homodimerization of e.g. HCs (if desired). Suitably in the context of the invention, an antibody chain assembly promoter promotes, supports, forces, or directs assembly of at least two antibody polypeptide chains (HC and LC) preferably in the presence of an additional antibody polypeptide chain (or additional polypeptide chains). Merely as an example, suitable HC-LC assembly promoters may promote, support, force, or direct (correct) assembly of at least two antibody polypeptide chains while, at the same time, avoiding assembly to other antibody polypeptide chains lacking an antibody chain assembly promoter or comprising a different HC-LC antibody chain assembly promoter.

In the context of the invention, said at least one moiety of the HC-LC assembly promoter (e.g. at least one amino acid) is encoded by the at least one coding sequence of nucleic acid sequence A and/or nucleic acid sequence B. As an example, two antibody chains comprising such an “HC-LC assembly promoter” may show an increased occurrence of correctly assembled antibody heavy chain and light chain under certain conditions, compared to naturally occurring antibody chains lacking such an “HC-LC assembly promoter”. An increased occurrence of correctly assembled antibody chains is suitably observed in the presence of other antibody polypeptide chains (e.g. lacking an assembly promoter).

It has to be understood that “correctly assembled” depends on the actual purpose, e.g. whether e.g. heterodimerization or homodimerization of heavy chains is preferred.

In a naturally occurring antibody or antibody chains, e.g. an IgG antibody, HCs and LCs are co-translationally translocated into the ER of a B-cell, and folding begins before the polypeptide chains are completely translated. Most IgGs assemble first as HC dimers to which LCs are added covalently via a disulphide bond between the CL and CH1 domains. Accordingly, a typical antibody heavy chain comprises a natural antibody heavy chain-light chain assembly sequence interface, forming a CH1-CL interface that mediates assembly. It has to be emphasized that such naturally occurring antibody heavy chain-light chain assembly interfaces are not comprised by the term “HC-LC assembly promoter” as used herein.

Merely as an example, an “HC-LC assembly promoter” may be derived from any naturally occurring antibody heavy chain assembly sequence, wherein at least one amino acid residue is mutated/changed/substituted to e.g. another amino acid residue. Further, the term “HC-LC assembly promoter” may have a sequence that is 100% identical to a naturally occurring antibody chain assembly sequence, wherein said “a HC-LC assembly promoter” is located in a position that does not occur in nature. Accordingly, the term “HC-LC“assembly promoter” has to be understood as “non-naturally occurring” in terms of the amino acid sequence or the position in an antibody heavy chain (specifically, “non-naturally occurring” has to be understood in comparison to wild-type or naturally occurring human antibody chains). Typically, an antibody HC-LC assembly promoter of the invention is configured to assemble to a LC-HC assembly promoter (located on an antibody light chain as defined herein).

Typically, a HC-LC assembly promoter as defined herein is located on a heavy chain and specifically interacts with a LC-HC assembly promoter on a light chain (as further specified below) to promote specific assembly of LCs to HCs.

According to preferred embodiments, the at least one HC-LC assembly promoter is located in the constant region of HC-A and/or HC-B. Suitably, at least one HC-LC assembly promoter is located in the constant region of HC-A and at least one HC-LC assembly promoter is located in the constant region of HC-B. Suitably, respective HC-LC assembly promoters are selected to allow specific assembly of LC-A to HC-A and LC-B to HC-B.

According to preferred embodiments, the at least one HC-LC assembly promoter is located in the Fab region of HC-A and/or HC-B. Suitably, at least one HC-LC assembly promoter is located in the Fab region of HC-A and one HC-LC assembly promoter is located in the Fab region of HC-B. Suitably, respective HC-LC assembly promoters are selected to allow specific assembly of LC-A to HC-A and LC-B to HC-B.

According to preferred embodiments, the at least one HC-LC assembly promoter is located in the CH1 domain region of HC-A and/or HC-B. Suitably, at least one HC-LC assembly promoter is located in the CH1 domain of HC-A and one HC-LC assembly promoter is located in the CH1 domain of HC-B. Suitably, respective HC-LC assembly promoters are selected to allow specific assembly of LC-A to HC-A and LC-B to HC-B.

According to preferred embodiments, the at least one HC-LC assembly promoter comprises at least one amino acid substitution in an amino acid sequence of the HC-LC assembly interface. In particular, the at least one HC-LC assembly promoter comprises at least one amino acid substitution in an amino acid sequence of the CH1-CL interface.

According to preferred embodiments, the at least one HC-LC assembly promoter comprises or consists of at least one selected from steric assembly element, electrostatic steering assembly element, SEED assembly element, DEEK assembly element, interchain disulfides assembly element, or any combination thereof.

Suitably, the at least one HC-LC assembly promoter comprises at least one steric assembly element, wherein the steric assembly element comprises a modification selected from at least one knob-modification and/or at least one hole modification.

According to preferred embodiments, the at least one coding sequence of nucleic acid sequence A encodes at least one HC-LC assembly promoter, and the at least one coding sequence of nucleic acid sequence B encodes at least one HC-LC assembly promoter. Suitably, the HC-LC assembly promoters are located in the CH1 domain, being a part of the HC(CH1)-LC(CL) assembly interface (CH1-CL interface). Suitably, the HC-LC assembly promoters located in the HC(CH1)-LC(CL) assembly interface are selected from at least one knob-modification and/or at least one hole modification. Suitably, HC-LC assembly promoters as defined above interact with LC-HC assembly promoters of antibody light chains (as described below).

In embodiments, the nucleic acid set of the composition additionally comprises
c) nucleic acid sequence C comprising at least one coding sequence encoding at least one LC-A, or a fragment or variant thereof, and/or
d) nucleic acid sequence D comprising at least one coding sequence encoding at least one LC-B), or a fragment or variant thereof.

The term “nucleic acid sequence C” as used herein has to be understood as any type of nucleic acid sequence, including DNA or RNA sequences, provided that said nucleic acid sequence comprises at least one coding sequence encoding at least one antibody light chain A (LC-A), or a fragment or variant thereof. Nucleic acid sequence C is part of the nucleic acid sequence set encoding an antibody. Said “nucleic acid sequence C” may be located on a separate nucleic acid molecule (e.g. a DNA molecule or an RNA molecule) or may be located one nucleic acid molecule (e.g. a bicistronic—or multicistronic nucleic acid as defined herein) together with nucleic acid sequence A and/or together with an optional further nucleic acid sequence as defined herein. Accordingly, nucleic acid sequence C and nucleic acid sequence A, and, optionally, further nucleic acid sequences may be located on separate entities (e.g. different RNA or DNA molecules) or on the same entity (e.g. the same RNA molecule/the same DNA molecule).

The term “nucleic acid sequence D” as used herein has to be understood as any type of nucleic acid sequence, including DNA, RNA sequences, provided that said nucleic acid sequence comprises at least one coding sequence encoding at least one antibody light chain B (LC-B), or a fragment or variant thereof. Nucleic acid sequence D is part of the nucleic acid sequence set encoding an antibody. Said “nucleic acid sequence D” may be located on a separate nucleic acid molecule (e.g. a DNA molecule or an RNA molecule) or may be located one nucleic acid molecule (e.g. a bicistronic—or multicistronic nucleic acid as defined herein) together with nucleic acid sequence B and/or together with an optional further nucleic acid sequence as defined herein. Accordingly, the nucleic acid sequence B and nucleic acid sequence D, and, optionally, further nucleic acid sequences may be located on separate entities (e.g. different RNA or DNA molecules) or on the same entity (e.g. the same RNA molecule/the same DNA molecule). Suitably, the antibody light chain encoded by nucleic acid sequence C and/or nucleic acid sequence D is selected or derived from a κ light chain or a λ light chain.

According to preferred embodiments, the at least one coding sequence of nucleic acid sequence C and/or nucleic acid sequence D encodes at least one light chain-heavy chain (LC-HC) assembly promoter.

The term “light chain-heavy chain assembly promoter” or “LC-HC assembly promoter” as used herein relates to a moiety (e.g. an amino acid) that promotes, supports, forces, or directs assembly of at least one antibody light chain and at least one antibody heavy chain (herein, provided by the nucleic acid sequence set). In the context of the invention, such a moiety is typically at least one amino acid capable of promoting, supporting, forcing, or directing a certain assembly of the at least two antibody polypeptide chains. Preferably, in the context of the invention, such an amino acid substitution is a substitution that does not occur naturally, suitably, a substitution that does not occur naturally in human antibody chains.

For example, an “LC-HC assembly promoter” may be located on an antibody light chain A and/or on an antibody light chain B to promote, support, force, or direct an assembly between the two antibody chains, e.g. to promote, support, force, or direct a heterodimerization (if desired) or a homodimerization of e.g. HCs (if desired). Suitably in the context of the invention, an antibody chain assembly promoter promotes, supports, forces, or directs assembly of at least two antibody polypeptide chains (LC and HC) preferably in the presence of an additional antibody polypeptide chain (or additional polypeptide chains). Merely as an example, suitable LC-HC assembly promoters may promote, support, force, or direct (correct) assembly of at least two antibody polypeptide chains while, at the same time, avoiding assembly to other antibody polypeptide chains lacking an antibody chain assembly promoter or comprising a different LC-HC antibody chain assembly promoter.

In the context of the invention, said at least one moiety of the LC-HC assembly promoter (e.g. at least one amino acid) is encoded by the at least one coding sequence of nucleic acid sequence C and/or nucleic acid sequence D. As an example, two antibody chains comprising such an “LC-HC assembly promoter” may show an increased occurrence of correctly assembled antibody heavy chain and light chain under certain conditions, compared to naturally occurring antibody chains lacking such an “LC-HC assembly promoter”. An increased occurrence of correctly assembled antibody chains is suitably observed in the presence of other antibody polypeptide chains (e.g. lacking an assembly promoter).

In a naturally occurring antibody or antibody chains, e.g. an IgG antibody, LCs and HCs are co-translationally translocated into the ER of a B-cell, and folding begins before the polypeptide chains are completely translated. Most IgGs assemble first as HC dimers to which LCs are added covalently via a disulphide bond between the CL and CH1 domains. Accordingly, a typical antibody light chain comprises a natural antibody light chain-heavy chain assembly sequence interface, forming a CL-CH1 interface that mediates assembly. It has to be emphasized that such naturally occurring antibody light chain-heavy chain assembly interfaces are not comprised by the term “LC-HC assembly promoter” as used herein.

Merely as an example, an “LC-HC assembly promoter” may be derived from any naturally occurring antibody chain assembly sequence, wherein at least one amino acid residue is mutated/changed/substituted to e.g. another amino acid residue. Further, the term “LC-HC assembly promoter” may have a sequence that is 100% identical to a naturally occurring antibody chain assembly sequence, wherein said “LC-HC assembly promoter” is located in a position that does not occur in nature. Accordingly, the term “LC-HC assembly promoter” has to be understood as “non-naturally occurring” in terms of the amino acid sequence or the position in an antibody heavy chain (specifically, “non-naturally occurring” has to be understood in comparison to wild-type or naturally occurring human antibody chains). Typically, an antibody LC-HC assembly promoter of the invention is configured to assemble to a HC-LC assembly promoter (located on an antibody heavy chain as defined herein). Typically, a LC-HC assembly promoter as defined herein is located on a light chain and specifically interacts with a HC-LC assembly promoter on a heavy chain (as further specified herein) to promote specific assembly of LCs to HCs.

According to preferred embodiments, the at least one LC-HC assembly promoter is located in the constant region of LC-A and/or LC-B. Suitably, at least one LC-HC assembly promoter is located in the constant region of LC-A and at least one LC-HC assembly promoter is located in the constant region of LC-B. Suitably, respective LC-HC assembly promoters are selected to allow specific assembly of LC-A to HC-A and LC-B to HC-B.

According to preferred embodiments, the at least one LC-HC assembly promoter is located in the Fab region of LC-A and/or LC-B. Suitably, at least one LC-HC assembly promoter is located in the Fab region of LC-A and at least one LC-HC assembly promoter is located in the Fab region of LC-B. Suitably, respective LC-HC assembly promoters are selected to allow specific assembly of LC-A to HC-A and LC-B to HC-B.

According to preferred embodiments, the at least one LC-HC assembly promoter is located in the CL domain of LC-A and/or LC-B. Suitably, at least one LC-HC assembly promoter is located in the CL domain of LC-A and at least one LC-HC assembly promoter is located in the CL domain of LC-B. Suitably, respective LC-HC assembly promoters are selected to allow specific assembly of LC-A to HC-A and LC-B to HC-B.

According to preferred embodiments, the at least one LC-HC assembly promoter comprises at least one amino acid substitution in an amino acid sequence of the LC-HC assembly interface. Accordingly, at least one LC-HC assembly promoter comprises at least one amino acid substitution in an amino acid sequence of the LC-HC assembly interface of LC-A and at least one LC-HC assembly promoter comprises at least one amino acid substitution in an amino acid sequence of the LC-HC assembly interface of LC-B. Suitably, respective LC-HC assembly promoters comprise amino acid substitutions to allow specific assembly of LC-A to HC-A and LC-B to HC-B.

According to preferred embodiments, the at least one LC-HC assembly promoter comprises or consists of at least one selected from steric assembly element, electrostatic steering assembly element, SEED assembly element, DEEK assembly element, interchain disulfides assembly element, or any combination thereof.

According to preferred embodiments, the at least one coding sequence of nucleic acid sequence C encodes at least one LC-HC assembly promoter and the at least one coding sequence of nucleic acid sequence D encodes at least one LC-HC assembly promoter.

In preferred embodiments, the nucleic acid sequence set of the composition comprises
a) nucleic acid sequence A comprising at least one coding sequence encoding
   at least one HC-A, or a fragment or variant thereof,
   at least one HC-HC assembly promoter, and
   at least one HC-LC assembly promoter;
b) nucleic acid sequence B comprising at least one coding sequence encoding
   at least one HC-B, or a fragment or variant thereof,
   at least one HC-HC assembly promoter, and
   at least one HC-LC assembly promoter;
c) nucleic acid sequence C comprising at least one coding sequence encoding
   at least one LC-A, or a fragment or variant thereof, and
   at least one LC-HC assembly promoter;
d) nucleic acid sequence D comprising at least one coding sequence encoding
   at least one LC-B, or a fragment or variant thereof, and
   at least one LC-HC assembly promoter.

In preferred embodiments, the composition of the invention comprises n different nucleic acid sequence sets encoding at least one antibody or a fragment or variant thereof (as defined herein), wherein n is an integer of 2 to 100. In preferred embodiments, n is an integer of 2 to 50. In more preferred embodiments, n is an integer of 2 to 20. In specific preferred embodiments, n may be selected from e.g. 2, 3, 4, 5, 6, 7, 8, 9, 10, 11, 12, 13, 14, 15, 16, 17, 18, 19, 20.

Accordingly, administration of the composition comprising n nucleic acid sequence sets to a cell or to a subject leads to expression of n assembled antibodies in said cell or subject, wherein n is an integer of 2 to 100. In preferred embodiments, n is an integer of 2 to 50. In more preferred embodiments, n is an integer of 2 to 20. In specific preferred embodiments, n may be selected from e.g. 2, 3, 4, 5, 6, 7, 8, 9, 10, 11, 12, 13, 14, 15, 16, 17, 18, 19, 20.

In particularly preferred embodiments, in vivo administration of the composition comprising n nucleic acid sequence sets to a human subject leads to expression of n assembled antibodies in said subject, wherein n is an integer of 2 to 10. In preferred embodiments, n is an integer of 2 to 5. In specific preferred embodiments, n may be selected from e.g. 2, 3, 4, or 5.

The term “assembled antibody” typically refers to an antibody comprising at least two antibody chains that are assembled and linked (e.g. via disulphide bridges). Suitably, an “assembled antibody” is assembled as such the desired function is achieved (e.g. in case of bispecific antibodies, an assembled antibody comprises two different heavy chains). Accordingly, an “assembled antibody” may be understood as correctly assembled, that is that the at least two antibody heavy chains (or fragments thereof) are assembled in the desired configuration to exert the desired function (binding to the desired antigen or antigens, triggering the desired function via e.g. Fc receptors). Accordingly, an “assembled antibody” can be understood as a correctly assembled antibody, or a correctly assembled and functional antibody. In the context of the invention, correct assembly is supported, forced, or directed by the at least one antibody chain assembly promoter (e.g. HC-HC assembly promoters, HC-LC assembly promoters, LC-HC assembly promoters).

In preferred embodiments, administration of the composition to a cell or to a subject leads to expression of at least two assembled antibodies (or fragment or variant) in said cell or subject, optionally to expression of 2 to 40, preferably 2, 3, 4, 5, 6, 7, 8, 9, 10, 11, 12, 13, 14, 15, 16, 17, 18, 19, or 20 assembled antibodies in said cell or subject, wherein preferably at least about 50%, at least about 60%, at least about 70%, at least about 75%, at least about 80%, at least about 85%, at least about 90%, at least about 95%, or at least about 100% of the expressed at least two antibodies are assembled antibodies (that is, a correctly assembled antibodies as defined herein). Suitably, the subject is a human subject. Preferably, mass spectrometry (MS) can be used to determine the percentage of assembled antibodies and misassembled antibodies

In preferred embodiments, administration of the composition to a cell or to a subject leads to expression of at least two assembled antibodies (or fragment or variant), optionally to expression of 2 to 40, preferably 2, 3, 4, 5, 6, 7, 8, 9, 10, 11, 12, 13, 14, 15, 16, 17, 18, 19, or 20 assembled antibodies in said cell or subject, wherein preferably less than about 50%, less than about 40%, less than about 30%, less than about 20%, less than about 10%, less than about 5%, or about 0%, preferably less than about 10% of the produced antibodies are misassembled antibodies (that is, not correctly assembled antibodies). Suitably, the subject is a human subject. Preferably, mass spectrometry (MS) can be used to determine the percentage of assembled antibodies and misassembled antibodies

In preferred embodiments, administration of the composition to a cell or to a subject leads to expression of at least two assembled antibodies (or fragment or variant) in the presence of at least one different antibody chain (e.g. provided by the m additional nucleic acid sequences as defined below) wherein, preferably, more than about 50%, 60%, 70%, 75%, 80%, 90%, 95%, preferably more than about 90% of the produced antibodies are correctly assembled. Suitably, the subject is a human subject. Preferably, mass spectrometry (MS) can be used to determine the percentage of assembled antibodies and misassembled antibodies

In preferred embodiments, administration of the composition to a cell or to a subject leads to expression of at least one assembled antibody (or fragment or variant) in the presence of at least one different antibody chain (e.g. provided by the m additional nucleic acid sequences as defined below), wherein preferably less than about 50%, 40%, 30%, 20%, 10%, 5%, preferably less than about 10% of the produced antibodies are misassembled. Suitably, the subject is a human subject.

In preferred embodiments, the composition comprises m additional nucleic acid sequences comprising at least one coding sequence encoding at least one antibody or a fragment of an antibody or a variant of an antibody.

Accordingly, in embodiments, the composition may comprise n different nucleic acid sequence sets as defined above, and may additionally comprise m additional nucleic acid sequences.

In preferred embodiments, the at least one antibody or a fragment or variant thereof encoded by the m additional nucleic acid sequences is a heavy chain of an antibody or a fragment or variant thereof, and/or a light chain of an antibody or a fragment or variant thereof.

Preferably, the at least one antibody or a fragment or variant thereof encoded by the m additional nucleic acid sequences is a heavy chain of an antibody or a fragment or variant thereof, and/or a light chain of an antibody or a fragment or variant thereof and does not comprise an antibody chain assembly promoter preferably as described in the context of the invention.

Notably, the term “does not comprise a antibody chain assembly promoter” as described in the context of the invention” has not to be understood as a light chain and/or heavy chain that is lacking (naturally occurring) assembly interfaces. Accordingly, the heavy chain of an antibody or a fragment or variant thereof, and/or a light chain of an antibody provided by the m nucleic acid sequences may comprise antibody chain assembly interfaces. However, said (naturally occurring) assembly interfaces do not assemble with any one of the antibody chain assembly promoters as described in the context of the invention.

In preferred embodiments, the at least one antibody or antibody fragment or variant thereof encoded by the m additional nucleic acid sequences is derived or selected from a monoclonal antibody or fragments thereof, a chimeric antibody or fragments thereof, a human antibody or fragments thereof, a humanized antibody or fragments thereof, an intrabody or fragments thereof, or a single chain antibody or fragments thereof, or a nanobody or fragments thereof.

In preferred embodiments, the at least one antibody or antibody fragment or variant thereof encoded by the m additional nucleic acid sequences is derived or selected from IgG1, IgG2, IgG3, IgG4, IgD, IgA1, IgA2, IgE, IgM, IgNAR, hclgG, BiTE, diabody, DART, TandAb, scDiabody, sc-Diabody-CH3, Diabody-CH3, Triple Body, mini antibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, Tandem scFv-Fc, Fab, Fab′, Fc, Facb, pFc′, Fd, Fv or scFv antibody fragment, scFv-Fc, scFab-Fc. Preferred in that context is IgG1, scFv-Fc and scFab-Fc.

In preferred embodiments, the at least one antibody or antibody fragment or variant thereof encoded by the m additional nucleic acid sequences specifically recognizes and/or binds to at least one target. In particularly preferred embodiments, said at least one target is an epitope or antigen.

In preferred embodiments, the at least one antibody or antibody fragment encoded by the m additional nucleic acid sequences specifically recognizes and/or binds to at least one target selected from at least one tumor antigen or epitope, at least one antigen or epitope of a pathogen, at least one viral antigen or epitope, at least one bacterial antigen or epitope, at least one protozoan antigen or epitope, at least one antigen or epitope of a cellular signalling molecule, at least one antigen or epitope of a component of the immune system, or any combination thereof.

In particularly preferred embodiments, the composition comprises m additional nucleic acid sequences encoding at least one antibody or a fragment or variant of an antibody, wherein the at least one antibody or antibody fragment specifically recognizes and/or binds to at least one target selected from at least one antigen or epitope of a pathogen, preferably a virus or a bacterium.

In preferred embodiments of the composition, the at least one antibody or antibody fragment encoded by the m additional nucleic acid sequences is derived or selected from a monospecific or a multispecific antibody or fragment or variant thereof, preferably wherein the multispecific antibody is derived or selected from a bispecific, trispecific, tetraspecific, pentaspecific, or a hexaspecific antibody or a fragment or variant thereof.

In preferred embodiments, the at least one antibody or antibody fragment encoded by the m additional nucleic acid sequences is derived or selected from an antibody heavy chain. Preferably, antibody heavy chains are selected from IgG1, IgG2, IgG3, IgG4, IgD, IgA1, IgA2, IgE, or IgM, or an allotype, an isotype, or mixed isotype or a fragment or variant of any of these, preferably IgG1 and/or IgG3.

In preferred embodiments, the at least one antibody heavy chain encoded by the m additional nucleic acid sequences is derived or selected from an antibody heavy chain of IgG, or an allotype or an isotype thereof, preferably an antibody heavy chain of IgG1 or an allotype or an isotype thereof.

In preferred embodiments of the composition, the antibody heavy chain of IgG encoded by the m additional nucleic acid sequences, preferably IgG1, is selected from G1m17, G1m3, G1m1 and G1m2, G1m27, G1m28, nG1m17, nG1m1, or any combination thereof. Suitably, the antibody heavy chain of IgG (provided by the m additional nucleic acid sequences), preferably IgG1, is selected from the allotype G1m3,1 (R120, D12/L14).

In preferred embodiments, the at least one antibody or antibody fragment encoded by the m additional nucleic acid sequences is derived or selected from an antibody heavy chain selected from IgG3. Selecting the m additional nucleic acid sequences from IgG3 may have the advantage that the HCs do not assemble with IgG1 HCs from the n different nucleic acid sequence sets.

In preferred embodiments, the at least one antibody or antibody fragment encoded by the m additional nucleic acid sequences is derived or selected from an antibody light chain. Preferably, antibody light chain is selected from a κ light chain or a λ light chain.

In preferred embodiments, the composition additionally comprises m additional nucleic acid sequences comprising at least one coding sequence encoding at least one antibody or a fragment of an antibody or a variant of an antibody. In such embodiments, the composition may comprise m additional nucleic acid sequences comprising at least one coding sequence encoding an antibody or a fragment of an antibody or a variant of an antibody, and n different nucleic acid sequence sets is derived or selected from any one as defined in the context of the first aspect.

In preferred embodiments of the composition, the m additional nucleic acid sequences of the composition encode one heavy chain (or a fragment or variant thereof) and, optionally, one light chain (or a fragment or variant thereof).

In particularly preferred embodiments, the composition comprises
(i) n different nucleic acid sequence sets encoding at least one antibody or a fragment or variant thereof as defined herein, and, additionally
(ii) m additional nucleic acid sequences comprising at least one coding sequence encoding at least one antibody or a fragment of an antibody or a variant of an antibody.

In preferred embodiments in that context, m may be an integer of 1 to 200, 1 to 100, 1 to 50, 1 to 20, or 1 to 10. In preferred embodiments, m is an integer of 1 to 20, for example 1, 2, 3, 4, 5, 6, 7, 8, 9, 10, 11, 12, 13, 14, 15, 16, 17, 18, 19, or 20.

In particularly preferred embodiments, the m additional nucleic acid sequences provide coding sequences for at least one heavy chain and at least one light chain. In such embodiments m is preferably 2. In such embodiments, the m additional nucleic acid sequences encode one functional antibody (comprising heavy and light chains).

In preferred embodiments in that context, n may be an integer of 1 to 200, 1 to 100, 1 to 50. In preferred embodiments, n is an integer of 1 to 20, for example 1, 2, 3, 4, 5, 6, 7, 8, 9, 10, 11, 12, 13, 14, 15, 16, 17, 18, 19, or 20.

In that context, it is preferred that n+m is an integer of at least 2. Suitably, n+m is an integer of 2 to 400, 2 to 200, 2 to 100, or 2 to 50. In preferred embodiments, n+m is an integer of 2 to 40, preferably 2 to 20. In specific preferred embodiments n+m is selected from 2, 3, 4, 5, 6, 7, 8, 9, 10, 11, 12, 13, 14, 15, 16, 17, 18, 19, or 20.

In particularly preferred embodiments, the composition comprises, up to 4 nucleic acid sequence sets selected from
(i) nucleic acid sequence set encoding HC-A and HC-B, comprising an assembly promoter pair HC-HC-PP3, and/or
(ii) nucleic acid sequence set encoding HC-A and HC-B, comprising an assembly promoter pair HC-HC-PP4, and/or
(iii) nucleic acid sequence set encoding HC-A and HC-B, comprising an assembly promoter pair HC-HC-PP5, and/or
(iv) nucleic acid sequence set encoding HC-A and HC-B, comprising an assembly promoter pair HC-HC-PP18, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP3, and
(ii) nucleic acid sequence set encoding HC-A and HC-B, comprising an assembly promoter pair HC-HC-PP4, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP3, and
(ii) nucleic acid sequence set encoding HC-A and HC-B, comprising an assembly promoter pair HC-HC-PP5, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP3, and
(ii) nucleic acid sequence set encoding HC-A and HC-B, comprising an assembly promoter pair HC-HC-PP18, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP4, and
(ii) nucleic acid sequence set encoding HC-A and HC-B, comprising an assembly promoter pair HC-HC-PP5, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP4, and
(ii) nucleic acid sequence set encoding HC-A and HC-B, comprising an assembly promoter pair HC-HC-PP18, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP5, and
(ii) nucleic acid sequence set encoding HC-A and HC-B, comprising an assembly promoter pair HC-HC-PP18, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP3, and
(ii) nucleic acid sequence set encoding HC-A and HC-B, comprising an assembly promoter pair HC-HC-PP4, and
(ii) nucleic acid sequence set encoding HC-A and HC-B, comprising an assembly promoter pair HC-HC-PP5, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP3, and
(ii) nucleic acid sequence set encoding HC-A and HC-B, comprising an assembly promoter pair HC-HC-PP4, and
(ii) nucleic acid sequence set encoding HC-A and HC-B, comprising an assembly promoter pair HC-HC-PP18, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP3, and
(ii) nucleic acid sequence set encoding HC-A and HC-B, comprising an assembly promoter pair HC-HC-PP5, and
(ii) nucleic acid sequence set encoding HC-A and HC-B, comprising an assembly promoter pair HC-HC-PP18, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP4, and
(ii) nucleic acid sequence set encoding HC-A and HC-B, comprising an assembly promoter pair HC-HC-PP5, and
(ii) nucleic acid sequence set encoding HC-A and HC-B, comprising an assembly promoter pair HC-HC-PP18, optionally, wherein the composition comprises m additional nucleic acid sequences encoding at least one antibody or a fragment of an antibody or a variant of an antibody.

In particularly preferred embodiments, the composition comprises,
(i) nucleic acid sequence set encoding HC-A and HC-B, comprising an assembly promoter pair HC-HC-PP3, and
(ii) nucleic acid sequence set encoding HC-A and HC-B, comprising an assembly promoter pair HC-HC-PP4, and
(iii) nucleic acid sequence set encoding HC-A and HC-B, comprising an assembly promoter pair HC-HC-PP5, and
(iv) nucleic acid sequence set encoding HC-A and HC-B, comprising an assembly promoter pair HC-HC-PP18, wherein the composition comprises m additional nucleic acid sequences encoding at least one antibody or a fragment of an antibody or a variant of an antibody.

In particularly preferred embodiments, administration of the composition to a cell or to a subject leads to expression of at least two (correctly) assembled antibodies, optionally to expression of 2 to 40, preferably 2, 3, 4, 5, 6, 7, 8, 9, 10, 11, 12, 13, 14, 15, 16, 17, 18, 19, or 20 assembled antibodies in said cell or subject, wherein, preferably, at least about 70%, at least about 75%, at least about 80%, at least about 85%, at least about 90%, at least about 95%, or about 100% of the expressed antibodies are correctly assembled antibodies. Preferably, the administration is an in vivo administration to a human subject. Preferably, mass spectrometry (MS) can be used to determine the percentage of assembled antibodies and misassembled antibodies.

In particularly preferred embodiments, administration of the composition to a cell or to a subject leads to expression of at least two (correctly) assembled antibodies, optionally to expression of 2 to 40, preferably 2, 3, 4, 5, 6, 7, 8, 9, 10, 11, 12, 13, 14, 15, 16, 17, 18, 19, or 20 assembled antibodies in said cell or subject, wherein, preferably, less than about 30%, less than about 25%, less than about 20%, less than about 15%, less than about 10%, less than about 5%, or about 0% of the expressed antibodies are mis-assembled antibodies. Preferably, the administration is an in vivo administration to a human subject. Preferably, mass spectrometry (MS) can be used to determine the percentage of assembled antibodies and misassembled antibodies.

In such embodiments, suitably, at least one antibody may be encoded by the m additional nucleic acid sequences of the composition.

According to various preferred embodiments, nucleic acid sequences of the composition may additionally encode at least one heterologous peptide or protein element.

Suitably, the at least one heterologous peptide or protein element may promote or improve secretion of the encoded antibody or antibody fragment (e.g. via secretory signal sequences), promote or improve anchoring of the encoded antibody or antibody fragment in the plasma membrane (e.g. via transmembrane elements), promote or improve formation of antibody or antibody complexes (e.g. via multimerization domains or clustering elements). In addition, a nucleic acid sequence of the composition may additionally encode peptide linker elements, self-cleaving peptides, immunologic adjuvant sequences or dendritic cell targeting sequences.

Suitable multimerization domains may be selected from the list of amino acid sequences according to SEQ ID NOs: 1116-1167 of WO2017/081082, or fragments or variants of these sequences. Suitable transmembrane elements may be selected from the list of amino acid sequences according to SEQ ID NOs: 1228-1343 of WO2017/081082, or fragments or variants of these sequences. Suitable peptide linkers may be selected from the list of amino acid sequences according to SEQ ID NOs: 1509-1565 of the patent application WO2017/081082, or fragments or variants of these sequences.

Suitable self-cleaving peptides may be selected from the list of amino acid sequences according to SEQ ID NOs: 1434-1508 of the patent application WO2017/081082, or fragments or variants of these sequences. Suitable secretory signal peptides may be selected from the list of amino acid sequences according to SEQ ID NOs: 1-1115 and SEQ ID NO: 1728 of published PCT patent application WO2017/081082, or fragments or variants of these sequences

In preferred embodiments, nucleic acid sequences of the composition may additionally encode at least one heterologous signal peptide to promote or improve the secretion of the encoded antibodies.

In embodiments, the heavy chain encoding nucleic acid sequence (for example, nucleic acid sequence A and/or B) and the light chain encoding nucleic acid sequence (for example, nucleic acid sequence C and/or D) encoding the respective assembled antibody are comprised in the composition in a w/w ratio ranging between about 10:1 to 1:10 (e.g., between about 9:1 to 1:9, 8:1 to 1:8, 7:1 to 1:7, 6:1 to 1:6, 5:1 to 1:5, 4:1 to 1:4, 3:1 to 1:3, or 2:1 to 1:2). In particular, about 9:1, 8:1, 7:1, 6:1, 5:1, 4:1, 3:1, 2:1 or 1:1.

In embodiments, the heavy chain encoding nucleic acid sequence (for example, nucleic acid sequence A and/or B) and the light chain encoding nucleic acid sequence (for example, nucleic acid sequence C and/or D) encoding the respective assembled antibody are comprised in the composition in a molar ratio ranging between approximately 10:1 to 1:10 (e.g., between approximately 9:1 to 1:9, 8:1 to 1:8, 7:1 to 1:7, 6:1 to 1:6, 5:1 to 1:5, 4:1 to 1:4, 3:1 to 1:3, or 2:1 to 1:2). In particular, about 9:1, 8:1, 7:1, 6:1, 5:1, 4:1, 3:1, 2:1 or 1:1.

In preferred embodiments, the composition of the first aspect is for in vivo expression of two different correctly assembled antibodies. In more preferred embodiments, the composition of the first aspect is for in vivo expression of three different correctly assembled antibodies. In even more preferred embodiments, the composition of the first aspect is for in vivo expression of four different correctly assembled antibodies. In particularly preferred embodiments, the composition of the first aspect is for in vivo expression of five different correctly assembled antibodies

Nucleic Acid Sequence Features and Embodiments

In the following, suitable features and embodiments referring to nucleic acid sequence A, B, C, and/or D of the n nucleic acid sequence set, and the m additional nucleic acid sequences are provided and described in detail (e.g. type of nucleic acid, structure of nucleic acid, elements of nucleic acid, modification of nucleic acid etc.). Notably, said features defining nucleic acid sequences of the first aspect (that is, the composition) may also apply to the nucleic acid sequence set of the second aspect.

In preferred embodiments of the first aspect, nucleic acid sequence A, B, C, and/or D of the n nucleic acid sequence set, and, optionally, the m additional nucleic acid sequence, is a monocistronic nucleic acid, a bicistronic nucleic acid, or multicistronic nucleic acid.

Preferably, nucleic acid sequence A, B, C, and/or D of the n nucleic acid sequence set, and, optionally, the m additional nucleic acid sequence is an artificial nucleic acid sequence as defined herein.

In embodiments, nucleic acid sequence A, B, C, and/or D of the n nucleic acid sequence set, and, optionally, the m additional nucleic acid sequence, is monocistronic and the coding sequence of said nucleic acid sequence encodes at least two different peptides or proteins. Accordingly, said coding sequence may encode at least two, three, four, five, six, seven, eight and more antibody chains as defined herein, linked with or without an amino acid linker sequence, wherein said linker sequence can comprise rigid linkers, flexible linkers, cleavable linkers, or a combination thereof. For example, a monocistronic nucleic acid may comprise a coding sequence encoding HC-A linked with or without an amino acid linker sequence to (at least a fragment of) LC-A. Likewise, a monocistronic nucleic acid may comprise a coding sequence encoding HC-B linked with or without an amino acid linker sequence to (at least a fragment of) LC-B. The m additional nucleic acid sequences may also be a monocistronic nucleic acid comprising a coding sequence encoding (at least a fragment of) one heavy chain linked with or without an amino acid linker sequence to (at least a fragment of) one light chain.

In embodiments, nucleic acid sequence A, B, C, and/or D of the n nucleic acid sequence set, and, optionally, the m additional nucleic acid sequence, may be bicistronic or multicistronic and comprises at least two coding sequences. Said at least two coding sequences suitably encode two or more different antibody chains as specified herein. Accordingly, the coding sequences in a bicistronic or multicistronic nucleic acid suitably encodes distinct proteins or peptides as defined herein or fragments variants thereof. Preferably, the coding sequences in said bicistronic or multicistronic constructs may be separated by at least one IRES (internal ribosomal entry site) sequence. Thus, the term “encoding two or more antibody chains” may mean, without being limited thereto, that the bicistronic or multicistronic nucleic acid encodes e.g. at least two, three, four, five, six or more (preferably different) antibody chains.

For example, a bicistronic nucleic acid construct of the invention may comprise nucleic acid sequence A (encoding at least a fragment of HC-A) and nucleic acid sequence C (encoding at least a fragment of LC-A), wherein, optionally, the respective coding sequences are separated by at least one IRES.

For example, a bicistronic nucleic acid construct of the invention may comprise nucleic acid sequence B (encoding at least a fragment of HC-B) and nucleic acid sequence D (encoding at least a fragment of LC-B), wherein, optionally, the respective coding sequences are separated by at least one IRES.

For example, the m additional nucleic acid sequences my be a bicistronic nucleic acid construct comprising a nucleic acid sequence encoding at least one heavy chain and nucleic acid sequence encoding at least one light chain, wherein, optionally, the respective coding sequences are separated by at least one IRES.

In that context, suitable IRES sequences may be selected from the list of nucleic acid sequences according to SEQ ID NOs: 1566-1662 of the patent application WO2017/081082, or fragments or variants of these sequences. In this context, the disclosure of WO2017/081082 relating to IRES sequences is herewith incorporated by reference.

In preferred embodiments, the nucleic acid sequence set of the composition comprises at least two monocistronic nucleic acid constructs, optionally, comprising at least four monocistronic nucleic acid constructs. In such embodiments, each monocistronic nucleic acid may comprise one nucleic acid sequence selected from nucleic acid sequence A, B, and, optionally C, and D.

In preferred embodiments, the nucleic acid sequence set of the composition comprises at least two bicistronic nucleic acid constructs. In such embodiments, each bicistronic nucleic acid may comprise two nucleic acid sequences selected from nucleic acid sequence A, B, and, optionally C, and D.

It has to be understood that, in the context of the invention, certain combinations of coding sequences (provided by nucleic acid sequence A, B, C, and/or D as defined herein, optionally, the m additional nucleic acid sequences) may be generated by any combination of monocistronic, bicistronic, and/or multicistronic nucleic acid to obtain a nucleic acid sequence composition encoding (assembled) antibodies as defined herein.

In preferred embodiments, nucleic acid sequence A, B, C, and/or D of the n nucleic acid sequence set, and, optionally, the m additional nucleic acid sequence, is an artificial nucleic acid, e.g. an artificial DNA or an artificial RNA.

In preferred embodiments, nucleic acid sequence A, B, C, and/or D of the n nucleic acid sequence set, and, optionally, the m additional nucleic acid sequence, e.g. the DNA or RNA, is a modified and/or stabilized nucleic acid, preferably a modified and/or stabilized artificial nucleic acid.

According to preferred embodiments, nucleic acid sequence A, B, C, and/or D of the n nucleic acid sequence set, and, optionally, the m additional nucleic acid sequence, may thus be provided as a “stabilized artificial nucleic acid” or “stabilized coding nucleic acid” that is to say a nucleic acid showing improved resistance to in vivo degradation and/or a nucleic acid showing improved stability in vivo, and/or a nucleic acid showing improved translatability in vivo.

In the following, specific suitable modifications/adaptations in this context are described which are suitable to “stabilize” the nucleic acid. Preferably, the nucleic acid sequences of the present invention may be provided as a “stabilized RNA”, “stabilized coding RNA”, “stabilized DNA” or “stabilized coding DNA”.

In the following, suitable modifications are described that are capable of “stabilizing” the nucleic acid of nucleic acid sequence A, B, C, and/or D of the n nucleic acid sequence set, and, optionally, the m additional nucleic acid sequence.

In preferred embodiments, nucleic acid sequence A, B, C, and/or D (of the nucleic acid sequence set) and, optionally, the m additional nucleic acid sequence has a half-life of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 day or at least 14 days (e.g. upon in vivo administration of the composition). When transfected into mammalian host cells or administered to an organism, nucleic acid sequence A, B, C, and/or D (of the n nucleic acid sequence set) and, optionally, the m additional nucleic acid sequence, comprising a codon modified coding sequence has a stability of greater than 18, 24, 36, 48, 60, 72 hours and are capable of being expressed by the mammalian host cell (e.g. a muscle cell, lung cell) or organism.

When transfected into mammalian host cells or administered to an organism, nucleic acid sequence A, B, C, and/or D (of the n nucleic acid sequence set) and, optionally, the m additional nucleic acid sequence comprising modified or stabilized coding sequence is translated into protein, wherein the amount of protein is at least comparable to, or preferably at least 10% more than, or at least 20% more than, or at least 30% more than, or at least 40% more than, or at least 50% more than, or at least 100% more than, or at least 200% or more than the amount of protein obtained by a nucleic acid sequence comprising a non-modified or non-stabilized coding sequence.

In preferred embodiments, the at least one coding sequence of nucleic acid sequence A, B, C, and/or D (of the n nucleic acid sequence set) and, optionally, the m additional nucleic acid sequence is a codon modified coding sequence. Suitably, the amino acid sequence encoded by the at least one codon modified coding sequence is not being modified compared to the amino acid sequence encoded by the corresponding wild type or reference coding sequence.

In preferred embodiments, the at least one coding sequence of nucleic acid sequence A, B, C, and/or D (of the n nucleic acid sequence set) and, optionally, the m additional nucleic acid sequence is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof.

In embodiments, nucleic acid sequence A, B, C, and/or D (of the n nucleic acid sequence set) and, optionally, the m additional nucleic acid sequence may be modified, wherein the C content of the at least one coding sequence may be increased, preferably maximized, compared to the C content of the corresponding wild type or reference coding sequence (herein referred to as “C maximized coding sequence”). The amino acid sequence encoded by the C maximized coding sequence of the nucleic acid is preferably not modified compared to the amino acid sequence encoded by the respective wild type or reference coding sequence. The generation of a C maximized nucleic acid sequences may suitably be carried out using a modification method according to WO2015/062738. In this context, the disclosure of WO2015/062738 is included herewith by reference.

In preferred embodiments, nucleic acid sequence A, B, C, and/or D (of the n nucleic acid sequence set) and, optionally, the m additional nucleic acid sequence may be modified, wherein the G/C content of the at least one coding sequence may be optimized compared to the G/C content of the corresponding wild type or reference coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the nucleic acid is preferably not modified as compared to the amino acid sequence encoded by the respective wild type or reference coding sequence. The generation of a G/C content optimized nucleic acid sequence (RNA or DNA) may be carried out using a method according to WO2002/098443. In this context, the disclosure of WO2002/098443 is included in its full scope in the present invention.

In preferred embodiments, nucleic acid sequence A, B, C, and/or D (of the n nucleic acid sequence set) and, optionally, the m additional nucleic acid sequence may be modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the nucleic acid is preferably modified such that the frequency of the codons encoding the same amino acid corresponds to the naturally occurring frequency of that codon according to the human codon usage. For example, in the case of the amino acid Ala, the wild type or reference coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table 2). Accordingly, such a procedure (as exemplified for Ala) is applied for each amino acid encoded by the coding sequence of the nucleic acid to obtain sequences adapted to human codon usage.

TABLE 2
Human codon usage table with frequencies indicated for each amino acid

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| lle | ATT | 0.35 | Thr | ACA | 0.27 |
| lle | ATC* | 0.52 | Thr | ACT | 0.23 |
| Lys | AAG* | 0.60 | Thr | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0.42 |
| Leu | CTC | 0.20 | Tyr | TAC* | 0.58 |
| Met | ATG* | 1 | Stop | TGA* | 0.61 |
| Asn | AAT | 0.44 | Stop | TAG | 0.17 |
| Asn | AAC* | 0.56 | Stop | TAA | 0.22 |

*most frequent human codon

In embodiments, nucleic acid sequence A, B, C, and/or D (of the n nucleic acid sequence set) and, optionally, the m additional nucleic acid sequence may be modified, wherein the G/C content of the at least one coding sequence may be modified compared to the G/C content of the corresponding wild type or reference coding sequence (herein referred to as “G/C content modified coding sequence”). In this context, the terms “G/C optimization” or “G/C content modification” relate to a nucleic acid that comprises a modified, preferably an increased number of guanosine and/or cytosine nucleotides as compared to the corresponding wild type or reference coding sequence. Such an increased number may be generated by substitution of codons containing adenosine or thymidine nucleotides by codons containing guanosine or cytosine nucleotides. Advantageously, nucleic acid sequences having an increased G/C content are more stable or show a better expression than sequences having an increased A/U. The amino acid sequence encoded by the nucleic acid sequence is preferably not modified as compared to the amino acid sequence encoded by the respective wild type or reference sequence. Preferably, the G/C content of the coding sequence of the nucleic acid is increased by at least 10%, 20%, 30%, preferably by at least 40% compared to the G/C content of the coding sequence of the corresponding wild type or reference nucleic acid sequence.

In embodiments, nucleic acid sequence A, B, C, and/or D (of the n nucleic acid sequence set) and, optionally, the m additional nucleic acid sequence may be modified, wherein the codon adaptation index (CAI) may be increased or preferably maximised in the at least one coding sequence (herein referred to as “CAI maximized coding sequence”). It is preferred that all codons of the wild type or reference nucleic acid sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein (see Table 2, most frequent human codons are marked with asterisks). Suitably, the nucleic acid comprises at least one coding sequence, wherein the codon adaptation index (CAI) of the at least one coding sequence is at least 0.5, at least 0.8, at least 0.9 or at least 0.95. Most preferably, the codon adaptation index (CAI) of the at least one coding sequence is 1 (CAI=1). For example, in the case of the amino acid Ala, the wild type or reference coding sequence may be adapted in a way that the most frequent human codon “GCC” is always used for said amino acid. Accordingly, such a procedure (as exemplified for Ala) may be applied for each amino acid encoded by the coding sequence of the nucleic acid to obtain CAI maximized coding sequences.

In embodiments, nucleic acid sequence A, B, C, and/or D (of the n nucleic acid sequence set) and, optionally, the m additional nucleic acid sequence may be modified by altering the number of A and/or U nucleotides in the nucleic acid sequence with respect to the number of A and/or U nucleotides in the original nucleic acid sequence (e.g. the wild type or reference sequence). Preferably, such an AU alteration is performed to modify the retention time of the individual nucleic acids in the composition, to allow co-purification using a HPLC method, and/or to allow analysis of the obtained nucleic acid composition. Such a method is described in detail in published PCT application WO2019092153A1. The disclosure relating to claims 1 to 70 of WO2019092153A1 herewith incorporated by reference.

In particularly preferred embodiments, the at least one coding sequence of nucleic acid sequence A, B, C, and/or D (of the n nucleic acid sequence set) and, optionally, the m additional nucleic acid sequence is a codon modified coding sequence, wherein the codon modified coding sequence is selected a G/C optimized coding sequence, a human codon usage adapted coding sequence, or a G/C modified coding sequence.

In preferred embodiments, nucleic acid sequence A, B, C, and/or D (of the n nucleic acid sequence set) and, optionally, the m additional nucleic acid sequence comprises at least one untranslated region (UTR).

In preferred embodiments, nucleic acid sequence A, B, C, and/or D (of the n nucleic acid sequence set) and, optionally, the m additional nucleic acid sequence comprises at least one protein-coding region (“coding sequence” or “cds”) as defined herein, and at least one 5′-UTR and/or at least one 3-UTR.

Notably, UTRs may harbor regulatory sequence elements that determine nucleic acid, e.g. RNA turnover, stability, and localization. Moreover, UTRs may harbor sequence elements that enhance translation. In medical application of nucleic acid sequences (including DNA and RNA), translation of the nucleic acid into at least one peptide or protein is of paramount importance to therapeutic efficacy. Certain combinations of 3-UTRs and/or 5′-UTRs may enhance the expression of operably linked coding sequences encoding the HC and LCs of the invention. Nucleic acid molecules harboring said UTR combinations advantageously enable rapid and transient expression of the encoded antibody after administration to a subject. Furthermore, suitable UTRs may be selected to reduce or minimize intrinsic immunostimulatory properties of the nucleic acid sequences.

Suitably, nucleic acid sequence A, B, C, and/or D (of the n nucleic acid sequence set) and, optionally, the m additional nucleic acid sequence comprises at least one heterologous 5-UTR and/or at least one heterologous 3′-UTR. Said heterologous 5′-UTRs or 3′-UTRs may be derived from naturally occurring genes or may be synthetically engineered. In preferred embodiments, the nucleic acid, preferably the RNA comprises at least one coding sequence as defined herein operably linked to at least one (heterologous) 3′-UTR and/or at least one (heterologous) 5′-UTR.

In preferred embodiments, nucleic acid sequence A, B, C, and/or D (of the n nucleic acid sequence set) and, optionally, the m additional nucleic acid sequence, e.g. the RNA or DNA, comprises at least one 3′-UTR, preferably at least one heterologous 3′-UTR.

Preferably, nucleic acid sequence A, B, C, and/or D (of the n nucleic acid sequence set) and, optionally, the m additional nucleic acid sequence comprises a 3′-UTR, which may be derivable from a gene that relates to an RNA with enhanced half-life (i.e. that provides a stable RNA).

In some embodiments, a 3′-UTR comprises one or more of a polyadenylation signal, a binding site for proteins that affect a nucleic acid stability of location in a cell, or one or more miRNA or binding sites for miRNAs. Accordingly, miRNA, or binding sites for miRNAs as defined herein may be removed from the 3′-UTR or may be introduced into the 3′-UTR in order to tailor the expression of the nucleic acid, e.g. the DNA or RNA to desired cell types or tissues.

In preferred embodiments, nucleic acid sequence A, B, C, and/or D (of the nucleic acid sequence set) and, optionally, the m additional nucleic acid sequence comprises at least one heterologous 3′-UTR, wherein the at least one heterologous 3′-UTR comprises a nucleic acid sequence derived or selected from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes. Suitably, the at least one heterologous 3′-UTR is selected from a sequence according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 23-38 or a fragment or a variant of any of these. Particularly preferred nucleic acid sequences in that context can be derived from published PCT application WO2019/077001A1, in particular, claim 9 of WO2019/077001A1. The corresponding 3′-UTR sequences of claim 9 of WO2019/077001A1 are herewith incorporated by reference (e.g., SEQ ID NOs: 23-34 of WO2019/077001A1, or fragments or variants thereof).

In preferred embodiments, nucleic acid sequence A, B, C, and/or D (of the nucleic acid sequence set) and, optionally, the m additional nucleic acid sequence comprises a 3′-UTR derived from a PSMB3 gene. Said 3′-UTR derived from a PSMB3 gene may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 23 or 24 or a fragment or a variant thereof.

In other embodiments, nucleic acid sequence A, B, C, and/or D (of the n nucleic acid sequence set) and, optionally, the m additional nucleic acid sequence may comprise a 3′-UTR as described in WO2016/107877, the disclosure of WO2016/107877 relating to 3′-UTR sequences herewith incorporated by reference. Suitable 3′-UTRs are SEQ ID NOs: 1-24 and SEQ ID NOs: 49-318 of WO2016/107877, or fragments or variants of these sequences. In other embodiments, the nucleic acid comprises a 3′-UTR as described in WO2017/036580, the disclosure of WO2017/036580 relating to 3′-UTR sequences herewith incorporated by reference. Suitable 3′-UTRs are SEQ ID NOs: 152-204 of WO2017/036580, or fragments or variants of these sequences. In other embodiments, the nucleic acid comprises a 3′-UTR as described in WO2016/022914, the disclosure of WO2016/022914 relating to 3′-UTR sequences herewith incorporated by reference. Particularly preferred 3′-UTRs are nucleic acid sequences according to SEQ ID NOs: 20-36 of WO2016/022914, or fragments or variants of these sequences.

In preferred embodiments, nucleic acid sequence A, B, C, and/or D (of the n nucleic acid sequence set) and, optionally, the m additional nucleic acid sequence, e.g. the RNA or DNA, comprises at least one 5′-UTR, preferably at least one heterologous 5′-UTR.

Preferably, nucleic acid sequence A, B, C, and/or D (of the n nucleic acid sequence set) and, optionally, the m additional nucleic acid sequence comprises a 5′-UTR, which may be derivable from a gene that relates to an RNA with enhanced half-life (i.e. that provides a stable RNA).

In some embodiments, a 5′-UTR comprises one or more of a binding site for proteins that affect an RNA stability or RNA location in a cell, or one or more miRNA or binding sites for miRNAs. Accordingly, miRNA or binding sites for miRNAs as defined above may be removed from the 5′-UTR or introduced into the 5′-UTR in order to tailor the expression of the nucleic acid to desired cell types or tissues.

In preferred embodiments, nucleic acid sequence A, B, C, and/or D (of the n nucleic acid sequence set) and, optionally, the m additional nucleic acid sequence comprises at least one heterologous 5′-UTR, wherein the at least one heterologous 5′-UTR comprises a nucleic acid sequence derived or selected from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes. Suitably, the at least one heterologous 5′-UTR is selected from a sequence according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 1-22 or a fragment or a variant of any of these. Particularly preferred nucleic acid sequences in that context can be selected from published PCT application WO2019/077001A1, in particular, claim 9 of WO2019/077001A1. The corresponding 5′-UTR sequences of claim 9 of WO2019/077001A1 are herewith incorporated by reference (e.g., SEQ ID NOs: 1-20 of WO2019/077001A1, or fragments or variants thereof).

In preferred embodiments, nucleic acid sequence A, B, C, and/or D (of the n nucleic acid sequence set) and, optionally, the m additional nucleic acid sequence comprises a 5′-UTR derived or selected from a HSD17B4 gene, wherein said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1 or 2 or a fragment or a variant thereof.

In other embodiments, nucleic acid sequence A, B, C, and/or D (of the n nucleic acid sequence set) and, optionally, the m additional nucleic acid sequence may comprises a 5′-UTR as described in WO2013/143700, the disclosure of WO2013/143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013/143700, or fragments or variants of these sequences. In other embodiments, the nucleic acid comprises a 5′-UTR as described in WO2016/107877, the disclosure of WO2016/107877 relating to 5′-UTR sequences herewith incorporated by reference. Particularly preferred 5′-UTRs are nucleic acid sequences according to SEQ ID NOs: 25-30 and SEQ ID NOs: 319-382 of WO2016/107877, or fragments or variants of these sequences. In other embodiments, the nucleic acid comprises a 5′-UTR as described in WO2017/036580, the disclosure of WO2017/036580 relating to 5′-UTR sequences herewith incorporated by reference. Particularly preferred 5′-UTRs are nucleic acid sequences according to SEQ ID NOs: 1-151 of WO2017/036580, or fragments or variants of these sequences. In other embodiments, the nucleic acid comprises a 5′-UTR as described in WO2016/022914, the disclosure of WO2016/022914 relating to 5′-UTR sequences herewith incorporated by reference. Particularly preferred 5′-UTRs are nucleic acid sequences according to SEQ ID NOs: 3-19 of WO2016/022914, or fragments or variants of these sequences.

In preferred embodiments, nucleic acid sequence A, B, C, and/or D (of the n nucleic acid sequence set) and, optionally, the m additional nucleic acid sequence comprises at least one coding sequence, wherein said coding sequence is operably linked to a 3′-UTR and/or a 5′-UTR selected from the following 5′-UTR/3′-UTR combinations: (HSD17B4/PSMB3), (NDUFA4/PSMB3), (SLC7A3/PSMB3), (NOSIP/PSMB3), (MP68/PSMB3), (UBQLN2/RPS9), (ASAH1/RPS9), (HSD17B4/RPS9), (HSD17B4/CASP1), (NOSIP/COX6B1), (NDUFA4/RPS9), (NOSIP/NDUFA1), (NDUFA4/COX6B1), (NDUFA4/NDUFA1), (ATP5A1/PSMB3), (Rpl31/PSMB3), (ATP5A1/CASP1), (SLC7A3/GNAS), (HSD17B4/NDUFA1), (Slc7a3/Ndufa1), (TUBB4B/RPS9), (RPL31/RPS9), (MP68/RPS9), (NOSIP/RPS9), (ATP5A1/RPS9), (ATP5A1/COX6B1), (ATP5A1/GNAS), (ATP5A1/NDUFA1), (HSD17B4/COX6B1), (HSD17B4/GNAS), (MP68/COX6B1), (MP68/NDUFA1), (NDUFA4/CASP1), (NDUFA4/GNAS), (NOSIP/CASP1), (RPL31/CASP1), (RPL31/COX6B1), (RPL31/GNAS), (RPL31/NDUFA1), (Slc7a3/CASP1), (SLC7A3/COX6B1), (SLC7A3/RPS9), (RPL32/ALB7), (RPL32/ALB7), or (α-globin gene/−).

In particularly preferred embodiments, nucleic acid sequence A, B, C, and/or D (of the n nucleic acid sequence set) and, optionally, the m additional nucleic acid sequence comprises at least one coding sequence as defined herein, wherein said coding sequence is operably linked to a HSD17B4 5′-UTR and a PSMB3 3′-UTR (HSD17B4/PSMB3).

In embodiments, the A/U (A/T) content in the environment of the ribosome binding site of the nucleic acid sequence A, B, C, and/or D (of the n nucleic acid sequence set) and, optionally, the m additional nucleic acid sequence may be increased compared to the A/U (A/T) content in the environment of the ribosome binding site of its respective wild type or reference nucleic acid. This modification (an increased A/U (A/T) content around the ribosome binding site) increases the efficiency of ribosome binding to the nucleic acid, e.g. to an RNA. An effective binding of the ribosomes to the ribosome binding site in turn has the effect of an efficient translation the nucleic acid.

Accordingly, in a particularly preferred embodiment, nucleic acid sequence A, B, C, and/or D (of the n nucleic acid sequence set) and, optionally, the m additional nucleic acid sequence comprises a ribosome binding site, also referred to as “Kozak sequence” identical to or at least 80%, 85%, 90%, 95% identical to any one of the sequences SEQ ID NOs: 41 or 42, or fragments or variants thereof.

In preferred embodiments, nucleic acid sequence A, B, C, and/or D (of the n nucleic acid sequence set) and, optionally, the m additional nucleic acid sequence comprises at least one poly(N) sequence, e.g. at least one poly(A) sequence, at least one poly(U) sequence, at least one poly(C) sequence, or combinations thereof.

In preferred embodiments, nucleic acid sequence A, B, C, and/or D (of the n nucleic acid sequence set) and, optionally, the m additional nucleic acid sequence comprises, preferably the RNA comprises at least one poly(A) sequence.

Suitably, the poly(A) sequence comprises about 10 to about 500 adenosine nucleotides, about 10 to about 200 adenosine nucleotides, about 30 to about 200 adenosine nucleotides, or about 30 to about 100 adenosine nucleotides. Suitably, the length of the poly(A) sequence may be at least about or even more than about 10, 50, 64, 75, 80, 90, 100, 200, 300, 400, or 500 adenosine nucleotides. In preferred embodiments, the poly(A) sequence comprises about 64 adenosine nucleotides (A64). In other preferred embodiments, the poly(A) sequence comprises about 75 adenosine nucleotides (A75). In other preferred embodiments, the poly(A) sequence comprises about 80 adenosine nucleotides (A80). In other preferred embodiments, the poly(A) sequence comprises about 90 adenosine nucleotides (A90). In other preferred embodiments, the poly(A) sequence comprises about 100 adenosine nucleotides (A100). In other embodiments, the poly(A) sequence comprises about 150 adenosine nucleotides (A150).

The poly(A) sequence as defined herein may be located directly at the 3′ terminus of nucleic acid sequence A, B, C, and/or D (of the n nucleic acid sequence set) and, optionally, the m additional nucleic acid sequence. In such embodiments, the 3′-terminal nucleotide (that is the last 3′-terminal nucleotide in the polynucleotide chain) is the 3′-terminal A nucleotide of the at least one poly(A) sequence. The term “directly located at the 3′ terminus” has to be understood as being located exactly at the 3′ terminus—in other words, the 3′ terminus of the nucleic acid consists of a poly(A) sequence terminating with an A nucleotide.

In embodiments where the nucleic acid sequence A, B, C, and/or D (of the n nucleic acid sequence set) and, optionally, the m additional nucleic acid sequence is an RNA, the poly(A) sequence of the nucleic acid is preferably obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using commercially available polyadenylation kits and corresponding protocols known in the art, or alternatively, by using immobilized poly(A)polymerases e.g. using a methods and means as described in WO2016/174271.

The nucleic acid sequence A, B, C, and/or D (of the n nucleic acid sequence set) and, optionally, the m additional nucleic acid sequence is an RNA, may comprise a poly(A) sequence obtained by enzymatic polyadenylation, wherein the majority of nucleic acid molecules comprise about 100 (+/−20) to about 500 (+/−50), preferably about 250 (+/−25) adenosine nucleotides.

In embodiments, nucleic acid sequence A, B, C, and/or D (of the n nucleic acid sequence set) and, optionally, the m additional nucleic acid sequence comprises a poly(A) sequence derived from a template DNA and additionally comprises at least one poly(A) sequence generated by enzymatic polyadenylation, e.g. as described in WO2016/091391.

In embodiments, nucleic acid sequence A, B, C, and/or D (of the n nucleic acid sequence set) and, optionally, the m additional nucleic acid sequence comprises at least one polyadenylation signal.

In embodiments, nucleic acid sequence A, B, C, and/or D (of the n nucleic acid sequence set) and, optionally, the m additional nucleic acid sequence comprises at least one poly(C) sequence as defined herein. In preferred embodiments, nucleic acid sequence A, B, C, and/or D (of the nucleic acid sequence set) and, optionally, the m additional nucleic acid sequence comprises at least one poly(C) sequence, wherein the poly(C) sequence comprises about 10 to about 100 cytosine nucleotides, preferably about 10 to about 40 cytosine nucleotides. In particularly preferred embodiments, the poly(C) sequence comprises about 30 cytosine nucleotides.

In preferred embodiments, nucleic acid sequence A, B, C, and/or D (of the n nucleic acid sequence set) and, optionally, the m additional nucleic acid sequence comprises at least one histone stem-loop (hSL) or histone stem loop structure as defined herein.

According to a further preferred embodiment, nucleic acid sequence A, B, C, and/or D (of the n nucleic acid sequence set) and, optionally, the m additional nucleic acid sequence comprises at least one histone stem-loop sequence derived from at least one of the specific formulae (Ia) or (IIa) of the patent application WO2012/019780.

In preferred embodiments, the least one histone stem-loop comprises or consists a nucleic acid sequence identical or at least 70%, 80%, 90%, 91%, 92%, 93%, 94%, 95%, 96%, 97%, 98%, or 99% identical to SEQ ID NOs: 39 or 40, or fragments or variants thereof.

In embodiments, in particular in embodiments that relate to RNA, nucleic acid sequence A, B, C, and/or D (of the n nucleic acid sequence set) and, optionally, the m additional nucleic acid sequence comprises a 3-terminal sequence element. Said 3-terminal sequence element comprises a poly(A) sequence and, optionally a histone-stem-loop sequence and, optionally, a poly(C) sequence. Accordingly, nucleic acid sequence A, B, C, and/or D (of the nucleic acid sequence set) and, optionally, the m additional nucleic acid sequence comprises at least one 3-terminal sequence element comprising or consisting a nucleic acid sequence being identical or at least 70%, 80%, 90%, 91%, 92%, 93%, 94%, 95%, 96%, 97%, 98%, or 99% identical to SEQ ID NOs: 45-74, or a fragment or variant thereof.

In various embodiments, in particular in embodiments that relate to RNA, nucleic acid sequence A, B, C, and/or D (of the n nucleic acid sequence set) and, optionally, the m additional nucleic acid sequence may comprise a 5′-terminal sequence element according to SEQ ID NOs: 43 or 44, or a fragment or variant thereof. Such a 5′-terminal sequence element comprises e.g. a binding site for T7 RNA polymerase. Further, the first nucleotide of said 5-terminal start sequence may preferably comprise a 2′O methylation, e.g. 2′O methylated guanosine or a 2′O methylated adenosine.

Preferably, nucleic acid sequence A, B, C, and/or D (of the n nucleic acid sequence set) and, optionally, the m additional nucleic acid sequence, e.g. the RNA or DNA, typically comprises about 50 to about 20000 nucleotides, or about 500 to about 10000 nucleotides, or about 1000 to about 10000 nucleotides, or preferably about 1000 to about 5000 nucleotides, or even more preferably about 2000 to about 5000 nucleotides.

In embodiments, nucleic acid sequence A, B, C, and/or D (of the n nucleic acid sequence set) and, optionally, the m additional nucleic acid sequence is a DNA or an RNA.

In embodiments, the DNA is a plasmid DNA or a linear coding DNA construct, wherein the DNA comprises or consists of the nucleic acid elements as defined herein (e.g. including coding sequences, UTRs, poly(A/T), polyadenylation signal, a promoter).

In embodiments, nucleic acid sequence A, B, C, and/or D (of the n nucleic acid sequence set) and, optionally, the m additional nucleic acid sequence is a DNA expression vector. Such a DNA expression vector may be selected from the group consisting of a bacterial plasmid, an adenovirus, a poxvirus, a parapoxivirus (ORF virus), a vaccinia virus, a fowlpox virus, a herpes virus, an adeno-associated virus (AAV), an alphavirus, a lentivirus, a lambda phage, a lymphocytic choriomeningitis virus and a Listeria sp, Salmonella sp. Suitably, the DNA may also comprise a promoter that is operably linked to the respective coding sequence of nucleic acid sequence A, B, C, and/or D (of the nucleic acid sequence set) and, optionally, the m additional nucleic acid sequence. The promoter operably linked to the coding sequence can be e.g. a promoter from a virus or from a human gene. The promoter can also be a tissue specific promoter, such as a muscle or skin specific promoter, natural or synthetic.

In embodiments, nucleic acid sequence A, B, C, and/or D (of the n nucleic acid sequence set) and, optionally, the m additional nucleic acid sequence is an adenovirus based vector. Such an adenovirus based vector may comprise at least one coding sequence encoding at least one antibody as defined herein. In the context of the invention, any suitable adenovirus based vector may be used such as those described in WO2005/071093 or WO2006/048215.

Suitably, the adenovirus based vector used is a simian adenovirus, thereby avoiding dampening of the immune response after administration by pre-existing antibodies to common human entities such as AdHu5. Suitable simian adenovirus vectors include AdCh63 (see WO/2005/071093) or AdCh68 but others may also be used. Suitably the adenovirus vector will have the E1 region deleted, rendering it replication-deficient in human cells. Other regions of the adenovirus such as E3 and E4 may also be deleted.

In preferred embodiments, nucleic acid sequence A, B, C, and/or D (of the n nucleic acid sequence set) is not a plasmid DNA and, optionally, the m additional nucleic acid sequence is not a plasmid DNA.

In preferred embodiments, nucleic acid sequence A, B, C, and/or D (of the n nucleic acid sequence set) and, optionally, the m additional nucleic acid sequence is an RNA. In preferred embodiments, all nucleic acid sequences e.g. A, B, C, and/or D (of the nucleic acid sequence set) and, optionally, the m additional nucleic acid sequence are RNA constructs.

Preferably, the nucleic acid sequence, e.g. the RNA typically comprises about 50 to about 20000 nucleotides, or about 500 to about 10000 nucleotides, or about 1000 to about 10000 nucleotides, or preferably about 1000 to about 5000 nucleotides, or even more preferably about 2000 to about 5000 nucleotides.

According to preferred embodiments, nucleic acid sequence A, B, C, and/or D (of the n nucleic acid sequence set) and, optionally, the m additional nucleic acid sequence is an RNA, preferably a coding RNA.

In preferred embodiments, the (coding) RNA is selected from an mRNA, a (coding) self-replicating RNA, a (coding) circular RNA, a (coding) viral RNA, or a (coding) replicon RNA.

In preferred embodiments, nucleic acid sequence A, B, C, and/or D (of the n nucleic acid sequence set) and, optionally, the m additional nucleic acid sequence is an mRNA. In preferred embodiments, all nucleic acid sequences e.g. A, B, C, and/or D (of the nucleic acid sequence set) and, optionally, the m additional nucleic acid sequence are mRNA constructs.

In the context of the invention, nucleic acid sequence A, B, C, and/or D (of the n nucleic acid sequence set) and, optionally, the m additional nucleic acid sequence as defined herein are translated into at least two (functional) assembled antibodies after administration (e.g. after administration to a subject, e.g. a human subject). Accordingly, nucleic acid sequence A, B, C, and/or D (of the nucleic acid sequence set) and, optionally, the m additional nucleic acid sequence, preferably the RNA, more preferably the mRNA, is for therapeutic purpose. Accordingly, the nucleic acid sequences of the composition are for therapeutic application.

Suitably, nucleic acid sequence A, B, C, and/or D (of the n nucleic acid sequence set) and, optionally, the m additional nucleic acid sequence, preferably RNA, may be modified by the addition of a 5′-cap structure, which preferably stabilizes the RNA and/or enhances expression of the encoded antibody (or antibody chain) and/or reduces the stimulation of the innate immune system (after administration to a subject). A 5′-cap structure is of particular importance in embodiments where the nucleic acid is an RNA, in particular a linear coding RNA, e.g. a linear mRNA or a linear coding replicon RNA.

Accordingly, in preferred embodiments, nucleic acid sequence A, B, C, and/or D (of the n nucleic acid sequence set) and, optionally, the m additional nucleic acid sequence, comprises a 5′-cap structure. In embodiments, a 5′-cap structure may suitably be selected from m7G, cap0, cap1, cap2, a modified cap0 or a modified cap1 structure. A 5′-cap (cap0 or cap1) structure may be formed in chemical RNA synthesis or in RNA in vitro transcription (co-transcriptional capping) using cap analogues.

In preferred embodiments, nucleic acid sequence A, B, C, and/or D (of the n nucleic acid sequence set) and, optionally, the m additional nucleic acid sequence, comprises a cap1 structure.

In preferred embodiments, the 5′-cap structure may suitably be added co-transcriptionally using tri-nucleotide cap analogue as defined herein, preferably in an RNA in vitro transcription reaction as defined herein.

In preferred embodiments, the cap1 is formed using co-transcriptional capping using tri-nucleotide cap analogues m7G(5′)ppp(5′)(2′OMeA)pG or m7G(5′)ppp(5′)(2′OMeG)pG. A preferred cap1 analogues in that context is m7G(5′)ppp(5′)(2′OMeA)pG.

In other embodiments, the 5′-cap structure is formed via enzymatic capping using capping enzymes (e.g. vaccinia virus capping enzymes and/or cap-dependent 2′-0 methyltransferases) to generate cap0 or cap1 or cap2 structures. The 5′-cap structure (cap0 or cap1) may be added using immobilized capping enzymes and/or cap-dependent 2′-0 methyltransferases using methods and means disclosed in WO2016/193226.

In preferred embodiments, about 70%, 75%, 80%, 85%, 90%, 95% of the nucleic acid species (in particular the RNA species) of nucleic acid sequence A, B, C, and/or D (of the n nucleic acid sequence set) and, optionally, the m additional nucleic acid sequence, comprises a cap1 structure as determined using a capping assay. In such embodiments, it is preferred that less than about 20%, 15%, 10%, 5%, 4%, 3%, 2%, 1% of the nucleic acid species (in particular the RNA species) of nucleic acid sequence A, B, C, and/or D (of the nucleic acid sequence set) and, optionally, the m additional nucleic acid sequence is uncapped.

In other preferred embodiments, about 70%, 75%, 80%, 85%, 90%, 95% of the nucleic acid species (in particular the RNA species) of nucleic acid sequence A, B, C, and/or D (of the n nucleic acid sequence set) and, optionally, the m additional nucleic acid sequence, comprises a cap0 structure as determined using a capping assay. In such embodiments, it is preferred that less than about 20%, 15%, 10%, 5%, 4%, 3%, 2%, 1% of the nucleic acid species (in particular the RNA species) of nucleic acid sequence A, B, C, and/or D (of the nucleic acid sequence set) and, optionally, the m additional nucleic acid sequence is uncapped.

For determining the presence/absence of a cap0 or a cap1 structure, a capping assays as described in published PCT application WO2015/101416, in particular, as described in claims 27 to 46 of published PCT application WO2015/101416 can be used. Other capping assays that may be used to determine the presence/absence of a cap0 or a cap1 structure of an RNA are described in PCT/EP2018/08667, or published PCT applications WO2014/152673 and WO2014/152659.

In preferred embodiments, nucleic acid sequence A, B, C, and/or D (of the n nucleic acid sequence set) and, optionally, the m additional nucleic acid sequence comprises an m7G(5′)ppp(5′)(2′OMeA) cap structure. In such embodiments, the nucleic acid, e.g. the RNA comprises a 5-terminal m7G cap, and an additional methylation of the ribose of the adjacent nucleotide of m7GpppN, in that case, a 2′O methylated Adenosine. Preferably, about 70%, 75%, 80%, 85%, 90%, 95% of the RNA (species) of the composition comprises such a cap1 structure as determined using a capping assay.

In other preferred embodiments, nucleic acid sequence A, B, C, and/or D (of the n nucleic acid sequence set) and, optionally, the m additional nucleic acid sequence comprises an m7G(5′)ppp(5′)(2′OMeG) cap structure. In such embodiments, the nucleic acid, e.g. the RNA comprises a 5-terminal m7G cap, and an additional methylation of the ribose of the adjacent nucleotide, in that case, a 2′O methylated guanosine. Preferably, about 70%, 75%, 80%, 85%, 90%, 95% of nucleic acid species (in particular RNA species) comprise such a cap1 structure as determined using a capping assay.

Accordingly, the first nucleotide of such a nucleic acid sequence, that is, the nucleotide downstream of the m7G(5′)ppp structure, may be a 2′O methylated guanosine or a 2′O methylated adenosine.

According to embodiments, nucleic acid sequence A, B, C, and/or D (of the n nucleic acid sequence set) and, optionally, the m additional nucleic acid sequence is a modified nucleic acid, preferably a modified RNA, wherein the modification refers to chemical modifications comprising backbone modifications as well as sugar modifications or base modifications.

A modified nucleic acid sequence may comprise nucleotide analogues/modifications, e.g. backbone modifications, sugar modifications or base modifications. A backbone modification in the context of the invention is a modification, in which phosphates of the backbone of the nucleotides of the RNA are chemically modified. A sugar modification in the context of the invention is a chemical modification of the sugar of the nucleotides of the RNA. Furthermore, a base modification in the context of the invention is a chemical modification of the base moiety of the nucleotides of the RNA. In this context, nucleotide analogues or modifications are preferably selected from nucleotide analogues which are applicable for transcription and/or translation.

In particularly preferred embodiments, the nucleotide analogues/modifications which may be incorporated into a modified nucleic acid, preferably the modified RNA of nucleic acid sequence A, B, C, and/or D are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

In some embodiments, the at least one modified nucleotide is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Particularly preferred in that context are pseudouridine (Lp), N1-methylpseudouridine (m1ψ), 5-methylcytosine, and 5-methoxyuridine.

Accordingly, in embodiments, nucleic acid sequence A, B, C, and/or D (of the n nucleic acid sequence set) and, optionally, the m additional nucleic acid sequence, preferably the RNA, comprises at least one modified nucleotide.

In some embodiments, essentially all, e.g. essentially 100% of the uracil in the coding sequence of nucleic acid sequence A, B, C, and/or D (of the n nucleic acid sequence set) and, optionally, the m additional nucleic acid sequence have a chemical modification, preferably a chemical modification is in the 5-position of the uracil.

Incorporating modified nucleotides such as e.g. pseudouridine (ψ), N1-methylpseudouridine (m1ψ), 5-methylcytosine, and/or 5-methoxyuridine into the coding sequence of nucleic acid sequence A, B, C, and/or D (of the n nucleic acid sequence set) and, optionally, the m additional nucleic acid sequence comprises may be advantageous as unwanted innate immune responses (upon administration of the nucleic acid sequence or pharmaceutical composition) may be adjusted or reduced (if required).

In embodiments, nucleic acid sequence A, B, C, and/or D (of the n nucleic acid sequence set) and, optionally, the m additional nucleic acid sequence, preferably the RNA comprises at least one coding sequence comprising at least one modified nucleotide. Preferably, the at least one modified nucleotide selected from pseudouridine (y) and/or N1-methylpseudouridine (m1ψ). In embodiments, all uracil nucleotides are replaced by pseudouridine (Ly) nucleotides and/or N1-methylpseudouridine (m1ψ) nucleotides, optionally all uracil nucleotides are replaced by pseudouridine (ψ) nucleotides and/or N1-methylpseudouridine (m1ψ) nucleotides.

In embodiments, nucleic acid sequence A, B, C, and/or D (of the n nucleic acid sequence set) and, optionally, the m additional nucleic acid sequence is an RNA, wherein the RNA may be prepared using any method known in the art, including chemical synthesis such as e.g. solid phase RNA synthesis, as well as in vitro methods, such as RNA in vitro transcription reactions. Accordingly, in a preferred embodiment, the RNA is obtained by RNA in vitro transcription.

Accordingly, in embodiments, nucleic acid sequence A, B, C, and/or D (of the n nucleic acid sequence set) and, optionally, the m additional nucleic acid sequence is preferably an in vitro transcribed RNA (that is, an RNA generated by a process of “RNA in vitro transcription” as defined herein).

In the context of producing a nucleic acid-based therapeutics, it may be required to provide GMP-grade nucleic acid, e.g. a GMP grade RNA or DNA. GMP-grade RNA or DNA may be produced using a manufacturing process approved by regulatory authorities. Accordingly, in a particularly preferred embodiment, RNA production is performed under current good manufacturing practice (GMP), implementing various quality control steps on DNA and RNA level, preferably according to WO2016/180430. In preferred embodiments, the RNA of the composition is a GMP-grade RNA, particularly a GMP-grade mRNA. Accordingly, an RNA of the composition for expression of at least two different antibodies in a cell comprising is preferably a GMP grade RNA.

The obtained RNA products of the composition are preferably purified using PureMessenger® (CureVac, Tubingen, Germany; RP-HPLC according to WO2008/077592) and/or tangential flow filtration (as described in WO2016/193206) and/or oligo d(T) purification (see WO2016/180430). Optionally, the obtained RNA products of the composition may be purified using a purification method for dsRNA removal, e.g. a cellulose-based purification method.

In a further preferred embodiment, the nucleic acid of the composition or the composition as such is lyophilized (e.g. according to WO2016/165831 or WO2011/069586) to yield a temperature stable nucleic acid composition as defined herein (e.g. RNA or DNA). The nucleic acid of the composition or the composition as such may also be dried using spray-drying or spray-freeze drying (e.g. according to WO2016/184575 or WO2016/184576) to yield a temperature stable nucleic acid powder.

Accordingly, in the context of manufacturing and purifying nucleic acid of the composition, in particular RNA, the disclosures of WO2017/109161, WO2015/188933, WO2016/180430, WO2008/077592, WO2016/193206, WO2016/165831, WO2011/069586, WO2016/184575, and WO2016/184576 are incorporated herewith by reference.

In preferred embodiments, nucleic acid sequence A, B, C, and/or D (of the n nucleic acid sequence set) and, optionally, the m additional nucleic acid sequence is a dried nucleic acid, particularly a dried RNA. Accordingly, the composition of the invention is a dried composition. The term “dried nucleic acid” as used herein has to be understood as nucleic acid that has been lyophilized, or spray-dried, or spray-freeze dried as defined above to obtain a temperature stable dried RNA (powder). The term “dried composition” as used herein has to be understood as a composition as defined herein that has been lyophilized, or spray-dried, or spray-freeze dried as defined above to obtain a temperature stable composition.

In preferred embodiments, nucleic acid sequence A, B, C, and/or D (of the n nucleic acid sequence set) and, optionally, the m additional nucleic acid sequence is a purified nucleic acid, particularly a purified RNA.

In preferred embodiments, nucleic acid sequence A, B, C, and/or D (of the n nucleic acid sequence set) and, optionally, the m additional nucleic acid sequence is a purified RNA, preferably a purified mRNA.

It has to be understood that the nucleic acid of the composition as defined herein (e.g. “dried RNA” as defined herein, “purified RNA” as defined herein, “GMP-grade RNA” as defined herein) may have superior stability characteristics (in vitro, in vivo) and improved efficiency (e.g. better translatability of e.g. the RNA in vivo) and are therefore particularly suitable for a medical purpose, e.g. a pharmaceutical composition as defined herein.

In various embodiments, nucleic acid sequence A, B, C, and/or D (of the n nucleic acid sequence set) and, optionally, the m additional nucleic acid sequence, preferably the RNA, comprises, preferably in 5′- to 3′-direction, the following elements:
A) 5′-cap structure, preferably as specified herein;
B) 5′-terminal start element, preferably as specified herein;
C) optionally, a 5′-UTR, preferably as specified herein;
D) a ribosome binding site, preferably as specified herein;
E) at least one coding sequence, preferably as specified herein;
F) 3-UTR, preferably as specified herein;
G) optionally, poly(A) sequence, preferably as specified herein;
H) optionally, poly(C) sequence, preferably as specified herein;
I) optionally, histone stem-loop structure or sequence, preferably as specified herein;
J) optionally, 3-terminal sequence element, preferably as specified herein.

In preferred embodiments, nucleic acid sequence A, B, C, and/or D (of the n nucleic acid sequence set) and, optionally, the m additional nucleic acid sequence, preferably the RNA, comprises the following elements, preferably in 5′- to 3-direction:
A) 5′-cap structure, preferably selected from m7G(5′), m7G(5′)ppp(5′)(2′OMeA), or m7G(5′)ppp(5′)(2′OMeG);
B) 5′-terminal start element, preferably selected from SEQ ID NOs: 43 or fragments or variants thereof;
C) optionally, a 5′-UTR derived from a HSD17B4 gene;
D) a ribosome binding site, preferably selected from SEQ ID NOs: 42 or fragments or variants thereof;
E) at least one coding sequence as specified herein, preferably a codon optimized coding sequence;
F) 3′-UTR derived from a 3′-UTR of a PSMB3 gene or an alpha-globin gene (“muag”);
G) optionally, poly(A) sequence comprising about 30 to about 500 adenosines;
H) optionally, poly(C) sequence comprising about 10 to about 100 cytosines;
I) optionally, histone stem-loop, preferably selected from SEQ ID NOs: 40;
J) optionally, 3-terminal sequence element, preferably as specified herein.

In particularly preferred embodiments nucleic acid sequence A, B, C, and/or D (of the n nucleic acid sequence set) and, optionally, the m additional nucleic acid sequence, preferably the RNA, comprises the following elements in 5′- to 3′-direction:
A) 5′-cap1 structure as defined herein;
B) 5′-UTR, preferably derived from a HSD17B4 gene, preferably according to SEQ ID NO: 2;
C) at least one coding sequence selected as specified herein, preferably a codon optimized coding sequence;
D) 3′-UTR, preferably derived from a PSMB3 gene, preferably according to SEQ ID NO: 24;
E) optionally, a histone stem-loop, preferably selected from SEQ ID NOs: 40;
F) at least one poly(A) sequence comprising about 100 A nucleotides, preferably representing the 3′ terminus.

In preferred embodiments of the first aspect, the composition comprises n nucleic acid sequence sets encoding at least one antibody or a fragment or variant thereof, wherein the n different nucleic acid sequence sets comprise
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody heavy chain B (HC-B), or a fragment or variant thereof,
wherein the at least one coding sequence of the nucleic acid sequence A and/or the nucleic acid sequence B encodes at least one antibody chain assembly promoter, wherein the composition is for expression of at least two assembled antibodies in vivo. Optionally, the composition comprises m additional nucleic acid sequences comprising at least one coding sequence encoding at least one antibody or a fragment of an antibody or a variant of an antibody.

In preferred embodiments of the first aspect, the composition comprises n RNA sequence sets encoding at least one antibody or a fragment or variant thereof, wherein the n different RNA sequence sets comprise
a) RNA sequence A comprising at least one coding sequence encoding at least one antibody heavy chain A (HC-A), or a fragment or variant thereof, and
b) RNA sequence B comprising at least one coding sequence encoding at least one antibody heavy chain B (HC-B), or a fragment or variant thereof,
wherein the at least one coding sequence of the RNA sequence A and/or the RNA sequence B encodes at least one antibody chain assembly promoter, wherein the composition is for expression of at least two assembled antibodies in vivo. Optionally, the composition comprises m additional nucleic acid sequences comprising at least one coding sequence encoding at least one antibody or a fragment of an antibody or a variant of an antibody.

In preferred embodiments of the first aspect, the composition comprises n nucleic acid sequence sets encoding at least one antibody or a fragment or variant thereof, wherein the n different nucleic acid sequence sets comprise
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody heavy chain B (HC-B), or a fragment or variant thereof,
wherein the at least one coding sequence of the nucleic acid sequence A and/or the nucleic acid sequence B encodes at least one antibody chain assembly promoter,
wherein antibody heavy chain A (HC-A) and antibody heavy chain B (HC-B) comprises at least one HC-HC assembly promoter pair comprising the following amino acid substitutions:
   HC-HC-PP3: S354C, T366W on HC-A; Y349C, T366S, L368A, Y407V on HC-B
   HC-HC-PP4: S364H, F405A on HC-A; Y349T, T394F on HC-B
   HC-HC-PP5: T350V, L351Y, F405A, Y407V on HC-A; T350V, T366L, K392L, T394W on HC-B
   HC-HC-PP18: Y349S, T366M, K370Y, K409V on HC-A; E/D356G, E357D, S364Q, Y407A on HC-B,

preferably, wherein the composition is for expression of at least two assembled antibodies in vivo. Optionally, the composition comprises m additional nucleic acid sequences comprising at least one coding sequence encoding at least one antibody or a fragment of an antibody or a variant of an antibody.

In preferred embodiments of the first aspect, the composition comprises n RNA sequence sets encoding at least one antibody or a fragment or variant thereof, wherein the n different RNA sequence sets comprise
a) RNA sequence A comprising at least one coding sequence encoding at least one antibody heavy chain A (HC-A), or a fragment or variant thereof, and
b) RNA sequence B comprising at least one coding sequence encoding at least one antibody heavy chain B (HC-B), or a fragment or variant thereof,
wherein the at least one coding sequence of the RNA sequence A and/or the RNA sequence B encodes at least one antibody chain assembly promoter,
wherein antibody heavy chain A (HC-A) and antibody heavy chain B (HC-B) comprises at least one HC-HC assembly promoter pair comprising the following amino acid substitutions:
   HC-HC-PP3: S354C, T366W on HC-A; Y349C, T366S, L368A, Y407V on HC-B
   HC-HC-PP4: S364H, F405A on HC-A; Y349T, T394F on HC-B
   HC-HC-PP5: T350V, L351Y, F405A, Y407V on HC-A; T350V, T366L, K392L, T394W on HC-B
   HC-HC-PP18: Y349S, T366M, K370Y, K409V on HC-A; E/D356G, E357D, S364Q, Y407A on HC-B,
wherein the composition is for expression of at least two assembled antibodies in vivo. Optionally, the composition comprises m additional nucleic acid sequences comprising at least one coding sequence encoding at least one antibody or a fragment of an antibody or a variant of an antibody.

Formulation and Complexation Features and Embodiments

In preferred embodiments, the composition of the invention comprises at least one pharmaceutically acceptable carrier or pharmaceutically acceptable excipient.

Accordingly, the composition of the invention is preferably a pharmaceutical composition.

The term “pharmaceutically acceptable carrier” or “pharmaceutically acceptable excipient” as used herein preferably includes the liquid or non-liquid basis of the composition for administration. If the composition is provided in liquid form, the carrier may be water, e.g. pyrogen-free water; isotonic saline or buffered (aqueous) solutions, e.g. phosphate, citrate etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, NaI, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂, Cal₂, CaBr₂, CaCO₃, CaSO₄, Ca(OH)₂.

Furthermore, organic anions of the aforementioned cations may be in the buffer. Accordingly, in embodiments, the pharmaceutical composition may comprise pharmaceutically acceptable carriers or excipients using one or more pharmaceutically acceptable carriers or excipients to e.g. increase stability, increase cell transfection, permit the sustained or delayed, increase the translation of encoded proteins in vivo, and/or alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the composition are capable of being mixed with the at least one nucleic acid sequence A, B, C, and/or D, optionally, a plurality of nucleic acids of the composition, in such a manner that no interaction occurs, which would substantially reduce the biological activity or the pharmaceutical effectiveness of the composition under typical use conditions (e.g., intramuscular or intradermal administration). Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The at least one pharmaceutically acceptable carrier or excipient of the pharmaceutical composition may preferably be selected to be suitable for systemic or local administration to a human subject.

Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Pharmaceutical compositions of the present invention is suitably a sterile composition and/or a pyrogen-free composition.

In a preferred embodiments, nucleic acid sequence A, B, C, and/or D (of the n nucleic acid sequence set) and, optionally, the m additional nucleic acid sequence is complexed or associated with further compound to obtain a formulated composition. A formulation in that context may have the function of a transfection agent. A formulation in that context may also have the function of protecting the nucleic acid from degradation.

In embodiments, nucleic acid sequence A, B, C, and/or D (of the n nucleic acid sequence set) and, optionally, the m additional nucleic acid sequence are formulated separately. Accordingly, nucleic acid sequence A, B, C, and/or D (of the n nucleic acid sequence set) and, optionally, the m additional nucleic acid sequence are formulated (complexed/associated) as separate entities. The formulation/complexation of the nucleic acid sequences may be the same or may be different. Suitably formulations are further specified herein and comprise e.g. complexation or associated one or more cationic or polycationic compounds to e.g. obtain a liposome or LNP formulation, or polymers (e.g. peptide based polymers).

In preferred embodiments, nucleic acid sequence A, B, C, and/or D (of the n nucleic acid sequence set) and, optionally, the m additional nucleic acid sequence are co-formulated. Accordingly, nucleic acid sequence A, B, C, and/or D (of the n nucleic acid sequence set) and, optionally, the m additional nucleic acid sequence are formulated (complexed/associated) as one entity. In these embodiments, the formulation/complexation of the nucleic acid sequences is the same (e.g. all nucleic acid sequences of the composition encapsulated in LNPs). Suitably formulations are further specified herein and comprise e.g. complexation or associated one or more cationic or polycationic compounds to e.g. obtain a liposome or LNP formulation, or polymers (e.g. peptide based polymers).

In embodiments, some nucleic acid sequences of the composition are co-formulated, and some nucleic acid sequences of the composition are formulated separately.

In preferred embodiments, nucleic acid sequence A, B, C, and/or D (of the n nucleic acid sequence set) and, optionally, the m additional nucleic acid sequence are co-formulated to increase the probability that all nucleic acid sequences of the composition are present in one particle/formulation to ensure that the nucleic acid sequences of the composition are up taken by the same cell (upon administration). A co-formulation of the nucleic acid sequences of the composition is advantageous for the production of correctly assembled antibodies (upon administration to a cell).

In preferred embodiments, nucleic acid sequence A, B, C, and/or D (of the n nucleic acid sequence set) and, optionally, the m additional nucleic acid sequence is complexed or associated with or at least partially complexed or partially associated with one or more cationic or polycationic compound. Complexation/association (“formulation”) to cationic or polycationic compounds as defined herein facilitates the uptake of the nucleic acid sequences of the composition into cells.

In preferred embodiments, the one or more cationic or polycationic compound (for complexation/encapsulation/formulation of nucleic acid sequence A, B, C, and/or D (of the n nucleic acid sequence set) and, optionally, the m additional nucleic acid sequence) is selected from a cationic or polycationic polymer, cationic or polycationic polysaccharide, cationic or polycationic lipid, cationic or polycationic protein, cationic or polycationic peptide, or any combinations thereof.

Cationic or polycationic compounds, being particularly preferred in this context may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, pIsl, FGF, Lactoferrin, Transportan, Buforin-2, Bac715-24, SynB, SynB(1), pVEC, hCT-derived peptides, SAP, or histones.

Further preferred cationic or polycationic compounds, which can be used as transfection or complexation agent may include cationic polysaccharides, for example chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

Preferred cationic or polycationic proteins or peptides that may be used for complexation of nucleic acid sequence A, B, C, and/or D (of the n nucleic acid sequence set) and, optionally, the m additional nucleic acid sequence can be derived from formula (Arg)l(Lys)m(His)n(Orn)o(Xaa)x of the patent application WO2009/030481 or WO2011/026641, the disclosure of WO2009/030481 or WO2011/026641 relating thereto incorporated herewith by reference.

In preferred embodiments, nucleic acid sequence A, B, C, and/or D (of the n nucleic acid sequence set) and, optionally, the m additional nucleic acid sequence is complexed, or at least partially complexed, with at least one cationic or polycationic proteins or peptides preferably selected from an amino acid sequence identical or at least 70%, 80%, 90%, 91%, 92%, 93%, 94%, 95%, 96%, 97%, 98%, or 99% identical to SEQ ID NOs: 75 to 79, or any combinations thereof.

According to various embodiments, the composition of the present invention comprises at least one nucleic acid set as defined herein, and, optionally, m additional nucleic acid sequence as defined herein, and a polymeric carrier.

The term “polymeric carrier” as used herein is intended to refer to a compound that facilitates transport and/or complexation of another compound (e.g. cargo nucleic acid of the composition). A polymeric carrier is typically a carrier that is formed of a polymer. A polymeric carrier may be associated to its cargo (e.g. DNA, or RNA of the composition) by covalent or non-covalent interaction. A polymer may be based on different subunits, such as e.g. a copolymer.

Suitable polymeric carriers in that context may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PEGylated PLL and polyethylenimine (PEI), dithiobis(succinimidylpropionate) (DSP), Dimethyl-3,3′-dithiobispropionimidate (DTBP), poly(ethylene imine) biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly(p-aminoester), poly(4-hydroxy-L-proine ester) (PHP), poly(allylamine), poly(α-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), Poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histine modified PLL, SS-PAEI, poly(p-aminoester), PHP, PAGA, PLGA, PPZ, PPE, PPA and PPE-EA.

In some embodiments, the polymeric carrier comprises PEI. In some embodiments, PEI is branched PEI. PEI may be a branched PEI of a molecular weight ranging from 10 to 40kDA, e.g., 25 kDa. In some embodiments, PEI is linear PEI. In some embodiments, the PEI nanoparticle that has a mean diameter of or less than about 60 nm (e.g., of or less than about 55 nm, of or less than about 50 nm, of or less than about 45 nm, of or less than about 40 nm, of or less than about 35 nm, of or less than about 30 nm, or of or less than about 25 nm). Suitable nanoparticles may be in the range of 25 nm to 60 nm, e.g. 30 nm to 50 nm. As used herein, the mean diameter may be represented by the z-average as determined by dynamic light scattering as commonly known in the art.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are preferably crosslinked by disulfide bonds (via —SH groups).

In this context, polymeric carriers according to formula {(Arg)l(Lys)m(His)n(Orn)o(Xaa)x(Cys)y} and formula Cys,{(Arg)l(Lys)m(His)n(Orn)o(Xaa)x)Cys2 of the patent application WO2012/013326 are preferred, the disclosure of WO2012/013326 relating thereto incorporated herewith by reference.

In embodiments, the polymeric carrier used to complex nucleic acid sequence A, B, C, and/or D (of the n nucleic acid sequence set) and, optionally, the m additional nucleic acid sequence may be derived from a polymeric carrier molecule according formula (L-P¹-S-[S-P²-S].-S—P³-L) of the patent application WO2011/026641, the disclosure of WO2011/026641 relating thereto incorporated herewith by reference.

In embodiments, the polymeric carrier compound is formed by, or comprises or consists of the peptide elements CysArg12Cys (SEQ ID NO: 75) or CysArg12 (SEQ ID NO: 76) or TrpArg12Cys (SEQ ID NO: 77). In particularly preferred embodiments, the polymeric carrier compound consists of a (R₁₂C)—(R₁₂C) dimer, a (WR₁₂C)—(WR₁₂C) dimer, or a (CR₁₂)—(CR₁₂C)—(CR₁₂) trimer, wherein the individual peptide elements in the dimer (e.g. (WR12C)), or the trimer (e.g. (CR12)), are preferably connected via —SH groups.

In embodiments, nucleic acid sequence A, B, C, and/or D (of the nucleic acid sequence set) and, optionally, the m additional nucleic acid sequence is complexed or associated with a polyethylene glycol/peptide polymer comprising HO-PEG5000-S-(S-CHHHHHHRRRRHHHHHHC-S-)7-S-PEG5000-OH (SEQ ID NO: 78 as peptide monomer), HO-PEG5000-S-(S-CHHHHHHRRRRHHHHHHC-S-)4-S-PEG5000-OH (SEQ ID NO: 78 as peptide monomer), HO-PEG5000-S-(S-CGHHHHHRRRRHHHHHGC-S-)7-S-PEG5000-OH (SEQ ID NO: 79 as peptide monomer) and/or a polyethylene glycol/peptide polymer comprising HO-PEG5000-S-(S-CGHHHHHRRRRHHHHHGC-S-)4-S-PEG5000-OH (SEQ ID NO: 79 of the peptide monomer).

In other embodiments, the composition comprises nucleic acid sequence A, B, C, and/or D (of the n nucleic acid sequence set) and, optionally, the m additional nucleic acid sequence, wherein nucleic acid sequence A, B, C, and/or D (of the nucleic acid sequence set) and, optionally, the m additional nucleic acid sequence is complexed or associated with polymeric carriers and, optionally, with at least one lipid component as described in WO2017/212008A1, WO2017/212006A1, WO2017/212007A1, and WO2017/212009A1. In this context, the disclosures of WO2017/212008A1, WO2017/212006A1, WO2017/212007A1, and WO2017/212009A1 are herewith incorporated by reference.

In preferred embodiments, the polymeric carrier is a peptide polymer, preferably a polyethylene glycol/peptide polymer as defined above, and comprises a lipid component, preferably a lipidoid component.

In preferred embodiments, the composition comprises a lipid component or a lipidoid component.

A lipidoid (or lipidoit) is a lipid-like compound, i.e. an amphiphilic compound with lipid-like physical properties. The lipidoid is preferably a compound, which comprises two or more cationic nitrogen atoms and at least two lipophilic tails. In contrast to many conventional cationic lipids, the lipidoid may be free of a hydrolysable linking group, in particular linking groups comprising hydrolysable ester, amide or carbamate groups. The cationic nitrogen atoms of the lipidoid may be cationisable or permanently cationic, or both types of cationic nitrogens may be present in the compound. In the context of the present invention, the term lipid is considered to also encompass lipidoids.

In preferred embodiments, nucleic acid sequence A, B, C, and/or D (of the n nucleic acid sequence set) and, optionally, the m additional nucleic acid sequence is complexed or associated with a polymeric carrier, preferably with a polyethylene glycol/peptide polymer as defined above, and a lipidoid component.

Suitably, the lipidoid component is cationic, which means that it is cationisable or permanently cationic. In one embodiment, the lipidoid is cationisable, i.e. it comprises one or more cationisable nitrogen atoms, but no permanently cationic nitrogen atoms. In another embodiment, at least one of the cationic nitrogen atoms of the lipidoid is permanently cationic. Optionally, the lipidoid comprises two permanently cationic nitrogen atoms, three permanently cationic nitrogen atoms, or even four or more permanently cationic nitrogen atoms.

In some embodiments, the lipidoid may comprise a aggregation reducing moiety, and/or a polymer moiety, e.g. a PEG moiety.

In a preferred embodiment, the lipidoid component may be any one selected from the lipidoids provided in table of page 50-54 of published PCT patent application WO2017/212009A1, the specific lipidoids provided in said table, and the specific disclosure relating thereto herewith incorporated by reference.

In preferred embodiments, the lipidoid component may be any one selected from 3-C12-OH, 3-C12-OH-cat, 3-C12-amide, 3-C12-amide monomethyl, 3-C12-amide dimethyl, RevPEG(10)-3-C12-OH, RevPEG(10)-DLin-pAbenzoic, 3C12amide-TMA cat., 3C12amide-DMA, 3C12amide-NH2, 3C12amide-OH, 3C12Ester-OH, 3C12 Ester-amin, 3C12Ester-DMA, 2C12Amid-DMA, 3C12-lin-amid-DMA, 2C12-sperm-amid-DMA, or 3C12-sperm-amid-DMA (see table of published PCT patent application WO2017/212009A1 (pages 50-54)). Particularly preferred are 3-C12-OH or 3-C12-OH-cat.

Further suitable lipidoid components may be derived from published PCT patent application WO2010/053572. In particular, lipidoids derivable from claims 1 to 297 of published PCT patent application WO2010/053572 may be used in the context of the invention, e.g. incorporated into the peptide polymer as described herein, or e.g. incorporated into the lipid nanoparticle (as described below). Accordingly, claims 1 to 297 of published PCT patent application WO2010/053572, and the specific disclosure relating thereto, is herewith incorporated by reference.

In preferred embodiments, the polyethylene glycol/peptide polymer optionally comprising a lipidoid component as specified above (e.g. 3-C12-OH or 3-C12-OH-cat), is used to complex the at least one nucleic acid to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid. In that context, the disclosure of published PCT patent application WO2017/212009A1, in particular claims 1 to 10 of WO2017/212009A1, and the specific disclosure relating thereto is herewith incorporated by reference.

In preferred embodiments, nucleic acid sequence A, B, C, and/or D (of the n nucleic acid sequence set) and, optionally, the m additional nucleic acid sequence is complexed, encapsulated, partially encapsulated, or associated with one or more lipids (e.g. cationic lipids and/or neutral lipids), thereby forming lipid-based carriers including liposomes, lipid nanoparticles (LNPs), lipoplexes, and/or nanoliposomes.

The term “lipid-based carriers” encompass lipid based delivery systems for RNA that comprise one or more lipid components (e.g. an aggregation reducing lipid, a cationic lipid, etc.). A lipid-based carrier may additionally comprise other components suitable for encapsulating/incorporating e.g. an RNA including a cationic or polycationic polymer, a cationic or polycationic polysaccharide, a cationic or polycationic protein, a cationic or polycationic peptide, or any combinations thereof. The term “lipid-based carriers” encompasses artificial lipid-based carrier system and does not comprise natural systems including virus particles etc.

In preferred embodiments, nucleic acid sequence A, B, C, and/or D (of the n nucleic acid sequence set) and, optionally, the m additional nucleic acid sequence is complexed, encapsulated, partially encapsulated, or associated with one or more lipids (e.g. cationic lipids and/or neutral lipids), thereby forming lipid nanoparticles (LNPs).

In embodiments, nucleic acid sequence A, B, C, and/or D (of the n nucleic acid sequence set) and, optionally, the m additional nucleic acid sequence are formulated in separate liposomes, lipid nanoparticles (LNP), lipoplexes, and/or nanoliposomes In embodiments, nucleic acid sequence A, B, C, and/or D (of the n nucleic acid sequence set) and, optionally, the m additional nucleic acid sequence are co-formulated (in any formulation or complexation agent defined herein).

In preferred embodiments, nucleic acid sequence A, B, C, and/or D (of the n nucleic acid sequence set) and, optionally, the m additional nucleic acid sequence are co-formulated in liposomes, lipid nanoparticles (LNP), lipoplexes, and/or nanoliposomes.

In embodiments, nucleic acid sequences of the composition that encode for one antibody species (that is, e.g., the components of one nucleic acid sequence set) are co-formulated (e.g. LNP). In such embodiments it may additionally be advantageous to separately formulate different nucleic acid sequence sets.

For example, if one nucleic acid sequence set encodes for Antibody A (e.g. comprising HC-HC-PP3), one nucleic acid sequence set encodes for Antibody B (e.g. comprising HC-HC-PP4), one nucleic acid sequence set encodes for Antibody C (e.g. comprising HC-HC-PP5), and one nucleic acid sequence set encodes for Antibody D (e.g. comprising HC-HC-PP18) etc., it may be advantageous to generate different co-formulations for antibody A (formulation A), antibody B (formulation B), antibody C (formulation C), and antibody D (formulation D) etc. The co-formulation of the components of the nucleic acid sequence sets (and the additional separate formulation of different nucleic acid sequence sets) may further increase the correct assembly in particular for in vivo applications.

The liposomes, lipid nanoparticles (LNPs), lipoplexes, and/or nanoliposomes—incorporated nucleic acid (e.g. DNA or RNA) may be completely or partially located in the interior space of the liposomes, lipid nanoparticles (LNPs), lipoplexes, and/or nanoliposomes, within the lipid layer/membrane, or associated with the exterior surface of the lipid layer/membrane. The incorporation of a nucleic acid into liposomes/LNPs is also referred to herein as “encapsulation” wherein the nucleic acid, e.g. the RNA is entirely contained within the interior space of the liposomes, lipid nanoparticles (LNPs), lipoplexes, and/or nanoliposomes. The purpose of incorporating nucleic acid into liposomes, lipid nanoparticles (LNPs), lipoplexes, and/or nanoliposomes is to protect the nucleic acid, preferably RNA from an environment which may contain enzymes or chemicals or conditions that degrade nucleic acid and/or systems or receptors that cause the rapid excretion of the nucleic acid. Moreover, incorporating nucleic acid, preferably RNA into liposomes, lipid nanoparticles (LNPs), lipoplexes, and/or nanoliposomes may promote the uptake of the nucleic acid, and hence, may enhance the therapeutic effect of the nucleic acid of the n nucleic acid sequence set (nucleic acid sequence A, B, C, and/or D) and, optionally, the m additional nucleic acid sequence. Accordingly, incorporating a nucleic acid of the composition into liposomes, lipid nanoparticles (LNPs), lipoplexes, and/or nanoliposomes may be particularly suitable for production of correctly assembled antibodies (upon administration). In this context, the terms “complexed” or “associated” refer to the essentially stable combination of nucleic acid with one or more lipids into larger complexes or assemblies without covalent binding.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of a nucleic acid, e.g. an RNA. For example, a liposome, a lipid complex, a lipoplex and the like are within the scope of a lipid nanoparticle (LNP).

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter.

LNPs of the invention can be characterized as microscopic vesicles having, optionally, an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphiphilic polymers and surfactants (e.g., polymerosomes, niosomes, etc.). In the context of the present invention, an LNP typically serves to transport the nucleic acid sequence set (nucleic acid sequence A, B, C, and/or D) and, optionally, the m additional nucleic acid sequence to a target tissue.

In preferred embodiments, nucleic acid sequence A, B, C, and/or D (of the n nucleic acid sequence set) and, optionally, the m additional nucleic acid sequence is complexed with one or more lipids thereby forming lipid nanoparticles (LNP), liposomes, nanoliposomes, lipoplexes. Preferably, LNPs (liposomes, nanoliposomes, lipoplexes) are particularly suitable for systemic or local administration, e.g. intravenous, intramuscular, intradermal, or pulmonary administration.

In preferred embodiments, the liposomes, lipid nanoparticles (LNP), lipoplexes, and/or nanoliposomes comprises at least one cationic or cationizable lipid.

LNPs (or liposomes, nanoliposomes, lipoplexes) typically comprise a cationic lipid and one or more excipients selected from neutral lipids, charged lipids, steroids and aggregation reducing lipids, preferably polymer conjugated lipids (e.g. PEGylated lipid). The nucleic acid of the composition may be encapsulated in the lipid portion of the LNP or an aqueous space enveloped by some or the entire lipid portion of the LNP. The nucleic acid (e.g. RNA, DNA) or a portion thereof may also be associated and complexed with the LNP. An LNP may comprise any lipid capable of forming a particle to which the nucleic acids are attached, or in which the one or more nucleic acids are encapsulated. Preferably, the LNP comprising nucleic acids comprises one or more cationic lipids, and one or more stabilizing lipids. Stabilizing lipids include neutral lipids and aggregation reducing lipids, preferably polymer conjugated lipids (e.g. PEGylated lipids).

The term “aggregation reducing lipid” refers to a molecule comprising both a lipid portion and a moiety suitable of reducing or preventing aggregation of the lipid-based carriers encapsulating the RNA in a composition. Under storage conditions, the lipid-based carriers may undergo charge-induced aggregation, a condition which can be undesirable for the stability of the composition. Therefore, it can be desirable to include a lipid compound which can reduce aggregation, for example by sterically stabilizing the lipid-based carriers. Such a steric stabilization may occur when a compound having a sterically bulky but uncharged moiety that shields or screens the charged portions of a lipid-based carriers from close approach to other lipid-based carriers in the composition. In the context of the invention, stabilization of the lipid-based carriers is achieved by including lipids which may comprise a lipid bearing a sterically bulky group which, after formation of the lipid-based carrier, is preferably located on the exterior of the lipid-based carrier. Suitable aggregation reducing groups include hydrophilic groups, e.g. polymers, such as poly(oxyalkylenes), e.g., a poly(ethylene glycol) or poly(propylene glycol). Lipids comprising a polymer as aggregation reducing group are herein referred to as “polymer conjugated lipid”.

The cationic lipid of an LNP (or liposomes, nanoliposomes, lipoplexes) may be cationisable, i.e. the lipid becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Such lipids (for liposomes, lipid nanoparticles (LNP), lipoplexes, and/or nanoliposomes) include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)-N,N-dimethyl-2,2-di((9Z,12Z)-octadeca-9,12-dienyl)tetrahydro-3aH-cyclopenta[d] [1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-N,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010/053572 (and particularly, CI 2-200 described at paragraph [00225]) and WO2012/170930, both of which are incorporated herein by reference, HGT4003, HGT5000, HGTS001, HGT5001, HGT5002 (see US20150140070A1).

In some embodiments, the lipid is selected from the group consisting of 98N12-5, C12-200, and ckk-E12.

In embodiments, the cationic lipid of the liposomes, lipid nanoparticles (LNP), lipoplexes, and/or nanoliposomes may be an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1.2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In embodiments, the cationic lipid of the liposomes, lipid nanoparticles (LNP), lipoplexes, and/or nanoliposomes may an amino alcohol lipidoid.

Amino alcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531A1, hereby incorporated by reference.

In another embodiment, suitable lipids can also be the compounds as disclosed in WO2015/074085A1 (i.e. ATX-001 to ATX-032 or the compounds as specified in claims 1-26), U.S. Appl. Nos. 61/905,724 and Ser. No. 15/614,499 or U.S. Pat. Nos. 9,593,077 and 9,567,296 hereby incorporated by reference in their entirety.

In other embodiments, suitable cationic lipids can also be the compounds as disclosed in WO2017/117530A1 (i.e. lipids 13, 14, 15, 16, 17, 18, 19, 20, or the compounds as specified in the claims), hereby incorporated by reference in its entirety.

In preferred embodiments, ionizable or cationic lipids may also be selected or derived from the lipids disclosed in WO2018/078053A1 (i.e. lipids derived from formula I, II, and III of WO2018/078053A1, or lipids as specified in claims 1 to 12 of WO2018/078053A1), the disclosure of WO2018/078053A1 hereby incorporated by reference in its entirety. In that context, lipids disclosed in Table 7 of WO2018/078053A1 (e.g. lipids derived from formula 1-1 to 1-41) and lipids disclosed in Table 8 of WO2018/078053A1 (e.g. lipids derived from formula II-1 to 11-36) may be suitably used in the context of the invention. Accordingly, formula 1-1 to formula 1-41 and formula II-1 to formula II-36 of WO2018/078053A1, and the specific disclosure relating thereto, are herewith incorporated by reference.

In preferred embodiments, cationic lipids may be selected or derived from formula III of published PCT patent application WO2018/078053A1. Accordingly, formula III of WO2018/078053A1, and the specific disclosure relating thereto, are herewith incorporated by reference.

In particularly preferred embodiments, nucleic acid sequence A, B, C, and/or D (of the n nucleic acid sequence set) and, optionally, the m additional nucleic acid sequence is complexed with one or more lipids thereby forming LNPs (or liposomes, nanoliposomes, lipoplexes), wherein the cationic lipid of the LNP is selected or derived from structures III-1 to III-36 of Table 9 of published PCT patent application WO2018/078053A1. Accordingly, formula III-1 to III-36 of WO2018/078053A1, and the specific disclosure relating thereto, are herewith incorporated by reference.

In particularly preferred embodiments, nucleic acid sequence A, B, C, and/or D (of the n nucleic acid sequence set) and, optionally, the m additional nucleic acid sequence is complexed with one or more lipids thereby forming liposomes, lipid nanoparticles (LNP), lipoplexes, and/or nanoliposomes, preferably LNPs, wherein the liposomes, lipid nanoparticles (LNP), lipoplexes, and/or nanoliposomes, preferably the LNPs comprise a cationic lipid according to formula III-3 of Table 9 of published PCT patent application WO2018/078053A1, preferably lipid ALC-0315.

Other suitable (cationic or ionizable) lipids are disclosed in WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836, US2014/0039032 and WO2017/112865. In that context, the disclosures of WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836 and US2014/0039032 and WO2017/112865 specifically relating to (cationic) lipids suitable for LNPs (or liposomes, nanoliposomes, lipoplexes) are incorporated herewith by reference.

In certain embodiments, the cationic lipid as defined herein, more preferably cationic lipid compound III-3 of Table 9 of published PCT patent application WO2018/078053A1 (ALC-0315), is present in the LNP (or liposomes, nanoliposomes, lipoplexes) in an amount from about 30 to about 95 mole percent, relative to the total lipid content of the LNP. If more than one cationic lipid is incorporated within the LNP, such percentages apply to the combined cationic lipids.

In embodiments, the cationic lipid is present in the LNP (or liposomes, nanoliposomes, lipoplexes) in an amount from about 30 mol % to about 70 mol %. In one embodiment, the cationic lipid is present in the LNP (or liposomes, nanoliposomes, lipoplexes) in an amount from about 40 mol % to about 60 mol % mole percent, such as about 40, 41, 42, 43, 44, 45, 46, 47, 48, 49, 50, 51, 52, 53, 54, 55, 56, 57, 58, 59 or 60 mol %, respectively. In embodiments, the cationic lipid is present in the LNP (or liposomes, nanoliposomes, lipoplexes) in an amount from about 47 mol % to about 48 mol %, wherein about 47.7 mol % are preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP (or liposomes, nanoliposomes, lipoplexes). In further embodiments, the LNPs (or liposomes, nanoliposomes, lipoplexes) comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to nucleic acid (e.g. coding RNA or DNA) is from about 3 to about 15, such as from about 5 to about 13 or from about 7 to about 11.

In embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7.

LNPs (or liposomes, nanoliposomes, lipoplexes) can comprise two or more (different) cationic lipids as defined herein. Cationic lipids may be selected to contribute to different advantageous properties. For example, cationic lipids that differ in properties such as amine pKa, chemical stability, half-life in circulation, half-life in tissue, net accumulation in tissue, or toxicity can be used in the LNP (or liposomes, nanoliposomes, lipoplexes). In particular, the cationic lipids can be chosen so that the properties of the mixed-LNP are more desirable than the properties of a single-LNP of individual lipids.

The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the nucleic acid which is used as cargo. The N/P ratio may be calculated on the basis that, for example, 1 ug RNA typically contains about 3 nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

In vivo characteristics and behavior of LNPs (or liposomes, nanoliposomes, lipoplexes) can be modified by addition of a hydrophilic polymer coating, e.g. polyethylene glycol (PEG), to the LNP surface to confer steric stabilization.

Furthermore, LNPs (or liposomes, nanoliposomes, lipoplexes) can be used for specific targeting by attaching ligands (e.g. antibodies, peptides, and carbohydrates) to its surface or to the terminal end of the attached PEG chains (e.g. via PEGylated lipids or PEGylated cholesterol).

In preferred embodiments, the liposomes, lipid nanoparticles (LNP), lipoplexes, and/or nanoliposomes of the composition comprise at least one aggregation reducing lipid, preferably a polymer conjugated lipid, e.g. a PEG conjugated lipid.

The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

In certain embodiments, the LNP (or liposomes, nanoliposomes, lipoplexes) comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as w-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In preferred embodiments, the PEGylated lipid is preferably selected or derived from formula (IV) of published PCT patent application WO2018/078053A1. Accordingly, PEGylated lipids selected or derived from formula (IV) of published PCT patent application WO2018/078053A1, and the respective disclosure relating thereto, are herewith incorporated by reference.

In a preferred embodiments, nucleic acid sequence A, B, C, and/or D (of the n nucleic acid sequence set) and, optionally, the m additional nucleic acid sequence of the pharmaceutical composition is complexed with one or more lipids thereby forming LNPs (or liposomes, nanoliposomes, lipoplexes), wherein the LNP comprises an aggregation reducing lipids, preferably a polymer conjugated lipid, more preferably a PEGylated lipid, wherein the PEGylated lipid is preferably selected or derived from formula (IVa) of published PCT patent application WO2018/078053A1. Accordingly, PEGylated lipid derived from formula (IVa) of published PCT patent application WO2018/078053A1, and the respective disclosure relating thereto, is herewith incorporated by reference.

In a preferred embodiment, nucleic acid sequence A, B, C, and/or D (of the n nucleic acid sequence set) and, optionally, the m additional nucleic acid sequence, is complexed with one or more lipids thereby forming lipid nanoparticles (or liposomes, nanoliposomes, lipoplexes), wherein the LNP (or liposomes, nanoliposomes, lipoplexes) comprises an aggregation reducing lipids, preferably a polymer conjugated lipid, more preferably a PEGylated lipid/PEG lipid.

In preferred embodiments, said PEG lipid or PEGylated lipid is selected or derived from formula (IVa) of WO2018/078053A1 (formula IVa of WO2018/078053A1 herewith incorporated by reference), wherein n of lipid according to formula IVa has a mean value ranging from about 30 to about 60, such as about 30±2, 32±2, 34±2, 36±2, 38±2, 40±2, 42±2, 44±2, 46±2, 48±2, 50±2, 52±2, 54±2, 56±2, 58±2, or 60±2. In a most preferred embodiment n is about 49 or n is about 45.

Further examples of PEG-lipids suitable in that context are provided in US2015/03761 15A1 and WO2015/199952, each of which is incorporated by reference in its entirety.

In some embodiments, LNPs (or liposomes, nanoliposomes, lipoplexes) include less than about 3, 2, or 1 mole percent of PEG or PEG-modified lipid, based on the total moles of lipid in the LNP. In further embodiments, LNPs (or liposomes, nanoliposomes, lipoplexes) comprise from about 0.1% to about 20% of the PEG-modified lipid on a molar basis, e.g., about 0.5 to about 10%, about 0.5 to about 5%, about 10%, about 5%, about 3.5%, about 3%, about 2.5%, about 2%, about 1.5%, about 1%, about 0.5%, or about 0.3% on a molar basis (based on 100% total moles of lipids in the LNP). In preferred embodiments, LNPs (or liposomes, nanoliposomes, lipoplexes) comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP). In various embodiments, the molar ratio of the cationic lipid to the PEGylated lipid ranges from about 100:1 to about 25:1.

In various preferred embodiments, the aggregation reducing lipid, preferably the polymer conjugated lipid does not comprise a polyethylene glycol (PEG). According to preferred embodiments, the liposomes, lipid nanoparticles (LNP), lipoplexes, and/or nanoliposomes of the composition comprise a PEG-free polymer conjugated lipid.

In preferred embodiments, the LNP (or liposomes, nanoliposomes, lipoplexes) comprises one or more additional lipids, which stabilize the formation of particles during their formulation or during the manufacturing process (e.g. neutral lipid and/or one or more steroid or steroid analogue).

In preferred embodiments, nucleic acid sequence A, B, C, and/or D (of the n nucleic acid sequence set) and, optionally, the m additional nucleic acid sequence is complexed with one or more lipids thereby forming lipid nanoparticles (or liposomes, nanoliposomes, lipoplexes), wherein the LNP (or liposomes, nanoliposomes, lipoplexes) comprises one or more neutral lipid and/or one or more steroid or steroid analogue.

Suitable stabilizing lipids include neutral lipids and anionic lipids. The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

In embodiments, the LNP (or liposome, nanoliposome, lipoplex) comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1 carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In some embodiments, the LNPs (or liposomes, nanoliposomes, lipoplexes) comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In preferred embodiments, the neutral lipid is 1,2-distearoyl-sn-glycero-3-phosphocholine (DSPC). Suitably, the molar ratio of the cationic lipid to DSPC may be in the range from about 2:1 to about 8:1.

In preferred embodiments, the steroid is cholesterol. Suitably, the molar ratio of the cationic lipid to cholesterol may be in the range from about 2:1 to about 1:1. In some embodiments, the cholesterol may a polymer-conjugated cholesterol, e.g. a PEGylated cholesterol.

The sterol can be about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle, liposomes, nanoliposomes, or lipoplex).

Preferably, lipid LNPs (or liposomes, nanoliposomes, lipoplexes) of the composition comprise:
(a) nucleic acid sequence A, B, C, and/or D (of the nucleic acid sequence set) and, optionally, the m additional nucleic acid sequence, (b) a cationic lipid, (c) an aggregation reducing agent (e.g. a polymer conjugated lipid or PEG-modified lipid), (d) optionally a non-cationic lipid (e.g. a neutral lipid), and (e) optionally, a sterol (e.g. cholesterol).

In some embodiments, the cationic lipids (as defined above), non-cationic lipids (as defined above), cholesterol (as defined above), and/or PEG-modified lipids (as defined above) may be combined at various relative molar ratios. For example, the ratio of cationic lipid to non-cationic lipid to cholesterol-based lipid to PEGylated lipid may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20:10, 40:30:25:5 or 40:32:20:8, 40:32:25:3 or 40:33:25:2, respectively.

In particularly preferred embodiments, nucleic acid sequence A, B, C, and/or D (of the n nucleic acid sequence set) and, optionally, the m additional nucleic acid sequence is complexed with one or more lipids thereby forming lipid nanoparticles (or liposomes, nanoliposomes, lipoplexes), wherein the LNP (or liposome, nanoliposome, lipoplex) comprises
(i) at least one cationic or cationizable lipid, preferably as defined herein;
(ii) at least one neutral lipid, preferably as defined herein;
(iii) at least one steroid or steroid analogue, preferably as defined herein; and
(iv) at least one aggregation reducing lipids, preferably a polymer conjugated lipid, e.g. a PEG-lipid as defined herein.

In equally preferred embodiments, nucleic acid sequence A, B, C, and/or D (of the n nucleic acid sequence set) and, optionally, the m additional nucleic acid sequence is complexed with one or more lipids thereby forming lipid nanoparticles (or liposomes, nanoliposomes, lipoplexes), wherein the LNP (or liposome, nanoliposome, lipoplex) comprises
a) at least one cationic or cationizable lipid, preferably wherein the lipid is not ALC-0315;
b) at least one neutral lipid, preferably as defined herein;
c) at least one steroid or steroid analogue, preferably as defined herein; and
d) at least one polymer conjugated lipid, preferably wherein the polymer conjugated lipid is not a PEG-lipid

In particularly preferred embodiments, nucleic acid sequence A, B, C, and/or D (of the n nucleic acid sequence set) and, optionally, the m additional nucleic acid sequence is complexed with one or more lipids thereby forming lipid nanoparticles (LNP), wherein the LNP comprises (i) to (iv) in a molar ratio of about 20-60% cationic lipid:5-25% neutral lipid:25-55% sterol; 0.5-15% aggregation reducing lipid, preferably polymer conjugated lipid.

In one preferred embodiment, the lipid nanoparticle (or liposome, nanoliposome, lipoplex) comprises: a cationic lipid with formula (III) of WO2018/078053A1 and/or PEG lipid with formula (IV) of WO2018/078053A1, optionally a neutral lipid, preferably 1,2-distearoyl-sn-glycero-3-phosphocholine (DSPC) and optionally a steroid, preferably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In a particular preferred embodiment, the composition comprises nucleic acid sequence A, B, C, and/or D (of the n nucleic acid sequence set) and, optionally, the m additional nucleic acid sequence, comprises lipid nanoparticles (LNPs), which have a molar ratio of approximately 50:10:38.5:1.5, preferably 47.5:10:40.8:1.7 or more preferably 47.4:10:40.9:1.7 (i.e. proportion (mol %) of cationic lipid, DSPC, cholesterol and an aggregation reducing lipids, preferably polymer conjugated lipid, e.g. PEG-lipid (preferably PEG-lipid).

The total amount of nucleic acid in the lipid nanoparticles may vary and is defined depending on the e.g. nucleic acid to total lipid w/w ratio. In one embodiment of the invention the nucleic acid, in particular the RNA to total lipid ratio is less than 0.06 w/w, preferably between 0.03 w/w and 0.04 w/w.

In some embodiments, the lipid nanoparticles (or liposomes, nanoliposomes, lipoplexes) are composed of only three lipid components, namely imidazole cholesterol ester (ICE), 1,2-dioleoyl-sn-glycero-3-phosphoethanolamine (DOPE), and 1,2-dimyristoyl-sn-glycerol, methoxypolyethylene glycol (DMG-PEG-2K) In preferred embodiments, the lipid nanoparticle (or liposomes, nanoliposomes, lipoplexes) of the composition comprises a cationic lipid, a steroid, a neutral lipid, and an aggregation reducing lipids, preferably a polymer conjugated lipid, more preferably a pegylated lipid. Preferably, the polymer conjugated lipid is a pegylated lipid or PEG-lipid. In a specific embodiment, lipid nanoparticles comprise a cationic lipid resembled by the cationic lipid COATSOME® SS-EC (former name: SS-33/4PE-15; NOF Corporation, Tokyo, Japan), in accordance with the following formula

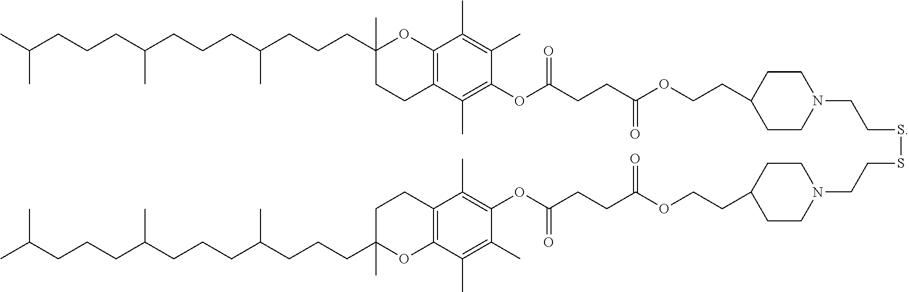 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

As described further below, those lipid nanoparticles are termed “GN01”.

Furthermore, in a specific embodiment, the GN01 lipid nanoparticles comprise a neutral lipid being resembled by the structure 1,2-diphytanoyl-sn-glycero-3-phosphoethanolamine (DPhyPE):

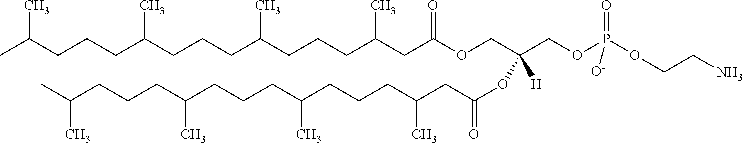 [SMILES: CC(C)CCCC(C)CCCC(C)CCCC(C)CC(=O)OC[C@H](COP(=O)([O-])OCC[NH3+])OC(=O)CC(C)CCCC(C)CCCC(C)CCCC(C)C]

Furthermore, in a specific embodiment, the GN01 lipid nanoparticles comprise a polymer conjugated lipid, preferably a pegylated lipid, being 1,2-dimyristoyl-rac-glycero-3-methoxypolyethylene glycol 2000 (DMG-PEG 2000) having the following structure:

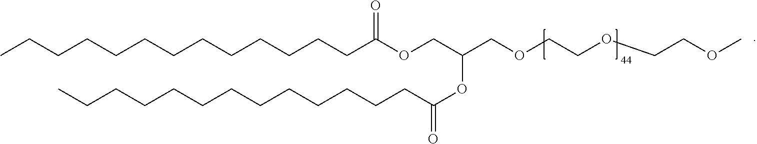

As used in the art, “DMG-PEG 2000” is considered a mixture of 1,2-DMG PEG2000 and 1,3-DMG PEG2000 in ˜97:3 ratio.

Accordingly, GN01 lipid nanoparticles (GN01-LNPs) according to one of the preferred embodiments comprise a SS-EC cationic lipid, neutral lipid DPhyPE, cholesterol, and the aggregation reducing lipids, preferably the polymer conjugated lipid (e.g. pegylated lipid) 1,2-dimyristoyl-rac-glycero-3-methoxypolyethylene glycol (PEG-DMG).

In a preferred embodiment, the GN01 LNPs comprise:
(a) cationic lipid SS-EC (former name: SS-33/4PE-15; NOF Corporation, Tokyo, Japan) at an amount of 45-65 mol %;
(b) cholesterol at an amount of 25-45 mol %;
(c) DPhyPE at an amount of 8-12 mol %; and
(d) PEG-DMG 2000 at an amount of 1-3 mol %;

each amount being relative to the total molar amount of all lipidic excipients of the GN01 lipid nanoparticles.

In a further preferred embodiment, the GN01 lipid nanoparticles as described herein comprises 59 mol % cationic lipid, 10 mol % neutral lipid, 29.3 mol % steroid and 1.7 mol % aggregation reducing lipids, preferably polymer conjugated lipid, e.g. pegylated lipid. In a most preferred embodiment, the GN01 lipid nanoparticles as described herein comprise 59 mol % cationic lipid SS-EC, 10 mol % DPhyPE, 29.3 mol % cholesterol and 1.7 mol % DMG-PEG 2000.

The amount of the cationic lipid relative to that of the nucleic acid in the GN01 lipid nanoparticle may also be expressed as a weight ratio. For example, the GN01 lipid nanoparticles comprise the at least one nucleic acid, preferably the at least one RNA at an amount such as to achieve a lipid to RNA weight ratio in the range of about 20 to about 60, or about 10 to about 50. In other embodiments, the ratio of cationic lipid to nucleic acid or RNA is from about 3 to about 15, such as from about 5 to about 13, from about 4 to about 8 or from about 7 to about 11. In a very preferred embodiment of the present invention, the total lipid/RNA mass ratio is about 40 or 40, i.e. about 40 or 40 times mass excess to ensure RNA encapsulation. Another preferred RNA/lipid ratio is between about 1 and about 10, about 2 and about 5, about 2 and about 4, or preferably about 3.

Further, the amount of the cationic lipid may be selected taking the amount of the nucleic acid cargo such as the nucleic acid cargo (e.g. RNA) compound into account. In one embodiment, the N/P ratio can be in the range of about 1 to about 50. In another embodiment, the range is about 1 to about 20, about 1 to about 10, about 1 to about 5. In one preferred embodiment, these amounts are selected such as to result in an N/P ratio of the GN01 lipid nanoparticles or of the composition in the range from about 10 to about 20. In a further very preferred embodiment, the N/P is 14 (i.e. 14 times mol excess of positive charge to ensure nucleic acid encapsulation).

In a preferred embodiment, GN01 lipid nanoparticles comprise 59 mol % cationic lipid COATSOME® SS-EC (former name: SS-33/4PE-15 as apparent from the examples section; NOF Corporation, Tokyo, Japan), 29.3 mol % cholesterol as steroid, 10 mol % DPhyPE as neutral lipid/phospholipid and 1.7 mol % DMG-PEG 2000 as polymer conjugated lipid. A further inventive advantage connected with the use of DPhyPE is the high capacity for fusogenicity due to its bulky tails, whereby it is able to fuse at a high level with endosomal lipids. For “GN01”, N/P (lipid to nucleic acid, e.g. RNA mol ratio) preferably is 14 and total lipid/RNA mass ratio preferably is 40 (m/m).

In other embodiments, nucleic acid sequence A, B, C, and/or D (of the n nucleic acid sequence set) and, optionally, the m additional nucleic acid sequence is complexed with one or more lipids thereby forming lipid nanoparticles (or liposomes, nanoliposomes, lipoplexes), wherein the LNP (or liposomes, nanoliposomes, lipoplexes) comprises
I. at least one cationic lipid;
Ii. at least one neutral lipid;
Iii. at least one steroid or steroid analogue; and
Iiii. at least one PEG-lipid as defined herein,
wherein the cationic lipid is DLin-KC2-DMA (50 mol %) or DLin-MC3-DMA (50 mol %), the neutral lipid is DSPC (10 mol %), the PEG lipid is PEG-DOMG (1.5 mol %) and the structural lipid is cholesterol (38.5 mol %).

In other embodiments, nucleic acid sequence A, B, C, and/or D (of the n nucleic acid sequence set) and, optionally, the m additional nucleic acid sequence is complexed with one or more lipids thereby forming lipid nanoparticles (LNP), wherein the LNP comprises SS15/Chol/DOPE (or DOPC)/DSG-5000 at mol % 50/38.5/10/1.5.

In other embodiments, nucleic acid sequence A, B, C, and/or D (of the n nucleic acid sequence set) and, optionally, the m additional nucleic acid sequence may be formulated in liposomes, e.g. in liposomes as described in WO2019/222424, WO2019/226925, WO2019/232095, WO2019/232097, or WO2019/232208, the disclosure of WO2019/222424, WO2019/226925, WO2019/232095, WO2019/232097, or WO2019/232208 relating to liposomes or lipid-based carrier molecules herewith incorporated by reference.

In various embodiments, the carrier of the composition that suitably encapsulates nucleic acid sequence A, B, C, and/or D (of the n nucleic acid sequence set) and, optionally, the m additional nucleic acid sequence, in particular the LNPs have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic and/or non-inflammatory. As used herein, the mean diameter may be represented by the z-average as determined by dynamic light scattering as commonly known in the art.

The polydispersity index (PDI) of the nanoparticles (e.g. LNPs) is typically in the range of 0.1 to 0.5. In a particular embodiment, a PDI is below 0.2. Typically, the PDI is determined by dynamic light scattering.

In another preferred embodiment of the invention the nanoparticles (e.g. LNPs) have a hydrodynamic diameter in the range from about 50 nm to about 300 nm, or from about 60 nm to about 250 nm, from about 60 nm to about 150 nm, or from about 60 nm to about 120 nm, respectively.

In another preferred embodiment of the invention the nanoparticles (e.g. LNPs) have a hydrodynamic diameter in the range from about 50 nm to about 300 nm, or from about 60 nm to about 250 nm, from about 60 nm to about 150 nm, or from about 60 nm to about 120 nm, respectively.

According to various suitable embodiments, suitable carriers of the composition may include polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semisynthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic poly conjugates).

In other embodiments, the nucleic acid sequences of the composition may be formulated in amphiphilic macromolecules (AMs). AMs comprise biocompatible amphiphilic polymers which have an alkylated sugar backbone covalently linked to poly(ethylene glycol). In aqueous solution, the AMs self-assemble to form micelles. Non-limiting examples of methods of forming AMs and AMs are described in US Patent Publication No. US20130217753, the contents of which are herein incorporated by reference in its entirety.

In other embodiments, the nucleic acid sequences of the composition may be formulated in inorganic nanoparticles (U.S. Pat. No. 8,257,745, herein incorporated by reference in its entirety). The inorganic nanoparticles may include, but are not limited to, clay substances that are water swellable. As a non-limiting example, the inorganic nanoparticle may include synthetic smectite clays which are made from simple silicates (See e.g., U.S. Pat. Nos. 5,585,108 and 8,257,745 each of which are herein incorporated by reference in their entirety).

In other embodiments, the nucleic acid sequences of the composition may be formulated in water-dispersible nanoparticle comprising a semiconductive or metallic material (U.S. Pub. No. 20120228565; herein incorporated by reference in its entirety) or formed in a magnetic nanoparticle (U.S. Pub. No. 20120265001 and 20120283503; each of which is herein incorporated by reference in its entirety). The water-dispersible nanoparticles may be hydrophobic nanoparticles or hydrophilic nanoparticles.

In other embodiments, the nucleic acid sequences of the composition may be formulated in high density lipoprotein-nucleic acid particles. As a non-limiting example, the particles may comprise a nucleic acid component and a polypeptide comprising a positively charged region which associates with the nucleic acid component as described in U.S. Pat. No. 8,734,853, the contents of which is herein incorporated by reference in its entirety.

In other embodiments, the nucleic acid sequences of the composition may be formulated in a micelle or coated on a micelle for delivery, or may be encapsulated into any hydrogel known in the art which may form a gel when injected into a subject, or may be formulated in and/or delivered using a nanolipogel.

In other embodiments, the nucleic acid sequences of the composition may be formulated in exosomes. The exosomes may be loaded with the nucleic acid of the composition and delivered to cells, tissues and/or organisms. As a non-limiting example, the nucleic acid may be loaded in exosomes described in International Publication No. WO2013084000, herein incorporated by reference in its entirety. In embodiments, the exosome are obtained from cells that have been induced to undergo oxidative stress such as, but not limited to, the exosomes described in International Patent Publication No. WO2014028763, the contents of which are herein incorporated by reference in its entirety.

Accordingly, the pharmaceutically acceptable carrier as used herein preferably includes the liquid or non-liquid basis of the inventive composition. If the inventive composition is provided in liquid form, the carrier will be water, typically pyrogen-free water; isotonic saline or buffered (aqueous) solutions, e.g. phosphate, citrate etc. buffered solutions. Preferably, Ringer- or Ringer-Lactate solution as described in WO2006/122828 is used as a liquid basis for the composition for use according to the invention.

As outlined above, in embodiments, the composition described herein may be lyophilized in order to improve storage stability of the composition. A lyoprotectant for lyophilization and/or spray (freeze) drying may be selected from trehalose, sucrose, mannose, dextran and inulin. A preferred lyoprotectant is sucrose, optionally comprising a further lyoprotectant. A further preferred lyoprotectant is trehalose, optionally comprising a further lyoprotectant.

Accordingly, in preferred embodiments, the composition is a lyophilized composition, a spray-dried composition, or a spray-freeze dried composition, optionally comprising at least one pharmaceutically acceptable lyoprotectant.

In preferred embodiments, the composition of the first aspect comprises (i) at least one, preferably n nucleic acid sequence set (nucleic acid sequence A, B, C, and/or D) as defined herein, and, optionally, (ii) m additional nucleic acid sequences, wherein said nucleic acid sequences are formulated and/or complexed as defined above, wherein administration of the composition to a cell or to a subject leads to expression of at least two assembled antibodies in said cell or subject, wherein, preferably, at least about 70%, at least about 75%, at least about 80%, at least about 85%, at least about 90%, at least about 95%, or at least about 100% of the expressed at least two antibodies are (correctly) assembled antibodies.

Preferably, administration of the composition to a cell leads to expression of at least two assembled antibodies in the same cell, wherein, preferably, at least about 70%, at least about 75%, at least about 80%, at least about 85%, at least about 90%, at least about 95%, or at least about 100% of the expressed at least two antibodies are (correctly) assembled antibodies.

In embodiments where the composition comprises RNA, the composition comprises at least one antagonist of at least one RNA sensing pattern recognition receptor. Such an antagonist may preferably be co-formulated in lipid-based carriers as defined herein.

Suitable antagonist of at least one RNA sensing pattern recognition receptor are disclosed in PCT patent application PCT/EP2020/072516, the full disclosure herewith incorporated by reference. In particular, the disclosure relating to suitable antagonist of at least one RNA sensing pattern recognition receptors as defined in any one of the claims 1 to 94 of PCT/EP2020/072516 are incorporated.

In preferred embodiments, the composition comprises at least one antagonist of at least one RNA sensing pattern recognition receptor selected from a Toll-like receptor, preferably TLR7 and/or TLR8.

In embodiments, the at least one antagonist of at least one RNA sensing pattern recognition receptor is selected from a nucleotide, a nucleotide analog, a nucleic acid, a peptide, a protein, a small molecule, a lipid, or a fragment, variant or derivative of any of these.

In preferred embodiments, the at least one antagonist of at least one RNA sensing pattern recognition receptor is a single stranded oligonucleotide, preferably a single stranded RNA Oligonucleotide.

In embodiments, the antagonist of at least one RNA sensing pattern recognition receptor is a single stranded oligonucleotide that comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-212 of PCT/EP2020/072516, or fragments of any of these sequences.

A particularly preferred antagonist of at least one RNA sensing pattern recognition receptor in the context of the invention is 5′-GAG CGmG CCA-3′ (SEQ ID NO: 85 of PCT/EP2020/072516), or a fragment thereof.

In preferred embodiments of the first aspect, the composition comprises n nucleic acid sequence sets encoding at least one antibody or a fragment or variant thereof, wherein the n different nucleic acid sequence sets comprise
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody heavy chain B (HC-B), or a fragment or variant thereof,
wherein the at least one coding sequence of the nucleic acid sequence A and/or the nucleic acid sequence B encodes at least one antibody chain assembly promoter, wherein the composition is for expression of at least two assembled antibodies in vivo. Optionally, the composition comprises m additional nucleic acid sequences comprising at least one coding sequence encoding at least one antibody or a fragment of an antibody or a variant of an antibody. In such embodiments, the nucleic acid sequence A, B, C, and/or D and, optionally, the m additional nucleic acid sequence are complexed or associated with one or more lipids, thereby forming LNPs that comprise or consist of
   i. at least one cationic or cationizable lipid;
   ii. at least one a neutral lipid;
   iii. at least one a steroid or steroid analogue;
   iv. at least one aggregation reducing lipids, preferably polymer conjugated lipid.

In preferred embodiments of the first aspect, the composition comprises n RNA sequence sets encoding at least one antibody or a fragment or variant thereof, wherein the n different RNA sequence sets comprise
a) RNA sequence A comprising at least one coding sequence encoding at least one antibody heavy chain A (HC-A), or a fragment or variant thereof, and
b) RNA sequence B comprising at least one coding sequence encoding at least one antibody heavy chain B (HC-B), or a fragment or variant thereof,
wherein the at least one coding sequence of the RNA sequence A and/or the RNA sequence B encodes at least one antibody chain assembly promoter, wherein the composition is for expression of at least two assembled antibodies in vivo. Optionally, the composition comprises m additional nucleic acid sequences comprising at least one coding sequence encoding at least one antibody or a fragment of an antibody or a variant of an antibody.

In such embodiments, the RNA sequence A, B, C, and/or D and, optionally, the m additional RNA sequence are complexed or associated with one or more lipids, thereby forming LNPs that comprise or consist of
i. at least one cationic or cationizable lipid;
ii. at least one a neutral lipid;
iii. at least one a steroid or steroid analogue;
iv. at least one aggregation reducing lipids, preferably polymer conjugated lipid.

In preferred embodiments of the first aspect, the composition comprises n nucleic acid sequence sets encoding at least one antibody or a fragment or variant thereof, wherein the n different nucleic acid sequence sets comprise
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody heavy chain B (HC-B), or a fragment or variant thereof,
wherein the at least one coding sequence of the nucleic acid sequence A and/or the nucleic acid sequence B encodes at least one antibody chain assembly promoter,
wherein antibody heavy chain A (HC-A) and antibody heavy chain B (HC-B) comprises at least one HC-HC assembly promoter pair comprising the following amino acid substitutions:
   HC-HC-PP3: S354C, T366W on HC-A; Y349C, T366S, L368A, Y407V on HC-B
   HC-HC-PP4: S364H, F405A on HC-A; Y349T, T394F on HC-B
   HC-HC-PP5: T350V, L351Y, F405A, Y407V on HC-A; T350V, T366L, K392L, T394W on HC-B
   HC-HC-PP18: Y349S, T366M, K370Y, K409V on HC-A; E/D356G, E357D, S364Q, Y407A on HC-B,

preferably, wherein the composition is for expression of at least two assembled antibodies in vivo. Optionally, the composition comprises m additional nucleic acid sequences comprising at least one coding sequence encoding at least one antibody or a fragment of an antibody or a variant of an antibody.

In such embodiments, the nucleic acid sequence A, B, C, and/or D and, optionally, the m additional nucleic acid sequence are complexed or associated with one or more lipids, thereby forming LNPs that comprise or consist of
i. at least one cationic or cationizable lipid;
ii. at least one a neutral lipid;
iii. at least one a steroid or steroid analogue;
iv. at least one aggregation reducing lipids, preferably polymer conjugated lipid.

In preferred embodiments of the first aspect, the composition comprises n RNA sequence sets encoding at least one antibody or a fragment or variant thereof, wherein the n different RNA sequence sets comprise
a) RNA sequence A comprising at least one coding sequence encoding at least one antibody heavy chain A (HC-A), or a fragment or variant thereof, and
b) RNA sequence B comprising at least one coding sequence encoding at least one antibody heavy chain B (HC-B), or a fragment or variant thereof,
wherein the at least one coding sequence of the RNA sequence A and/or the RNA sequence B encodes at least one antibody chain assembly promoter,
wherein antibody heavy chain A (HC-A) and antibody heavy chain B (HC-B) comprises at least one HC-HC assembly promoter pair comprising the following amino acid substitutions:
   HC-HC-PP3: S354C, T366W on HC-A; Y349C, T366S, L368A, Y407V on HC-B
   HC-HC-PP4: S364H, F405A on HC-A; Y349T, T394F on HC-B
   HC-HC-PP5: T350V, L351Y, F405A, Y407V on HC-A; T350V, T366L, K392L, T394W on HC-B
   HC-HC-PP18: Y349S, T366M, K370Y, K409V on HC-A; E/D356G, E357D, S364Q, Y407A on HC-B,
wherein the composition is for expression of at least two assembled antibodies in vivo. Optionally, the composition comprises m additional nucleic acid sequences comprising at least one coding sequence encoding at least one antibody or a fragment of an antibody or a variant of an antibody. In such embodiments, the RNA sequence A, B, C, and/or D and, optionally, the m additional RNA sequence are complexed or associated with one or more lipids, thereby forming LNPs that comprise or consist of
   i. at least one cationic or cationizable lipid;
   ii. at least one a neutral lipid;
   iii. at least one a steroid or steroid analogue;
   iv. at least one aggregation reducing lipids, preferably polymer conjugated lipid.

Nucleic Acid Sequence Set

In a second aspect, the present invention relates inter alia to a nucleic acid sequence set that encodes an antibody, or a fragment of an antibody, or a variant of an antibody. Notably, features and embodiments described in the context of the composition of the first aspect (the composition comprising at least one nucleic acid sequence set) may likewise be applied to the nucleic acid set of the second aspect.

In the following, particularly preferred embodiments of the nucleic acid sequence set of the second aspect are provided as an item list. These items are preferred embodiments, and have to be read in conjunction with definitions also provided in the context of the first aspect.

Item 1: A nucleic acid sequence set encoding an antibody or a fragment or variant of an antibody, comprising
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody chain heavy B (HC-B), or a fragment or variant thereof,
wherein the at least one coding sequence of the nucleic acid sequence A and/or the nucleic acid sequence B encodes at least one antibody chain assembly promoter.

Preferably the nucleic acid sequence set of Item 1 is selected from any one of the nucleic acid sequence sets as described in the context of the first aspect.

Item 2: Nucleic acid sequence set of Item 1, wherein the at least one antibody chain assembly promoter is a moiety that promotes, supports, forces, or directs assembly of at least two antibody chains, preferably wherein the moiety comprises at least one amino acid in a position that does not occur naturally, or amino acid sequence that does not occur naturally.

Item 3: Nucleic acid sequence set of Item 1 or 2, wherein the at least one antibody chain assembly promoter is a moiety that prevents or reduces assembly of HC-A and/or HC-B to a wild-type (unmodified) antibody heavy chain, preferably to a wild-type (unmodified) antibody heavy chain selected or derived from a human.

Item 4: Nucleic acid sequence set of Item 1 to 3, wherein the antibody or antibody fragment or variant thereof is derived or selected from a monoclonal antibody or fragments thereof, a chimeric antibody or fragments thereof, a human antibody or fragments thereof, a humanized antibody or fragments thereof, an intrabody or fragments thereof, a single chain antibody or fragments thereof.

Item 5: Nucleic acid sequence set of Item 1 to 4, wherein the antibody or antibody fragment or variant thereof encoded by the nucleic acid set is derived or selected from an IgG1, IgG2, IgG3, IgG4, IgD, IgA1, IgA2, IgE, IgM, IgNAR, hclgG, BiTE, diabody, DART, VHH or VNAR-Fragment, TandAb, scDiabody; sc-Diabody-CH3, Diabody-CH3, Triple Body, mini antibody, minibody, nanobody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, Tandem scFv-Fc, Fab, Fab′, Fc, Facb, pFc′, Fd, Fv, scFv antibody fragment scFv-Fc, or scFab-Fc, preferably IgG1, IgG3, scFv-Fc or scFab-Fc.

Item 6: Nucleic acid sequence set of Item 1 to 5, wherein the antibody or antibody fragment specifically recognizes and/or binds to at least one target. In preferred embodiments, a target may be selected from at least one epitope or at least one antigen.

Item 7: Nucleic acid sequence set of Item 1 to 6, wherein the antibody or antibody fragment encoded by the nucleic acid set specifically recognizes and/or binds to at least one target selected from at least one tumor antigen or epitope, at least one antigen or epitope of a pathogen, at least one viral antigen or epitope, at least one bacterial antigen or epitope, at least one protozoan antigen or epitope, at least one antigen or epitope of a cellular signalling molecule, at least one antigen or epitope of a component of the immune system, at least one antigen or epitope of an intracellular protein, or any combination thereof. Preferably, the at least one antibody or antibody fragment specifically recognizes and/or binds to at least one antigen or epitope of a pathogen (e.g. bacteria or virus).

Item 8: Nucleic acid sequence set of Item 1 to 7, wherein the nucleic acid sequence set encodes an antibody or a fragment or variant of an antibody, wherein antibody or antibody fragment is derived or selected from a monospecific antibody or fragment or variant thereof, or a multispecific antibody or fragment or variant thereof.

Item 9: Nucleic acid sequence set of Item 1 to 8, wherein the nucleic acid sequence set encodes an antibody or a fragment or variant of an antibody, wherein the multispecific antibody is derived or selected from a bispecific, trispecific, tetraspecific, pentaspecific, or a hexaspecific antibody or a fragment or variant of any of these.

Item 10: Nucleic acid sequence set of Item 1 to 9, wherein the nucleic acid sequence set encodes at least one antibody heavy chain A and at least one antibody heavy chain B, wherein heavy chain A and/or heavy chain B is derived or selected from antibody heavy chains selected from IgG1, IgG2, IgG3, IgG4, IgD, IgA1, IgA2, IgE, or IgM, or an allotype, an isotype, or mixed isotype or a fragment or variant of any of these. Preferably the at least one HC-A and/or the at least one HC-B is derived or selected from antibody heavy chains selected from IgG1 and/or IgG3.

Item 11: Nucleic acid sequence set of Item 1 to 10, wherein the at least one HC-A and/or the at least one HC-B is derived or selected from an antibody heavy chain of IgG, or an allotype or an isotype thereof, preferably an antibody heavy chain of IgG1 or an allotype or an isotype thereof.

Item 12: Nucleic acid sequence set of Item 1 to 11, wherein the at least one HC-A and/or the at least one HC-B is derived or selected from an antibody heavy chain of IgG, preferably an antibody heavy chain of IgG1 or an allotype or an isotype thereof, wherein the antibody heavy chain of IgG, preferably IgG1, is selected from G1m17, G1m3, G1m1 and G1m2, G1m27, G1m28, nG1m17, nG1 m1, or any combination thereof.

Item 13: Nucleic acid sequence set of Item 11 or 12, wherein the antibody heavy chain IgG, preferably IgG1 is selected or is derived from allotype G1m3,1 (R120, D12/L14).

Item 14: Nucleic acid sequence set of Item 1 to 13, wherein the at least one antibody chain assembly promoter is a heavy chain-heavy chain (HC-HC) assembly promoter and/or a heavy chain-light chain (HC-LC) assembly promoter.

Item 15: Nucleic acid sequence set of Item 14, wherein the at least one HC-HC assembly promoter is located in the constant region of antibody heavy chain A and/or antibody heavy chain B. Preferably, at least one HC-HC assembly promoter is located in the constant region of antibody heavy chain A and antibody heavy chain B.

Item 16: Nucleic acid sequence set of Item 14 or 15, wherein the at least one HC-HC assembly promoter is located in the Fc region of antibody heavy chain A and/or antibody heavy chain B. Preferably, at least one HC-HC assembly promoter is located in the Fc region of antibody heavy chain A and antibody heavy chain B.

Item 17: Nucleic acid sequence set of Item 14 to 16, wherein the at least one HC-HC assembly promoter is located in the CH3 domain of antibody heavy chain A and/or antibody heavy chain B. Preferably, at least one HC-HC assembly promoter is located in the CH3 domain of antibody heavy chain A and antibody heavy chain B.

Item 18: Nucleic acid sequence set of Item 14 to 17, wherein the at least one HC-HC assembly promoter comprises at least one amino acid substitution in an amino acid sequence of a CH3-CH3 assembly interface of antibody heavy chain A and/or antibody heavy chain B.

Item 19: Nucleic acid sequence set of Item 14 to 18, wherein the at least one HC-HC assembly promoter comprises or consists of at least one selected from steric assembly element, electrostatic element assembly element, SEED assembly element, DEEK assembly element, interchain disulfides assembly element, or any combination thereof. In particularly preferred embodiments, the at least one HC-HC assembly promoter comprises or consists of at least one steric assembly element. In particularly preferred embodiments, the at least one HC-HC assembly promoter does not comprises or consists of at least one electrostatic steering assembly element.

Item 20: Nucleic acid sequence set of Item 14 to 19, wherein the at least one HC-HC assembly promoter comprises at least one amino acid substitution in the CH3 region.

Item 21: Nucleic acid sequence set of Item 14 to 20, wherein the at least one HC-HC assembly promoter comprises or consists of at least one steric assembly element.

Item 22: Nucleic acid sequence set of Item 21, wherein the at least one steric assembly element comprises a modification selected from at least one knob-modification and/or at least one hole modification.

Item 23: Nucleic acid sequence set of Item 22, wherein the at least one steric assembly element as specified herein comprises a modification selected from at least one knob-modification wherein, preferably, the at least one knob-modification is at least one amino acid substitution in a CH3-CH3 assembly interface.

Item 24: Nucleic acid sequence set of Item 22, wherein the at least one steric assembly element as specified herein comprises a modification selected from at least one hole-modification wherein, preferably, the at least one hole-modification is at least one amino acid substitution in a CH3-CH3 assembly interface.

Item 25: Nucleic acid sequence set of Item 14 to 22, wherein the at least one coding sequence of nucleic acid sequence A encodes at least one HC-HC assembly promoter and the at least one coding sequence of nucleic acid sequence B encodes at least one HC-HC assembly promoter.

Item 26: Nucleic acid sequence set of Item 25, wherein the at least one HC-HC assembly promoter of HC-A comprises at least one knob-modification and the at least one HC-HC assembly promoter of HC-B comprises at least one hole modification.

Item 27: Nucleic acid sequence set of Item 14 to 26, wherein HC-A and HC-B comprise at least one HC-HC assembly promoter pair comprising the following amino acid substitutions (numbering according to EU numbering of the CH3 domain; see also Table 1 of the first aspect):
HC-HC-PP 1: T366Y on HC-A; Y407T on HC-B
HC-HC-PP 2: T366W on HC-A; 366S, L368A, Y407V on HC-B
HC-HC-PP 3: S354C, T366W on HC-A; Y349C, T366S, L368A, Y407V on HC-B
HC-HC-PP 4: S364H, F405A on HC-A; Y349T, T394F on HC-B
HC-HC-PP 5: T350V, L351Y, F405A, Y407V on HC-A; T350V, T366L, K392L, T394W on HC-B
HC-HC-PP 6: K409D on HC-A; D399K on HC-B
HC-HC-PP 7: K409D on HC-A; D399R on HC-B
HC-HC-PP 8: K409E on HC-A; D399R on HC-B
HC-HC-PP 9: K409E on HC-A; D399K on HC-B
HC-HC-PP 10: K392D, K409D on HC-A; E/D356K, D399K on HC-B
HC-HC-PP 11: D221E, P228E, L368E on HC-A; D221R, P228R, K409R on HC-B
HC-HC-PP 12: K360E, K409W on HC-A; Q347R, D399V, F405T on HC-B
HC-HC-PP 13: Y349C, K360E, K409W on HC-A; Q347R, S354C, D399V, F405T on HC-B
HC-HC-PP 14: L351 L/K, T366K on HC-A; Y349D/E, R355D/E on HC-B
HC-HC-PP 15: L351L/K, T366K on HC-A; Y349D/E, L351D/E, R355D/E, L368D/E (only one) on HC-B
HC-HC-PP 16: F405L on HC-A; K409R on HC-B
HC-HC-PP 17: K360D, D399M, Y407A on HC-A; E345R, Q347R, T366V, K409V on HC-B
HC-HC-PP 18: Y349S, T366M, K370Y, K409V on HC-A; E/D356G, E357D, S364Q, Y407A on HC-B

Item 28A: Nucleic acid sequence set of Item 14 to 27, wherein HC-A and HC-B comprise at least one HC-HC assembly promoter pair comprising the following amino acid substitutions (numbering according to EU numbering of the CH3 domain; see also Table 1 of the first aspect):
HC-HC-PP3: S354C, T366W on HC-A; Y349C, T366S, L368A, Y407V on HC-B
HC-HC-PP4: S364H, F405A on HC-A; Y349T, T394F on HC-B
HC-HC-PP5: T350V, L351Y, F405A, Y407V on HC-A; T350V, T366L, K392L, T394W on HC-B
HC-HC-PP18: Y349S, T366M, K370Y, K409V on HC-A; E/D356G, E357D, S364Q, Y407A on HC-B

Item 28B: Nucleic acid sequence set of Item 14 to 28A, antibody heavy chain A (HC-A) and antibody heavy chain B (HC-B) encoded by the nucleic acid sequence set comprises at least one HC-HC assembly promoter pair comprising the following amino acid sequence stretch in the CH3 domain, being identical or at least 90%, 95%, 96%, 97%, 98%, 99% identical to the following amino acid sequences:
HC-HC-PP3: SEQ ID NO: 104 on HC-A; SEQ ID NO: 105 on HC-B
HC-HC-PP4: SEQ ID NO: 106 on HC-A; SEQ ID NO: 107 on HC-B
HC-HC-PP5: SEQ ID NO: 108 on HC-A; SEQ ID NO: 109 on HC-B
HC-HC-PP18: SEQ ID NO: 110 on HC-A; SEQ ID NO: 111 on HC-B

Item 29: Nucleic acid sequence set of Item 1 to 28, wherein the coding sequence of nucleic acid sequence A additionally encodes at least one fragment selected or derived from an antibody light chain A (LC-A) or a variant thereof and/or wherein the coding sequence of nucleic acid sequence B additionally encodes at least one fragment selected or derived from an antibody light chain B (LC-B) or a variant thereof.

Item 30: Nucleic acid sequence set of Item 29, wherein the at least one LC-A and/or the at least one LC-B is selected or derived from a κ light chain or λ light chain or a fragment or variant thereof.

Item 31: Nucleic acid sequence set of Item 29 or 30, wherein the at least one LC-A fragment or variant is N-terminally or C-terminally fused to HC-A, preferably fused to the variable region of HC-A, and/or wherein the at least one LC-B fragment or variant is N-terminally or C-terminally fused to HC-B, preferably fused to the variable region of HC-B.

Item 32: Nucleic acid sequence set of Item 29 to 31, wherein the LC-A fragment or variant is a variable region of an antibody light chain or a fragment thereof and/or wherein the LC-B fragment or variant is a variable region of an antibody light chain or a fragment thereof.

Item 33: Nucleic acid sequence set of Item 29 to 32, wherein a variable region of LC-A is fused to the variable region of HC-A, optionally via a linker peptide element, and/or wherein a variable region of LC-B is fused to the variable region of HC-B, optionally via a linker peptide element.

In preferred embodiments, the nucleic acid sequence set of any one of the preceding Items comprises
a) nucleic acid sequence A comprising at least one coding sequence encoding
   at least one HC-A, or a fragment or variant thereof, and
   at least one HC-HC assembly promoter as defined herein, and
   at least one LC-A, or a fragment or variant thereof,

preferably, wherein the variable region of LC-A is fused to the variable region of HC-A;
b) nucleic acid sequence B comprising at least one coding sequence encoding
   at least one HC-B, or a fragment or variant thereof, and
   at least one HC-HC assembly promoter as defined herein, and
   at least one LC-B, or a fragment or variant thereof,

preferably, wherein the variable region of LC-B is fused to the variable region of HC-B.

Item 34: Nucleic acid sequence set of Item 1 to 33, wherein at least one antibody chain assembly promoter of nucleic acid sequence A and/or the nucleic acid sequence B is selected from a heavy chain-light chain (HC-LC) assembly promoter.

Item 35: Nucleic acid sequence set of Item 34, wherein the at least one HC-LC assembly promoter is located in the constant region of HC-A and/or HC-B.

Item 36: Nucleic acid sequence set of Item 34 or 35, wherein the at least one HC-LC assembly promoter is located in the Fab region of HC-A and/or HC-B.

Item 37: Nucleic acid sequence set of Item 34 to 36, wherein the at least one HC-LC assembly promoter is located in the CH1 domain of HC-A and/or HC-B.

Item 38: Nucleic acid sequence set of Item 34 to 37, wherein the at least one HC-LC assembly promoter comprises at least one amino acid substitution in an amino acid sequence of the HC-LC assembly interface.

Item 39: Nucleic acid sequence set of Item 34 to 38, wherein the at least one HC-LC assembly promoter comprises or consists of at least one selected from steric assembly element, electrostatic steering assembly element, SEED assembly element, DEEK assembly element, interchain disulfides assembly element, or any combination thereof.

Item 40: Nucleic acid sequence set of Item 1 to 39, wherein the nucleic acid sequence set additionally comprises,
c) nucleic acid sequence C comprising at least one coding sequence encoding at least one LC-A, or a fragment or variant thereof, and/or
d) nucleic acid sequence D comprising at least one coding sequence encoding at least one LC-B), or a fragment or variant thereof.

Item 41: Nucleic acid sequence set of Item 40, wherein the antibody light chain encoded by nucleic acid sequence C and/or nucleic acid sequence D is selected or derived from a κ light chain or a λ light chain.

Item 42: Nucleic acid sequence set of Item 40 or 41, wherein the at least one coding sequence of nucleic acid sequence C and/or nucleic acid sequence D encodes at least one light chain-heavy chain (LC-HC) assembly promoter.

Item 43: Nucleic acid sequence set of Item 42, wherein the at least one LC-HC assembly promoter is located in the constant region of LC-A and/or LC-B.

Item 44: Nucleic acid sequence set of Item 42 or 43, wherein the at least one LC-HC assembly promoter is located in the Fab region of LC-A and/or LC-B.

Item 45: Nucleic acid sequence set of Item 42 or 44, wherein the at least one LC-HC assembly promoter is located in the CL domain of LC-A and/or LC-B.

Item 46: Nucleic acid sequence set of Item 42 or 45, wherein the at least one LC-HC assembly promoter comprises at least one amino acid substitution in an amino acid sequence of the LC-HC assembly interface.

Item 47: Nucleic acid sequence set of Item 42 or 46, wherein the at least one LC-HC assembly promoter comprises or consists of at least one selected from steric assembly element, electrostatic steering assembly element, SEED assembly element, DEEK assembly element, interchain disulfides assembly element, or any combination thereof.

In preferred embodiments, the nucleic acid sequence set of any one of the preceding Items comprises
a) nucleic acid sequence A comprising at least one coding sequence encoding
   at least one HC-A, or a fragment or variant thereof,
   at least one HC-HC assembly promoter, and
   at least one HC-LC assembly promoter;
b) nucleic acid sequence B comprising at least one coding sequence encoding
   at least one HC-B, or a fragment or variant thereof,
   at least one HC-HC assembly promoter, and
   at least one HC-LC assembly promoter;
c) nucleic acid sequence C comprising at least one coding sequence encoding
   at least one LC-A, or a fragment or variant thereof, and
   at least one LC-HC assembly promoter;
d) nucleic acid sequence D comprising at least one coding sequence encoding
   at least one LC-B, or a fragment or variant thereof, and
   at least one LC-HC assembly promoter.

Item 48: Nucleic acid sequence set of Item 1 to 47, wherein administration of the nucleic acid sequence set to a cell or to a subject leads to (i) expression of at least one HC-A, or a fragment or variant thereof, and (ii) expression of at least one HC-B, or a fragment or variant thereof, and, optionally (iii) expression of at least one LC-A, or a fragment or variant thereof, and, optionally (iv) expression of at least one LC-B, or a fragment or variant thereof in said cell or said subject. Suitably, the subject is a human subject.

Item 49: Nucleic acid sequence set of Item 1 to 48, wherein administration of the nucleic acid sequence set to a cell or to a subject leads to expression one assembled antibody in said cell or subject, preferably, wherein at least about 70%, at least about 75%, at least about 80%, at least about 85%, at least about 90%, at least about 95%, or at least about 100% of the expressed antibody is a correctly assembled antibody. Preferably, mass spectrometry (MS) can be used to determine the percentage of assembled antibodies and misassembled antibodies

Item 50: Nucleic acid sequence set of Item 1 to 49, wherein nucleic acid sequence A, B, C, and/or D is a monocistronic nucleic acid, a bicistronic nucleic acid, or multicistronic nucleic acid.

Item 51: Nucleic acid sequence set of Item 1 to 50, wherein the at least one coding sequence of nucleic acid sequence A, B, C, and/or D is a codon modified coding sequence, preferably wherein the amino acid sequence encoded by the at least one codon modified coding sequence is not being modified compared to the amino acid sequence encoded by the corresponding wild type or reference coding sequence.

Item 52: Nucleic acid sequence set of Item 51, wherein the codon modified coding sequence is selected from C maximized coding sequence, CAI maximized coding sequence, human codon usage adapted coding sequence, G/C content modified coding sequence, and G/C optimized coding sequence, or any combination thereof.

Item 53: Nucleic acid sequence set of Item 51 or 52, wherein the codon modified coding sequence is a G/C optimized coding sequence, a human codon usage adapted coding sequence, or a G/C content modified coding sequence.

Item 54: Nucleic acid sequence set of Item 1 to 53, wherein nucleic acid sequence A, B, C, and/or D comprises at least one untranslated region, preferably at least one heterologous untranslated region (UTR).

Item 55: Nucleic acid sequence set of Item 54, wherein the at least one heterologous untranslated region is selected from at least one heterologous 5′-UTR and/or at least one heterologous 3′-UTR.

Item 56: Nucleic acid sequence set of Item 55, wherein the at least one heterologous 3′-UTR comprises or consists a nucleic acid sequence selected or derived from a 3′-UTR of a gene selected from PSMB3, ALB7, alpha-globin, CASP1, COX6B1, GNAS, NDUFA1 and RPS9, or from a homolog, a fragment or a variant of any one of these genes.

Item 57: Nucleic acid sequence set of Item 55, wherein the at least one heterologous 5′-UTR comprises or consists of a nucleic acid sequence selected or derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

Item 58: Nucleic acid sequence set of Item 1 to 57, wherein nucleic acid sequence A, B, C, and/or D comprises at least one poly(A) sequence, preferably comprising about 30 to about 200 adenosine nucleotides.

Item 59: Nucleic acid sequence set of Item 1 to 58, wherein nucleic acid sequence A, B, C, and/or D comprises at least one poly(C) sequence, preferably comprising about 10 to about 40 cytosine nucleotides.

Item 60: Nucleic acid sequence set of Item 1 to 59, wherein nucleic acid sequence A, B, C, and/or D comprises at least one histone stem-loop or histone stem-loop structure.

Item 61: Nucleic acid sequence set of Item 1 to 60, wherein nucleic acid sequence A, B, C, and/or D is a DNA or an RNA.

Item 62: Nucleic acid sequence set of Item 1 to 61, wherein nucleic acid sequence A, B, C, and/or D is a coding RNA.

Item 63: Nucleic acid sequence set of Item 62, wherein the coding RNA is an mRNA, a self-replicating RNA, a circular RNA, or a replicon RNA, preferably mRNA.

Item 64: Nucleic acid sequence set of Item 1 to 63, wherein nucleic acid sequence A, B, C, and D are mRNA constructs.

Item 65: Nucleic acid sequence set of Item 1 to 64, wherein nucleic acid sequence A, B, C, and D comprises a 5′-cap structure, preferably m7G, cap0, cap1, cap2, a modified cap0 or a modified cap1 structure.

Item 66: Nucleic acid sequence set of Item 1 to 65, wherein nucleic acid sequence A, B, C, and D comprises at least one modified nucleotide preferably selected from pseudouridine (ψ) and/or N1-methylpseudouridine (m1ψ).

Item 67: Nucleic acid sequence set of Item 1 to 66, wherein nucleic acid sequence A, B, C, and/or D is formulated separately.

Item 68: Nucleic acid sequence set of Item 1 to 66, wherein nucleic acid sequence A, B, C, and/or D are co-formulated.

Item 69: Nucleic acid sequence set of Item 1 to 68, wherein nucleic acid sequence A, B, C, and/or D is complexed or associated with or at least partially complexed or partially associated with one or more cationic or polycationic compound.

Item 70: Nucleic acid sequence set of Item 69, wherein the one or more cationic or polycationic compound is selected from a cationic or polycationic polymer, cationic or polycationic polysaccharide, cationic or polycationic lipid, cationic or polycationic protein, cationic or polycationic peptide, or any combinations thereof.

Item 71: Nucleic acid sequence set of Item 69 or 70, wherein the one or more cationic or polycationic peptides are selected from SEQ ID NOs: 75 to 79 peptides for complexation, or any combinations thereof.

Item 72: Nucleic acid sequence set of Item 69 to 71, wherein the cationic or polycationic polymer is a polyethylene glycol/peptide polymer comprising HO-PEG5000-S-(S-CHHHHHHRRRRHHHHHHC-S-)7-S-PEG5000-OH (SEQ ID NO: 75 of the peptide monomer) and/or wherein the cationic or polycationic polymer is a polyethylene glycol/peptide polymer comprising HO-PEG5000-S-(S-CGHHHHHRRRRHHHHHGC-S-)4-S-PEG5000-OH (SEQ ID NO: 79 of the peptide monomer), preferably comprising a lipid component or a lipidoid component.

Item 72: Nucleic acid sequence set of Item 1 to 72, wherein nucleic acid sequence A, B, C, and/or D is complexed or associated with one or more lipids, thereby forming lipid-based carrier including liposomes, lipid nanoparticles (LNP), lipoplexes, and/or nanoliposomes, preferably lipid nanoparticles (LNP).

Item 73: Nucleic acid sequence set of Item 1 to 72, wherein nucleic acid sequence A, B, C, and/or D is formulated in separate liposomes, lipid nanoparticles (LNP), lipoplexes, and/or nanoliposomes.

Item 74: Nucleic acid sequence set of Item 1 to 72, wherein nucleic acid sequence A, B, C, and/or D are co-formulated in liposomes, lipid nanoparticles (LNP), lipoplexes, and/or nanoliposomes.

Item 75: Nucleic acid sequence set of Item 72 to 74, wherein the liposomes, lipid nanoparticles (LNP), lipoplexes, and/or nanoliposomes comprises at least one cationic or cationizable lipid.

Item 76: Nucleic acid sequence set of Item 72 to 75, wherein the liposomes, lipid nanoparticles (LNP), lipoplexes, and/or nanoliposomes comprises at least one aggregation reducing lipid, preferably polymer conjugated lipid, e.g.

PEG conjugated lipid.

Item 77: Nucleic acid sequence set of Item 72 to 76, wherein the liposomes, lipid nanoparticles (LNP), lipoplexes, and/or nanoliposomes comprises one or more neutral lipids and/or one or more steroid or steroid analogues.

Item 78: Nucleic acid sequence set of Item 72 to 77, wherein the liposome, lipid nanoparticle (LNP), lipoplex, and/or nanoliposome, preferably the LNP comprises or consists of
i. at least one cationic or cationizable lipid;
ii. at least one a neutral lipid;
iii. at least one a steroid or steroid analogue;
iv. at least one aggregation reducing lipid, preferably polymer conjugated lipid, e.g. a PEG-lipid,

preferably wherein (i) to (iv) are in a molar ratio of about 20-60% cationic or cationizable lipid, 5-25% neutral lipid, 25-55% sterol, and 0.5-15% polymer-conjugated lipid.

Item 78: Nucleic acid sequence set of Item 1 to 78, wherein administration to a cell or to a subject leads to expression of one assembled antibody in said cell or subject, wherein, preferably, at least about 70%, at least about 75%, at least about 80%, at least about 85%, at least about 90%, at least about 95%, or at least about 100% of the expressed antibody is (correctly) assembled. Preferably, mass spectrometry (MS) can be used to determine the percentage of assembled antibodies and misassembled antibodies Item 79: Nucleic acid sequence set of Item 1 to 78, suitable for administration to a cell or a subject and/or suitable for a medical application.

Item 80: Nucleic acid sequence set of Item 1 to 79, suitable for in vivo administration to a human subject.

Combination of Nucleic Acid Sequence Sets

In a third aspect, the present invention relates inter alia to combination of nucleic acid sequence sets, wherein each set encodes an antibody, or a fragment of an antibody, or a variant of an antibody. Notably, features and embodiments described in the context of the composition of the first aspect (the composition comprising n nucleic acid sequence sets), and the nucleic acid set of the second aspect may likewise be applied to the combination of the third aspect.

Item 80: Combination comprising n different nucleic acid sequence sets according to Items 1 to 79 of the second aspect or compositions of the first aspect, wherein the n nucleic acid sequence sets or compositions are separate entities and, optionally, administered as n separate entities, preferably wherein n is an integer of 1 to 20, preferably 2 to 10, e.g. 2, 3, 4, 5, 6, 7, 8, 9, 10, 11, 12, 13, 14, 15, 16, 17, 18, 19, 20.

Item 81: Combination of item 80, wherein each of the n separate entities comprise a different HC-HC promoter pair selected from HC-HC PP1, HC-HC PP2, HC-HC PP3, HC-HC PP4, HC-HC PP5, HC-HC PP6, HC-HC PP7, HC-HC PP8, HC-HC PP9, HC-HC PP10, HC-HC PP11, HC-HC PP12, HC-HC PP13, HC-HC PP14, HC-HC PP15, HC-HC PP16, HC-HC PP17, or HC-HC PP18.

Item 82: Combination of item 80 or 81, wherein each of the n separate entities comprise a different HC-HC promoter pair selected from HC-HC PP3, HC-HC PP4, HC-HC PP5, or HC-HC PP18, preferably wherein n is 2, 3 or 4.

Item 83: Combination of item 80 to 82, wherein the combination comprises m additional nucleic acid sequences (as defined in the first aspect) or compositions as separate entity, comprising at least one coding sequence encoding at least one antibody or a fragment of an antibody or a variant of an antibody.

Item 84: Combination of Item 80 to 83, wherein administration of the combination to a cell or to a subject leads to expression of at least two assembled antibodies in said cell or subject, wherein, preferably, at least about 70%, at least about 75%, at least about 80%, at least about 85%, at least about 90%, at least about 95%, or at least about 100% of the n expressed antibodies are (correctly) assembled. Preferably, mass spectrometry (MS) can be used to determine the percentage of assembled antibodies and misassembled antibodies.

In embodiments, the components of the combination (the individual nucleic acid sequence sets) may be formulated as separate entities and/or administered as separate entities which may further improve the expression of (correctly) assembled antibodies preferably in vivo.

Kit or Kit of Parts:

In a fourth aspect, the present invention provides a kit or kit of parts, preferably comprising at least one composition of the first aspect, and/or at least one nucleic acid sequence set of the second aspect, optionally comprising at least one liquid vehicle for solubilising, and, optionally, technical instructions providing information on administration and dosage of the kit components. Further, the kit or kit of parts may comprise the individual components of the combination of the third aspect.

Notably, embodiments relating to the first, second, and third aspect of the invention are likewise applicable to embodiments of the fourth aspect of the invention, and certain embodiments relating to the fourth aspect of the invention are likewise applicable to embodiments of the first, second, and third aspect of the invention.

In preferred embodiments, the kit or the kit of parts comprises:
(a) at least one first component selected from a composition of the first aspect and/or at least one nucleic acid sequence set of the second aspect;
(b) optionally, at least one second component selected from an antibody or antibody fragment;
(c) optionally, a liquid vehicle for solubilising (a) and/or (b), and optionally technical instructions providing information on administration and dosage of the components.

The kit or kit of parts may further comprise additional components as described in the context of the composition of the first aspect or the nucleic acid set of the second aspect, in particular, pharmaceutically acceptable carriers, excipients, buffers and the like.

The technical instructions of said kit or kit of parts may comprise information about administration and dosage and patient groups. Such kits, preferably kits of parts, may be applied e.g. for any of the applications or medical uses mentioned herein.

Preferably, the individual components of the kit or kit of parts may be provided in lyophilised or spray-dried form.

The kit may further contain as a part a vehicle (e.g. pharmaceutically acceptable buffer solution) for solubilising the first component, and/or the second component.

In preferred embodiments, the kit or kit of parts comprises Ringer- or Ringer lactate solution.

In preferred embodiments, the kit or kit of parts comprise an injection needle, a microneedle, an injection device, a catheter, an implant delivery device, or a micro cannula, or an inhalation device.

Any of the above kits may be used in applications or medical uses as defined in the context of the invention.

Medical Uses:

A further aspect relates to the first medical use of the provided composition, nucleic acid sequence set, combination and/or kit or kit of parts.

Embodiments and features described herein (in the context of the “medical use” or “further medical use”) are also applicable to method of treatments as further outlined below. Likewise, embodiments and features described in the context of the “method of treatment” are also applicable to first medical use and the further medical uses as described herein.

In preferred embodiments, the invention provides a composition as defined in the context of the first aspect, the nucleic acid sequence set as defined in the context of the second aspect, the combination as defined in the context of the third aspect, and/or the kit or kit of parts as defined in the context of the fourth aspect for use as a medicament.

In preferred embodiments, the composition, combination, or kit for use as a medicament comprises n nucleic acid sequence sets encoding at least one antibody or a fragment or variant thereof, wherein the n different nucleic acid sequence sets comprise
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody heavy chain B (HC-B), or a fragment or variant thereof,
wherein the at least one coding sequence of the nucleic acid sequence A and/or the nucleic acid sequence B encodes at least one antibody chain assembly promoter, wherein the composition is for expression of at least two assembled antibodies in vivo. Optionally, the composition comprises m additional nucleic acid sequences comprising at least one coding sequence encoding at least one antibody or a fragment of an antibody or a variant of an antibody.

In such embodiments, the nucleic acid sequence A, B, C, and/or D and, optionally, the m additional nucleic acid sequence are preferably complexed or associated with one or more lipids, thereby forming LNPs that comprise or consist of
i. at least one cationic or cationizable lipid;
ii. at least one a neutral lipid;
iii. at least one a steroid or steroid analogue;
iv. at least one aggregation reducing lipids, preferably polymer conjugated lipid.

In preferred embodiments, the composition, combination, or kit for use as a medicament comprises n RNA sequence sets encoding at least one antibody or a fragment or variant thereof, wherein the n different RNA sequence sets comprise
a) RNA sequence A comprising at least one coding sequence encoding at least one antibody heavy chain A (HC-A), or a fragment or variant thereof, and
b) RNA sequence B comprising at least one coding sequence encoding at least one antibody heavy chain B (HC-B), or a fragment or variant thereof,
wherein the at least one coding sequence of the RNA sequence A and/or the RNA sequence B encodes at least one antibody chain assembly promoter, wherein the composition is for expression of at least two assembled antibodies in vivo. Optionally, the composition comprises m additional nucleic acid sequences comprising at least one coding sequence encoding at least one antibody or a fragment of an antibody or a variant of an antibody.

In such embodiments, the RNA sequence A, B, C, and/or D and, optionally, the m additional RNA sequence are preferably complexed or associated with one or more lipids, thereby forming LNPs that comprise or consist of
i. at least one cationic or cationizable lipid;
ii. at least one a neutral lipid;
iii. at least one a steroid or steroid analogue;
iv. at least one aggregation reducing lipids, preferably polymer conjugated lipid.

In preferred embodiments, the composition, combination, or kit for use as a medicament comprises n nucleic acid sequence sets encoding at least one antibody or a fragment or variant thereof, wherein the n different nucleic acid sequence sets comprise
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody heavy chain B (HC-B), or a fragment or variant thereof,
wherein the at least one coding sequence of the nucleic acid sequence A and/or the nucleic acid sequence B encodes at least one antibody chain assembly promoter,
wherein antibody heavy chain A (HC-A) and antibody heavy chain B (HC-B) comprises at least one HC-HC assembly promoter pair comprising the following amino acid substitutions:
   HC-HC-PP3: S354C, T366W on HC-A; Y349C, T366S, L368A, Y407V on HC-B
   HC-HC-PP4: S364H, F405A on HC-A; Y349T, T394F on HC-B
   HC-HC-PP5: T350V, L351Y, F405A, Y407V on HC-A; T350V, T366L, K392L, T394W on HC-B
   HC-HC-PP18: Y349S, T366M, K370Y, K409V on HC-A; E/D356G, E357D, S364Q, Y407A on HC-B,

preferably, wherein the composition is for expression of at least two assembled antibodies in vivo. Optionally, the composition comprises m additional nucleic acid sequences comprising at least one coding sequence encoding at least one antibody or a fragment of an antibody or a variant of an antibody.

In such embodiments, the nucleic acid sequence A, B, C, and/or D and, optionally, the m additional nucleic acid sequence are preferably complexed or associated with one or more lipids, thereby forming LNPs that comprise or consist of
i. at least one cationic or cationizable lipid;
ii. at least one a neutral lipid;
iii. at least one a steroid or steroid analogue;
iv. at least one aggregation reducing lipids, preferably polymer conjugated lipid.

In preferred embodiments, the composition, combination, or kit for use as a medicament comprises n RNA sequence sets encoding at least one antibody or a fragment or variant thereof, wherein the n different RNA sequence sets comprise
a) RNA sequence A comprising at least one coding sequence encoding at least one antibody heavy chain A (HC-A), or a fragment or variant thereof, and
b) RNA sequence B comprising at least one coding sequence encoding at least one antibody heavy chain B (HC-B), or a fragment or variant thereof,
wherein the at least one coding sequence of the RNA sequence A and/or the RNA sequence B encodes at least one antibody chain assembly promoter,
wherein antibody heavy chain A (HC-A) and antibody heavy chain B (HC-B) comprises at least one HC-HC assembly promoter pair comprising the following amino acid substitutions:
   HC-HC-PP3: S354C, T366W on HC-A; Y349C, T366S, L368A, Y407V on HC-B
   HC-HC-PP4: S364H, F405A on HC-A; Y349T, T394F on HC-B
   HC-HC-PP5: T350V, L351Y, F405A, Y407V on HC-A; T350V, T366L, K392L, T394W on HC-B
   HC-HC-PP18: Y349S, T366M, K370Y, K409V on HC-A; E/D356G, E357D, S364Q, Y407A on HC-B,
wherein the composition is for expression of at least two assembled antibodies in vivo. Optionally, the composition comprises m additional nucleic acid sequences comprising at least one coding sequence encoding at least one antibody or a fragment of an antibody or a variant of an antibody. In such embodiments, the RNA sequence A, B, C, and/or D and, optionally, the m additional RNA sequence are complexed or associated with one or more lipids, thereby forming LNPs that comprise or consist of
   i. at least one cationic or cationizable lipid;
   ii. at least one a neutral lipid;
   iii. at least one a steroid or steroid analogue;
   iv. at least one aggregation reducing lipids, preferably polymer conjugated lipid.

In particular, said composition, nucleic acid sequence set, combination and/or kit or kit of parts may be used for human medical purposes and/or for veterinary medical purposes, preferably for human medical purposes.

Without whishing to be bound to theory, composition, nucleic acid sequence set, combination and/or kit or kit of parts may be advantageously used for human medical purposes and/or for veterinary medical purposes, preferably for human medical purposes as the thereby provided nucleic acid sequences generate at least two, preferably multiple correctly assembled antibodies. The fact that upon administration, correctly assembled antibodies are produced may advantageously reduce the risk of unwanted side effects (due to off-target binding of mis-assembled antibody species).

In particular, said composition, nucleic acid sequence set, combination and/or kit or kit of parts is for use as a medicament for human medical purposes, wherein said composition, nucleic acid sequence set, combination and/or kit or kit of parts may be particularly suitable for young infants, newborns, immunocompromised recipients, as well as pregnant and breast-feeding women and elderly people.

Further aspects relate to second and further medical uses of the provided composition, nucleic acid kit, combination and/or kit or kit of parts.

Embodiments and features described herein (in the context of the “further medical uses”) are also applicable to method of treatments as outlined below.

Accordingly, the composition, nucleic acid sequence set, combination, and/or kit or kit of the present invention, may be used for the treatment, prophylaxis or therapy of any disorder, disease, or condition which can be treated or prevented by use of an antibody, in particular cancer, cardiovascular diseases, neurological diseases, infectious diseases, autoimmune diseases, virus diseases, bacterial diseases, genetic diseases or disorder and diseases or disorders related thereto.

In preferred embodiments, the invention provides a composition as defined in the context of the first aspect, a nucleic acid sequence set as defined in the context of the second aspect, a combination as defined in the context of the third aspect, and/or a kit or kit of parts as defined in the context of the fourth aspect for use in the treatment or prophylaxis of an infection with a pathogen, for use in the treatment or prophylaxis of a cardiovascular disease or condition, for use in the treatment or prophylaxis of a neurological disease or condition, for use in the treatment or prophylaxis of an infectious disease or condition, for use in the treatment or prophylaxis of an autoimmune diseases or condition, for use in the treatment or prophylaxis of cancer or tumour disease or condition, for use in the treatment or prophylaxis of an eye or ophthalmic disease or condition, for use in the treatment or prophylaxis of a lung or pulmonary disease or condition, for use in the treatment or prophylaxis of a neurological disease or condition, for use in the treatment or prophylaxis of a genetic disease or condition, or for use in the treatment or prophylaxis of a lung disease or condition.

As used herein, the term “cancer” refers to the broad class of disorders and malignancies characterized by hyper proliferative cell growth, either in vitro (e.g., transformed cells) or in vivo. Conditions which can be treated or prevented by the compositions and methods of the invention include, e.g., a variety of neoplasms, including benign or malignant tumours, a variety of hyperplasias, or the like. Compositions and methods of the invention can achieve the inhibition and/or reversion of undesired hyper proliferative cell growth involved in such conditions.

Infectious diseases are typically caused by pathogenic microorganisms, such as bacteria, viruses, parasites or fungi. Infectious diseases can usually be spread, directly or indirectly, from one person to another.

The term “cardiovascular disease” as used herein typically includes any disorders/diseases of the cardiovascular system. Specific examples of cardiovascular diseases include coronary heart disease, arteriosclerosis, apoplexy and hypertension.

The term “neurological disease” as used herein typically includes disorders/diseases of the nervous system. Specific examples of neurological diseases include Alzheimer's disease, amyotrophic lateral sclerosis, dystonia, epilepsy, multiple sclerosis and Parkinson's disease.

The term “autoimmune disease” as used herein typically refers to a pathological state rising from an abnormal immune response of the body to substances and tissues that are normally present in the body.

In preferred embodiments, the invention provides a composition as defined in the context of the first aspect, a nucleic acid sequence set as defined in the context of the second aspect, a combination as defined in the context of the third aspect, and/or a kit or kit of parts as defined in the context of the fourth aspect for use in the treatment or prophylaxis of an infection with a pathogen (e.g. passive vaccination), preferably wherein the pathogen is a virus or a bacterium.

In preferred embodiments, the invention relates to a composition as defined in the context of the first aspect, the nucleic acid sequence set as defined in the context of the second aspect, the combination as defined in the context of the third aspect, and/or the kit or kit of parts as defined in the context of the fourth aspect for use in treatment or prophylaxis of a disease or condition (preferably as defined herein), wherein administration to a cell or to a subject leads to expression of at least two assembled antibodies in said cell or subject, wherein, preferably, at least about 70%, at least about 75%, at least about 80%, at least about 85%, at least about 90%, at least about 95%, or at least about 100% of the expressed at least two antibodies are (correctly) assembled antibodies. Preferably, mass spectrometry (MS) can be used to determine the percentage of assembled antibodies and misassembled antibodies In preferred embodiments, the invention relates to a composition as defined in the context of the first aspect, the nucleic acid sequence set as defined in the context of the second aspect, the combination as defined in the context of the third aspect, and/or the kit or kit of parts as defined in the context of the fourth aspect for use as a chronic medical treatment.

The term “chronic medical treatment” relates to treatments that require the administration more than once, for example once or more than once a day, once or more than once a week, once or more than once a month.

In preferred embodiments, applying or administering of the combination of the first aspect, the composition of the second aspect, or the kit or kit of parts of the third aspect is performed more than once, for example once or more than once a day, once or more than once a week, once or more than once a month (as defined herein).

Administration may be orally, parenterally, by inhalation spray, topically, rectally, nasally, buccally, vaginally or via an implanted reservoir. The term parenteral, as used herein, includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, intraocular, intravitreal, subretinal, intratumoral.

In preferred embodiments, the step of applying or administering is subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, intraocular, intravitreal, subretinal, intranasal or intratumoral.

In particularly preferred embodiments, the step of applying or administering is intravenous, intramuscular or intrapulmonal.

In embodiments where different nucleic acid sequence sets are to be administered as separate entities, the step of applying or administering may be at different injection sites for each entity. Alternatively, in embodiments where different nucleic acid sequence sets are to be administered as separate entities, the step of applying or administering may be at a different injection regimen or time-staggered. That procedure may improve the correct assembly of antibodies in vivo as each antibody (provided by an nucleic acid sequence set) may be administered as a separate entity.

In preferred embodiments, applying or administering of the combination of the first aspect, the composition of the second aspect, or the kit or kit of parts of the third aspect leads to expression of at least two assembled antibodies, wherein said at least two assembled antibodies are detectable at least about 6 hours, 12 hours, 24 hours, 36 hours, 48 hours, 60 hours, 72 hours, 96 hours, 120 hours, 144 hours, 156 hours, 168 hours, or 180 hours post-administration (e.g., post single administration).

In some embodiments, applying or administering of the combination of the first aspect, the composition of the second aspect, or the kit or kit of parts of the third aspect leads to expression of at least two assembled antibodies, wherein said at least two assembled antibodies are detectable at least about 1 day, 2 days, 3 days, 4 days, 5 days, 6 days, 7 days, 8 days, 9 days, 10 days, 11 days, 12 days, 13 days, 14 days, 15 days, 20 days, 22 days, 25 days, or 30 days post-administration (e.g., post single administration).

In some embodiments, applying or administering of the combination of the first aspect, the composition of the second aspect, or the kit or kit of parts of the third aspect leads to expression of at least two assembled antibodies, wherein said at least two assembled antibodies are detectable at least about 0.5 weeks, 1 week, 1.5 weeks, 2 weeks, 2.5 weeks, 3 weeks, 3.5 weeks, 4 weeks, 4.5 weeks, 5 weeks, 5.5 weeks, 6 weeks, 6.5 weeks, 7 weeks, 7.5 weeks, or 8 weeks post-administration (e.g., post single administration). In some embodiments, the systemic expression of the antibody is detectable at least about 1 month, 2 months, 3 months, or 4 months post-administration (e.g., post single administration).

In some embodiments, applying or administering of the combination of the first aspect, the composition of the second aspect, or the kit or kit of parts of the third aspect leads to expression of at least two assembled antibodies in target cells or tissues, wherein said target cells or tissues may be selected from hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

Methods of Treatment:

A further aspect of the present invention relates to a method of treating or preventing a disease, disorder, or condition.

Embodiments described above (in the context of the first medical use and the further medical uses) are also applicable to methods of treatment as described herein.

In particular, said composition, nucleic acid sequence set, combination and/or kit or kit of parts may be used in a method for human medical purposes and/or for veterinary medical purposes, preferably for human medical purposes.

In preferred embodiments, the invention provides a method of treating or preventing a disorder or condition, wherein the method comprises applying or administering to a subject in need thereof a composition as defined in the context of the first aspect, a nucleic acid sequence set as defined in the context of the second aspect, a combination as defined in the context of the third aspect, and/or a kit or kit of parts as defined in the context of the fourth aspect.

Accordingly, the composition, nucleic acid sequence set, combination and/or kit or kit of the present invention, may be used in a method of treating or preventing a disorder or condition, wherein the disorder or condition can be any disorder, disease, or condition which can be treated or prevented by use of an antibody, in particular cancer, cardiovascular diseases, neurological diseases, infectious diseases, autoimmune diseases, virus diseases, bacterial diseases, genetic diseases or disorder and diseases or disorders related thereto.

In preferred embodiments, the invention provides a method of treating or preventing a disorder or condition, wherein the method comprises applying or administering to a subject in need thereof a composition as defined in the context of the first aspect, a nucleic acid sequence set as defined in the context of the second aspect, a combination as defined in the context of the third aspect, and/or a kit or kit of parts as defined in the context of the fourth aspect, wherein the disorder or condition is an infection with a pathogen, a cardiovascular disease or condition, a neurological disease or condition, an infectious disease or condition, an autoimmune diseases or condition, a cancer or tumour disease or condition, an eye or ophthalmic disease or condition, a lung or pulmonary disease or condition, a neurological disease or condition, a genetic disease or condition, or a lung disease or condition.

In preferred embodiments, the invention relates to a method of treating or preventing a disorder or condition, wherein the method comprises applying or administering to a subject in need thereof a composition as defined in the context of the first aspect, a nucleic acid sequence set as defined in the context of the second aspect, a combination as defined in the context of the third aspect, and/or a kit or kit of parts as defined in the context of the fourth aspect, wherein administration to a cell or to a subject leads to expression of at least two assembled antibodies in said cell or subject, wherein, preferably, at least about 70%, at least about 75%, at least about 80%, at least about 85%, at least about 90%, at least about 95%, or at least about 100% of the expressed at least two antibodies are (correctly) assembled antibodies. Preferably, mass spectrometry (MS) can be used to determine the percentage of assembled antibodies and misassembled antibodies In preferred embodiments, the subject in need thereof is a mammalian subject, preferably a human subject. In specific embodiments, the subject in need thereof is a young infant human subject, a newborn human subject, immunocompromised human subject, a pregnant human subject, a breast-feeding human subject, or an elderly human subject.

In preferred embodiments, the method of treatment is a chronic medical treatment. Accordingly, applying or administering is performed more than once, for example once or more than once a day, once or more than once a week, once or more than once a month (as defined herein).

In preferred embodiments, the method of treatment comprises a step of applying or administering to a subject, wherein applying or administering may be orally, parenterally, by inhalation spray, topically, rectally, nasally, buccally, vaginally, via an implanted reservoir, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, intraocular, intravitreal, subretinal, intranasal or intratumoral administration.

In particularly preferred embodiments, the step of applying or administering is intravenous, intramuscular or intrapulmonal.

In embodiments where different nucleic acid sequence sets are to be administered as separate entities, the step of applying or administering may be at different injection sites for each entity. Alternatively, in embodiments where different nucleic acid sequence sets are to be administered as separate entities, the step of applying or administering may be at a different injection regimen or time-staggered. That procedure may improve the correct assembly of antibodies in vivo as each antibody (provided by an nucleic acid sequence set) may be administered as a separate entity.

Methods for Expressing or Producing at Least Two Nucleic Acid Encoded Antibodies:

A further aspect relates to a method expressing or producing at least two nucleic acid encoded antibodies in an organ or tissue.

A method for expressing at least two nucleic acid encoded antibodies in an organ or tissue in a subject, comprising administering or applying to a subject a composition as defined in the context of the first aspect, a nucleic acid sequence set as defined in the context of the second aspect, a combination as defined in the context of the third aspect, and/or a kit or kit of parts as defined in the context of the fourth aspect.

In preferred embodiments, administering or applying leads to expression of at least two assembled antibodies in an organ or tissue in a subject, wherein, preferably, at least about 70%, at least about 75%, at least about 80%, at least about 85%, at least about 90%, at least about 95%, or at least about 100% of the expressed at least two antibodies are (correctly) assembled antibodies. Preferably, mass spectrometry (MS) can be used to determine the percentage of assembled antibodies and misassembled antibodies In preferred embodiments, the method for expressing does not involve a purification step of the expressed antibodies. In preferred embodiments, the method for expressing does not involve a harvesting step of the expressed antibodies (e.g. harvesting from a cell, e.g. a bacterium or a cell culture).

In preferred embodiments, the method for expressing is an in vivo method for expressing at least two correctly assembled antibodies.

In preferred embodiments, the nucleic acid encoded antibodies are not provided by plasmid DNA. In preferred embodiments, the nucleic acid encoded antibodies are provided by RNA, preferably mRNA.

A further aspect relates to an in vitro method for the production of at least two nucleic acid encoded antibodies in a cell.

In preferred embodiments, the in vitro method of producing at least two nucleic acid encoded antibodies comprises a step of
(i) applying or administering a composition of the first aspect, a nucleic acid sequence set of the second aspect, a combination of the third aspect, a or a kit or kit of parts of the fourth aspect to allow expression of at least two assembled antibodies in said cell, and, optionally, a step of
(ii) isolating and/or purifying the expressed assembled antibodies, wherein the method is an in vitro, in situ, or ex vivo method.

In particularly preferred embodiments, the nucleic acid sequences used in the method are RNA sequences preferably mRNA sequences.

In preferred embodiments, administering or applying leads to expression of at least two assembled antibodies in said cell, wherein, preferably, at least about 70%, at least about 75%, at least about 80%, at least about 85%, at least about 90%, at least about 95%, or at least about 100% of the expressed at least two antibodies are (correctly) assembled antibodies. Preferably, mass spectrometry (MS) can be used to determine the percentage of assembled antibodies and misassembled antibodies

In preferred embodiments, the cell is a cell line suitable for the production of therapeutic antibodies. For example, a mammalian host cell line including (without limiting) NSO murine myeloma cells, PER. C6® human cells, and Chinese hamster ovary (CHO) cells. In other embodiments, the cell is a yeast cell, or a bacterial cell. For example, S. cerevisiae, P. pastoris, E. coli etc.

The obtained in vitro produced antibodies may be isolated from the cells and may be purified using typical antibody purification methods known in the art (e.g. affinity purification, chromatography, filtration, centrifugation, dialysis etc.

A further aspect relates to an a method for reducing the production of HC-HC by-products as defined herein in RNA encoded antibody mixtures as defined herein for in vitro or in vivo applications by introducing different HC-HC promoter pairs into the respective heavy chains, preferably wherein the HC-HC promoter pairs are selected from HC-HC PP1, HC-HC PP2, HC-HC PP3, HC-HC PP4, HC-HC PP5, HC-HC PP6, HC-HC PP7, HC-HC PP8, HC-HC PP9, HC-HC PP10, HC-HC PP11, HC-HC PP12, HC-HC PP13, HC-HC PP14, HC-HC PP15, HC-HC PP16, HC-HC PP17, or HC-HC PP18, more preferably from HC-HC PP3, HC-HC PP4, HC-HC PP5, or HC-HC PP18.

### BRIEF DESCRIPTION OF LISTS AND TABLES

Table 1: Preferred HC-HC assembly promoters and promoter pairs of the invention
Table A: CH3-CH3 assembly regions of preferred HC-HC promoters of the invention
Table 2: Human codon usage table with frequencies indicated for each amino acid
Table 3: Overview of mRNA constructs used in Examples section
Table 4: Overview of compositions used in Example 2 (and partly in Example 4)
Table 5: Results of the analysis of Example 2
Table 6: Overview of compositions used in Example 3 (and partly in Example 5)
Table 7: Results of the analysis of Example 3
Table 8: Overview of compositions used in Example 6

### BRIEF DESCRIPTION OF THE DRAWINGS

FIG. 1: FIG. 1A shows an exemplary IgG antibody that comprises HC-A comprising one exemplary HC-HC assembly promoter in the CH3 domain (1) and HC-B comprising an HC-HC assembly promoter in the CH3 domain (2). Both promoters (1,2) are compatible, interact, and promote specific assembly. Accordingly, (1) and (2) are exemplary HC-HC assembly promoter pairs in the context of the invention. Grey: light chains; black: heavy chains.

FIG. 1B shows an exemplary IgG antibody that comprises HC-A comprising a HC-HC assembly promoter (1) and a (wild type or non-modified) HC that does not comprise a compatible promoter in the CH3 domain. The configuration shown in B is an undesired misassembled by-product that could theoretically occur if co-expressed in the same cell (indicated by an “X”). In the context of the invention, the formation of misassembled by-products is prevented or reduced to allow co-expression of at least two assembled antibodies in the same cell.

FIG. 2: Assembly options for 1 common light chain (grey) and 3 different heavy chains (black) of Example 2. The AA configuration shows an assembled wild type (non-modified) IgG, the BC configuration shows an assembled IgG comprising an HC-HC assembly promoter pair. The other configurations (AB, AC, BB, CC) are undesired misassembled by-products that could theoretically occur if co-expressed in the same cell (indicated by an “X”). In the context of the invention, the formation of misassembled by-products is prevented or reduced to allow co-expression of at least two correctly assembled antibodies in the same cell. For larger view of the antibody elements (domains etc.), compare with FIG. 1.

FIG. 3: Exemplary mass spectrometry results. The Figure shows the deconvoluted mass spectra for wt IgG (top), IgG with HC-HC_18 (middle) and composition ID 3 (wt IgG and IgG with HC-HC_PP18; bottom) of Example 2. They were generated by summing up the individual mass spectra over the elution time of the Fc-dimers and subsequent deconvolution by means of the MaxEnt algorithm. Identity of the protein species were confirmed by comparing the experimentally determined masses with the theoretically calculated masses. In the bottom panel, the two main peaks could be ascribed to the desired assembled antibodies wt IgG (AA; HC-HC-configurations see Table 4) and IgG with HC-HC_18 (BC) by reference to their theoretical molecular weight 50140.2 and 49977.1 as well as to their main peaks in the top and middle panel, respectively. The given percentages reflect the relative amounts of the assembled wt IgG and IgG with HC-HC_PP18 within the antibody mixture as shown in Table 5. Notably, no misassembled species could be detected at the theoretical molecular weights for the undesired HC-HC configurations.

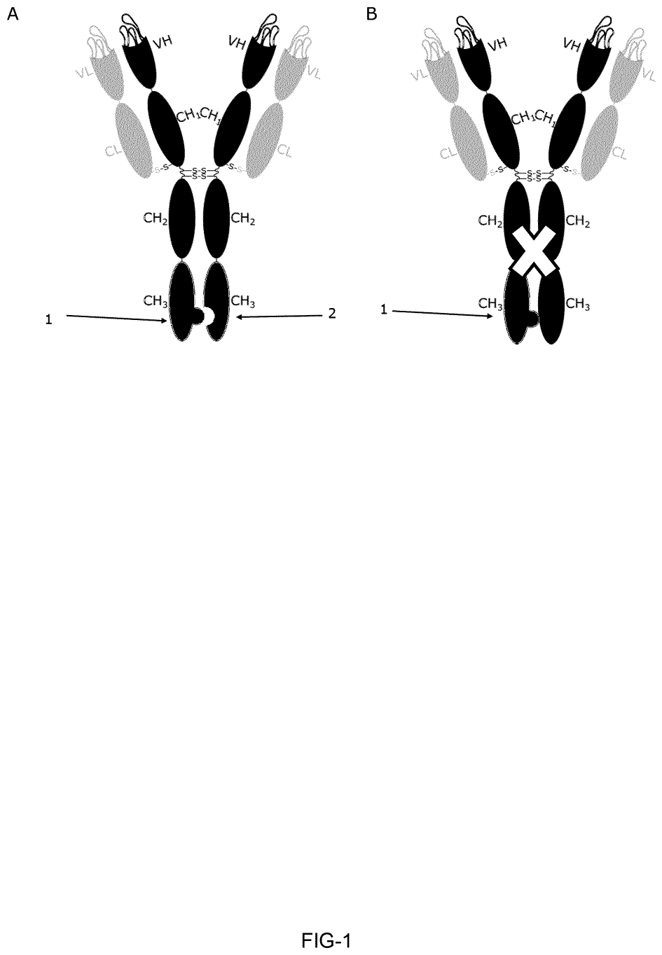

### EXAMPLES

The following examples are given to enable those skilled in the art to more clearly understand and to practice the present invention. The present invention is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below.

### Example 1: Preparation of DNA and RNA Constructs and Compositions

1.1. Preparation of DNA and RNA Constructs:

DNA sequences encoding different antibody chains were prepared and used for subsequent RNA in vitro transcription reactions. Said DNA sequences were prepared by modifying wild type (reference) encoding DNA sequences for respective antibody parts by introducing a G/C optimized or modified coding sequence for stabilization and expression optimization. Sequences were introduced into a pUC derived DNA vector to comprise stabilizing 3-UTR sequences and 5′-UTR sequences, additionally comprising a stretch of adenosines (see Table 3). The obtained plasmid DNA constructs were transformed and propagated in bacteria using common protocols known in the art. Eventually, the plasmid DNA constructs were extracted, purified, and used for subsequent RNA in vitro transcription.

1.2. RNA In Vitro Transcription from Plasmid DNA Templates:

DNA plasmids prepared according to section 1.1 were enzymatically linearized using a restriction enzyme and used for DNA dependent RNA in vitro transcription using T7 RNA polymerase in the presence of a nucleotide mixture (ATP/GTP/CTP/UTP) and cap analog. The obtained RNA constructs were purified using RP-HPLC (PureMessenger®, CureVac AG, Tübingen, Germany; WO2008/077592) and used for in vitro and in vivo experiments. The generated RNA sequences/constructs are provided in Table 3 with the encoded protein indicated therein.

TABLE 3
Overview of mRNA constructs encoding Influenza B antibody chains used in Examples

| RNA | | SEQ ID NO: | SEQ ID NO: |
|---|---|---|---|
| ID | Name | Protein | RNA |
| R8534 | LC mRNA product | 80 | 92 |
| R8535 | HC mRNA product (not comprising an assembly promoter) | 81 | 93 |
| R8544 | HC mRNA product comprising an assembly promoter: | 82 | 94 |
| | HC-HC_PP3_HC-A mRNA | | |
| R8545 | HC mRNA product comprising an assembly promoter: | 83 | 95 |
| | HC-HC_PP3_HC-B mRNA | | |
| R8536 | HC mRNA product comprising an assembly promoter: | 84 | 96 |
| | HC-HC_PP4_HC-A mRNA | | |
| R8537 | HC mRNA product comprising an assembly promoter: | 85 | 97 |
| | HC-HC_PP4_HC-B mRNA | | |
| R8538 | HC mRNA product comprising an assembly promoter: | 86 | 98 |
| | HC-HC_PP5_HC-A mRNA | | |
| R8539 | HC mRNA product comprising an assembly promoter: | 87 | 99 |
| | HC-HC_PP5_HC-B mRNA | | |
| R8540 | HC mRNA product comprising an assembly promoter: | 88 | 100 |
| | HC-HC_PP16_HC-A mRNA | | |
| R8541 | HC mRNA product comprising an assembly promoter: | 89 | 101 |
| | HC-HC_PP16_HC-B mRNA | | |
| R8542 | HC mRNA product comprising an assembly promoter: | 90 | 102 |
| | HC-HC_PP18_HC-A mRNA | | |
| R8543 | HC mRNA product comprising an assembly promoter: | 91 | 103 |
| | HC-HC_PP18_HC-B mRNA | | |

### Example 2: In Vitro Expression Analysis of Antibodies Comprising HC-HC Assembly Promoter Pairs and an Unmodified Antibody

The goal of the experiment was to identify antibody assembly promoter pairs that allow for the production of two correctly assembled antibodies in the same cell (in this experiment: one unmodified IgG that does not comprise assembly promotors in the presence of an antibody comprising an assembly promotor pair). Further, it was a goal of the experiment that the two correctly assembled antibodies are produced without generating undesired by-products (e.g. mismatching of antibody chains).

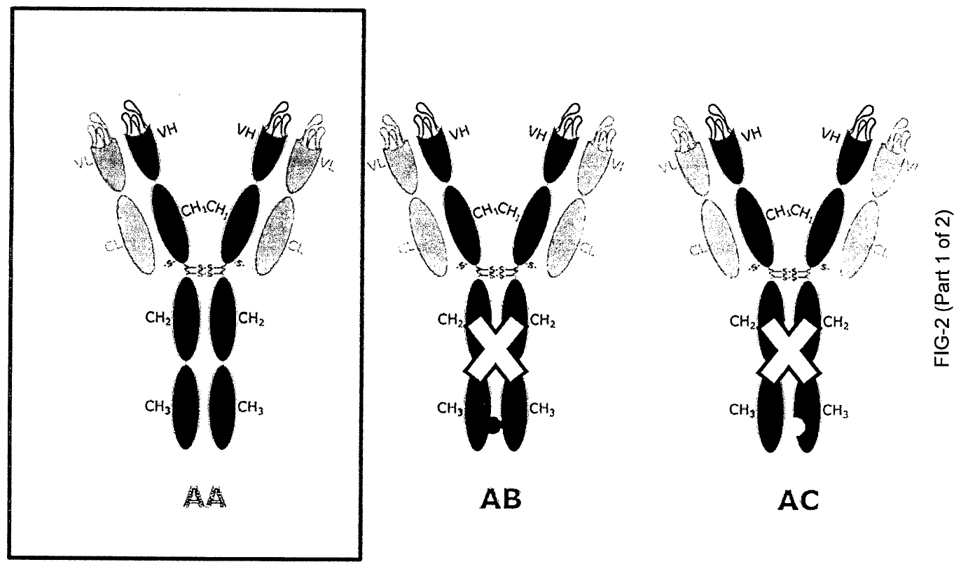

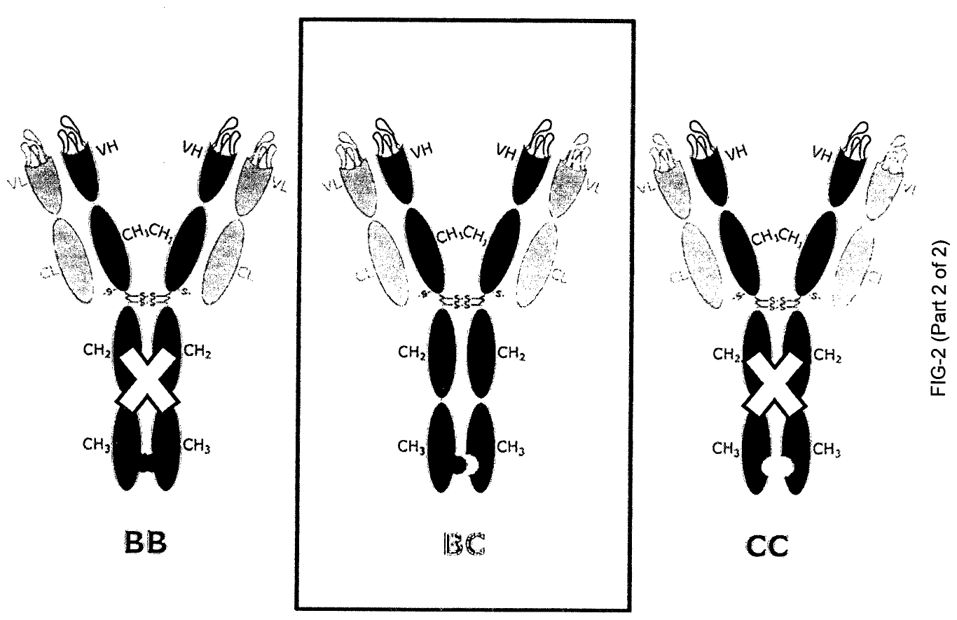

For the in vitro analysis, a composition comprising mRNA encoding an Influenza B antibody (HC: R8535 “Chain A”+LC: R8534) was combined with a nucleic acid sequence set encoding Influenza B antibody heavy chains comprising HO-HO assembly promoter elements (“Chain B” and “Chain C” with the same light chain R8534). The compositions were used for transfection of cells as further outlined below. The compositions used for transfection are provided in Table 4. Also provided in Table 4 are the two desired HO-HG configurations, and the undesired HO-HO configurations of potential misassembled species or by-products. The conditions of the experiment are further illustrated in FIG. 2.

TABLE 4
Overview of compositions used in Example 2 (and partly in Example 4)

| | | Desired | Undesired |
|---|---|---|---|
| | mRNA | assembled | Mis-assembled |
| Composition | sequences | antibodies | antibodies |
| ID | comprised in the composition | HC configuration | HC configuration |
| 1 | LC mRNA product R8534 | AA | AB |
| | HC mRNA product R8535 (“Chain A”) | BC | BB |
| | HC-HC_PP4_HC-A mRNA R8536 (“Chain B”) | | CC |
| | HC-HC_PP4_HC-B mRNA R8537 (“Chain C”) | | AC |
| 2 | LC mRNA product R8534 | AA | AB |
| | HC mRNA product R8535 (“Chain A”) | BC | BB |
| | HC-HC_PP5_HC-A mRNA R8538 (“Chain B”) | | CC |
| | HC-HC_PP5_HC-B mRNA R8539 (“Chain C”) | | AC |
| 3 | LC mRNA product R8534 | AA | AB |
| | HC mRNA product R8535 (“Chain A”) | BC | BB |
| | HC-HC_PP18_HC-A mRNA R8542 (“Chain B”) | | CC |
| | HC-HC_PP18_HC-B mRNA R8543 (“Chain C”) | | AC |
| 4 | LC mRNA product R8534 | AA | AB |
| | HC mRNA product R8535 (“Chain A”) | BC | BB |
| | HC-HC_PP3_HC-A mRNA R8544 (“Chain B”) | | CC |
| | HC-HC_PP3_HC-B mRNA R8545 (“Chain C”) | | AC |

2.1: Cell Transfection with mRNA Constructs

135 μg of mRNA (total amount of composition; HC to LC ratio (w:w) 2:1 on mRNA level) was used for four separate transfection experiments (BHK cells using lipofectamine as transfection reagent). The compositions used for the transfection of cells are provided in Table 4.

2 days post transfection, the produced, secreted antibodies were purified from the BHK cell culture medium using a protein A plus agarose (Pierce Chromatography Cartridge; Thermo Fisher). The four purified antibody mixtures were subjected to further analysis as outlined in 2.2.

2.2: Mass-Spectrometry-Based Analysis of Antibody Assembly

12.5 μg antibody sample (four different samples obtained in step 2.1) was treated with 0.5 μl PNGaseF (R&D Systems, #9109-GH) and incubated over night at 37° C. to allow deglycosylation. Following deglycosylation, the sample was treated with 0.32 μl cysteine protease FabALACTICA (Genovis, #A0-AG1-020) to digest the antibodies above the hinge-region into a Fab′ fragments and Fc-dimer fragments. The enzymatic treatment reduced the MW of a full-length antibody (about 150 kDa plus glycan pattern) to an Fc portion of about 50 kDa without glycan pattern. Afterwards, the sample was analyzed using HPLC-MS to observe mass differences and to determine the relative amounts of assembled and misassembled antibodies.

2 μg of obtained, digested probe was chromatographically purified, desalted, and analyzed using RP-HPLC (Acquity BEH300 C4, 1 mm 50 mm, 1.7 pm) coupled to MS (QTOF mass spectrometer, MAXIS, Bruker Daltonics). The mass-spectra for each sample were recorded and the individual mass spectra over the elution time of the Fc-dimers summed up and subsequently deconvoluted by means of the MaxEnt algorithm.

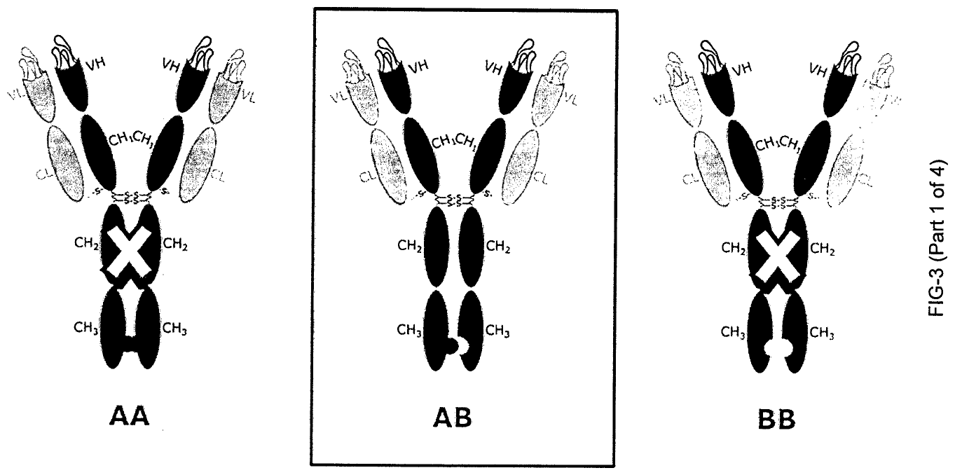

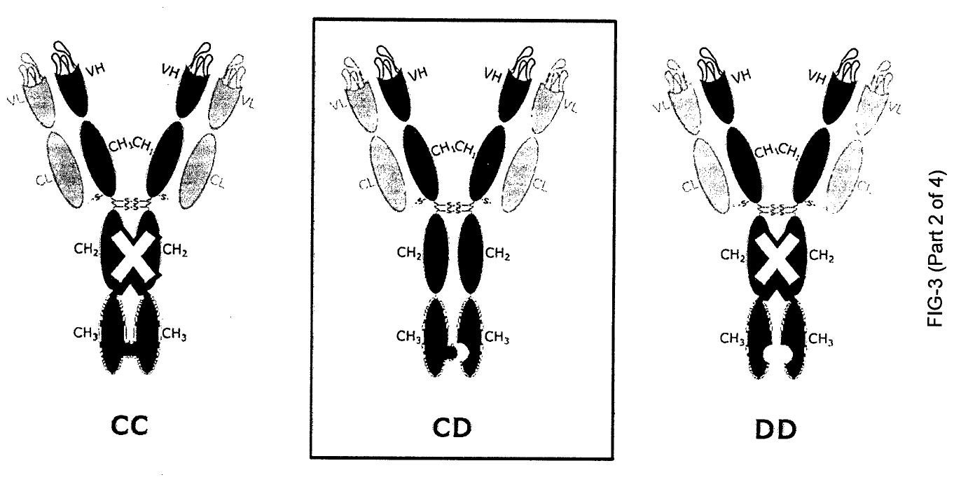

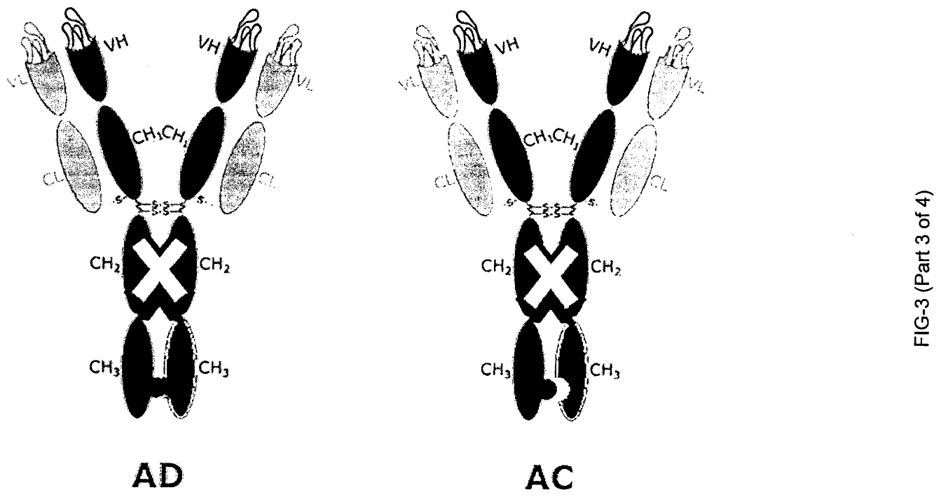

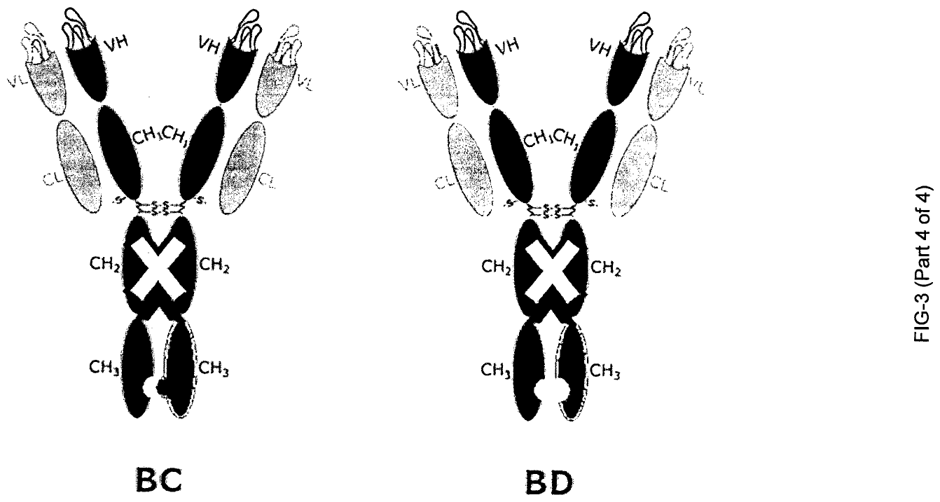

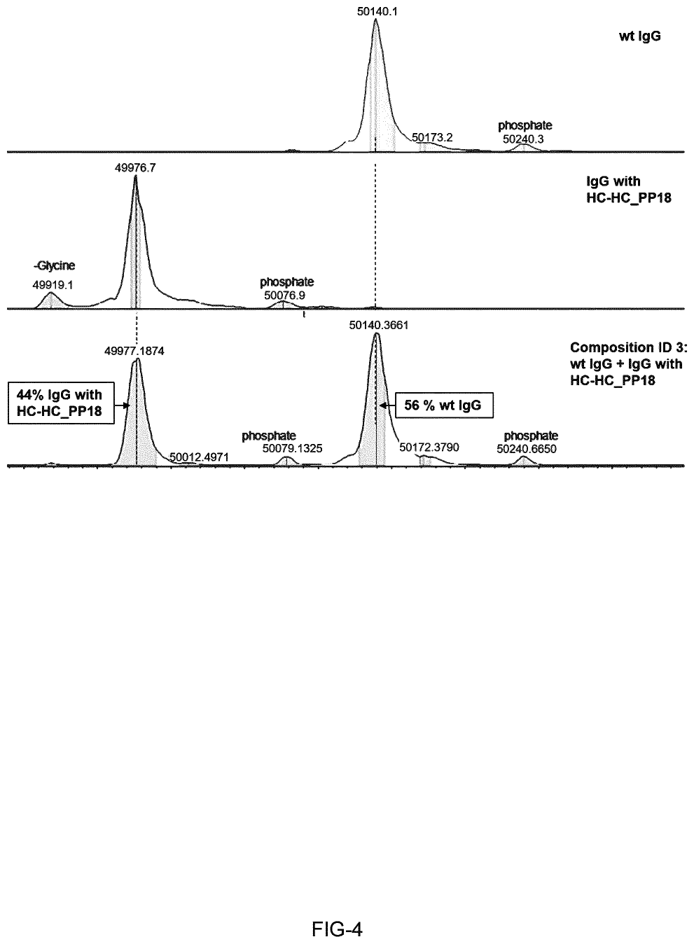

Subsequently, the determined mass for each identified protein species in each of the four samples was compared to the theoretically expected mass to confirm the identity of the respective protein species. Lastly, the relative amounts of assembled and misassembled antibodies within the antibody mixture were calculated on the basis of the peak areas. The result of the analysis of Example 2 is summarized in Table 5. For further illustration, exemplary mass spectrometry results for Composition ID 3 of Example 2 are shown in FIG. 3.

TABLE 5
Results of the analysis of Example 2

| Composition | Encoded | Assembled species | | Mis-assembled species | | | |
|---|---|---|---|---|---|---|---|
| ID | antibodies | AA | BC | BB | CC | AB | AC |
| 1 | wt IgG | 48% | 31% | | | 21% | |
| | IgG with HC-HC_PP4 | | | | | | |
| 2 | wt IgG | 57% | 43% | | | | |
| | IgG with HC-HC_PP5 | | | | | | |
| 3 | wt IgG | 56% | 44% | | | | |
| | IgG with HC-HC_PP18 | | | | | | |
| 4 | wt IgG | 73% | 27% | | | | |
| | IgG with HC-HC_PP3 | | | | | | |

As shown in Table 5, in vitro administration of the compositions comprising nucleic acid sequences encoding an unmodified antibody (wt IgG) and, additionally, comprising nucleic acid sequence sets having HC-HC_PP4, HC-HC_PP5, HC-HC_PP18, or HC-HC_PP3 to a cell led to the simultaneous production of the desired assembled antibodies. Under the respective experimental conditions, the combination of wt IgG and IgG with HC-HC_PP4 (composition 1) led to the production of a small percentage of undesired, misassembled antibody species (“AB”) in the presence of wt IgG HCs.

The data clearly shows that the tested HC-HC assembly promoter pairs led to an increased production of correctly assembled antibodies, also in the presence of very similar but unmodified heavy chains (wt HC) of the same antibody type. The data demonstrates that by using antibody assembly promoter pairs according to the invention, it is possible to produce mixtures of correctly assembled antibody using nucleic acid compositions encoding said antibodies.

### Example 3: In Vitro Expression Analysis of Two Antibodies Comprising HC-HC Promoter Pairs

The goal of the experiment was to test the identified assembly promoter pairs of Example 2 against each other, and to identify assembly promoter pairs that are compatible with each other. Accordingly the goal was to find assembly promoter pairs that can be used in combination for producing correctly assembled antibody mixtures.

For the in vitro analysis, a composition comprising two nucleic acid sequence sets encoding Influenza B antibody heavy chains comprising different HC-HC assembly promoter pairs were combined with the same light chain (R8534) and administered in vitro to a cell to allow expression of the encoded antibodies.

The compositions used for transfection are provided in Table 6. Also provided therein are the two desired HC-HC configurations (AB and CD), and the undesired HC-HC configurations of potential misassembled species. The conditions of the experiment are further illustrated in FIG. 3.

TABLE 6
Overview of compositions used in Example 3 (and partly in Example 5)

| | | Desired | Undesired |
|---|---|---|---|
| | mRNA | assembled | Mis-assembled |
| Composition | sequences | antibodies | antibodies |
| ID | comprised in the composition | HC configuration | HC configuration |
| 5 | LC mRNA product R8534 | AB | AA, BB |
| | HC-HC_PP4_HC-A mRNA R8536 (“Chain A”) | CD | CC, DD |
| | HC-HC_PP4_HC-B mRNA R8537 (“Chain B”) | | AC, AD |
| | HC-HC_PP5_HC-A mRNA R8538 (“Chain C”) | | BC, BD |
| | HC-HC_PP5_HC-B mRNA R8539 (“Chain D”) | | |
| 6 | LC mRNA product R8534 | AB | AA, BB |
| | HC-HC_PP4_HC-A mRNA R8536 (“Chain A”) | CD | CC, DD |
| | HC-HC_PP4_HC-B mRNA R8537 (“Chain B”) | | AC, AD |
| | HC-HC_PP18_HC-A mRNA R8542 (“Chain C”) | | BC, BD |
| | HC-HC_PP18_HC-B mRNA R8543 (“Chain D”) | | |
| 7 | LC mRNA product R8534 | AB | AA, BB |
| | HC-HC_PP4_HC-A mRNA R8536 (“Chain A”) | CD | CC, DD |
| | HC-HC_PP4_HC-B mRNA R8537 (“Chain B”) | | AC, AD |
| | HC-HC_PP3_HC-A mRNA R8544 (“Chain C”) | | BC, BD |
| | HC-HC_PP3_HC-B mRNA R8545 (“Chain D”) | | |
| 8 | LC mRNA product R8534 | AB | AA, BB |
| | HC-HC_PP5_HC-A mRNA R8538 (“Chain A”) | CD | CC, DD |
| | HC-HC_PP5_HC-B mRNA R8539 (“Chain B”) | | AC, AD |
| | HC-HC_PP18_HC-A mRNA R8542 (“Chain C”) | | BC, BD |
| | HC-HC_PP18_HC-B mRNA R8543 (“Chain D”) | | |
| 9 | LC mRNA product R8534 | AB | AA, BB |
| | HC-HC_PP5_HC-A mRNA R8538 (“Chain A”) | CD | CC, DD |
| | HC-HC_PP5_HC-B mRNA R8539 (“Chain B”) | | AC, AD |
| | HC-HC_PP3_HC-A mRNA R8544 (“Chain C”) | | BC, BD |
| | HC-HC_PP3_HC-B mRNA R8545 (“Chain D”) | | |
| 10 | LC mRNA product R8534 | AB | AA, BB |
| | HC-HC_PP18_HC-A mRNA R8542 (“Chain A”) | CD | CC, DD |
| | HC-HC_PP18_HC-B mRNA R8543 (“Chain B”) | | AC, AD |
| | HC-HC_PP3_HC-A mRNA R8544 (“Chain C”) | | BC, BD |
| | HC-HC_PP3_HC-B mRNA R8545 (“Chain D”) | | |

3.1: Cell Transfection with mRNA Constructs

Composition 5-10 (HC to LC ratio (w:w) 2:1 on mRNA level) was used for six separate transfection experiments (each performed as described in section 2.1.).

3.2: Mass-Spectrometry-Based Analysis of Antibody Assembly

Sample preparation and analysis was performed according to section 2.2. The calculated mass for each protein species in each of the six samples was compared to the theoretical expected mass to observe mass differences and to determine the relative amounts of assembled and misassembled antibodies (raw data mass-spectrograms not shown). The result of the analysis is summarized in Table 7.

TABLE 7
Results of the analysis of Example 3

| Composition | Encoded | Assembled species | | Mis-assembled species | | | | | | | |
|---|---|---|---|---|---|---|---|---|---|---|---|
| ID | antibodies | AB | CD | AA | BB | CC | DD | AC | AD | BC | BD |
| 5 | IgG with HC-HC_PP4 | 47% | 46% | | | | | | 7% | | |
| | IgG with HC-HC_PP5 | | | | | | | | | | |
| 6 | IgG with HC-HC_PP4 | 44% | 56% | | | | | | | | |
| | IgG with HC-HC_PP18 | | | | | | | | | | |
| 7 | IgG with HC-HC_PP4 | 65% | 35% | | | | | | | | |
| | IgG with HC-HC_PP3 | | | | | | | | | | |
| 8 | IgG with HC-HC_PP5 | 42% | 58% | | | | | | | | |
| | IgG with HC-HC_PP18 | | | | | | | | | | |
| 9 | IgG with HC-HC_PP5 | 66% | 34% | | | | | | | | |
| | IgG with HC-HC_PP3 | | | | | | | | | | |
| 10 | IgG with HC-HC_PP18 | 70% | 30% | | | | | | | | |
| | IgG with HC-HC_PP3 | | | | | | | | | | |

As shown in Table 7, in vitro administration of the six compositions (composition 5 to 10) comprising two nucleic acid sequence sets having different HC-HC assembly promoter pairs to a cell led to the simultaneous production of the desired assembled antibodies. Under the respective experimental conditions, the combination of IgG with HC-HC_PP4 and IgG with HC-HC_PP5 led to the production of a small percentage of undesired, misassembled antibody species (7% “AD”).

The data clearly shows that most tested HC-HC assembly promoter pairs are compatible with each other, and that they can be combined. The data demonstrates that by using antibody assembly promoter pairs according to the invention, it is possible to produce mixtures of correctly assembled antibodies using nucleic acid compositions encoding said antibody mixture.

### Example 4: In Vivo Expression Analysis of Antibodies Comprising HC-HC Promoter Pairs and an Unmodified Antibody

The goal of the experiment was to evaluate whether the use of antibody assembly promotors allows the production of two correctly assembled antibodies in vivo (in that experiment: one unmodified Influenza B IgG1 that does not comprise assembly promotors in the presence of an Influenza B construct comprising assembly promotors).

For the in vivo analysis, composition ID 3 (see Table 4) comprising mRNA encoding an Influenza B antibody (HC: R8535+LC: R8534) and mRNA encoding the antibody heavy chain with HC-HC assembly promoter elements PP18 (with the same light chain R8534). The composition was used for in vivo administration as further outlined below.

Also provided in Table 4 are the two desired HC-HC configurations of Composition ID 3, and its undesired HC-HC configurations of potential misassembled species. The conditions of the experiment are further illustrated in FIG. 2.

4.1: Lipid Nanoparticle Formulation of mRNA Constructs

mRNA constructs were formulated in lipid nanoparticles (final mRNA concentration 0.2 mg/ml; HC to LC ratio (w:w) 2:1 on mRNA level). LNPs were prepared using a cationic lipid, a structural lipid, a PEG-lipid, and cholesterol. Lipid solution (in ethanol) was mixed with RNA solution (in aqueous buffer) using a T-connector. Obtained LNPs were re-buffered in a carbohydrate buffer via dialysis, and up-concentrated to a target concentration using TFF.

4.2: In Vivo Administration of LNP-Formulated mRNA and Preparation of Serum

A dose of 2 mg/kg LNP-formulation of composition ID 3 was injected intravenously into the tail vain of C57BL6 female mice. 48 hours after administration the animals were sacrificed, the blood collected and serum prepared.

4.3: Purification of mAb

The produced, secreted antibodies were purified from mouse serum using FPLC (HiTrap Protein G HP antibody purification column, #17040401, Cytiva) and anti-human IgG-Agarose (Sigma, #A3316). The purified antibody mixture was subjected to further analysis as outlined in section 4.4.

4.4: Mass-Spectrometry-Based Analysis of Antibody Assembly

12.5 μg antibody sample (obtained in step 4.4) was further processed and analyzed according to section 2.2.

4.5: Results

In vivo administration of formulated composition ID 3 comprising nucleic acid sequences encoding an unmodified antibody (wt IgG) and, additionally, comprising a nucleic acid sequence set having HC-HC PP18 led to the simultaneous production of the desired correctly assembled antibodies. Importantly, no Fc mispairing was detected in the MS analysis.

That shows that the used HC-HC assembly promoter pairs led to the production of correctly assembled antibodies in vivo, also in the presence of unmodified heavy chains with an almost identical protein sequence (wt HC). The data demonstrates that by using antibody assembly promoter pairs according to the invention, it is possible to produce assembled antibody mixtures using nucleic acid compositions encoding said antibodies for in vivo applications.

### Example 5: In Vivo Expression Analysis of Two Antibodies Comprising Two Different HC-HC Promoter Pairs

The goal of the experiment was to evaluate whether the use of two different antibody assembly promotor pairs allows the production of two correctly assembled antibodies in vivo.

For the in vivo analysis, a composition comprising two nucleic acid sequence sets encoding Influenza B antibody heavy chains comprising different HC-HC assembly promoter pairs were combined with the same light chain (R8534) and administered in vivo to allow expression of the encoded antibodies.

For the in vivo analysis, compositions ID 6 and ID 8 (see Table 6) were used. The composition was used for in vivo administration as further outlined below. Also provided in Table 6 are the two desired HC-HC configurations of Composition ID 6 and ID 8, and its undesired HC-HC configurations of potential misassembled species. The conditions of the experiment are further illustrated in FIG. 3.

5.1: Lipid Nanoparticle Formulation of mRNA Constructs

mRNA constructs were formulated in lipid nanoparticles (final mRNA concentration 0.2 mg/ml; HC to LC ratio (w:w) 2:1 on mRNA level). LNPs were prepared using a cationic lipid, a structural lipid, a PEG-lipid, and cholesterol. Lipid solution (in ethanol) was mixed with RNA solution (in aqueous buffer) using a T-connector. Obtained LNPs were re-buffered in a carbohydrate buffer via dialysis, and up-concentrated to a target concentration using TFF.

5.2: In Vivo Administration of LNP-Formulated mRNA and Preparation of Serum

A dose of 2 mg/kg LNP-formulation of composition ID 3 was injected intravenously into the tail vain of C57BL6 female mice. 48 hours after administration the animals were sacrificed, the blood collected and serum prepared.

5.3: Purification of mAb

The produced, secreted antibodies were purified from mouse serum using FPLC (HiTrap Protein G HP antibody purification column, #17040401, Cytiva) and anti-human IgG-Agarose (Sigma, #A3316). The purified antibody mixture was subjected to further analysis as outlined in section 5.4.

5.4: Mass-Spectrometry-Based Analysis of Antibody Assembly

12.5 μg antibody sample (obtained in step 4.4) was further processed and analyzed according to section 2.2.

5.5: Results

In vivo administration of the formulated compositions (composition 6 and 8) comprising two nucleic acid sequence sets having different HC-HC assembly promoter pairs to an animal led to the simultaneous production of the desired correctly assembled antibodies. Importantly, no Fc mispairing was detected in the MS analysis.

That shows that the used HC-HC assembly promoter pairs led to the production of correctly assembled antibodies in vivo, also in the presence of other promoter pairs. The data demonstrates that by using antibody assembly promoter pairs according to the invention, it is possible to produce mixtures of correctly assembled antibodies in vivo using nucleic acid compositions encoding said antibody mixture.

### Example 6: In Vivo Expression Analysis of Three Antibodies Comprising Different HC-HC Promoter Pairs

The goal of the experiment was to evaluate whether the in vivo administration of three Influenza B antibodies comprising different HC-HC promoter pairs would lead to the simultaneous production of the three desired correctly assembled antibodies in vivo.

For the in vivo analysis, a composition comprising three nucleic acid sequence sets encoding antibody heavy chains with three different HC-HC promoter pairs and a common light chain (R8534) were administered in vivo to allow expression of the encoded antibodies. The compositions that were used in this example are provided in Table 8. Also provided therein are the three desired HC-HC configurations (AB, CD, EF), and the undesired HC-HC configurations of potential misassembled species.

TABLE 8
Overview of compositions used in Example 6

| | | Desired | Undesired |
|---|---|---|---|
| | mRNA | assembled | Mis-assembled |
| Composition | sequences | antibodies | antibodies |
| ID | comprised in the composition | HC configuration | HC configuration |
| 11 | LC mRNA product R8534 | AB | AA, BB, CC, DD, |
| | HC-HC_PP3_HC-A mRNA R8544 (“Chain A”) | CD | EE, FF, AC, AD, |
| | HC-HC_PP3_HC-B mRNA R8545 (“Chain B”) | EF | AE, AF, BC, BD, |
| | HC-HC_PP4_HC-A mRNA R8536 (“Chain C”) | | BE, BF, CE, CF, |
| | HC-HC_PP4_HC-B mRNA R8537 (“Chain D”) | | DE, DF |
| | HC-HC_PP18_HC-A mRNA R8542 (“Chain E”) | | |
| | HC-HC_PP18_HC-B mRNA R8543 (“Chain F”) | | |
| 12 | LC mRNA product R8534 | AB | AA, BB, CC, DD, |
| | HC-HC_PP3_HC-A mRNA R8544 (“Chain A”) | CD | EE, FF, AC, AD, |
| | HC-HC_PP3_HC-B mRNA R8545 (“Chain B”) | EF | AE, AF, BC, BD, |
| | HC-HC_PP5_HC-A mRNA R8538 (“Chain C”) | | BE, BF, CE, CF, |
| | HC-HC_PP5_HC-B mRNA R8539 (“Chain D”) | | DE, DF |
| | HC-HC_PP18_HC-A mRNA R8542 (“Chain E”) | | |
| | HC-HC_PP18_HC-B mRNA R8543 (“Chain F”) | | |

6.1: Lipid Nanoparticle Formulation of mRNA Constructs

mRNA constructs were formulated according to section 4.1.

6.2: In Vivo Administration of LNP-Formulated mRNA and Preparation of Serum

2 mg/kg LNP-formulation of composition ID 11 or 12 were used according to section 4.2.

6.3: Purification of mAb

The produced, secreted antibodies in the serum of mice that were administered composition ID 11 or 12 were purified according to section 4.3. The two purified antibody mixtures were subjected to further analysis as outlined in 6.4.

6.4: Mass-Spectrometry-Based Analysis of Antibody Assembly

12.5 μg antibody sample of the two antibody mixtures (obtained in step 6.3) was further processed and analyzed according to section 2.2.

6.5 Results

In vivo administration of formulated compositions ID 11 or 12 comprising three nucleic acid sequence sets encoding antibody heavy chains with three different HC-HC assembly promoter pairs and a common light chain (R8534) led to the simultaneous and specific production of the three desired correctly assembled antibodies. Importantly, no Fc mispairing was detected. Importantly, no Fc mispairing was detected in the MS analysis.

The data demonstrates that by using antibody assembly promoter pairs according to the invention, it is possible to produce assembled antibody mixtures of three antibodies using nucleic acid compositions encoding said antibodies in vivo.

### Example 7: In Vivo Antibody Levels in Dependence on the Number of Different HC in the Nucleic Acid Composition

The goal of the experiment was to evaluate whether increasing numbers of different HC with or without HC-HC assembly promotor pairs in nucleic acid compositions would negatively affect antibody levels upon in vivo administration.

To this end, LNP-formulated Composition ID 3 (3 HC), 6/8 (4 HC) and 11/12 (6 HC) were administered in vivo with a total mRNA amount of 2 mg/kg per mouse in all cases followed by serum collection after 48h as specified in Examples 4, 5, 6, respectively. Subsequently, mouse sera from individual mice were analyzed using an ELISA detecting human IgG (Goat Anti-Human IgG, #2044-01, SouthernBiotech as coating antibody and Goat Anti-Human IgG Biotin #109065088, Dianova, as detection antibody),

In addition to the results described in Examples 4-6 (all five tested compositions led to the specific assembly of the desired antibodies in vivo without misassembled species being detected), the ELISA results showed that increasing numbers of different HC with or without HC-HC assembly promotor pairs in nucleic acid compositions did not negatively affect antibody levels in vivo. Accordingly, it has been demonstrated that also nucleic acid compositions encoding for multiple antibodies lead to sufficient antibody levels in vivo.

### Summary of the Findings (Examples 1 to 7)

As shown in Example 2, the tested HC-HC assembly promoter pairs support specific in vitro assembly of an antibody in the presence of antibody chains that lack HC-HC assembly promoters (IgG wt). Accordingly, one HC-HC assembly promotor pair can also be combined with IgG wt to generate nucleic acid compositions encoding antibody mixtures of two assembled antibodies.

In addition, as shown in Example 3, the tested HC-HC assembly promoter pairs support specific in vitro assembly of an antibody, also in the presence of antibody chains that comprise a different HC-HC assembly promoter pair. Accordingly, two HC-HC assembly promotor pairs can also be combined to generate nucleic acid compositions encoding antibody mixtures of two assembled antibodies.

In addition, as described in Example 4, the tested HC-HC assembly promoter pairs support specific in vivo assembly of an antibody in the presence of antibody chains that lack HC-HC assembly promoters (IgG wt). Accordingly, one HC-HC assembly promotor pair can also be combined with IgG wt to generate nucleic acid compositions for in vivo administration encoding antibody mixtures of two assembled antibodies.

In addition, as described in Example 5, the tested HC-HC assembly promoter pairs support specific in vivo assembly of an antibody, also in the presence of other antibody chains that comprise a different HC-HC assembly promoter pair. Accordingly, two HC-HC assembly promotor pairs can also be combined to generate nucleic acid compositions for in vivo administration encoding antibody mixtures of two assembled antibodies.

In addition, as described in Example 6, the tested HC-HC assembly promoter pairs support specific in vivo assembly of an antibody, also in the presence of other antibody chains that comprise two different HC-HC assembly promoter pairs. Accordingly, three HC-HC assembly promotor pairs can also be combined to generate nucleic acid compositions for in vivo administration encoding antibody mixtures of three assembled antibodies.

In addition, as described in Example 7, increasing numbers of different HC did not negatively affect antibody production in vivo. Accordingly, HC-HC assembly promotor pairs can also be combined with wt IgG or other HC-HC assembly promotor pairs to generate nucleic acid compositions for in vivo administration encoding antibody mixtures of multiple correctly assembled antibodies without hampering in vivo antibody production.

Accordingly, as shown herein, by employing HC-HC assembly promoter pairs, an antibody mixture of up to five correctly assembled antibodies (IgG with HC-HC_PP3, IgG with HC-HC PP4, IgG with HC-HC_PP5, IgG with HC-HC_PP18, wt IgG) can be produced upon in vitro and/or in vivo administration of a composition of the invention.

The inventive concept exemplified herein can potentially be expanded, and further HC-HC assembly promoters as disclosed in the specification can be used to generate nucleic acid compositions encoding a plurality of different assembled antibodies (e.g. 5, 6, 7, 8, 9, 10, 20 or more assembled antibodies).

Summarizing the above, the data demonstrates that the production of a plurality of fully (correctly) assembled antibodies can be accomplished by delivering a nucleic acid composition encoding said plurality of antibodies, wherein at least one coding sequence of the nucleic acid sequences encodes at least one antibody chain assembly promoter.

1. A composition for expression of at least two antibodies in a cell or subject comprising
n nucleic acid sequence sets encoding at least one antibody or a fragment or variant thereof, wherein the n nucleic acid sequence sets comprise
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody heavy chain B (HC-B), or a fragment or variant thereof,
wherein the at least one coding sequence of nucleic acid sequence A and/or nucleic acid sequence B encodes at least one antibody chain assembly promoter.

2. Composition of claim 1, wherein the at least one antibody chain assembly promoter is a moiety that promotes, supports, forces, or directs assembly of at least two antibody chains, preferably wherein the moiety comprises at least one amino acid residue in a position that does not occur naturally, or at least one amino acid sequence that does not occur naturally.

3. Composition of claim 1 or 2, wherein the at least one antibody chain assembly promoter is a moiety that prevents or reduces assembly of HC-A and/or HC-B to a wild-type (unmodified) antibody heavy chain, preferably to a wild-type (unmodified) antibody heavy chain selected or derived from a human.

4. Composition of claim 1 to 3, wherein the at least one antibody or antibody fragment or variant thereof is derived or selected from a monoclonal antibody or fragments thereof, a chimeric antibody or fragments thereof, a human antibody or fragments thereof, a humanized antibody or fragments thereof, an intrabody or fragments thereof, a single chain antibody or fragments thereof.

5. Composition of claim 1 to 4, wherein the at least one antibody or antibody fragment or variant thereof is derived or selected from an IgG1, IgG2, IgG3, IgG4, IgD, IgA1, IgA2, IgE, IgM, IgNAR, hclgG, BiTE, diabody, DART, VHH or VNAR-Fragment, TandAb, scDiabody; sc-Diabody-CH3, Diabody-CH3, Triple Body, mini antibody, minibody, nanobody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, Tandem scFv-Fc, Fab, Fab′, Fc, Facb, pFc′, Fd, Fv, scFv antibody fragment, scFv-Fc, or scFab-Fc, preferably IgG1, IgG3, scFv-Fc or scFab-Fc

6. Composition of claim 1 to 5, wherein the at least one antibody or antibody fragment specifically recognizes and/or binds to at least one target, preferably an epitope or antigen.

7. Composition of any one of the preceding claims, wherein the at least one antibody or antibody fragment specifically recognizes and/or binds to at least one target selected from at least one tumor antigen or epitope, at least one antigen or epitope of a pathogen, at least one viral antigen or epitope, at least one bacterial antigen or epitope, at least one protozoan antigen or epitope, at least one antigen or epitope of a cellular signalling molecule, at least one antigen or epitope of a component of the immune system, at least one antigen or epitope of an intracellular protein, or any combination thereof, preferably the at least one antibody or antibody fragment specifically recognizes and/or binds to at least one antigen or epitope of a pathogen.

8. Composition of any one of the preceding claims, wherein the at least one antibody or antibody fragment is derived or selected from a monospecific antibody or fragment or variant thereof, or a multispecific antibody or fragment or variant thereof.

9. Composition of claim 8, wherein the multispecific antibody is derived or selected from a bispecific, trispecific, tetraspecific, pentaspecific, or a hexaspecific antibody or a fragment or variant of any of these.

10. Composition of any one of the preceding claims, wherein the at least one HC-A and/or the at least one HC-B is derived or selected from antibody heavy chains selected from IgG1, IgG2, IgG3, IgG4, IgD, IgA1, IgA2, IgE, or IgM, or an allotype, an isotype, or mixed isotype or a fragment or variant of any of these, preferably the at least one HC-A and/or the at least one HC-B is derived or selected from antibody heavy chains selected from IgG1 and/or IgG3.

11. Composition of any one of the preceding claims, wherein the at least one HC-A and/or the at least one HC-B is derived or selected from an antibody heavy chain of IgG, or an allotype or an isotype thereof, preferably an antibody heavy chain of IgG1 or an allotype or an isotype thereof.

12. Composition of claim 11, wherein the antibody heavy chain of IgG, preferably IgG1, is selected from G1m17, G1m3, G1m1 and G1m2, G1m27, G1m28, nG1m17, nG1 m1, or any combination thereof.

13. Composition of claim 11 or 12, wherein the antibody heavy chain of IgG, preferably IgG1, is selected from the allotype G1m3,1 (R120, D12/L14).

14. Composition of any one of the preceding claims, wherein the at least one antibody chain assembly promoter is a heavy chain-heavy chain (HC-HC) assembly promoter and/or a heavy chain-light chain (HC-LC) assembly promoter.

15. Composition of claim 14, wherein the at least one HC-HC assembly promoter is located in the constant region of HC-A and/or HC-B.

16. Composition of claim 14 or 15, wherein the at least one HC-HC assembly promoter is located in the Fc region of antibody heavy chain A and/or antibody heavy chain B.

17. Composition of claim 14 to 16, wherein the at least one HC-HC assembly promoter is located in the CH3 domain of antibody heavy chain A and/or antibody heavy chain B.

18. Composition of claim 14 to 17, wherein the at least one HC-HC assembly promoter comprises at least one amino acid substitution in an amino acid sequence of a CH3-CH3 assembly interface.

19. Composition of claim 14 to 18, wherein the at least one HC-HC assembly promoter comprises or consists of at least one selected from steric assembly element, electrostatic steering assembly element, SEED assembly element, DEEK assembly element, interchain disulfides assembly element, or any combination thereof.

20. Composition of claim 14 to 19, wherein the at least one HC-HC assembly promoter comprises or consists of at least one steric assembly element.

21. Composition of claim 20, wherein the at least one steric assembly element comprises a modification selected from at least one knob-modification and/or at least one hole modification.

22. Composition of claim 21, wherein the at least one knob-modification is at least one amino acid substitution, preferably located in a CH3-CH3 assembly interface.

23. Composition of claim 21, wherein the at least one hole-modification is at least one amino acid substitution, preferably located in a in a CH3-CH3 assembly interface.

24. Composition of claim 14 to 23, wherein the at least one coding sequence of nucleic acid sequence A encodes at least one HC-HC assembly promoter and the at least one coding sequence of nucleic acid sequence B encodes at least one HC-HC assembly promoter.

25. Composition of claim 24, wherein the at least one HC-HC assembly promoter of HC-A comprises at least one knob-modification and the at least one HC-HC assembly promoter of HC-B comprises at least one hole modification.

26. Composition of any one of the preceding claims, wherein HC-A and HC-B comprise at least one HC-HC assembly promoter pair comprising the following amino acid substitutions (numbering according to EU numbering of the CH3 domain):
HC-HC-PP1: T366Y on HC-A; Y407T on HC-B
HC-HC-PP2: T366W on HC-A; 366S, L368A, Y407V on HC-B
HC-HC-PP3: S354C, T366W on HC-A; Y349C, T366S, L368A, Y407V on HC-B
HC-HC-PP4: S364H, F405A on HC-A; Y349T, T394F on HC-B
HC-HC-PP5: T350V, L351Y, F405A, Y407V on HC-A; T350V, T366L, K392L, T394W on HC-B
HC-HC-PP6: K409D on HC-A; D399K on HC-B
HC-HC-PP7: K409D on HC-A; D399R on HC-B
HC-HC-PP8: K409E on HC-A; D399R on HC-B
HC-HC-PP9: K409E on HC-A; D399K on HC-B
HC-HC-PP10: K392D, K409D on HC-A; E/D356K, D399K on HC-B
HC-HC-PP11: D221E, P228E, L368E on HC-A; D221R, P228R, K409R on HC-B
HC-HC-PP12: K360E, K409W on HC-A; Q347R, D399V, F405T on HC-B
HC-HC-PP13: Y349C, K360E, K409W on HC-A; Q347R, S354C, D399V, F405T on HC-B
HC-HC-PP14: L351L/K, T366K on HC-A; Y349D/E, R355D/E on HC-B
HC-HC-PP15: L351L/K, T366K on HC-A; Y349D/E, L351D/E, R355D/E, L368D/E on HC-B
HC-HC-PP16: F405L on HC-A; K409R on HC-B
HC-HC-PP17: K360D, D399M, Y407A on HC-A; E345R, Q347R, T366V, K409V on HC-B
HC-HC-PP18: Y349S, T366M, K370Y, K409V on HC-A; E/D356G, E357D, S364Q, Y407A on HC-B

27. Composition of any one of the preceding claims, wherein antibody heavy chain A (HC-A) and antibody heavy chain B (HC-B) comprises at least one HC-HC assembly promoter pair comprising the following amino acid substitutions (numbering according to EU numbering of the CH3 domain):
HC-HC-PP3: S354C, T366W on HC-A; Y349C, T366S, L368A, Y407V on HC-B
HC-HC-PP4: S364H, F405A on HC-A; Y349T, T394F on HC-B
HC-HC-PP5: T350V, L351Y, F405A, Y407V on HC-A; T350V, T366L, K392L, T394W on HC-B
HC-HC-PP18: Y349S, T366M, K370Y, K409V on HC-A; E/D356G, E357D, S364Q, Y407A on HC-B

28. Composition of any one of the preceding claims, wherein antibody heavy chain A (HC-A) and antibody heavy chain B (HC-B) comprises at least one HC-HC assembly promoter pair comprising the following amino acid sequence preferably located in the CH3 domain:
HC-HC-PP3: SEQ ID NO: 104 on HC-A; SEQ ID NO: 105 on HC-B
HC-HC-PP4: SEQ ID NO: 106 on HC-A; SEQ ID NO: 107 on HC-B
HC-HC-PP5: SEQ ID NO: 108 on HC-A; SEQ ID NO: 109 on HC-B
HC-HC-PP18: SEQ ID NO: 112 on HC-A; SEQ ID NO: 113 on HC-B

29. Composition of any one of the preceding claims, wherein the coding sequence of nucleic acid sequence A additionally encodes at least one fragment selected or derived from an antibody light chain A (LC-A) or a variant thereof and/or wherein the coding sequence of nucleic acid sequence B additionally encodes at least one fragment selected or derived from an antibody light chain B (LC-B) or a variant thereof.

30. Composition of claim 29, wherein the at least one LC-A and/or the at least one LC-B is selected or derived from a κ light chain or λ light chain or a fragment or variant thereof.

31. Composition of claim 29 or 30, wherein the at least one LC-A fragment or variant is N-terminally or C-terminally fused to HC-A, preferably fused to the variable region of HC-A, and/or wherein the at least one LC-B fragment or variant is N-terminally or C-terminally fused to HC-B, preferably fused to the variable region of HC-B.

32. Composition of claim 29 to 31, wherein the LC-A fragment or variant is a variable region of an antibody light chain or a fragment thereof and/or wherein the LC-B fragment or variant is a variable region of an antibody light chain or a fragment thereof.

33. Composition of claim 29 to 32, wherein a variable region of LC-A is fused to the variable region of HC-A, optionally via a linker peptide element, and/or wherein a variable region of LC-B is fused to the variable region of HC-B, optionally via a linker peptide element.

34. Composition of any one of the preceding claims, wherein at least one antibody chain assembly promoter of nucleic acid sequence A and/or the nucleic acid sequence B is selected from a heavy chain-light chain (HC-LC) assembly promoter.

35. Composition of claim 34, wherein the at least one HC-LC assembly promoter is located in the constant region of HC-A and/or HC-B.

36. Composition of claim 34 or 35, wherein the at least one HC-LC assembly promoter is located in the Fab region of HC-A and/or HC-B.

37. Composition of claim 34 to 36, wherein the at least one HC-LC assembly promoter is located in the CH1 domain of HC-A and/or HC-B.

38. Composition of claim 34 to 37, wherein the at least one HC-LC assembly promoter comprises at least one amino acid substitution in an amino acid sequence of the HC-LC assembly interface.

39. Composition of claim 34 to 38, wherein the at least one HC-LC assembly promoter comprises or consists of at least one selected from steric assembly element, electrostatic steering assembly element, SEED assembly element, DEEK assembly element, interchain disulfides assembly element, or any combination thereof.

40. Composition of any one of the preceding claims, wherein the nucleic acid sequence set additionally comprises,
c) nucleic acid sequence C comprising at least one coding sequence encoding at least one LC-A, or a fragment or variant thereof, and/or
d) nucleic acid sequence D comprising at least one coding sequence encoding at least one LC-B, or a fragment or variant thereof.

41. Composition of claim 40, wherein the antibody light chain encoded by nucleic acid sequence C and/or nucleic acid sequence D is selected or derived from a κ light chain or a λ light chain.

42. Composition of claim 40 or 41, wherein the at least one coding sequence of nucleic acid sequence C and/or nucleic acid sequence D encodes at least one light chain-heavy chain (LC-HC) assembly promoter.

43. Composition of claim 42, wherein the at least one LC-HC assembly promoter is located in the constant region of LC-A and/or LC-B.

44. Composition of claim 42 or 43, wherein the at least one LC-HC assembly promoter is located in the Fab region of LC-A and/or LC-B.

45. Composition of claim 42 to 44, wherein the at least one LC-HC assembly promoter is located in the CL domain of LC-A and/or LC-B.

46. Composition of claim 42 to 45, wherein the at least one LC-HC assembly promoter comprises at least one amino acid substitution in an amino acid sequence of the LC-HC assembly interface.

47. Composition of claim 42 to 46, wherein the at least one LC-HC assembly promoter comprises or consists of at least one selected from steric assembly element, electrostatic steering assembly element, SEED assembly element, DEEK assembly element, interchain disulfides assembly element, or any combination thereof.

48. Composition of any one of the preceding claims, wherein n is an integer of 2 to 100, preferably an integer of 2 to 20, for example 2, 3, 4, 5, 6, 7, 8, 9, 10, 11, 12, 13, 14, 15, 16, 17, 18, 19, 20.

49. Composition of claims 1 to 47, wherein the composition comprises m additional nucleic acid sequences comprising at least one coding sequence encoding at least one antibody or a fragment of an antibody or a variant of an antibody, preferably wherein the at least one antibody or a fragment of an antibody or a variant of an antibody does not comprise an antibody chain assembly promoter.

50. Composition of claim 49, wherein the at least one antibody or a fragment or variant thereof encoded by the m additional nucleic acid sequences is a heavy chain of an antibody or a fragment or variant thereof, and/or a light chain of an antibody or a fragment or variant thereof.

51. Composition of claim 49 or 50, wherein the at least one antibody or antibody fragment or variant thereof is derived or selected from a monoclonal antibody or fragments thereof, a chimeric antibody or fragments thereof, a human antibody or fragments thereof, a humanized antibody or fragments thereof, an intrabody or fragments thereof, or a single chain antibody or fragments thereof, or a nanobody or fragments thereof.

52. Composition of claim 49 to 51, wherein the at least one antibody or antibody fragment or variant thereof is derived or selected from IgG1, IgG2, IgG3, IgG4, IgD, IgA1, IgA2, IgE, IgM, IgNAR, hclgG, BiTE, diabody, DART, TandAb; scDiabody; sc-Diabody-CH3, Diabody-CH3, Triple Body, mini antibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, Tandem scFv-Fc, Fab, Fab′, Fc, Facb, pFc′, Fd, Fv, scFv antibody fragment, scFv-Fc, or scFab-Fc.

53. Composition of claim 49 to 52, wherein the at least one antibody or antibody fragment specifically recognizes and/or binds to at least one target, preferably an epitope or antigen.

54. Composition of claim 49 to 53, wherein the at least one antibody or antibody fragment specifically recognizes and/or binds to at least one target selected from at least one tumor antigen or epitope, at least one antigen or epitope of a pathogen, at least one viral antigen or epitope, at least one bacterial antigen or epitope, at least one protozoan antigen or epitope, at least one antigen or epitope of a cellular signalling molecule, at least one antigen or epitope of a component of the immune system, or any combination thereof, preferably the at least one antibody or antibody fragment specifically recognizes and/or binds to at least one antigen or epitope of a pathogen.

55. Composition of claim 49 to 54, wherein the at least one antibody or antibody fragment is derived or selected from a monospecific or a multispecific antibody or fragment or variant thereof, preferably wherein the multispecific antibody is derived or selected from a bispecific, trispecific, tetraspecific, pentaspecific, or a hexaspecific antibody or a fragment or variant thereof.

56. Composition of claim 49 to 55, wherein the at least one antibody or antibody fragment is derived or selected from antibody heavy chains selected from IgG1, IgG2, IgG3, IgG4, IgD, IgA1, IgA2, IgE, or IgM, or an allotype, an isotype, or mixed isotype or a fragment or variant of any of these, preferably IgG1 and/or IgG3.

57. Composition of claim 49 to 56, wherein the at least one antibody or antibody fragment is derived or selected from a κ light chain or a λ light chain.

58. Composition of claim 49 to 57, wherein m is an integer of 1 to 10, preferably 1, 2, 3, 4, 5, 6, 7, 8, 9, 10.

59. Composition of claim 49 to 58, wherein n is an integer of 1 to 20, preferably 1, 2, 3, 4, 5, 6, 7, 8, 9, 10, 11, 12, 13, 14, 15, 16, 17, 18, 19, or 20.

60. Composition of any one of the preceding claims, wherein composition comprises up to four nucleic acid sequence sets selected from
(i) nucleic acid sequence comprising an assembly promoter pair HC-HC-PP3, and/or
(ii) nucleic acid sequence set comprising an assembly promoter pair HC-HC-PP4, and/or
(iii) nucleic acid sequence set comprising an assembly promoter pair HC-HC-PP5, and/or
(iv) nucleic acid sequence set comprising an assembly promoter pair HC-HC-PP18, optionally comprising m additional nucleic acid sequences encoding at least one antibody or a fragment or variant.

61. Composition of any one of the preceding claims, wherein administration of the composition to a cell or to a subject leads to expression of at least two assembled antibodies, optionally to expression of 2 to 40, preferably 2, 3, 4, 5, 6, 7, 8, 9, 10, 11, 12, 13, 14, 15, 16, 17, 18, 19, or 20 assembled antibodies in said cell or subject, wherein, preferably, at least about 70%, at least about 75%, at least about 80%, at least about 85%, at least about 90%, at least about 95%, or at least about 100% of the expressed antibodies are assembled antibodies.

62. Composition of any one of the preceding claims, wherein nucleic acid sequence A, B, C, and/or D and, optionally, the m additional nucleic acid sequences is a monocistronic nucleic acid, a bicistronic nucleic acid, or multicistronic nucleic acid.

63. Composition of any one of the preceding claims, wherein the at least one coding sequence of nucleic acid sequence A, B, C, and/or D and, optionally, the m additional nucleic acid sequence is a codon modified coding sequence, preferably wherein the amino acid sequence encoded by the at least one codon modified coding sequence is not being modified compared to the amino acid sequence encoded by the corresponding wild type or reference coding sequence.

64. Composition of claim 63, wherein the codon modified coding sequence is selected from C maximized coding sequence, CAI maximized coding sequence, human codon usage adapted coding sequence, G/C content modified coding sequence, and G/C optimized coding sequence, or any combination thereof.

65. Composition of claim 63 or 64, wherein the codon modified coding sequence is a G/C optimized coding sequence, a human codon usage adapted coding sequence, or a G/C content modified coding sequence.

66. Composition of any one of the preceding claims, wherein nucleic acid sequence A, B, C, and/or D and, optionally, the m additional nucleic acid sequence comprises at least one untranslated region.

67. Composition of claim 59, wherein the at least one untranslated region is selected from at least one heterologous 5′-UTR and/or at least one heterologous 3′-UTR.

68. Composition of claim 67, wherein the at least one heterologous 3′-UTR comprises or consists a nucleic acid sequence selected or derived from a 3′-UTR of a gene selected from PSMB3, ALB7, alpha-globin, CASP1, COX6B1, GNAS, NDUFA1 and RPS9, or from a homolog, a fragment or a variant of any one of these genes.

69. Composition of claim 67, wherein the at least one heterologous 5′-UTR comprises or consists of a nucleic acid sequence selected or derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

70. Composition of any one of the preceding claims, wherein nucleic acid sequence A, B, C, and/or D and, optionally, the m additional nucleic acid sequence comprises at least one poly(A) sequence, preferably comprising about 30 to about 200 adenosine nucleotides.

71. Composition of any one of the preceding claims, wherein nucleic acid sequence A, B, C, and/or D and, optionally, the m additional nucleic acid sequence comprises at least one poly(C) sequence, preferably comprising about 10 to about 40 cytosine nucleotides.

72. Composition of any one of the preceding claims, wherein nucleic acid sequence A, B, C, and/or D and, optionally, the m additional nucleic acid sequence comprises at least one histone stem-loop or histone stem-loop structure.

73. Composition of any one of the preceding claims, wherein nucleic acid sequence A, B, C, and/or D and, optionally, the m additional nucleic acid sequence is a DNA or an RNA.

74. Composition of any one of the preceding claims, wherein nucleic acid sequence A, B, C, and/or D and, optionally, the m additional nucleic acid sequence is a coding RNA.

75. Composition of claim 74, wherein the coding RNA is an mRNA, a self-replicating RNA, a circular RNA, or a replicon RNA, preferably mRNA.

76. Composition of any one of the preceding claims, wherein nucleic acid sequence A, B, C, and D and, optionally, the m additional nucleic acid sequence are mRNA constructs.

77. Composition of any one of the preceding claims, wherein nucleic acid sequence A, B, C, and/or D and, optionally, the m additional nucleic acid sequence comprises a 5′-cap structure, preferably m7G, cap0, cap1, cap2, a modified cap0 or a modified cap1 structure.

78. Composition of any one of the preceding claims, wherein nucleic acid sequence A, B, C, and/or D and, optionally, the m additional nucleic acid sequence comprises at least one modified nucleotide preferably selected from pseudouridine (4) and/or N1-methylpseudouridine (m1ψ).

79. Composition of any one of the preceding claims, comprising at least one pharmaceutically acceptable carrier or pharmaceutically acceptable excipient.

80. Composition of any one of the preceding claims, wherein nucleic acid sequence A, B, C, and/or D and, optionally, the m additional nucleic acid sequence are formulated separately.

81. Composition of any one of the preceding claims, wherein nucleic acid sequence A, B, C, and/or D and, optionally, the m additional nucleic acid sequence are co-formulated.

82. Composition of any one of the preceding claims, wherein nucleic acid sequence A, B, C, and/or D and, optionally, the m additional nucleic acid sequence is complexed or associated with or at least partially complexed or partially associated with one or more cationic or polycationic compound.

83. Composition of claim 83, wherein the one or more cationic or polycationic compound is selected from a cationic or polycationic polymer, cationic or polycationic polysaccharide, cationic or polycationic lipid, cationic or polycationic protein, cationic or polycationic peptide, or any combinations thereof.

84. Composition of claim 82 to 83, wherein the one or more cationic or polycationic peptides are selected from any one of the peptides according to SEQ ID NOs: 75 to 79 for complexation, or any combinations thereof.

85. Composition of claim 82 to 84, wherein the cationic or polycationic polymer is a polyethylene glycol/peptide polymer comprising HO-PEG5000-S-(S-CHHHHHHRRRRHHHHHHC-S-)7-S-PEG5000-OH (SEQ ID NO: 78 of the peptide monomer) and/or wherein the cationic or polycationic polymer is a polyethylene glycol/peptide polymer comprising HO-PEG5000-S-(S-CGHHHHHRRRRHHHHHGC-S-)4-S-PEG5000-OH (SEQ ID NO: 79 of the peptide monomer).

86. Composition of claim 82 to 85, wherein the composition comprises a lipid component or a lipidoid component.

87. Composition of any one of the preceding claims, wherein nucleic acid sequence A, B, C, and/or D and, optionally, the m additional nucleic acid sequence is complexed or associated with one or more lipids, thereby forming liposomes, lipid nanoparticles (LNP), lipoplexes, and/or nanoliposomes.

88. Composition of any one of the preceding claims, wherein nucleic acid sequence A, B, C, and/or D and, optionally, the m additional nucleic acid sequence is complexed or associated with one or more lipids thereby forming lipid nanoparticles (LNPs).

89. Composition of any one of the preceding claims, wherein nucleic acid sequence A, B, C, and/or D and, optionally, the m additional nucleic acid sequence are formulated in separate liposomes, lipid nanoparticles (LNP), lipoplexes, and/or nanoliposomes.

90. Composition of any one of the preceding claims, wherein nucleic acid sequence A, B, C, and/or D and, optionally, the m additional nucleic acid sequence are co-formulated in liposomes, lipid nanoparticles (LNP), lipoplexes, and/or nanoliposomes.

91. Composition of claim 87 to 90, wherein the liposomes, lipid nanoparticles (LNP), lipoplexes, and/or nanoliposomes comprises at least one cationic or cationizable lipid.

92. Composition of claim 87 to 91, wherein the liposomes, lipid nanoparticles (LNP), lipoplexes, and/or nanoliposomes comprises at least one aggregation reducing lipid, preferably at least one polymer conjugated lipid, e.g. a PEG conjugated lipid.

93. Composition of claim 87 to 92, wherein the liposomes, lipid nanoparticles (LNP), lipoplexes, and/or nanoliposomes comprises one or more neutral lipids and/or one or more steroid or steroid analogues.

94. Composition of claim 93, wherein the neutral lipid is 1,2-distearoyl-sn-glycero-3-phosphocholine (DSPC).

95. Composition of claim 93 or 94, wherein the steroid is cholesterol, preferably wherein the molar ratio of the cationic lipid to cholesterol is in the range from about 2:1 to about 1:1.

96. Composition of claim 87 to 95, wherein the liposome, lipid nanoparticle (LNP), lipoplex, and/or nanoliposome, preferably the LNP comprises or consists of
i. at least one cationic or cationizable lipid;
ii. at least one a neutral lipid;
iii. at least one a steroid or steroid analogue;
iv. at least one aggregation reducing lipid, preferably a polymer conjugated lipid, e.g. a PEG-lipid.

97. Composition of claim 96, wherein (i) to (iv) are in a molar ratio of about 20-60% cationic or cationizable lipid, 5-25% neutral lipid, 25-55% sterol, and 0.5-15% aggregation reducing lipid, preferably polymer-conjugated lipid.

98. Composition of any one of the preceding claims, wherein the composition is a lyophilized composition, a spray-dried composition, or a spray-freeze dried composition, optionally comprising at least one pharmaceutically acceptable lyoprotectant.

99. Composition any one of the preceding claims, wherein administration to a cell or to a subject leads to expression of at least two assembled antibodies in said cell or subject, wherein, preferably, at least about 70%, at least about 75%, at least about 80%, at least about 85%, at least about 90%, at least about 95%, or at least about 100% of the expressed at least two antibodies are (correctly) assembled antibodies.

100. A nucleic acid sequence set encoding at least one antibody or a fragment or variant thereof, comprising
a) nucleic acid sequence A comprising at least one coding sequence encoding at least one antibody heavy chain A (HC-A), or a fragment or variant thereof, and
b) nucleic acid sequence B comprising at least one coding sequence encoding at least one antibody heavy chain B (HC-B), or a fragment or variant thereof,
wherein the at least one coding sequence of the nucleic acid sequence A and/or the nucleic acid sequence B encodes at least one antibody chain assembly promoter, preferably wherein the nucleic acid sequence set is selected from any one of the nucleic acid sequence sets as defined in claims 1 to 47, optionally wherein the nucleic acid sequences are characterized by any one of the features as defined in claims 62 to 78.

101. A Kit or kit of parts, comprising at least one composition of claim 1 to 99, or at least one nucleic acid sequence set of claim 100, optionally comprising at least one liquid vehicle for solubilising, and, optionally, technical instructions providing information on administration and dosage of the kit components.

102. Composition of claim 1 to 99, a nucleic acid sequence set of claim 100, or a kit or kit of parts of claim 101, for use as a medicament.

103. Composition of claim 1 to 99, a nucleic acid sequence set of claim 100, or a kit or kit of parts of claim 101, for use in the treatment or prophylaxis of an infection with a pathogen, for use in the treatment or prophylaxis of a cardiovascular disease, for use in the treatment or prophylaxis of a neurological disease, for use in the treatment or prophylaxis of an infectious disease, for use in the treatment or prophylaxis of an autoimmune diseases, for use in the treatment or prophylaxis of cancer or tumour disease, for use in the treatment or prophylaxis of an eye or ophthalmic disease, for use in the treatment or prophylaxis of a lung or pulmonary disease, for use in the treatment or prophylaxis of a neurological disease, or for use in the treatment or prophylaxis of a genetic disease.

104. A method of treating or preventing a disorder or condition, wherein the method comprises applying or administering to a subject in need thereof a composition of claim 1 to 99, a nucleic acid sequence set of claim 100, or a kit or kit of parts of claim 101.

105. Method of treating or preventing a disorder of claim 104, wherein the disorder or condition is an infection with a pathogen, a cardiovascular disease, a neurological disease, an infectious disease, an autoimmune diseases, a cancer or tumour disease, an eye or ophthalmic disease, a lung or pulmonary disease, a neurological disease, or a genetic disease.

106. Method of treating or preventing a disorder of claim 104 or 105, wherein the subject in need is a mammalian subject, preferably a human subject.

107. A method of expressing at least two nucleic acid encoded antibodies in an organ or tissue in a subject, wherein the method comprises applying or administering a composition of claim 1 to 99, a nucleic acid sequence set of claim 100, or a kit or kit of parts of claim 101 to a subject.

108. Method of expressing of claim 107, wherein the method does not involve a harvesting step of the expressed antibodies or a purification step of the expressed antibodies.

109. Method of expressing of claim 107 or 108, wherein the method is an in vivo method for expressing at least two correctly assembled antibodies

110. A method of producing at least two nucleic acid encoded antibodies, wherein the method comprises a step of (i) applying or administering a composition of claim 1 to 99, a nucleic acid sequence set of claim 100, or a kit or kit of parts of claim 101 to allow expression of at least two assembled antibodies in a cell, and, optionally, a step of (ii) isolating and/or purifying the produced assembled antibodies, wherein the method is an in vitro, in situ, or ex vivo method.
